(19) United States
(12) Patent Application Publication
(10) Pub. No.: US 2009/0318438 A1
(43) Pub. Date: Dec. 24, 2009

(54) Heterocyclic compounds and their use as anticancer agents

(76) Inventors: Xiaoling Chen, Brooklyn, NY (US); Hai-Ying He, Brooklyn, NY (US); Joel Kawakami, Honolulu, HI (US); Alexander Kiselyov, San Diego, CA (US); Xiaohu Ouyang, Flushing, NY (US); Vatee Pattaropong, New Madrid, MO (US); Evgueni Piatnitski, King of Prussia, PA (US); Maria Carolina Tuma, Jersey City, NJ (US); John Kincaid, San Diego, CA (US)

Correspondence Address: IMCLON;Lerner, David, Littenberg, Krumholz & Mentlik, LLP, 600 South Avenue West, Westfield, NJ, 07090, US

(21) Appl. No.: 12/286,132

(22) Filed: Sep. 25, 2008

Related U.S. Application Data
(63) Continuation of application No. 10/564,267, filed on Oct. 10, 2008.
(60) Provisional application No. 60/485,963, filed on Jul. 9, 2003.

(51) Int. Cl. A61K 31/5377; A61K 31/444; C07D 401/04; A61K 31/496; C07D 413/14; A61P 35/00; C07D 405/00; C07D 401/14; A61K 31/4439

(52) U.S. Cl. 514/233.8; 514/340; 514/333; 514/338; 546/256; 546/272.4; 514/255.05

(57) ABSTRACT

The present invention relates to heterocyclic compounds that have anticancer activity, and pharmaceutical compositions that contain such compounds, methods of treating diseases and conditions in mammals using such compounds and composition and methods for their manufacture.

### CROSS-REFERENCE TO RELATED APPLICATIONS

This application is a continuation of U.S. application Ser. No. 10/564,267, filed on Jan. 9, 2006, the disclosure of which is incorporated herein by reference.

### FIELD OF THE INVENTION

The present invention encompasses heterocyclic compounds and derivatives thereof, pharmaceutical compositions containing the compounds, methods for making the compounds, and methods of treating cancer and/or ocular diseases by administering a therapeutically effective amount of the compounds to subjects in need of such treatment.

### BACKGROUND OF THE INVENTION

Malignant tumors, characterized by abnormal proliferation of neoplastic cells, are one of the most common diseases worldwide, and the subset of human cancer types amenable to curative treatment is rather small. Although there is tremendous progress in understanding the molecular events that lead to malignancy, there is still a high demand for the development of clinically innovative drugs that can effectively inhibit proliferation of cancer cells and cure human cancer.

Taxol is one of many antitumor agent developed in the past three decades, effective for treatment of ovarian and breast cancers, with a worldwide sale of USD 1.5 billion in 2002. Because taxol halts proliferation of cancer cells by acting on microtubules, taxol's success as a chemotherapeutic agent brought the focus back to the potential of microtubules as a potential target.

Microtubules are elements of the cell cytoskeleton that play a key role in cell division, shape and motility, as well as intracellular transport. Microtubules are highly dynamic structures formed by heterodimers of alpha and beta tubilin that assemble into polymers in a GTP-dependent manner. During cell division microtubules disassemble into soluble tubulin dimers, prior to their reassembly and formation of the mitotic spindle, a structure that provides segregation of replicated chromosomes to daughter cells. For proper cell division to occur, it is essential that microtubules are able to polymerize and depolymerize. Microtubules in the mitotic spindle are more dynamic than those in non-dividing cells, and thus can be targeted by agents that affect microtubule dynamics. By altering microtubule polymerization/depolymerization these agents affect mitotic spindle function, arrest dividing cells in the G2/M phase of the cell cycle, and ultimately lead to apoptotic cell death. As neoplastic cells have high proliferation rates, they can be targeted by these antimitotic agents. Compounds that bind to tubulin, interfere with microtubule dynamics and inhibit division of cancer cells and are indeed some of the most effective cancer therapeutic agents in use.

Clinically available compounds, such as taxol or vincristine, have been to known to have disadvantages, such as, (1) high toxicity, (2) marginal bioavailability and poor solubility, (3) complex synthesis or isolation procedures, and (4) development of drug resistance in patients. Therefore, synthetic low molecular weight compounds with oral bioavailability and high therapeutic index for first and second line therapy are desirable.

Because of their clinical potential, several synthetic molecules that bind to tubulin are currently being evaluated in preclinical or early clinical stage. Most notably, WO 01/22954, assigned to Asta Medica, discloses indole-3-glyoxylamide derivatives with antitumor activity. One compound, D-24851, has been shown to exert antitumor activity in vivo, shows efficacy toward MDR cells and lacks neurotoxicity. See, Cancer Research 61, 392, 2001. DE 10020852, assigned to Asta Medica, discloses 1H-indol-2-yl aryl ketones and related compounds as antitumor agents. Specifically, D64131 has been shown to be orally active, efficacious in xenograft models and showed no signs of toxicity. See, Cancer Research 62, 3113, 2002. South African publication ZA 2000000419, assigned to Abbott, discloses oxadiazoline derivatives as antiproliferative agents. A-204197 has shown to be effective against Taxol resistant cell lines. See, Cancer Research 61, 5480, 2001. U.S. Pat. No. 6,521,658, also assigned to Abbott, discloses certain sulfonamides as cell proliferation inhibitors. WO 02/39958, assigned to Tularik, discloses combination therapy using pentafluorobenzenesulfonamides and antineoplastic agents.

Besides their antitumorigenic effect by inhibition of proliferation of tumor cells, tubulin agents can also act as vascular disrupting agents (VDAs). The effect of tubulin agents on tumor endothelial cells may cause in a single dose the selective shutdown of tumor vasculature, depriving tumor cells of nutrients and oxygen, and causing tumor necrosis. See, Clin. Cancer Res., 10:415-27 (2004) or Cancer, 100:2491-9 (2004). Preclinical data have shown that some but not all tubulin small molecules have antivascular and antiangiogenic activities. While marketed drugs such as paclitaxel and vinblastine might have antiangiogenic actions in low doses, they only have vascular disrupting effects at maximum tolerated doses (MTD). Second generation small molecule tubulin agents such as combretastatin and its analogues, nevertheless, are effective at doses much lower than MTD. Combretastatin A-4 phosphate has been shown to change endothelial cell morphology, shut down tumor vasculature, and induce tumor necrosis in mouse tumor models. See, Cancer Research, 59, 1626 (1999). Tubulin agents have shown to have synergistic anticancer effect with existing therapies. See, Cancer Research, 59, 1626 (1999); Eur. J. Cancer, 40:284-90 (2004); Anticancer Res. 23:1619-23 (2003). Antivascular and anti-angiogenic actions of small molecule tubulin binding agents have also been demonstrated in clinical trials. See, Cancer Res. 63:1144-7 (2003); Clin. Cancer Res. 10:415-27 (2004). Tubulin small molecules VDAs additionally can be useful in the treatment of ocular diseases in which retinal neovascularization is pathological, such as age-dependent macular degeneration and diabetic retinopathy. See, Oncogene, 22: 6537-6548 (2003).

The desirability for novel and active compounds that may treat diseases associated with effects upon microtubules are of great interest as novel therapeutics.

### SUMMARY OF THE INVENTION

One embodiment of the invention encompasses compounds having Formula II:

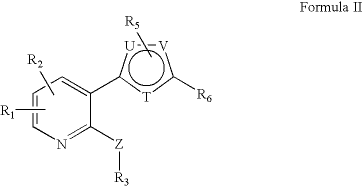

or pharmaceutically acceptable salts, stereoisomers, hydrates or pro-drugs thereof,
wherein,

the ring formed by T, U, V is

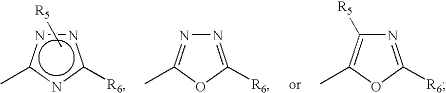

Z is O, S, nitro, or NR₄;

R₁, R₂, or R₅ each independently is:

1) hydrogen, hydroxyl, halo, nitro, or cyano;

2) C₁-C₈ alkyl;

3) C₂-C₈ alkenyl;

4) C₂-C₈ alkynyl;

5) C₁-C₈ alkoxy;

6) C₃-C₈ cycloalkyl or heterocyclyl;

7) C₄-C₈ cycloalkylalkyl or heterocyclylalkyl;

8) C₃-C₁₀ aryl;

9) C₅-C₁₀ aralkyl;

10) C₆-C₁₀ aryloxy;

11) NH₂, NHR₇, or NR₇R₇; or

12) —SO₂R₇,

wherein R₇ is independently H, hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally, R₁ and R₂ taken together form a ring structure including cycloalkyl, heterocyclyl, or aryl ring;

R₃ is:

1) hydrogen;

2) C₁-C₈ alkyl;

3) C₂-C₈ alkenyl;

4) C₂-C₈ alkynyl;

5) C₁-C₈ alkoxy;

6) C₃-C₁₀ cycloalkyl or heterocyclyl;

7) C₄-C₁₀ cycloalkylalkyl or heterocyclylalkyl;

8) C₃-C₁₀ aryl;

9) C₄-C₁₀ aralkyl;

10) carbonyl; or

11) —SO₂R₈, —CO₂R₈, —SR₈, or —SOR₈;

wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,

R₄ is:

1) hydrogen;

2) C₁-C₈ alkyl;

3) C₂-C₈ alkenyl;

4) C₂-C₈ alkynyl;

5) C₃-C₈ cycloalkyl or heterocyclyl;

6) C₄-C₈ cycloalkylalkyl or heterocyclylalkyl;

7) C₃-C₁₀ aryl;

8) C₅-C₁₀ aralkyl;

9) carbonyl; or

10) —SO₂R₁₂, or —SOR₁₂;

wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; optionally, R₃ and R₄ are taken together to form a C₄-C₆ heterocyclyl optionally substituted with R₁₃, or aryl; and

R₆ is:

1) C₁-C₈ alkyl;

2) C₂-C₈ alkenyl;

3) C₂-C₈ alkynyl;

4) C₁-C₈ alkoxy;

5) C₃-C₁₀ cycloalkyl or heterocyclyl;

6) C₄-C₁₀ cycloalkylalkyl or heterocyclylalkyl;

7) C₄-C₁₀ aryl;

8) C₅-C₁₀ aralkyl; or

9) NH₂, NHR₉ or NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄) alkyl], —N[(C₁-C₄)alkyl]₂, —NH(C₅-C₈ heterocyclylalkyl), —NH(C₆-C₈ aryl), or —NH(C₆-C₈ heterocyclyl).

Another embodiment of the invention encompasses compounds of Formula II wherein Z is O or NH. Yet another embodiment of the invention encompasses compounds of Formula II. Yet another embodiment of the invention encompasses compounds of Formula II wherein R₁, R₂, or R₅ is substituted with R₇, wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

Another embodiment of the invention encompasses compounds of Formula II wherein R₁ and R₂ taken together form a ring structure including cycloalkyl, heterocyclyl or aryl rings. Yet another embodiment of the invention encompasses compounds of Formula II, wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

Yet another embodiment of the invention encompasses compounds of Formula II, wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

Yet another embodiment of the invention encompasses compounds of Formula II, wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocycyl).

The invention also encompasses compounds of Formula III:

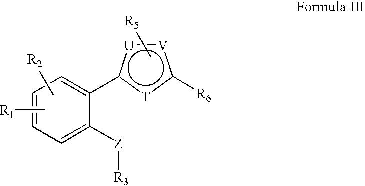

wherein,

the ring formed by T, U, V is

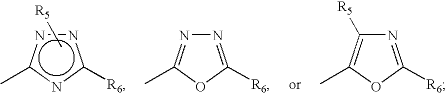

Z is O, S, nitro, or NR₄;

R₁, R₂, or R₅ each independently is:

1) hydrogen, hydroxyl, halo, nitro, or cyano;

2) C₁-C₈ alkyl;

3) C₂-C₈ alkenyl;

4) C₂-C₈ alkynyl;

5) C₁-C₈ alkoxy;

6) C₃-C₈ cycloalkyl or heterocyclyl;

7) C₄-C₈ cycloalkylalkyl or heterocyclylalkyl;

8) C₃-C₁₀ aryl;

9) C₅-C₁₀ aralkyl;

10) C₆-C₁₀ aryloxy;

1) NH₂, NHR₇, or NR₇R₇; or

12) —SO₂R₇,

wherein R₇ is independently H, hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally, R₁ and R₂ taken together form a ring structure including cycloalkyl, heterocyclyl, or aryl ring;

R₃ is:

1) hydrogen;

2) C₁-C₈ alkyl;

3) C₂-C₈ alkenyl;

4) C₂-C₈ alkynyl;

5) C₁-C₈ alkoxy;

6) C₃-C₁₀ cycloalkyl or heterocyclyl;

7) C₄-C₁₀ cycloalkylalkyl or heterocyclylalkyl;

8) C₃-C₁₀ aryl;

9) C₄-C₁₀ aralkyl;

10) carbonyl; or

11) —SO₂R₈, —CO₂R₈, —SR₈, or —SOR₈;

wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,

R₄ is:

1) hydrogen;

2) C₁-C₈ alkyl;

3) C₂-C₈ alkenyl;

4) C₂-C₈ alkynyl;

5) C₃-C₈ cycloalkyl or heterocyclyl;

6) C₄-C₈ cycloalkylalkyl or heterocyclylalkyl;

7) C₃-C₁₀ aryl;

8) C₅-C₁₀ aralkyl;

9) carbonyl; or

10) —SO₂R₁₂, or —SOR₁₂;

wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; optionally, R₃ and R₄ are taken together to form a C₄-C₆ heterocyclyl optionally substituted with R₁₃, or aryl; and

R₆ is:

1) C₁-C₈ alkyl;

2) C₂-C₈ alkenyl;

3) C₂-C₈ alkynyl;

4) C₁-C₈ alkoxy;

5) C₃-C₁₀ cycloalkyl or heterocyclyl;

6) C₄-C₁₀ cycloalkylalkyl or heterocyclylalkyl;

7) C₄-C₁₀ aryl;

8) C₅-C₁₀ aralkyl; or

9) NH₂, NHR₉ or NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄) alkyl], —N[(C₁-C₄)alkyl]₂, —NH(C₅-C₈ heterocyclylalkyl), —NH(C₆-C₈ aryl), or —NH(C₆-C₈ heterocyclyl).

Another embodiment of the invention encompasses compounds of Formula III, wherein Z is O or NR₄. Yet another embodiment of the invention encompasses compounds of Formula III, wherein R₁, R₂, or R₅ is substituted with R₇ wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

Another embodiment of the invention encompasses compounds of Formula III, wherein when taken together R₁ and R₂ form a ring structure including cycloalkyl, heterocyclyl, or aryl. Yet another embodiment of the invention encompasses compounds of Formula III, wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

Yet another embodiment of the invention encompasses compounds of Formula III, wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

Yet another embodiment of the invention encompasses compounds of Formula III, wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

The invention also encompasses methods for treating cancer comprising administering a therapeutically effective amount of a compound of Formula II to a subject in need of such treatment, wherein the compound of Formula II has the formula:

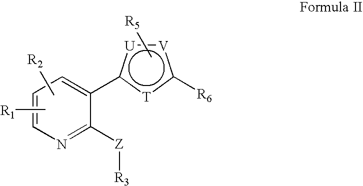

or pharmaceutically acceptable salts, stereoisomers, hydrates or pro-drugs thereof,
wherein,

the ring formed by T, U, V is

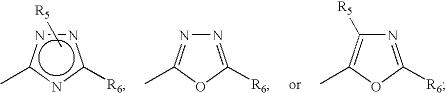

Z is O, S, nitro, or NR₄;

R₁, R₂, or R₅ each independently is:

1) hydrogen, hydroxyl, halo, nitro, or cyano;

2) C₁-C₆ alkyl;

3) C₂-C₆ alkenyl;

4) C₂-C₆ alkynyl;

5) C₁-C₆ alkoxy;

6) C₃-C₈ cycloalkyl or heterocyclyl;

7) C₄-C₈ cycloalkylalkyl or heterocyclylalkyl;

8) C₄-C₁₀ aryl;

9) C₄-C₁₀ aralkyl;

10) C₆-C₁₀ aryloxy;

1) NH₂, NHR₇, or NR₇R₇; or

12) —SO₂R₇,

wherein R₇ is independently H, hydroxyl, halo, C₁-C₄ alkyl optionally substituted with at least one R₁₀, C₁-C₄ alkoxy optionally substituted with at least one R₁₀, C₃-C₈ cycloalkyl optionally substituted with at least one R₁₀, C₄-C₈ heterocycloalkyl optionally substituted with at least one R₁₀, C₆-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂, wherein when taken together R₁ and R₂ form a ring structure including heterocyclyl or aryl rings;

R₃ is:

1) hydrogen;

2) C₁-C₆ alkyl;

3) C₂-C₆ alkenyl;

4) C₂-C₆ alkynyl;

5) C₁-C₆ alkoxy;

6) C₃-C₁₀ cycloalkyl or heterocyclyl;

7) C₄-C₁₀ cycloalkylalkyl or heterocyclylalkyl;

8) C₄-C₁₀ aryl;

9) C₄-C₁₀ aralkyl;

10) carbonyl; or

11) —SO₂R₈, —CO₂R₈, —SR₈, or —SOR₈;

wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,

R₄ is:

1) hydrogen;

2) C₁-C₆ alkyl;

3) C₂-C₆ alkenyl;

4) C₂-C₆ alkynyl;

5) C₃-C₈ cycloalkyl or heterocyclyl;

6) C₄-C₈ cycloalkylalkyl or heterocyclylalkyl;

7) C₄-C₁₀ aryl;

8) C₅-C₁₀ aralkyl;

9) carbonyl; or

10) —SO₂R₁₂, or —SOR₁₂;

wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₆-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; and

R₆ is:

1) C₁-C₆ alkyl;

2) C₂-C₆ alkenyl;

3) C₂-C₆ alkynyl;

4) C₁-C₆ alkoxy;

5) C₃-C₈ cycloalkyl or heterocyclyl;

6) C₄-C₈ cycloalkylalkyl or heterocyclylalkyl;

7) C₄-C₁₀ aryl;

8) C₅-C₁₀ aralkyl; or

9) —NH₂, —NH₉, or —NR₉R₉,

wherein R₉ is independently hydroxyl, halo, nitro, C₁-C₄ alkyl optionally substituted with at least one R₁₄, C₂-C₄ alkynyl optionally substituted with at least one R₁₄, C₁-C₄ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₆-C₁₀ aryl, C₄-C₉ heterocycloalkyl, —SO₂(C₆-C₁₀ aryl), NH₂, —NH[(C₁-C₄)alkyl], —N[(C₁-C₄) alkyl]₂, —NH(C₅-C₉ heterocyclylalkyl), —NH(C6-C₈ aryl), or —NH(C₆-C₈ heterocyclyl) or a pharmaceutically acceptable salt, hydrate or pro-drug thereof, in combination with a pharmaceutically acceptable carrier.

Another embodiment of the invention encompasses methods of treatment wherein in the compounds of Formula II Z is O or NH. Yet another embodiment of the invention encompasses methods of treatment wherein in the compounds of Formula II R₁, R₂, or R₅ is substituted with R₇, wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

Another embodiment of the invention encompasses methods of treatment wherein in the compounds of Formula II R₁ and R₂ taken together form a ring stricture including cycloalkyl, heterocyclyl or aryl.

Yet another embodiment of the invention encompasses methods of treatment wherein in the compounds of Formula II wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

Another embodiment of the invention encompasses methods of treatment wherein in the compounds of Formula II wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

Yet another embodiment of the invention encompasses methods of treatment wherein in the compounds of Formula II wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

Another embodiment of the invention encompasses methods of treatment wherein in the compounds of Formula II are administered in a dosage form which may be a tablet, caplet, troche, lozenge, dispersion, suspension, suppository, solution, capsule, or patch. Another embodiment of the invention encompasses methods of treatment wherein in the compounds of Formula II are administered in about 0.001 mg/kg to about 100 mg/kg. Yet another embodiment of the invention encompasses methods of treatment wherein in the compounds of Formula II are administered by oral administration.

### DETAILED DESCRIPTION OF THE INVENTION

The present invention encompasses heterocyclic compounds and derivatives thereof, pharmaceutical compositions containing the compounds, methods for making the compounds, methods of treating cancer, and methods of treating ocular diseases by administering a therapeutically effective amount of the compounds to subjects in need of such treatment. Not to be limited by theory, it is believed that the compounds of Formula I inhibit tubilin polymerization, and consequently cell division. Therefore, the compounds of Formula I may be used to treated diseases associated with the uncontrolled proliferation of cells. In particular, the invention encompasses heterocylic compounds having compounds of Formula I:

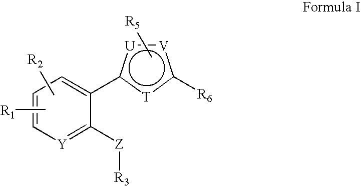

### DEFINITIONS

As used herein, the term “alkyl” refers to a saturated hydrocarbon radical having 1 to 6 carbon atoms. The allyl group may be straight, branched, substituted or unsubstituted. Alkyl groups include, but are not limited to, methyl, ethyl, propyl, isopropyl, butyl, or t-butyl.

As used herein, the term “alkenyl” refers to a non-aromatic hydrocarbon radical, which may be straight chain or branched, substituted or unsubstituted, having from 2 to 6 carbon atoms and at least one carbon to carbon double bond. Alkenyl groups include, but are not limited to, ethenyl, propenyl, butenyl, pentenyl, or 2-methylbutenyl.

As used herein, the term “alkynyl” as used herein refers to a hydrocarbon radical, which may be straight chained or branched, substituted or unsubstituted, having 2 to 6 carbon atoms and at least one carbon to carbon triple bond. Alkynyl groups include, but are not limited to, ethynyl, propynyl, or butynyl.

As used herein, the term “alkoxy” refers to a substituted or unsubstituted group including —O-alkyl, —O-alkenyl, —O-alkynyl group, —O-cycloalkyl, or —O-heterocyclyl, wherein alkyl, alkenyl, and alkynyl are as defined above and cycloalkyl and heterocyclyl are as defined below. Examples of alkoxy groups include, but are not limited to, methoxy, ethoxy, propoxy, isopropoxy, butoxy, isobutoxy, tertiary butoxy, pentoxy, isopentoxy, hexoxy, isohexoxy, allyloxy, propargyloxy, or vinyloxy.

As used herein, the term “cycloalkyl” refers to a cyclic hydrocarbon radical having 3 to 10 carbon atoms, which may be substituted or unsubstituted. Optionally, the cycloalkyl group may have at least one carbon to carbon double bond. Cycloalkyl groups include, but are not limited to cyclopropyl, cyclobutyl, cyclopentyl, cyclopentenyl, or cyclohexyl.

As used herein, the term “heterocyclyl” or “heterocycle” refers to cycloalkyl rings that include within the ring at least one nitrogen, oxygen, or sulfur atom, and optionally include one or two double bonds. The nitrogen and sulfur heteroatoms may optionally be oxidized, and the nitrogen heteroatom may optionally be quaternized. The term “heterocyclyl” also refers to dihydro and tetrahydro analogs of monocyclic or polycyclic aromatic rings having at least one nitrogen atom within the ring. The heterocyclic ring may be attached at any heteroatom or carbon atom, which results in the creation of a stable structure. The heterocycle ring can be substituted or unsubstituted including, but not limited to, aziridinyl, furanyl, isothiazolidinyl, isothiazolyl, isoxazolidinyl, isoxazolyl, morpholino, oxadiazolyl, oxazolidinyl, oxazolinyl, oxazolyl, piperidinyl, 4-piperidonyl, piperazinyl, pyranyl, pyrazolidinyl, pyrrolidinyl, quinuclidinyl, tetrahydrofuranyl, tetrahydrothienyl, tetrahydrothiophenyl, thiadiazoyl, thiazolidinyl, thiazolinyl, thiazolyl, thienyl, thiomorpholino, thiomorpholinyl sulfoxide, thiomorpholinyl sulfone, or thiophenyl.

As used herein, the term “aryl” refers to carbocyclic aromatic groups including, but not limited to, phenyl, naphthyl, or anthracyl. The term “aryl” also refers to monocyclic or polycyclic aromatic ring having at least one nitrogen atom within the ring. The nitrogen heteroatom may optionally be quaternized. The term “aryl” also refers to any bicyclic group in which a cycloalkyl or heterocycloalkyl ring is fused to a benzene ring, examples include, but are not limited to, azolyl, azepinyl, benzimidazolyl, benzofuranyl, benzoisothiazolyl, benzoisoxazolyl, benzooxazolyl, benzopyranyl, benzothiazolyl, benzothienyl, benzotriazole, benzoxazolyl, imidazolidinyl, imidazolyl, imidazopyridinyl, indolinyl, indolizinyl, indolyl, isoimidazolyl, isoindolyl, isoquinolinyl, pyrazinyl, pyrazolyl, pyridinyl, pyridazinyl, pyrimidinyl, pyrrolyl, quinolinyl, tetrazolyl, triazinyl, 1,2,3-triazolyl, or 1,2,4-triazolyl. An aryl ring may be unsubstituted or substituted with at least one suitable substituent.

As used herein, the term “cycloalkylalkyl” refers to a straight-chain alkyl, alkenyl or alkynyl group wherein one of the hydrogen atoms bonded to a terminal carbon is replaced with a cycloalkyl moiety, for example, —(CH₂)ₙ-cycloalkyl, wherein n=1-6.

As used herein, the term “heterocyclylalkyl” refers to a straight-chain alkyl, alkenyl or alkynyl group wherein one of the hydrogen atoms bonded to a terminal carbon is replaced with a cycloalkyl moiety, for example, —(CH₂)ₙ-heterocyclyl, wherein n=1-6.

As used herein, the term “aralkyl” refers to a straight-chain alkyl, alkenyl or alkynyl group wherein one of the hydrogen atoms bonded to a terminal carbon is replaced with an aryl or heteroaryl moiety. Typical aralkyl groups include, but are not limited to, benzyl, benzylidene, benzylidyne, benzenobenzyl, naphthenobenzyl and the like.

As used herein, the term “aryloxy group” refers to an —O-aryl or —O-heteroaryl, wherein aryl or heteroaryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy.”

As used herein, the term “halo” or “halogen” includes the halogen atoms fluorine, chlorine, bromine, or iodine.

When one or more chiral centers are present in the compounds of the present invention, the individual isomers, i.e., enantiomers, diastereomers, etc. and mixtures thereof (e.g., racemates, etc.) are intended to be encompassed by the formulae depicted herein. Also included are individual polymorphs of each compound of the present invention.

As used herein the terms “pharmaceutically acceptable salts” and “hydrates” refer to those salts and hydrated forms of the compound that would be apparent to those in the art, i.e., those which favorably affect the physical or pharmacokinetic properties of the compound, such as solubility, palatability, absorption, distribution, metabolism, or excretion. Other factors, more practical in nature, which those skilled in the art may take into account in the selection include the cost of the raw materials, ease of crystallization, yield, stability, solubility, hygroscopicity and flowability of the resulting bulk drug. Pharmaceutically acceptable salts may be prepared by the addition of an appropriate acid. Thus, the compound can be used in the form of salts derived from inorganic or organic acids. Examples include, but are not limited to, acetate, adipate, alginate, aspartate, benzoate, benzenesulfonate, bisulfate, butyrate, citrate, camphorate, camphorsulfonate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxy-ethanesulfonate, lactate, maleate, methanesulfonate, 2-naphthalenesulfonate, nicotinate, pamoate, pectinate, persulfate, 3-phenylpropionate, pivalate, propionate, succinate, tartrate, or undecanoate.

As used herein, the term “subject” refers to a mammal, preferably a human, but can also be an animal in need of veterinary treatment.

When a compound of the present invention is present as a salt or hydrate that is non-pharmaceutically acceptable, that compound can be converted under certain circumstances to a salt or hydrate form that is pharmaceutically acceptable in accordance with the present invention.

When the compound is negatively charged, it is balanced by a counterion, such as, an alkali metal cation such as sodium or potassium. Other suitable counterions include calcium, magnesium, zinc, ammonium, or alkylammonium cations, such as tetramethylammonium, tetrabutylammonium, choline, triethylhydroammonium, meglumine, triethanol-hydroammonium, and the like. An appropriate number of counterions are associated with the molecule to maintain overall charge neutrality. Likewise, when the compound is positively charged, e.g., protonated, an appropriate number of negatively charged counterions are present to maintain overall charge neutrality. These pharmaceutically acceptable salts are within the scope of the present invention.

Also included in the present invention are pharmaceutically acceptable salts of the compounds described within. Compounds disclosed herein which possess a sufficiently acidic functional group, a sufficiently basic functional group, or both, and accordingly can react with any of a number of organic or inorganic bases, or organic or inorganic acids, may form a salt. Acids commonly employed to form acid addition salts from compounds with basic groups are inorganic acids including, but are not limited to, hydrochloric acid, hydrobromic acid, hydroiodic acid, sulfuric acid, or phosphoric acid, and organic acids including, but are not limited to, para-toluenesulfonic acid, methanesulfonic acid, oxalic acid, para-bromophenyl-sulfonic acid, carbonic acid, succinic acid, citric acid, benzoic acid, or acetic acid. Examples of such salts include, but are not limited to, the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propiolate, decanoate, caprylate, acrylate, formate, isobutyrate, caproate, heptanoate, propionate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, napthalene-1-sulfonate, naphthalene-2-sulfonate, mandelate, and the like.

If the compound has an acidic proton, a salt may be formed by the addition of base to form a pharmaceutically acceptable base addition salt. Base salts include, but are not limited to, ammonium salts, alkali metal salts, alkaline earth metal, salts with organic bases, and salts with amino acids. Alkali metal salts include, but are not limited to, sodium or potassium salts; alkaline earth metal salts include, but are not limited to, calcium and magnesium salts; salts with organic bases include, but are not limited to, dicyclohexylamine salts, N-methyl-D-glucamine; and salts with amino acids include, but are not limited to, arginine, lysine, and the like.

The basic nitrogen-containing groups may be quaternized with agents such as lower alkyl halides, including, not limited to, methyl, ethyl, propyl, or butyl chloride, bromide, or iodide; dialkyl sulfates including, not limited to, dimethyl, diethyl, or dibutyl; and diamyl sulfates, long chain halides including, not limited to, decyl, lauryl, myristyl, or stearyl chlorides, bromides, or iodides; or aralkyl halides including, but not limited to, benzyl and phenethyl bromides and the like.

The presence of pharmaceutically acceptable salts within the scope of the present compounds is not intended to limit the compounds of the present invention to those that are synthetically prepared. The compounds of the present invention also include compounds that are converted within the body and prodrugs. As used herein, term “pro-drug” refers to a form of the compound of the present invention suitable for administration to a patient without undue toxicity, irritation, allergic response, and the like, and effective for their intended use. A pro-drug is transformed in vivo to yield the parent compound of the Formula I herein, for example by hydrolysis in blood. A thorough discussion is provided in T. Higuchi and V. Stella, Pro-drugs as Novel Delivery Systems Vol. 14 of the A. C. S. Symposium Series, and in Edward B. Roche, ed., Bioreversible Carriers in Drug Design, American Pharmaceutical Association and Pergamon Press, 1987.

The compounds of the present invention may have asymmetric centers and occur as racemates, mixtures of diastereomers, enantiomerically enhanced mixtures, or as individual enantiomers. All isomeric forms and/or polymorphs are included in the present invention.

One embodiment of the invention encompasses heterocyclic biaryl compounds having five or six membered rings wherein the rings optionally include at least one heteroatom which are useful in the treatment of cancer. Generally, the compounds of the invention are represented in Formula (I):

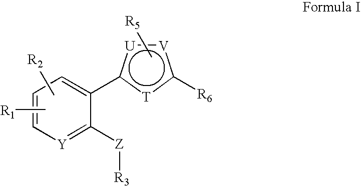

or pharmaceutically acceptable salts, stereoisomers, hydrates or pro-drugs thereof,
wherein,

Y is C or N;

T, U, V each independently is C, N, or O;

Z is O, S, nitro, or NR₄;

R₁, R₂, or R₅ each independently is:

1) hydrogen, hydroxyl, halo, nitro, or cyano;

2) alkyl, optionally substituted with at least one R₇;

3) alkenyl, optionally substituted with at least one R₇;

4) alkynyl, optionally substituted with at least one R₇;

5) alkoxy, optionally substituted with at least one R₇;

6) cycloalkyl or heterocyclyl, optionally substituted with at least one R₇;

7) cycloalkylalkyl or heterocyclylalkyl, optionally substituted with at least one R₇;

8) aryl, optionally substituted with at least one R₇;

9) aralkyl, optionally substituted with at least one R₇;

10) aryloxy, optionally substituted with at least one R₇;

11) NH₂, NHR₇, NR₇R₇;

12) —SO₂R₇; or

13) carbonyl, optionally substituted with at least one R₇;

wherein R₇ is independently H, hydroxyl, halo, alkyl optionally substituted with at least one R₁₀, alkoxy optionally substituted with at least one R₁₀, cycloalkyl optionally substituted with at least one R₁₀, heterocycloalkyl optionally substituted with at least one R₁₀, aryl optionally substituted with at least one R₁₀, NH₂, NHR₁₀, NR₁₀R₁₀ or SO₂R₁₀, wherein R₁₀ is independently halo, cyano, nitro, C₁-C₆ alkyl, C₁-C₆ alkoxy, or NH₂, wherein when taken together R₁ and R₂ form a ring structure including heterocyclyl or aryl rings;

R₃ is:

1) hydrogen;

2) alkyl, optionally substituted with at least one R₈;

3) alkenyl, optionally substituted with at least one R₈;

4) alkynyl, optionally substituted with at least one R₈;

5) alkoxy, optionally substituted with at least one R₈;

6) cycloalkyl or heterocyclyl, optionally substituted with at least one R₈;

7) aryl, optionally substituted with at least one R₈;

8) cycloalkylalkyl or heterocyclylalkyl, optionally substituted with at least one R₈;

9) aralkyl, optionally substituted with at least one R₈;

10) carbonyl, optionally substituted with at least one R₈; or

11) —SO₂R₈, —CO₂R₈, —SR₈, or —SOR₈;

wherein R₈ is independently H, halo, cyano, nitro, alkyl optionally substituted with at least one R₁₁, alkoxy optionally substituted with at least one R₁₁, cycloalkyl optionally substituted with at least one R₁₁, heterocyclyl optionally substituted with at least one R₁₁, aryl optionally substituted with at least one R₁₁, NH₂, NHR₁₁, NR₁₁R₁₁, or SO₂R₁₁, wherein R₁₁ is independently halo, cyano, nitro, C₁-C₆ alkyl, C₁-C₆ alkoxy, or NH₂,

wherein R₇ is independently H, hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally, R₁ and R₂ taken together form a ring structure including cycloalkyl, heterocyclyl, or aryl ring;

R₃ is:

1) hydrogen;

2) C₁-C₈ alkyl, optionally substituted with at least one R₈;

3) C₂-C₈ alkenyl, optionally substituted with at least one R₈;

4) C₂-C₈ alkynyl, optionally substituted with at least one R₈;

5) C₁-C₈ alkoxy, optionally substituted with at least one R₈;

6) C₃-C₁₀ cycloalkyl or heterocyclyl, optionally substituted with at least one R₈;

7) C₄-C₁₀ cycloalkylalkyl or heterocyclylalkyl, optionally substituted with at least one R₈;

8) C₃-C₁₀ aryl, optionally substituted with at least one R₈;

9) C₄-C₁₀ aralkyl, optionally substituted with at least one R₈;

10) carbonyl, optionally substituted with at least one R₈; or

11) —SO₂R₈, —CO₂R₈, —SR₈, or —SOR₈;

wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,

R₄ is:

1) hydrogen;

2) C₁-C₈ alkyl, optionally substituted with at least one R₁₂;

3) C₂-C₈ alkenyl, optionally substituted with at least one R₁₂;

4) C₂-C₈ alkynyl, optionally substituted with at least one R₁₂;

5) C₃-C₈ cycloalkyl or heterocyclyl, optionally substituted with at least one R₁₂;

6) C₄-C₈ cycloalkylalkyl or heterocyclylalkyl, optionally substituted with at least one R₁₂;

7) C₃-C₁₀ aryl, optionally substituted with at least one R₁₂;

8) C₅-C₁₀ aralkyl, optionally substituted with at least one R₁₂;

9) carbonyl, optionally substituted with at least one R₁₂; or

10) —SO₂R₁₂, or —SOR₁₂;

wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₉ heterocyclylalkyl, or NH₂; optionally, R₃ and R₄ are taken together to form a C₄-C₆ heterocyclyl optionally substituted with R₁₃, or aryl; and

R₆ is:

1) C₁-C₈ alkyl, optionally substituted with at least one R₉;

2) C₂-C₈ alkenyl, optionally substituted with at least one R₉;

3) C₂-C₈ alkynyl, optionally substituted with at least one R₉;

4) C₁-C₈ alkoxy, optionally substituted with at least one R₉;

5) C₃-C₁₀ cycloalkyl or heterocyclyl, optionally substituted with at least one R₉;

6) C₄-C₁₀ cycloalkylalkyl or heterocyclylalkyl, optionally substituted with at least one R₉;

7) C₄-C₁₀ aryl, optionally substituted with at least one R₉;

8) C₅-C₁₀ aralkyl, optionally substituted with at least one R₉; or

9) NH₂, NHR₉ or NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

A preferred embodiment of the invention encompasses compounds of Formula IIA:

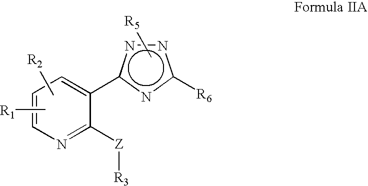

wherein,

Z is O or NR₄;

R₁, R₂, or R₅ each independently is:

1) hydrogen, hydroxyl, fluoro, chloro, bromo, nitro, or cyano;

2) C₁-C₈ alkyl, optionally substituted with at least one R₇;

3) C₁-C₆ alkoxy, optionally substituted with at least one R₇;

4) C₃-C₈ cycloalkyl or heterocyclyl, optionally substituted with at least one R₇;

5) C₄-C₁₀ aryl, optionally substituted with at least one R₇;

6) C₅-C₁₀ aralkyl, optionally substituted with at least one R₇; or

7) NH₂, NHR₇, or NR₇R₇,

wherein R₇ is independently hydroxyl, fluoro, chloro, bromo, C₁-C₆ alkyl optionally substituted with at least one R₁₀, C₁-C₆ alkoxy optionally substituted with at least one R₁₀, NH₂, NHR₁₀, NR₁₀R₁₀, or SO₂R₁₀, wherein R₁₀ is independently fluoro, bromo, chloro, cyano, nitro, C₁-C₄ alkyl, C₁-C₄ alkoxy, or NH₂;

R₃ is:

1) hydrogen;

2) C₃-C₁₀ cycloalkyl or heterocyclyl, optionally substituted with at least one R₈;

3) C₄-C₁₀ cycloalkylalkyl or heterocyclylalkyl, optionally substituted with at least one R₈;

4) C₄-C₁₀ aryl, optionally substituted with at least one R₈;

5) C₄-C₁₀ aralkyl, optionally substituted with at least one R₈; or

6) —SO₂R₈,

wherein R₈ is independently fluoro, chloro, bromo, nitro, C₁-C₄ alkyl optionally substituted with at least one R₁₁, C₁-C₄ alkoxy optionally substituted with at least one R₁₁, C₃-C₁₀ aryl optionally substituted with at least one R₁₁, NH₂, NHR₁₁, NR₁₁R₁₁, or SO₂R₁₁, wherein R₁₁ is independently fluoro, chloro, bromo, cyano, C₁-C₄ alkyl, C₁-C₄ alkoxy, C₆-C₉ aryl, C₃-C₈ aralkyl, or NH₂;

R₄ is:

1) hydrogen;

2) C₃-C₈ cycloalkyl or heterocyclyl, optionally substituted with at least one R₁₂;

3) C₄-C₈ cycloalkylalkyl or heterocyclylalkyl, optionally substituted with at least one R₁₂;

4) C₄-C₁₀ aryl, optionally substituted with at least one R₁₂;

5) C₅-C₁₀ aralkyl, optionally substituted with at least one R₁₂; or

6) —SO₂R₁₂,

wherein R₁₂ is independently chloro, bromo, nitro, C₁-C₆ alkyl optionally substituted with at least one R₁₃, C₁-C₆ alkoxy optionally substituted with at least one R₁₃, C₃-C₁₀ aryl optionally substituted with at least one R₁₃, NH₂, NHR₁₃, NR₁₃R₁₃, or SO₂R₁₃, optionally, R₃ and R₄ are taken together to form a C₄-C₆ heterocyclyl or aryl ring optionally substituted with R₁₃, wherein R₁₃ is independently fluoro, chloro, bromo, cyano, C₁-C₄ alkyl, C₁-C₄ alkoxy, C₃-C₉ aryl, or NH₂; and

R₆ is

1) C₁-C₈ alkoxy, optionally substituted with at least one R₉;

2) C₃-C₁₀ cycloalkyl or heterocyclyl, optionally substituted with at least one R₉;

3) C₄-C₁₀ cycloalkylalkyl or heterocyclylalkyl, optionally substituted with at least one R₉;

4) C₄-C₁₀ aryl, optionally substituted with at least one R₉;

5) C₅-C₁₀ aralkyl, optionally substituted with at least one R₉; or

6) NH₂, NHR₉ or NR₉R₉,

wherein R₉ is independently hydroxyl, fluoro, chloro, bromo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₈ cycloalkyl or heterocyclyl, optionally substituted with at least one R₁₄, C₄-C₈ cycloalkylalkyl or heterocyclylalkyl, optionally substituted with at least one R₁₄, C₄-C₁₀ aryl, optionally substituted with at least one R₁₄; C₅-C₁₀ aralkyl, optionally substituted with at least one R₁₄, —NH₂, —NHR₁₄, —NR₁₄R₁₄, or —SO₂R₁₄, wherein R₁₄ is independently fluoro, chloro, bromo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

In a most preferred embodiment, the compounds of the invention have Formula IIA wherein,

Z is O or NH;

R₁, R₂, or R₅ each independently is:

1) hydrogen, fluoro, chloro, or bromo;

2) C₁-C₆ alkyl, optionally substituted with at least one R₇; or

3) C₃-C₈ heterocyclyl, optionally substituted with at least one R₇,

wherein R₇ is independently fluoro, C₁-C₄ alkyl, NHR₁₀, or NR₁₀R₁₀, wherein R₁₀ is independently C₁-C₄ alkyl;

R₃ is:

1) C₄-C₁₀ heterocyclyl optionally substituted with at least one R₈;

2) C₄-C₁₀ aryl, optionally substituted with at least one R₈;

3) C₅-C₁₀ aralkyl, optionally substituted with at least one R₈; or

4) C₄-C₁₀ heterocyclylalkyl, optionally substituted with at least one R₈;

wherein R₈ is independently fluoro, chloro, bromo, nitro, C₁-C₄ alkyl, C₁-C₄ alkoxy, —SO₂CH₃, or —SO₂NH₂;

R₆ is

1) C₄-C₁₀ heterocyclyl, optionally substituted with at least one R₉;

2) C₅-C₁₀ heterocyclylalkyl, optionally substituted with at least one R₉;

3) C₄-C₁₀ aryl, optionally substituted with at least one R₉;

3) C₅-C₁₀ aralkyl, optionally substituted with at least one R₉; or

4) NHR₉ or NR₉R₉,

wherein R₉ is independently fluoro, chloro, bromo, cyano, C₁-C₄ alkyl, C₁-C₄ alkoxy optionally substituted with at least one R₁₄, C₄-C₈ heterocyclylalkyl optionally substituted with at least one R₁₄, C₄-C₁₀ aryl or heteroaryl optionally substituted with at least one R₁₄; C₅-C₁₀ aralkyl, optionally substituted with at least one R₁₄, —NHR₁₄, —NR₁₄R₁₄, or —SO₂(C₁-C₄ alkyl), wherein R₁₄ is independently fluoro, chloro, bromo, C₁-C₄ alkyl, —SO₂(C₆-C₁₀ aryl), —NH₂, —N(C₁-C₄ alkyl)₂, —NH(C₅-C₈ heterocyclylalkyl), —NH(C₆-C₈ aryl), or —NH(C₆-C₈ heterocyclyl).

Another preferred embodiment of the invention encompasses compounds of Formula IIB:

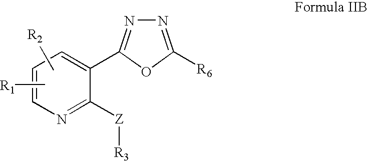

wherein

Z is O or NR₄;

R₁ or R₂ each independently is:

1) hydrogen, hydroxyl, fluoro, chloro, bromo, nitro, or cyano;

2) C₁-C₈ alkyl, optionally substituted with at least one R₇;

3) C₁-C₈ alkoxy, optionally substituted with at least one R₇;

4) C₃-C₈ cycloalkyl or heterocyclyl, optionally substituted with at least one R₇;

5) C₃-C₁₀ aryl, optionally substituted with at least one R₇;

6) C₅-C₁₀ aralkyl, optionally substituted with at least one R₇; or

7) NH₂, NHR₇, or NR₇R₇,

wherein R₇ is independently hydroxyl, fluoro, chloro, bromo, C₁-C₆ alkyl optionally substituted with at least one R₁₀, C₁-C₆ alkoxy optionally substituted with at least one R₁₀, NH₂, NHR₁₀, NR₁₀R₁₀, or SO₂R₁₀, wherein R₁₀ is independently fluoro, bromo, chloro, cyano, nitro, C₁-C₄ alkyl, C₁-C₄ alkoxy, or NH₂;

R₃ is:

1) hydrogen;

2) C₃-C₁₀ cycloalkyl or heterocyclyl, optionally substituted with at least one R₈;

3) C₄-C₁₀ cycloalkylalkyl or heterocyclylalkyl, optionally substituted with at least one R₈;

4) C₄-C₁₀ aryl, optionally substituted with at least one R₈;

5) C₄-C₁₀ aralkyl, optionally substituted with at least one R₈; or

6) —SO₂R₈,

wherein R₉ is independently fluoro, chloro, bromo, nitro, C₁-C₄ alkyl optionally substituted with at least one R₁₁, C₁-C₄ alkoxy optionally substituted with at least one R₁₁, C₆-C₁₀ aryl optionally substituted with at least one R₁₁, NH₂, NHR₁₁, NR₁₁R₁₁, or SO₂R₁₁, wherein R₁₁ is independently fluoro, chloro, bromo, cyano, C₁-C₄ alkyl, C₁-C₄ alkoxy, C₆-C₉ aryl, C₃-C₈ heterocyclyl, or NH₂;

R₄ is:

1) hydrogen;

2) C₃-C₈ cycloalkyl or heterocyclyl, optionally substituted with at least one R₁₂;

3) C₄-C₈ cycloalkylalkyl or heterocyclylalkyl, optionally substituted with at least one R₁₂;

4) C₄-C₁₀ aryl, optionally substituted with at least one R₁₂;

5) C₅-C₁₀ aralkyl, optionally substituted with at least one R₁₂; or

6) —SO₂R₁₂,

wherein R₁₂ is independently chloro, bromo, nitro, C₁-C₄ alkyl optionally substituted with at least one R₁₃, C₁-C₄ alkoxy optionally substituted with at least one R₁₃, C₆-C₁₀ aryl optionally substituted with at least one R₁₃, NH₂, NHR₁₃, NR₁₃R₁₃, or SO₂R₁₃, optionally, R₃ and R₄ are taken together to form a C₄-C₆ heterocyclyl ring optionally substituted with R₁₃, wherein R₁₃ is independently fluoro, chloro, bromo, cyano, C₁-C₄ alkyl, C₁-C₄ alkoxy, C₆-C₉ aryl, C₃-C₈ heterocyclylalkyl, or NH₂; and

R₆ is

1) C₁-C₈ alkoxy, optionally substituted with at least one R₉;

2) C₃-C₁₀ cycloalkyl or heterocyclyl, optionally substituted with at least one R₉;

3) C₄-C₁₀ cycloalkylalkyl or heterocyclylalkyl, optionally substituted with at least one R₉;

4) C₄-C₁₀ aryl, optionally substituted with at least one R₉;

5) C₅-C₁₀ aralkyl, optionally substituted with at least one R₉; or

6) NH₂, NHR₉ or NR₉R₉,

wherein R₉ is independently hydroxyl, fluoro, chloro, bromo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₈ cycloalkyl or heterocyclyl optionally substituted with at least one R₁₄, C₄-C₈ cycloalkylalkyl or heterocyclylalkyl optionally substituted with at least one R₁₄; C₄-C₁₀ aryl, optionally substituted with at least one R₁₄; C₅-C₁₀ aralkyl, optionally substituted with at least one R₁₄, —NH₂, —NHR₁₄, —NR₁₄R₁₄, or —SO₂R₁₄, wherein R₁₄ is independently fluoro, chloro, bromo, cyano, nitro, C₁-C₆ alkyl, C₁-C₆ alkoxy, C₄-C₉ heterocycloalkyl, C₆-C₁₀ aryl, —SO₂(C₆-C₁₀ aryl), —NH₂, —NH[(C₁-C₄)alkyl], —N[(C₁-C₄)alkyl]₂.

In a most preferred embodiment, the compounds of the invention have Formula IIB wherein,

Z is O or NH;

R₁, or R₂ each independently is:

1) hydrogen, fluoro, chloro, or bromo;

2) C₁-C₈ alkyl, optionally substituted with at least one R₇;

3) C₃-C₈ heterocyclyl, optionally substituted with at least one R₇; or

4) NHR₇ or NR₇R₇,

wherein R₇ is independently fluoro, C₁-C₄ alkyl, —NHR₁₀, or —NR₁₀R₁₀, wherein R₁₀ is independently C₁-C₄ alkyl;

R₃ is:

1) C₄-C₈ heterocyclylalkyl, optionally substituted with at least one R₈;

2) C₄-C₁₀ aryl, optionally substituted with at least one R₈;

3) C₄-C₁₀ heterocyclyl, optionally substituted with at least one R₈; or

4) C₄-C₁₀ aralkyl, optionally substituted with at least one R₈;

wherein R₈ is independently fluoro, chloro, bromo, C₁-C₄ alkyl, C₁-C₄ alkoxy, SO₂NH₂, or SO₂CH₃; and

R₆ is:

1) C₃-C₈ heterocyclyl, optionally substituted with at least one R₉;

2) C₄-C₁₀ aryl, optionally substituted with at least one R₉; or

3) —NHR₉ or —NR₉R₉,

wherein R₉ is independently fluoro, chloro, bromo, C₄-C₁₀ aryl, or —SO₂CH₃.

Another preferred embodiment of the invention encompasses compounds of Formula IIC:

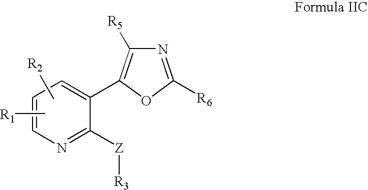

wherein

Z is O or NR₄;

R₁, R₂, or R₅ each independently is:

1) hydrogen, hydroxyl, fluoro, chloro, bromo, nitro, or cyano;

2) C₁-C₈ alkyl, optionally substituted with at least one R₇;

3) C₁-C₈ alkoxy, optionally substituted with at least one R₇;

4) C₃-C₈ cycloalkyl or heterocyclyl, optionally substituted with at least one R₇;

5) C₄-C₈ cycloalkylalkyl or heterocyclylalkyl, optionally substituted with at least one R₇;

6) C₃-C₁₀ aryl, optionally substituted with at least one R₇;

7) C₅-C₁₀ aralkyl, optionally substituted with at least one R₇; or

8) —NHR₇ or —NR₇R₇,

wherein R₇ is independently hydroxyl, fluoro, chloro, bromo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₆-C₁₀ aryl optionally substituted with at least one R₁₀, NH₂, NHR₁₀, NR₁₀R₁₀ or SO₂R₁₀, wherein R₁₀ is independently halo, cyano, nitro, C₁-C₄ alkyl, C₁-C₄ alkoxy, or NH₂, wherein when taken together R₁ and R₂ form a ring structure including cycloalkyl, heterocyclyl, or aryl;

R₃ is:

1) C₁-C₈ alkyl, optionally substituted with at least one R₈;

2) C₁-C₈ alkoxy, optionally substituted with at least one R₈;

3) C₃-C₈ cycloalkyl or heterocyclyl, optionally substituted with at least one R₈;

4) C₄-C₈ cycloalkylalkyl or heterocyclylalkyl, optionally substituted with at least one R₈;

5) C₄-C₁₀ aryl, optionally substituted with at least one R₈;

6) C₅-C₁₀ aralkyl, optionally substituted with at least one R₈; or

7) —SO₂R₈ or —SOR₈;

wherein R₈ is independently H, fluoro, chloro, bromo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, or NH₂,

R₄ is hydrogen or R₃; and

R₆ is:

1) C₃-C₈ cycloalkyl or heterocyclyl, optionally substituted with at least one R₉;

2) C₄-C₈ cycloalkylalkyl or heterocyclylalkyl, optionally substituted with at least one R₉;

3) C₄-C₁₀ aryl, optionally substituted with at least one R₉;

4) C₅-C₁₀ aralkyl, optionally substituted with at least one R₉; or

5) —NHR₉ or —NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl, optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₆-C₁₀ aryl, C₄-C₈ heterocycloalkyl, or NH₂.

A preferred embodiment of the invention encompasses compounds of Formula IIIA:

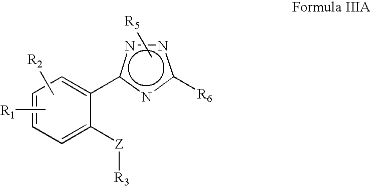

wherein,

Z is O or NR₄;

R₁, R₂, or R₅ each independently is:

1) hydrogen, fluoro, bromo, chloro, nitro, or cyano;

2) C₁-C₈ alkyl, optionally substituted with at least one R₇;

3) C₁-C₈ alkoxy, optionally substituted with at least one R₇;

4) C₃-C₈ cycloalkyl or heterocyclyl, optionally substituted with at least one R₇;

5) C₃-C₁₀ aryl, optionally substituted with at least one R₇;

6) C₅-C₁₀ aralkyl, optionally substituted with at least one R₇; or

7) NH₂, NHR₇, or NR₇R₇,

wherein R₇ is independently hydroxyl, fluoro, chloro, bromo, C₁-C₆ alkyl optionally substituted with at least one R₁₀, C₁-C₆ alkoxy optionally substituted with at least one R₁₀, NH₂, NHR₁₀, NR₁₀R₁₀, or SO₂R₁₀, wherein R₁₀ is independently fluoro, bromo, chloro, cyano, nitro, C₁-C₄ alkyl, C₁-C₄ alkoxy, or NH₂;

R₃ is:

1) C₃-C₈ cycloalkyl or heterocyclyl, optionally substituted with at least one R₈;

3) C₄-C₈ cycloalkylalkyl or heterocyclylalkyl, optionally substituted with at least one R₈;

4) C₄-C₁₀ aryl, optionally substituted with at least one R₈;

5) C₅-C₁₀ aralkyl, optionally substituted with at least one R₈; or

6) —SO₂R₈,

wherein R₈ is independently fluoro, chloro, bromo, nitro, C₁-C₄ alkyl optionally substituted with at least one R₁₁, C₁-C₄ alkoxy optionally substituted with at least one R₁₁, C₆-C₁₀ aryl optionally substituted with at least one R₁₁, NH₂, NHR₁₁, NR₁₁R₁₁, or SO₂R₁₁, wherein R₁₁ is independently fluoro, chloro, bromo, cyano, C₁-C₄ alkyl, C₁-C₄ alkoxy, C₆-C₁₀ aryl, C₃-C₈ heterocyclylalkyl, or NH₂;

R₄ is:

1) hydrogen;

2) C₃-C₈ cycloalkyl or heterocyclyl, optionally substituted with at least one R₁₂;

3) C₄-C₈ cycloalkylalkyl or heterocyclylalkyl, optionally substituted with at least one R₁₂;

4) C₄-C₁₀ aryl, optionally substituted with at least one R₁₂;

5) C₅-C₁₀ aralkyl, optionally substituted with at least one R₁₂; or

6) —SO₂R₁₂,

wherein R₁₂ is independently chloro, bromo, nitro, C₁-C₄ alkyl optionally substituted with at least one R₁₃, C₁-C₄ alkoxy optionally substituted with at least one R₁₃, C₆-C₁₀ aryl optionally substituted with at least one R₁₃, NH₂, NHR₃, NR₁₃R₁₃, or SO₂R₁₃, optionally, R₃ and R₄ are taken together to form a C₄-C₆ heterocyclyl or aryl ring optionally substituted with R₁₃, wherein R₁₃ is independently fluoro, chloro, bromo, cyano, C₁-C₄ alkyl, C₁-C₄ alkoxy, C₃-C₉ aryl, or NH₂; and

R₆ is

1) C₁-C₈ alkoxy, optionally substituted with at least one R₉;

2) C₃-C₁₀ cycloalkyl or heterocyclyl, optionally substituted with at least one R₉;

3) C₄-C₁₀ cycloalkylalkyl or heterocyclylalkyl, optionally substituted with at least one R₉;

4) C₄-C₁₀ aryl, optionally substituted with at least one R₉;

5) C₅-C₁₀ aralkyl, optionally substituted with at least one R₉; or

6) NH₂, NHR₉ or NR₉R₉,

wherein R₉ is independently hydroxyl, fluoro, chloro, bromo, nitro, C₁-C₆ alkyl optionally substituted with at least one R₁₄, C₂-C₆ alkynyl optionally substituted with at least one R₁₄, C₁-C₄ alkoxy optionally substituted with at least one R₁₄, C₃-C₈ cycloalkyl or heterocyclyl, optionally substituted with at least one R₁₄, C₄-C₈ cycloalkylalkyl or heterocyclylalkyl, optionally substituted with at least one R₁₄, C₄-C₁₀ aryl, optionally substituted with at least one R₁₄, C₅-C₁₀ aralkyl, optionally substituted with at least one R₁₄, —NH₂, —NHR₁₄, —NR₁₄R₁₄, or —SO₂R₁₄, wherein R₁₄ is independently fluoro, chloro, bromo, cyano, nitro, C₁-C₄ alkyl, C₁-C₄ alkoxy, C₄-C₉ heterocycloalkyl, C₆-C₁₀ aryl, C₄-C₈ heterocyclyl, —SO₂(C₆-C₁₀ aryl), —NH₂, —NH[(C₁-C₄)alkyl], or —N[(C₁-C₄)alkyl]₂.

In a most preferred embodiment, the compounds of the invention include those of Formula IIIA wherein,

Z is O or NH;

R₁, R₂, or R₅ each independently is:

1) hydrogen, fluoro, bromo, or chloro;

2) C₁-C₆ alkyl, optionally substituted with at least one R₇;

3) C₃-C₈ heterocyclyl, optionally substituted with at least one R₇; or

4) NHR₇ or NR₇R₇,

wherein R₇ is independently fluoro or C₁-C₄ alkyl;

R₃ is:

1) C₃-C₈ heterocyclyl, optionally substituted with at least one R₈;

2) C₄-C₈ heterocyclylalkyl, optionally substituted with at least one R₈;

3) C₄-C₁₀ aryl, optionally substituted with at least one R₈;

4) C₅-C₁₀ aralkyl, optionally substituted with at least one R₈; or

5) SO₂R₈,

wherein R₈ is independently fluoro, chloro, bromo, cyano, C₁-C₄ alkyl, C₁-C₄ alkoxy, or SO₂(C₆-C₁₀ aryl); and

R₆ is

1) C₄-C₁₀ heterocyclyl, optionally substituted with at least one R₉;

2) C₄-C₁₀ heterocyclylalkyl, optionally substituted with at least one R₉;

3) C₄-C₁₀ aryl, optionally substituted with at least one R₉;

4) C₅-C₁₀ aralkyl, optionally substituted with at least one R₉; or

5) NHR₉ or NR₉R₉,

wherein R₉ is independently fluoro, chloro, bromo, C₁-C₆ alkyl, C₂-C₆ alkynyl, C₁-C₆ alkoxy, C₄-C₁₀ heterocyclyl optionally substituted with at least one R₁₄, C₄-C₁₀ aryl optionally substituted with at least one R₁₄, or SO₂CH₃, wherein R₁₄ is independently fluoro, chloro, bromo, or C₁-C₄ alkoxy.

Another preferred embodiment of the invention encompasses compounds of Formula IIIB:

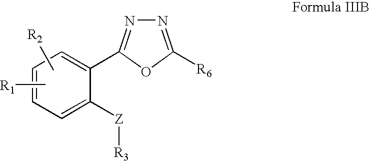

wherein,

Z is O or NR₄;

R₁ or R₂ each independently is:

1) hydrogen, hydroxyl, fluoro, chloro, bromo, nitro, or cyano;

2) C₁-C₈ alkyl, optionally substituted with at least one R₇;

3) C₁-C₈ alkoxy, optionally substituted with at least one R₇;

4) C₃-C₈ cycloalkyl or heterocyclyl, optionally substituted with at least one R₇;

5) C₄-C₈ cycloalkylalkyl or heterocyclylalkyl, optionally substituted with at least one R₇;

6) C₄-C₁₀ aryl, optionally substituted with at least one R₇;

7) C₅-C₁₀ aralkyl, optionally substituted with at least one R₇; or

8) —NHR₇ or —NR₇R₇,

wherein R₇ is independently hydroxyl, fluoro, chloro, bromo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₅-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally R₁ and R₂ are taken together to form a ring heterocyclyl or aryl ring;

R₃ is:

1) hydrogen;

2) C₁-C₈ alkyl, optionally substituted with at least one R₈;

3) C₁-C₈ alkoxy, optionally substituted with at least one R₈;

4) C₃-C₈ cycloalkyl or heterocyclyl, optionally substituted with at least one R₈;

5) C₄-C₈ cycloalkylalkyl or heterocyclylalkyl, optionally substituted with at least one R₈;

6) C₄-C₁₀ aryl, optionally substituted with at least one R₈;

7) C₅-C₁₀ aralkyl, optionally substituted with at least one R₈; or

8) —SO₂R₈,

wherein R₈ is independently fluoro, chloro, bromo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₅-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, or NH₂,

R₄ is hydrogen or R₃; and

R₆ is:

1) C₃-C₈ cycloalkyl or heterocyclyl, optionally substituted with at least one R₉;

2) C₄-C₈ cycloalkylalkyl or heterocyclylalkyl, optionally substituted with at least one R₉;

3) C₄-C₁₀ aryl, optionally substituted with at least one R₉;

4) C₅-C₁₀ aralkyl, optionally substituted with at least one R₉; or

5) —NHR₉ or —NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₄ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl, optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₆-C₁₀ aryl, C₄-C₈ heterocyclyl, or NH₂.

In a most preferred embodiment, the compounds of the invention include those of Formula IIIB wherein,

Z is O or NH;

R₁ or R₂ each independently is:

1) hydrogen, fluoro, chloro, or bromo;

2) C₁-C₈ alkyl, optionally substituted with at least one R₇;

3) C₃-C₈ cycloalkyl or heterocyclyl, optionally substituted with at least one R₇; or

4) NHR₇ or NR₇R₇,

wherein R₇ is independently fluoro, chloro, bromo, or C₁-C₄ alkyl;

R₃ is:

1) hydrogen;

2) C₁-C₈ alkyl, optionally substituted with at least one R₈;

3) C₁-C₈ alkoxy, optionally substituted with at least one R₈;

4) C₃-C₈ cycloalkyl or heterocyclyl, optionally substituted with at least one R₈;

5) C₄-C₈ cycloalkylalkyl or heterocyclylalkyl, optionally substituted with at least one R₈;

6) C₄-C₁₀ aryl, optionally substituted with at least one R₈;

7) C₅-C₁₀ aralkyl, optionally substituted with at least one R₈; or

8) —SO₂R₈,

wherein R₈ is independently fluoro, chloro, bromo, cyano, nitro, C₁-C₄ alkyl, C₁-C₄ alkoxy, C₆-C₁₀ aryl optionally substituted with at least one R₁₁, C₆-C₁₀ aralkyl optionally substituted with at least one R₁₁, wherein R₁₁ is independently fluoro, chloro, bromo, cyano, C₁-C₄ alkyl, or C₁-C₄ alkoxy; and

R₆ is:

1) C₃-C₈ cycloalkyl or heterocyclyl, optionally substituted with at least one R₉;

2) C₄-C₈ cycloalkylalkyl or heterocyclylalkyl, optionally substituted with at least one R₉;

3) C₄-C₁₀ aryl, optionally substituted with at least one R₉;

4) C₅-C₁₀ aralkyl, optionally substituted with at least one R₉; or

5) —NHR₉ or —NR₉R₉,

wherein R₉ is independently hydroxyl, fluoro, chloro, bromo, nitro, or C₆-C₁₀ aryl optionally substituted with at least one R₁₄, wherein R₁₄ is independently fluoro, chloro, bromo, C₁-C₄ alkoxy, or C₆-C₁₀ aryl.

Another preferred embodiment of the invention encompasses compounds of Formula IIIC:

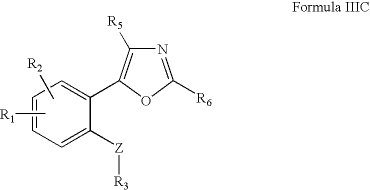

wherein

Z is O or NR₄;

R₁, R₂, or R₅ each independently is:

1) hydrogen, hydroxyl, fluoro, chloro, bromo, nitro, or cyano;

2) C₁-C₆ alkyl, optionally substituted with at least one R₇;

3) C₁-C₆ alkoxy, optionally substituted with at least one R₇;

4) C₃-C₈ cycloalkyl or heterocyclyl, optionally substituted with at least one R₇;

5) C₄-C₈ cycloalkylalkyl or heterocyclylalkyl, optionally substituted with at least one R₇;

6) C₄-C₁₀ aryl, optionally substituted with at least one R₇;

7) C₅-C₁₀ aralkyl, optionally substituted with at least one R₇; or

8) —NHR₇ or —NR₇R₇,

wherein R₇ is independently hydroxyl, fluoro, chloro, bromo, C₁-C₄ alkyl optionally substituted with at least one R₁₀, C₁-C₄ alkoxy optionally substituted with at least one R₁₀, C₃-C₈ cycloalkyl optionally substituted with at least one R₁₀, C₄-C₈ heterocycloalkyl optionally substituted with at least one R₁₀, C₆-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂, wherein when taken together R₁ and R₂ form a ring structure including heterocyclyl or aryl rings;

R₃ is:

1) C₁-C₆ alkyl, optionally substituted with at least one R₈;

2) C₁-C₆ alkoxy, optionally substituted with at least one R₈;

3) C₃-C₈ cycloalkyl or heterocyclyl, optionally substituted with at least one R₈;

4) C₄-C₈ cycloalkylalkyl or heterocyclylalkyl, optionally substituted with at least one R₈;

5) C₄-C₁₀ aryl, optionally substituted with at least one R₈;

6) C₅-C₁₀ aralkyl, optionally substituted with at least one R₈; or

7) —SO₂R₈ or —SOR₈;

wherein R₈ is independently fluoro, chloro, bromo, cyano, nitro, C₁-C₆ alkyl optionally substituted with at least one R₁₁, C₁-C₆ alkoxy optionally substituted with at least one R₁₁, C₃-C₈ cycloalkyl optionally substituted with at least one R₁₁, C₃-C₈ heterocyclyl optionally substituted with at least one R₁₁, C₅-C₁₀ aryl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, or NH₂,

R₄ is hydrogen or R₃; and

R₆ is:

1) C₃-C₈ cycloalkyl or heterocyclyl, optionally substituted with at least one R₉;

2) C₄-C₈ cycloalkylalkyl or heterocyclylalkyl, optionally substituted with at least one R₉;

3) C₄-C₁₀ aryl, optionally substituted with at least one R₉;

4) C₅-C₁₀ aralkyl, optionally substituted with at least one R₉; or

5) —NHR₉ or —NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl, optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₆-C₁₀ aryl, or NH₂.

In a most preferred embodiment, the compounds of the invention include those of Formula IIIC wherein,

Z is O or NH;

R₁, R₂, or R₅ each independently is:

1) hydrogen, hydroxyl, fluoro, chloro, bromo, nitro, or cyano;

2) C₁-C₆ alkyl, optionally substituted with at least one R₇;

3) C₁-C₆ alkoxy, optionally substituted with at least one R₇;

4) C₃-C₈ cycloalkyl or heterocyclyl, optionally substituted with at least one R₇;

5) C₄-C₈ cycloalkylalkyl or heterocyclylalkyl, optionally substituted with at least one R₇;

6) C₄-C₁₀ aryl, optionally substituted with at least one R₇;

7) C₅-C₁₀ aralkyl, optionally substituted with at least one R₇; or

8) —NHR₇ or —NR₇R₇,

wherein R₇ is independently H, hydroxyl, fluoro, chloro, bromo, C₁-C₄ alkyl optionally substituted with at least one R₁₀, C₁-C₄ alkoxy optionally substituted with at least one R₁₀, C₃-C₈ cycloalkyl optionally substituted with at least one R₁₀, C₄-C₈ heterocycloalkyl optionally substituted with at least one R₁₀, C₆-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂, wherein when taken together R₁ and R₂ form a ring structure including heterocyclyl or aryl ring;

R₃ is:

1) C₁-C₆ alkyl, optionally substituted with at least one R₈;

2) C₄-C₈ heterocyclyl, optionally substituted with at least one R₈; or

3) C₄-C₁₀ aryl, optionally substituted with at least one R₈,

wherein R₈ is independently fluoro, chloro, bromo, C₁-C₄ alkyl, or C₁-C₄ alkoxy; and

R₆ is:

1) —NHR₉ or —NR₉R₉,

wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl, optionally substituted with at least one R₁₄, —NH₁₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₅-C₁₀ aryl, or NH₂.

Even most preferably, the compounds of the invention encompass compounds of Formulas IIA, IIB, IIC, IIIA, IIIB, or IIIC, wherein Z is NH.

Compounds of Formula I may be made using a variety of synthetic pathways. For illustration purposes, applicants provide the following synthetic schemes, with the understanding that one skilled in the art may vary conditions and/or reagents without deviating from the described process.

Compounds of Formula I wherein the five membered ring is a triazole are made using the synthetic pathways illustrated in Schemes 1 and 2. Although the schemes illustrate a six membered ring with one substitution, a second substitution is well within the abilities of the ordinary skilled artisan. The reactions may be carried out consecutively, i.e., with intervening isolation and/or purification steps, or concurrently, i.e., the reaction mixture is carried forth in the reaction sequence without isolation and/or purification.

Compound A may be synthesized in at least two ways as illustrated by Scheme 1. In one case, the ester of 2-halobenzoate or 2-halonicotinate is reacted with a mono or di-substituted amine under basic conditions to form Compound A. An alternative, is to react an ester of 2-aminonicotinoate or 2-aminobenzoate, as illustrated a methyl ester, with a substituted aldehyde and a reducing agent, such as NaBH(OAc)₃, to yield Compound A. Thereafter, Compound A is reacted with a substituted hydrazine to yield Compound B.

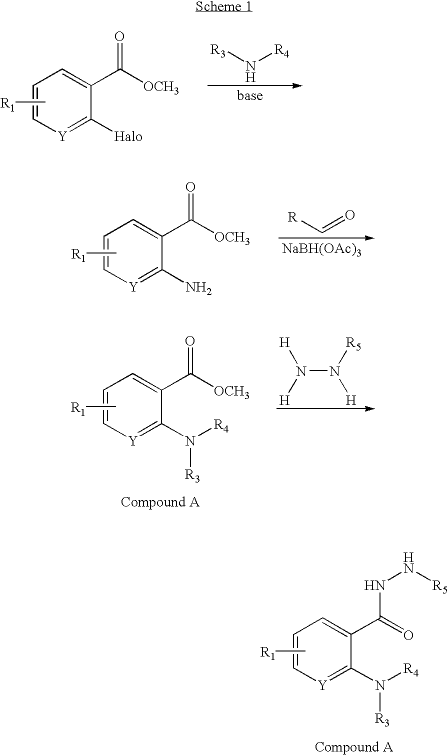

In a second sequence, a halomethane is allowed to react with a substituted thiourea which is then allowed to react with Compound B to yield compounds of Formula II, wherein the five-membered ring is a substituted or unsubstituted triazole. See Scheme 2. As the skilled artisan easily recognizes, the triazole may be substituted by using a substituted hydrazine or N,N′-disubstituted thiourea. In an alternative reaction sequence, Compound B is allowed to react with an amidine to form compounds of Formula I wherein the five-membered ring is a triazole.

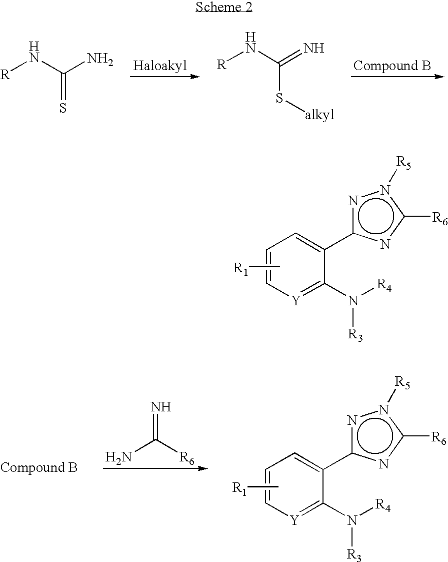

Compounds of Formula I wherein the five-membered ring is an oxadiazole are made using the synthetic pathways illustrated in Schemes 3 and 4. Compound A, synthesized as described above, is allowed to react with hydrazine to form Compound C.

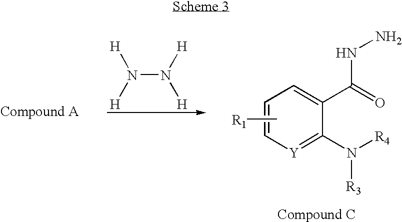

Thereafter, Compound C is allowed to react with an isothiocyanate to yield Compound D. Subsequently, Compound D is allowed to react with a coupling reagent, such as DCC, to yield compounds of Formula I, wherein the five-membered ring is an oxadiazole ring. See Scheme 4.

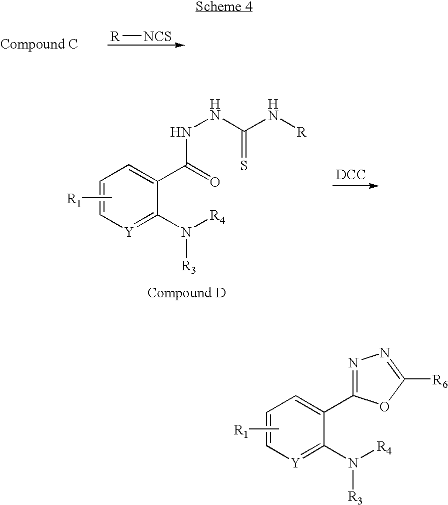

Compounds wherein the five-membered ring is an oxazole are made using the synthetic pathways illustrated in Scheme 5. In one case, a substitution reaction of an 2-halo-2′-nitroacetophenone with an azide to form Compound E, which is then allowed to react with an isothiocyanate to form Compound F. Hydrogenation of the nitro group into an amine (Compound G), followed by reaction with an aldehyde yields compounds of Formula I, wherein the five-membered ring is an oxazole.

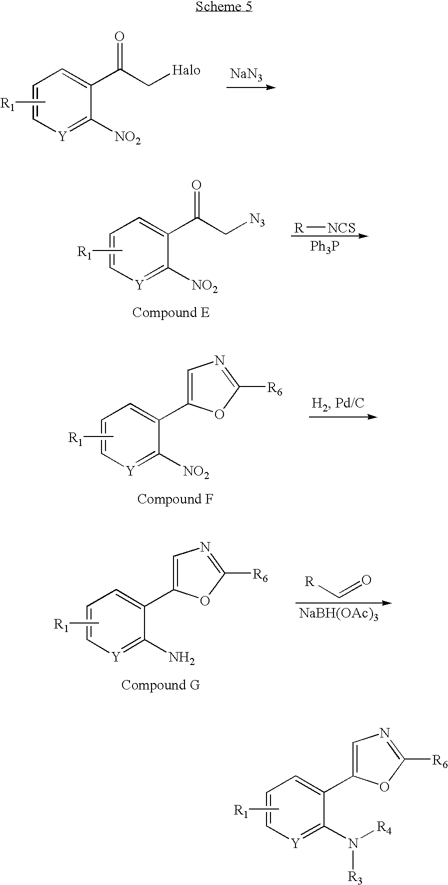

The pharmaceutical compositions of the invention comprise compounds of Formula I, or a pharmaceutically acceptable salt, solvate, hydrate, or clathrate thereof as an active ingredient, and may also contain a pharmaceutically acceptable carrier and optionally other therapeutic ingredients known to those skilled in the art. Preferred pharmaceutical compositions comprise at least one compound of Formula IIA, IIB, IIC, IIIA, IIIB, or IIIC.

Another aspect of the present invention relates to pharmaceutical compositions, which include at least one compound of the present invention as described herein (that is, a compound of Formula I) or a pharmaceutically acceptable salt, hydrate or pro-drug thereof, in combination with a pharmaceutically acceptable carrier.

Compositions of the invention are suitable for oral, mucosal (e.g., nasal, vaginal, or rectal), parenteral (e.g., subcutaneous, intravenous, bolus injection, intramuscular, or intraarterial), sublingual, transdermal, or buccal administration, although the most suitable route in any given case will depend on the nature and severity of the condition being treated. The compositions may be conveniently presented in unit dosage form and prepared by any of the methods well known in the part of pharmacy. Dosage forms include tablets, caplets, troches, lozenges, dispersions, suspensions, suppositories, solutions, capsules, soft elastic gelatin capsules, patches, and the like. Preferred dosage forms are those suitable for oral administration.

The compositions of the present invention may be employed in solid or liquid form including for example, powder or crystalline form, in solution or in suspension. The choice of carrier and the content of active compound in the carrier are generally determined in accordance with the solubility and chemical properties of the desired product, the particular mode of administration and the provisions to be observed in pharmaceutical practice. Thus, the carrier employed may be, for example, either a solid or liquid.

One method of administering a solid dosage form is to form solid compositions for rectal administration, which include suppositories formulated in accordance with known methods and containing at least one compound of the present invention. Examples of solid carriers include lactose, sucrose, talc, gelatin, agar, pectin, acacia, magnesium stearate, stearic acid and the like.

Examples of liquid carriers include syrup, peanut oil, olive oil, water and the like. For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. Injectable forms must be fluid to the extent they can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin.

The solutions of the salts of the products according to the invention are especially useful for administration by intramuscular or subcutaneous injection. Solutions of the active compound as a free base or pharmacologically acceptable salt can be prepared in water suitably mixed with a surfactant such as hydroxypropyl-cellulose. Dispersions can also be prepared in glycerol, liquid polyethylene glycols, and mixtures thereof and in oils. The aqueous solutions, also including solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Examples of injectable dosage forms include sterile injectable liquids, e.g., solutions, emulsions and suspensions. Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, methods of preparation may include vacuum drying and a freeze-dry technique that yields a powder of the active ingredient plus any additional desired ingredient from previously sterile-filtered solution thereof.

Examples of injectable solids include powders that are reconstituted, dissolved, or suspended in a liquid prior to injection. In injectable compositions, the carrier typically includes sterile water, saline or another injectable liquid, e.g., peanut oil for intramuscular injections. Also, various buffering agents, preservatives and the like can be included within the compositions of the present invention.

For oral administration, the active compound may be administered, for example, with an inert diluent or with an assimilable edible carrier, or it may be enclosed in hard or soft shell gelatin capsules, or it may be compressed into tablets, or it may be incorporated directly with the food of the diet, or may be incorporated with excipient and used in the form of ingestible tablets, buccal tablets, troches, capsules, elixirs, suspensions, syrups, wafers, and the like. Examples of oral solid dosage forms include tablets, capsules, troches, lozenges and the like. Examples of oral liquid dosage forms include solutions, suspensions, syrups, emulsions, soft gelatin capsules and the like. Carriers for oral use (solid or liquid) may include time delay materials known in the art, such as glyceryl monostearate or glyceryl distearate alone or with a wax. To prepare a capsule, it may be advantageous to use lactose and liquid carrier, such as high molecular weight polyethylene glycols.

Topical administration, in the form of gels (water or alcohol based), creams or ointments, for example, containing compounds of the invention may be used. Topical applications may be formulated in carriers such as hydrophobic or hydrophilic bases to form ointments, creams, lotions, in aqueous, oleaginous or alcoholic liquids to form paints or in dry diluents to form powders. Such topical formulations can be used for example, to treat ocular diseases as well as inflammatory diseases such as rheumatoid arthritis, psoriasis, contact dermatitis, delayed hypersensitivity reactions and the like.

Compounds of the invention may be also incorporated in a gel or matrix base for application in a patch, which would allow a controlled release of compound through transdermal barrier.

For administration by inhalation compounds of the invention may be dissolved or suspended in a suitable carrier for use in a nebulizer or a suspension or solution aerosol, or may be absorbed or adsorbed onto a suitable solid carrier for use in a dry powder inhaler.

Compositions according to the invention may also be formulated in a manner that resists rapid clearance from the vascular (arterial or venous) wall by convection and/or diffusion, thereby increasing the residence time of the viral particles at the desired site of action. A periadventitial depot comprising a compound according to the invention may be used for sustained release. One such useful depot for administering a compound according to the invention may be a copolymer matrix, such as ethylene-vinyl acetate, or a polyvinyl alcohol gel surrounded by a Silastic shell. Alternatively, a compound according to the invention may be delivered locally from a silicone polymer implanted in the adventitia.

An alternative approach for minimizing washout of a compound according to the invention during percutaneous, transvascular delivery comprises the use of nondiffusible, drug-eluting microparticles. The microparticles may be included a variety of synthetic polymers, such as polylactide for example, or natural substances, including proteins or polysaccharides. Such microparticles enable strategic manipulation of variables including total dose of drug and kinetics of its release. Microparticles can be injected efficiently into the arterial or venous wall through a porous balloon catheter or a balloon over stent, and are retained in the vascular wall and the periadventitial tissue for at least about two weeks. Formulations and methodologies for local, intravascular site-specific delivery of therapeutic agents are discussed in Reissen et al. (J. Am. Coll. Cardiol., 23: 1234-1244 (1994)).

A composition according to the invention may also comprise a hydrogel which is prepared from any biocompatible or non-cytotoxic (homo or hetero) polymer, such as a hydrophilic polyacrylic acid polymer that can act as a drug absorbing sponge. Such polymers have been described, for example, in application WO93/08845. Certain of them, such as, in particular, those obtained from ethylene and/or propylene oxide are commercially available.

Another embodiment of the invention provides for a compound according to the invention to be administered by means of perfusion balloons. These perfusion balloons, which make it possible to maintain a blood flow and thus to decrease the risks of ischaemia of the myocardium, on inflation of the balloon, also enable the compound to be delivered locally at normal pressure for a relatively long time, more than twenty minutes, which may be necessary for its optimal action.

Alternatively, a channeled balloon catheter (such as “channeled balloon angioplasty catheter”, Mansfield Medical, Boston Scientific Corp., Watertown, Mass.) may be used. This catheter includes a conventional balloon covered with a layer of 24 perforated channels that are perfused via an independent lumen through an additional infusion orifice. Various types of balloon catheters, such as double balloon, porous balloon, microporous balloon, channel balloon, balloon over stent and hydrogel catheters, all of which may be used to practice the invention, are disclosed in Reissen et al. (1994).

Another aspect of the present invention relates to a pharmaceutical composition including a compound according to the invention and poloxamer, such as Poloxamer 407, which is a non-toxic, biocompatible polyol, commercially available (e.g., from BASF, Parsippany, N.J.). A poloxamer impregnated with a compound according to the invention may be deposited for example, directly on the surface of the tissue to be treated, for example during a surgical intervention. Poloxamer possesses essentially the same advantages as hydrogel while having a lower viscosity. The use of a channel balloon catheter with a poloxamer impregnated with a compound according to the invention may be advantageous in that it may keep the balloon inflated for a longer period of time, while retaining the properties of facilitated sliding, and of site-specificity of the poloxamer.

The composition may also be administered to a patient via a stent device. In this embodiment, the composition is a polymeric material in which the compound of the invention is incorporated, which composition is applied to at least one surface of the stent device.

Polymeric materials suitable for incorporating the compound of the invention include polymers having relatively low processing temperatures such as polycaprolactone, poly(ethylene-co-vinyl acetate) or poly(vinyl acetate or silicone gum rubber and polymers having similar relatively low processing temperatures. Other suitable polymers include non-degradable polymers capable of carrying and delivering therapeutic drugs such as latexes, urethanes, polysiloxanes, styrene-ethylene/butylene-styrene block copolymers (SEBS) and biodegradable, bioabsorbable polymers capable of carrying and delivering therapeutic drugs, such as poly-DL-lactic acid (DL-PLA), and poly-L-lactic acid (L-PLA), polyorthoesters, polyiminocarbonates, aliphatic polycarbonates, and polyphosphazenes.

In addition to the active compound and the pharmaceutically acceptable carrier, the compositions of the present invention optionally contain one or more excipients that are conventional in the art. For example, excipients such as lactose, sodium citrate, calcium carbonate, dicalcium phosphate and disintegrating agents such as starch, alginic acids and certain complex silica gels combined with lubricants such as magnesium stearate, sodium lauryl sulfate and talc may be used for preparing tablets, troches, pills, capsules and the like.

Various other materials may be present as coatings or to otherwise modify the physical form of the dosage unit. For instance, tablets, pills, or capsules may be coated with shellac, sugar or both. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the active compound may be incorporated into sustained-release preparations and formulations.

The percentage of active ingredient in the compositions of the invention may be varied. Several unit dosage forms may be administered at about the same time. A suitable dose employed may be determined by a physician or qualified medical professional, and depends upon various factors including the desired therapeutic effect, the nature of the illness being treated, the route of administration, the duration of the treatment, and the condition of the patient, such as age, weight, general state of health and other characteristics, which can influence the efficacy of the compound according to the invention. In adults, doses are generally from about 0.001 to about 50, preferably about 0.001 to about 5, mg/kg body weight per day by inhalation; from about 0.01 to about 100, preferably 0.1 to 70, more preferably 0.5 to 10, mg/kg body weight per day by oral administration; from about 0.1 to about 150 mg applied externally; and from about 0.001 to about 10, preferably 0.01 to 10, mg/kg body weight per day by intravenous or intramuscular administration.

The compounds and compositions according to the invention may be administered as frequently as necessary as determined by a skilled practitioner in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. For other patients, it may be necessary to prescribe not more than one or two doses per day.

The compounds of the present invention may also be formulated for use in conjunction with other therapeutically active compounds or in connection with the application of therapeutic techniques to address pharmacological conditions, which may be ameliorated through the application of a compound according to the present invention.

One embodiment of the invention encompasses method of treating cancer using the compounds of the invention. The disclosed compounds can be used to treat subjects with cancer, including multi-drug resistant cancers. A cancer is resistant to a drug when it resumes a normal rate of tumor growth while undergoing treatment with the drug after the tumor had initially responded to the drug. The term “multi-drug resistant cancer” refers to cancer that is resistant to two or more drugs, typically five or more.

The disclosed compounds can be co-administered with other anticancer agents such as Taxol, Vincristine, Adriamycin, Etoposide, Doxorubicin, Dactinomycin, Mitomycin C, Bleomycin, Vinblastine, Cisplatin, Erbitux, Avastin, Irressa, and the like. Additionally, the disclosed compounds can be co-administered with bioactive anticancer agents such as kinase inhibitors, kinase receptors, antigenesis inhibitors, cell cycle inhibitors, cytotoxic targeting agents, signal transduction pathway inhibitors, and the like. The method can also be carried in combination with other cancer treatments such as surgery, radiation, and the like.

Moreover, the compounds of Formula I may be used for in vivo and in vitro investigative, diagnostic, or prophylactic methods, which are well known in the art.

The methods of the present invention encompass administration of a therapeutically effective amount of at least one compound of Formula I to a mammal in need of such treatment. As used herein, the term “administering” means delivering the compounds of the present invention to a mammal by any method that may achieve the result sought. The method may be, for example, orally, parenterally (intravenously or intramuscularly), topically, transdermally, or by inhalation. The term “mammal” as used herein is intended to include, but is not limited to, humans, laboratory animals, domestic pets and farm animals. The term “therapeutically effective amount” as used herein with respect to the treatment or prevention of cancer encompasses an amount of compound of the present invention that when administered to a mammal is effective in producing the desired therapeutic effect. For example, a desired effect is a tumor growth rate reduction to a rate less than untreated tumor growth rate. Preferably, wherein the tumor growth rate is reduced for about 20% to about 100%.

Different therapeutically effective amounts may be applicable for different diseases and conditions, as will be readily known by those of ordinary skill in the art. Similarly, amounts sufficient to treat or prevent such disorders, but insufficient to cause adverse effects associated with compounds of Formula I, are also encompassed by dosage amounts and dose frequency schedules.

The compounds of the invention were tested to determine biological activity using an in vitro tubulin polymerization assay, cell cycle analysis, and SRB cytotoxicity assay. The results of the assays are summarized in Tables 1-5.

Briefly, tubulin polymerization is a kinetic process that is temperature-dependent and requires GTP and was performed as follows. Soluble tubulin dimers polymerize into microtubules upon warming, and polymerization in vitro correlates with an increase in turbidity (measured at 340 nm). Lyophilized bovine tubulin (HTS Tubulin—97% tubulin, <3% MAPs—Cytoskeleton Inc.) was resuspended in G-PEM buffer (80 mM PIPES pH 7, 1 mM EGTA, 1 mM MgCl₂, 1 mM GTP, 5% glycerol) to a final concentration of 3 mg/ml and kept at 4° C. Compounds in 100× stock solutions in DMSO were dotted to pre-warmed 96-well plates (Corning Costar 3696), the plates were transferred to a 37° C. plate reader (SPECTRAmax Plus, Molecular Devices), cold tubulin was added to the wells, and the plates were shaken for mixing. The absorbance at 340 nm was determined at one minute intervals for 30 minutes. Kinetic curves with 30 points each were collected for each compound, and the dynamic range was between 0 and 0.4 OD units. The percentage inhibition values were calculated using the 30 minute data point and based on control samples (treated with 1% DMSO only). The assay is a modified version of the HTS kit sold by Cytoskeleton (1830 S. Acoma St., Denver, Colo.), adapted to maximize throughput and reduce time, without reduction in dynamic range or sensitivity, while retaining the ability to detect compounds that inhibit or enhance tubulin polymerization.

The cell cycle analysis was performed as follows. Cancer cells (A431, human epidermoid carcinoma cells) were maintained in culture in D-MEM media with 10% FBS and 1 mg/ml glutamate. Prior to experiment, cells were plated onto 6-well plates for a final density of 500,000 cells/well at the time of treatment. Cells were treated with the compounds of the invention at a concentration of about 0.01 to 1 μM final concentrations (final 0.1% DMSO) for 24 hours, then trypsinized, collected, rinsed in PBS (phosphate buffered saline), and fixed in 70% cold ethanol overnight at 4° C. The cells were then rinsed with PBS, resuspended in PBS with 0.2% Tween, RNAse was added (final 1 μg/ml), cells were incubated at 37° C. for 15 min, followed by addition of Propidium Iodide (final 50 μg/ml), and a 30 minute incubation at room temperature. DNA ploidy was analyzed using flow cytometers (Epics Excel, Beckman-Coulter, or Guava PCA-96, Guava Technologies) and mitotic arrest characterized by massive accumulation of cells in the G2/M phase of cell cycle.

The in vitro growth inhibition activity of the compounds was determined by the Sulphorhodamine B assay. (Skehan P, Storeng R, Scudiero D, Monks A, McMahon J, Vistica D, Warren J T, Bokesch H, Kenney S, Boyd M R. New calorimetric cytotoxicity assay for anticancer-drug screening. J Natl Cancer Inst 82, 1107-1112, 1990). Sulphorhodamine B binds to basic amino acids and stains proteins which can be eluted and detected spectrophotometrically by measuring absorbance at 515 nm. The absorbance was indicative of the total protein content of the cells fixed to the walls of the plate well at a given time by trichloroacetic acid, which is a measure of the viable cell concentration. The results of the assays are included in the following tables.

TABLE 1
Compounds of Formula IIA, pyridyl-triazoles.

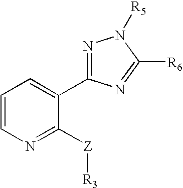

| Comp. | | | | Tubulin | FACS | SRB |
|---|---|---|---|---|---|---|
| No. | —Z—R₃ | R₆ | R₅ | Assay^{a} | Assay^{b} | Assay^{c} |
| 1 | 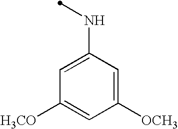 | 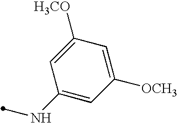 | H | 45 | 0.1 | 5.5 |
| 2 | 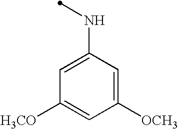 | 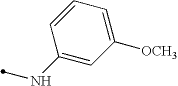 | H | 80 | 0.1 | 0.7 |
| 3 | 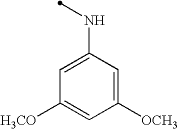 | 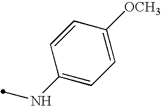 | H | 72 | 10 | 2.5 |
| 4 | 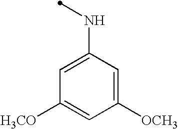 | 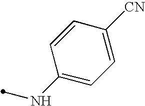 | H | 32 | 10 | 4 |
| 5 | 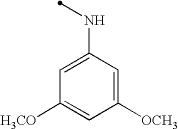 | 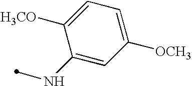 | H | 20 | 100 | 10 |
| 6 | 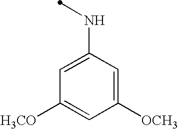 | 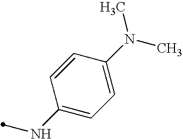 | H | 35 | 10 | 20 |
| 7 | 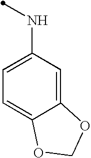 | 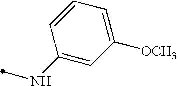 | H | 47 | 10 | 15 |
| 8 | 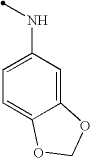 | 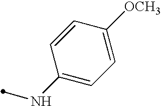 | H | 100 | | 0.3 |
| 9 | 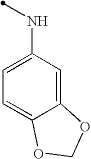 | 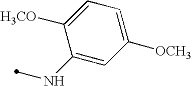 | H | 47 | | 25 |
| 10 | 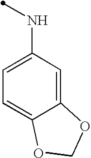 | 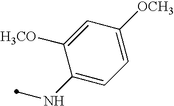 | H | 73 | | 20 |
| 11 | 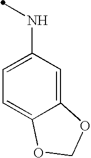 | 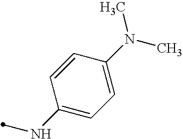 | H | 82 | 10 | 0.25 |
| 12 | 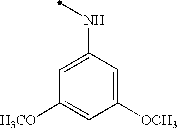 | 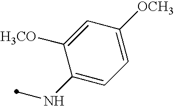 | H | 46 | | 20 |
| 13 | 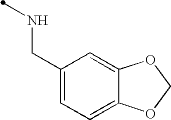 | 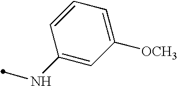 | H | 80 | 1 | 2 |
| 14 | 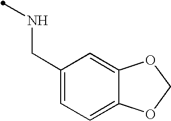 | 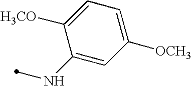 | H | 24 | 100 | 20 |
| 15 | 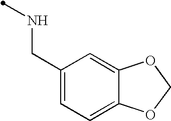 | 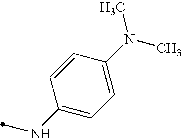 | H | 20 | 100 | 0.2 |
| 16 | 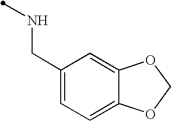 | 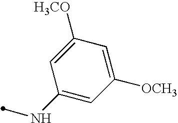 | H | 41 | | 2.5 |
| 17 | 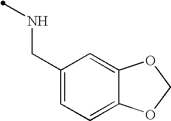 | 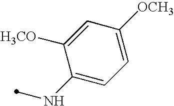 | H | 80 | 10 | 4 |
| 18 | 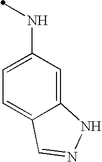 | 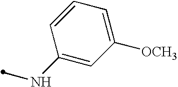 | H | 70 | 1 | 1.5 |
| 19 | 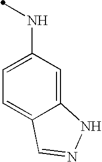 | 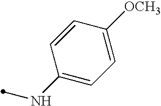 | H | 20 | | |
| 20 | 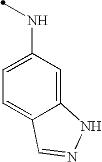 | 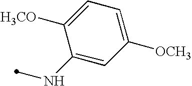 | H | 44 | 10 | 0.9 |
| 21 | 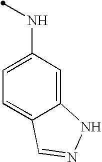 | 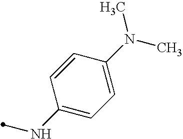 | H | 24 | 100 | 40 |
| 22 | 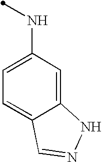 | 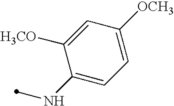 | H | 24 | | |
| 23 | 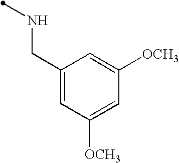 | 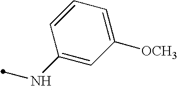 | H | 22 | | |
| 24 | 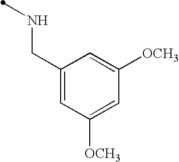 | 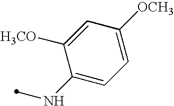 | H | 20 | | |
| 25 | 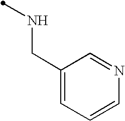 | 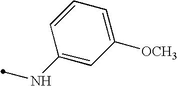 | H | 77 | 1 | 0.5 |
| 26 | 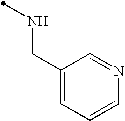 | 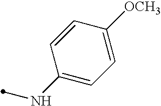 | H | 20 | | |
| 27 | 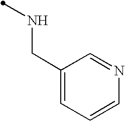 | 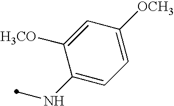 | H | 20 | | |
| 28 | 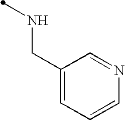 | 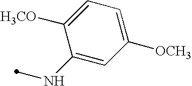 | H | 22 | | |
| 29 | 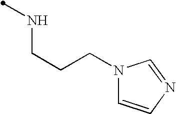 | 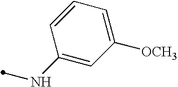 | H | 28 | | |
| 30 | 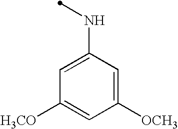 | 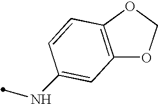 | H | 87 | 0.1 | 0.01 |
| 31 | 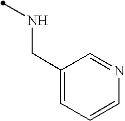 | 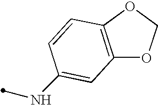 | H | 49 | 1 | 0.25 |
| 32 | 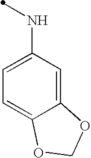 | 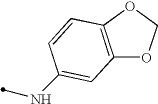 | H | 54 | 10 | |
| 33 | 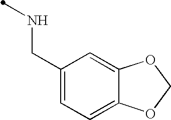 | 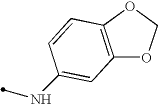 | H | 60 | | 10 |
| 34 | 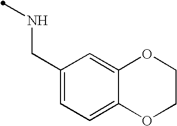 | 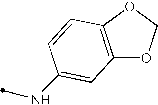 | H | 69 | | |
| 35 | 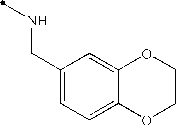 | 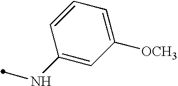 | H | 66 | | |
| 36 | 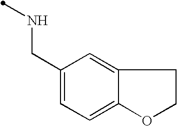 | 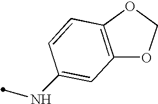 | H | 67 | | |
| 37 | 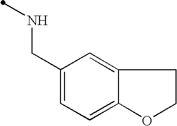 | 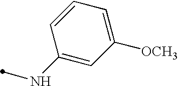 | H | 91 | | |
| 38 | 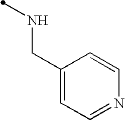 | 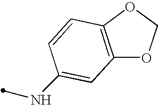 | H | 77 | 0.1 | |
| 39 | 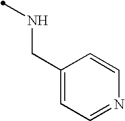 | 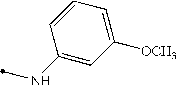 | H | 83 | 0.1 | |
| 40 | 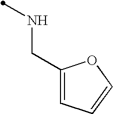 | 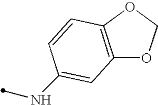 | H | 71 | 1 | |
| 41 | 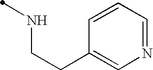 | 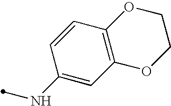 | H | 23 | | |
| 42 | 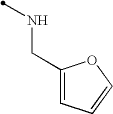 | 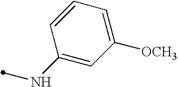 | H | 69 | 10 | |
| 43 | 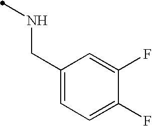 | 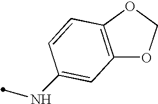 | H | 84 | 1 | |
| 44 | 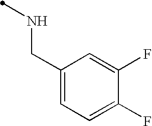 | 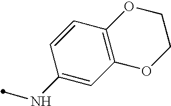 | H | 84 | 1 | |
| 45 | 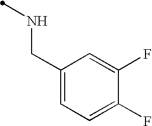 | 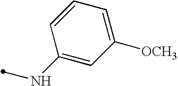 | H | 83 | 1 | |
| 46 | 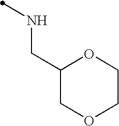 | 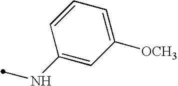 | H | 25 | | |
| 47 | 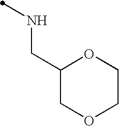 | 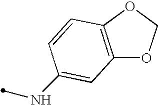 | H | 24 | | |
| 48 | 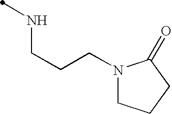 | 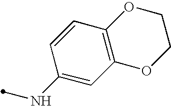 | H | 21 | | |
| 49 | 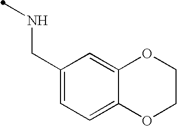 | 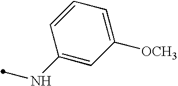 | H | 78 | 1 | |
| 50 | 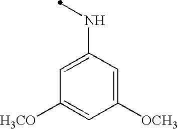 | 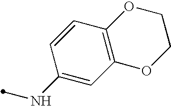 | H | 90 | 0.1 | |
| 51 | 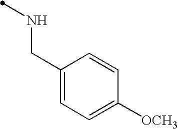 | 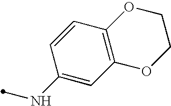 | H | 68 | 1 | |
| 52 | 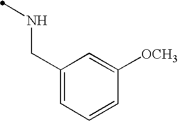 | 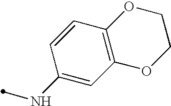 | H | 35 | | |
| 53 | 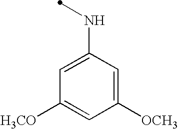 | 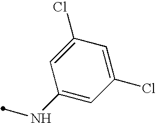 | H | 44 | | 6 |
| 54 | 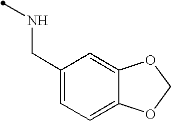 | 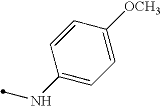 | H | 38 | 10 | 7.5 |
| 55 | 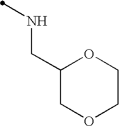 | 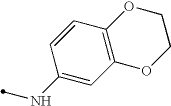 | H | 23 | | |
| 56 | 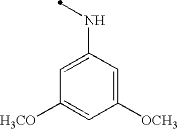 | 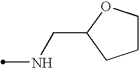 | H | 97 | 1 | |
| 57 | 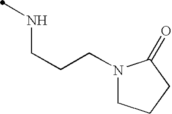 | 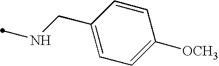 | H | 22 | | |
| 58 | 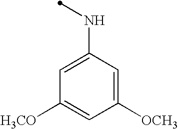 | 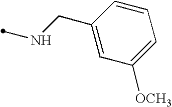 | H | 100 | 0.1 | |
| 59 | 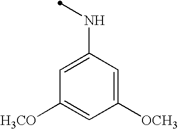 | 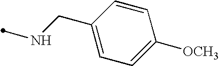 | H | 88 | 1 | |
| 60 | 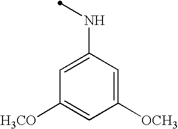 | 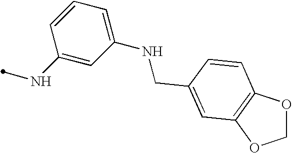 | H | 99 | | |
| 60a | 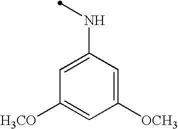 | 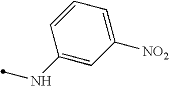 | H | 95 | 0.1 | |
| 60b | 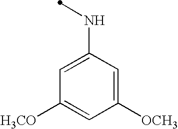 | 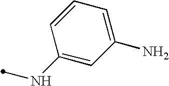 | H | 39 | | |
| 61 | 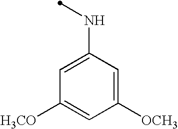 | 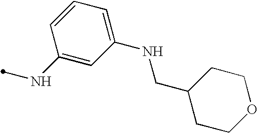 | H | 24 | | |
| 62 | 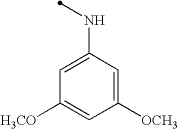 | 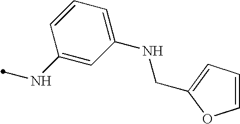 | H | 73 | | |
| 63 | 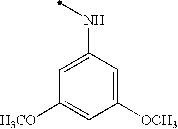 | 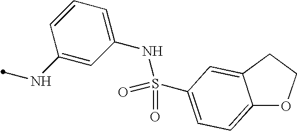 | H | 24 | | |
| 64 | 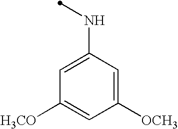 | 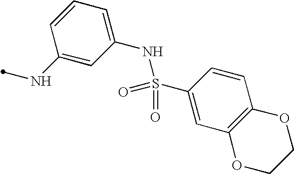 | H | 29 | | |
| 65 | 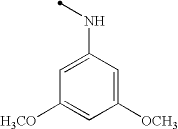 | 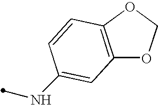 | CH₃ | 77 | | |
| 66 | 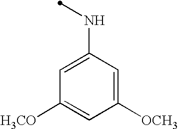 | 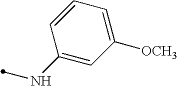 | CH₃ | 73 | | |
| 67 | 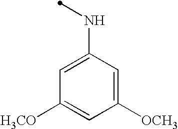 | 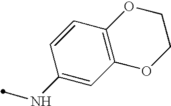 | CH₃ | 76 | | |
| 68 | 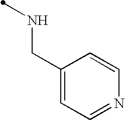 | 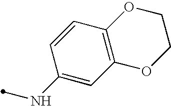 | CH₃ | 58 | | |
| 69 | 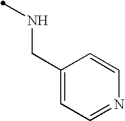 | 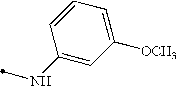 | CH₃ | 29 | | |
| 70 | 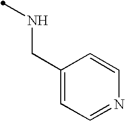 | 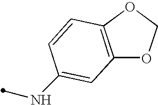 | CH₃ | 32 | | |
| 71 | 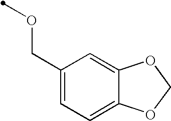 | 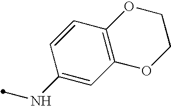 | H | 83 | 0.1 | |
| 72 | 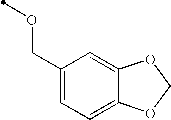 | 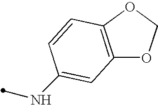 | H | 100 | 0.1 | |
| 73 | 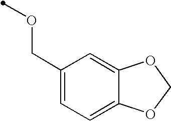 | 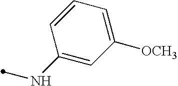 | H | 70 | 1 | |
| 74 | 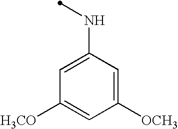 | 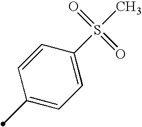 | H | 87 | 0.1 | |
| 75 | 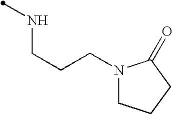 | 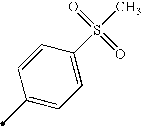 | H | 60 | | 80 |
| 76 | 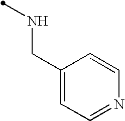 | 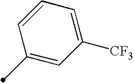 | H | 21 | | |
| 77 | 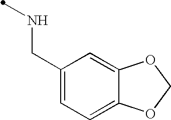 | 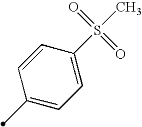 | H | 28 | 10 | 4.5 |
| 78 | 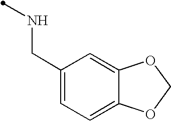 | 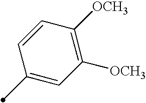 | H | 55 | 1 | 3 |
| 79 | 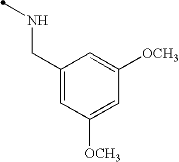 | 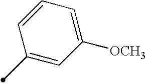 | H | 32 | | 20 |
| 80 | 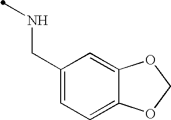 | 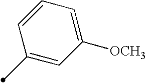 | H | 46 | | 8 |
| 81 | 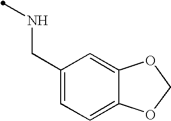 | 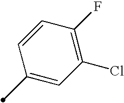 | H | 20 | | |
| 82 | 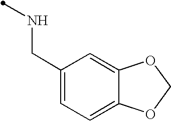 | 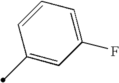 | H | 30 | | 20 |
| 83 | 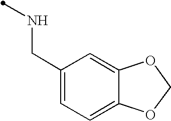 | 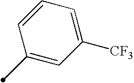 | H | 25 | | |
| 84 | 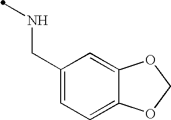 | 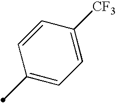 | H | 27 | | 2.5 |
| 85 | 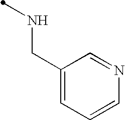 | 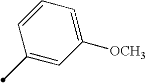 | H | 61 | 1 | 8 |
| 86 | 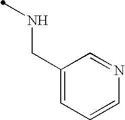 | 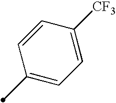 | H | 31 | | 2.2 |
| 87 | 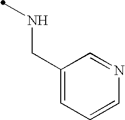 | 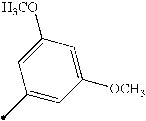 | H | 85 | | 30 |
| 88 | 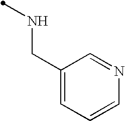 | 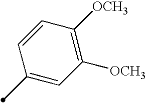 | H | 29 | 10 | 1.2 |
| 89 | 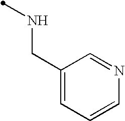 | 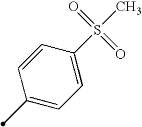 | H | 85 | | 5 |
| 90 | 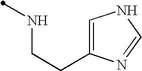 | 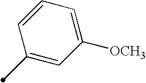 | H | 25 | | |
| 91 | 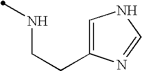 | 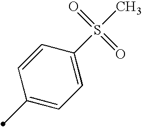 | H | 29 | | |
| 92 | 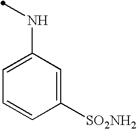 | 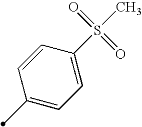 | H | 74 | 10 | |
| 93 | 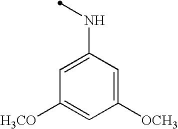 | 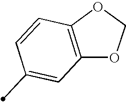 | H | 28 | | |
| 94 | 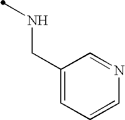 | 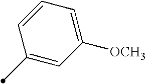 | H | 43 | | |
| 95 | 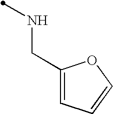 | 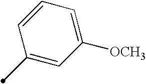 | H | 41 | | |
| 96 | 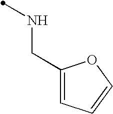 | 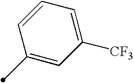 | H | 20 | | |
| 97 | 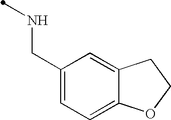 | 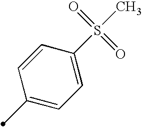 | H | 77 | 1 | |
| 98 | 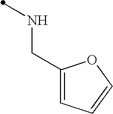 | 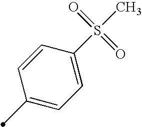 | H | 83 | 1 | |
| 99 | 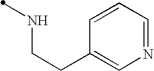 | 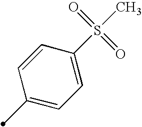 | H | 47 | | |
| 100 | 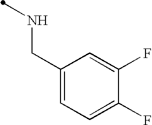 | 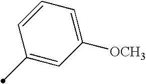 | H | 70 | 10 | |
| 101 | 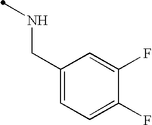 | 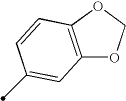 | H | 66 | 1 | |
| 102 | 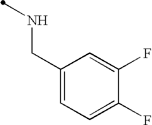 | 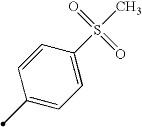 | H | 88 | 0.1 | |
| 103 | 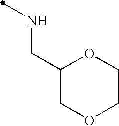 | 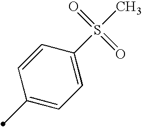 | H | 80 | 1 | |
| 104 | 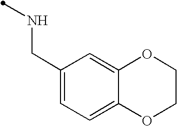 | 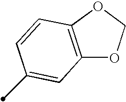 | H | 21 | | |
| 105 | 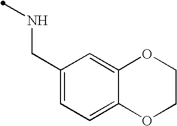 | 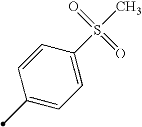 | H | 91 | 1 | |
| 106 | 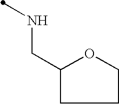 | 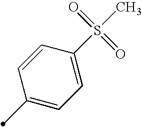 | H | 79 | 0.1 | |
| 107 | 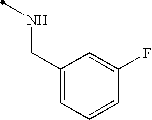 | 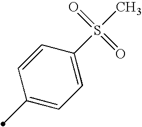 | H | 37 | | |
| 108 | 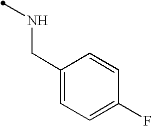 | 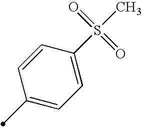 | H | 91 | 0.1 | |
| 109 | 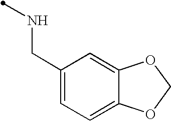 | 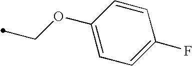 | H | 41 | | 30 |
| 110 | 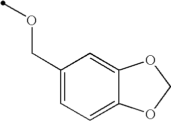 | 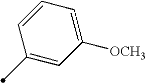 | H | 20 | | |

^{a}Measured as a percent inhibition at 10 μM based on 30 minute data.
^{b}Measured as concentration in μM required to achieve inhibition in FACS assay.
^{c}Measured as μM required to inhibit tumor cell growth by 50%.

TABLE 2
Compounds of Formula IIIA, phenyl-triazoles.

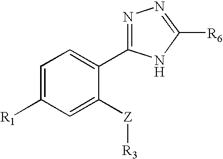

| Comp. | | | | Tubulin | FACS | SRB |
|---|---|---|---|---|---|---|
| No. | —Z—R₃ | —R₆ | —R₁ | Assay^{a} | Assay^{b} | Assay^{c} |
| 111 | 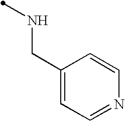 | 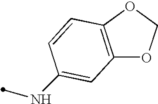 | H | 70 | | 10 |
| 112 | 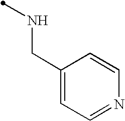 | 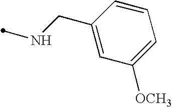 | H | 49 | | |
| 113 | 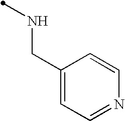 | 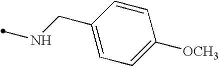 | H | 24 | | |
| 114 | 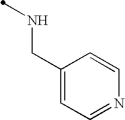 | 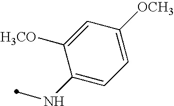 | H | 24 | | |
| 115 | 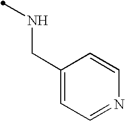 | 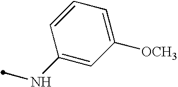 | H | 79 | 1 | 1.5 |
| 116 | 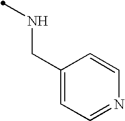 | 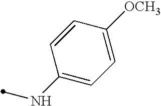 | H | 20 | | |
| 117 | 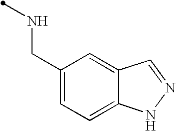 | 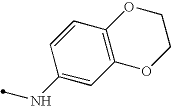 | H | 84 | 1 | |
| 118 | 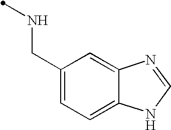 | 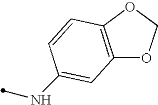 | H | 41 | | |
| 119 | 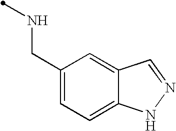 | 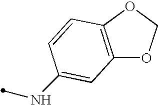 | H | 76 | 1 | |
| 120 | 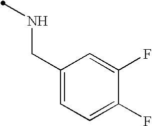 | 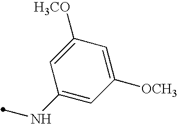 | H | 63 | | |
| 121 | 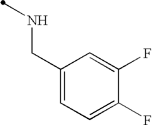 | 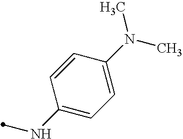 | H | 23 | | |
| 122 | 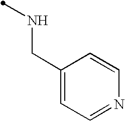 | 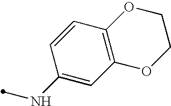 | H | 84 | 1 | |
| 123 | 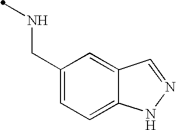 | 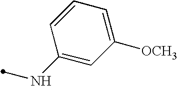 | H | 75 | 1 | |
| 124 | 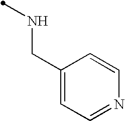 | 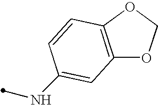 | CF₃ | 93 | 0.1 | |
| 124b | —NO₂ | 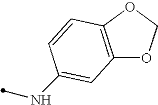 | CF₃ | 26 | | |
| 124c | —NH₂ | 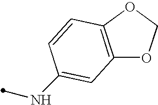 | CF₃ | 61 | | |
| 125 | 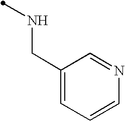 | 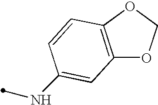 | CF₃ | 96 | | |
| 126 | 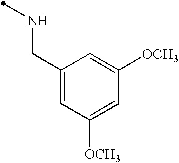 | 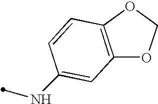 | CF₃ | 93 | | |
| 127 | 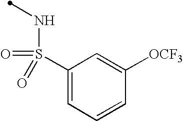 | 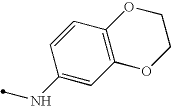 | H | 61 | 10 | |
| 127a | —NO₂ | 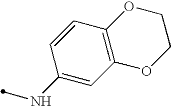 | H | 22 | | |
| 127b | —NH₂ | 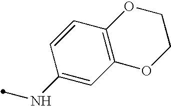 | H | 20 | | |
| 128 | 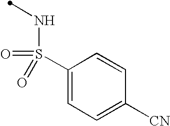 | 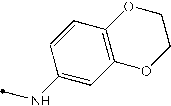 | H | 42 | | |
| 129 | 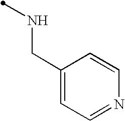 | 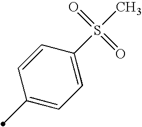 | H | 84 | 0.1 | 0.025 |
| 130 | 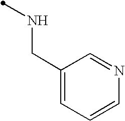 | 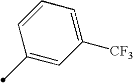 | H | 47 | | 2.75 |
| 131 | 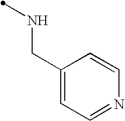 | 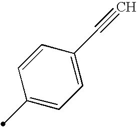 | H | 46 | 0.1 | 0.06 |
| 132 | 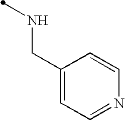 | 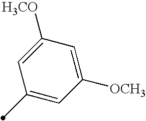 | H | 46 | | 100 |
| 133 | 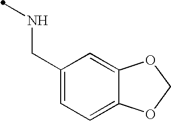 | 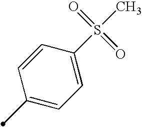 | H | 86 | 1 | |
| 134 | 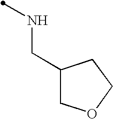 | 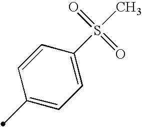 | H | 34 | | |
| 135 | 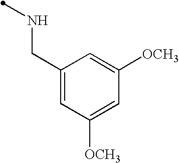 | 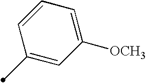 | H | 23 | | |
| 136 | 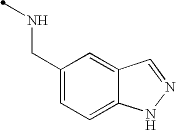 | 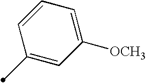 | H | 32 | 10 | |
| 137 | 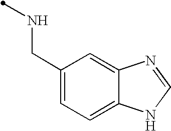 | 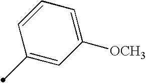 | H | 28 | | |
| 138 | 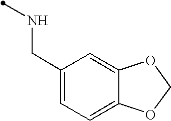 | 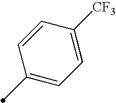 | H | 28 | | |
| 139 | 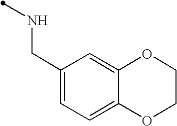 | 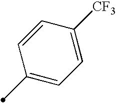 | H | 21 | | |
| 140 | 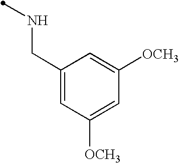 | 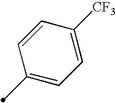 | H | 20 | | |
| 141 | 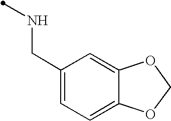 | 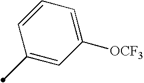 | H | 88 | 0.1 | |
| 142 | 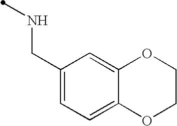 | 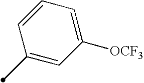 | H | 20 | | |
| 143 | 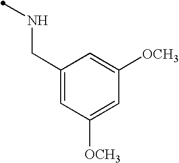 | 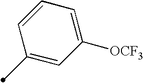 | H | 46 | | |
| 144 | 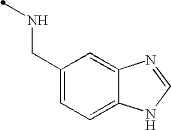 | 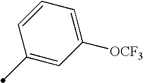 | H | 86 | | |
| 145 | 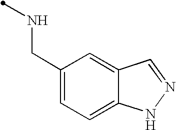 | 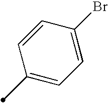 | H | 58 | 0.1 | |
| 146 | 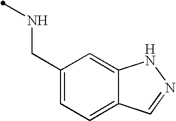 | 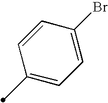 | H | 39 | | |
| 147 | 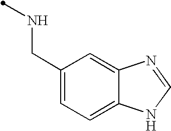 | 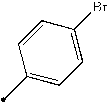 | H | 43 | | |
| 148 | 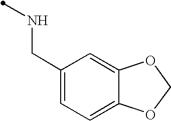 | 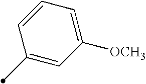 | H | 71 | 10 | |
| 149 | 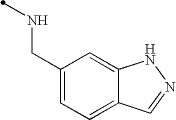 | 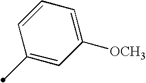 | H | 26 | | |
| 150 | 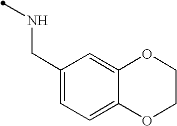 | 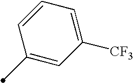 | H | 24 | | |
| 151 | 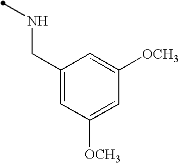 | 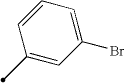 | H | 42 | | |
| 152 | 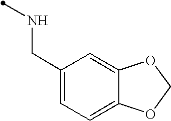 | 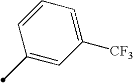 | H | 73 | | |
| 153 | 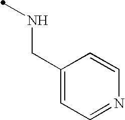 | 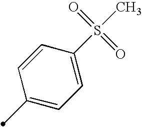 | CF₃ | 54 | | |
| 154 | 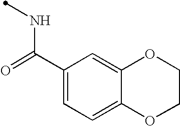 | 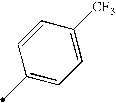 | H | 23 | | |

^{a}Measured as a percent inhibition at 10 μM based on 30 minute data.
^{b}Measured as concentration in μM required to achieve inhibition in FACS assay.
^{c}Measured as μM required to inhibit tumor cell growth by 50%.

TABLE 3
Compounds of Formula IIB, pyridyl-oxadiazoles.

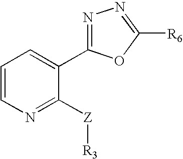

| Comp. | | | Tubulin | FACS | SRB |
|---|---|---|---|---|---|
| No. | —Z—R₃ | —R₆ | Assay^{a} | Assay^{b} | Assay^{c} |
| 155 | 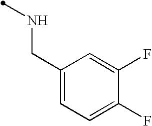 | 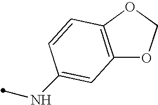 | 91 | 1 | |
| 156 | 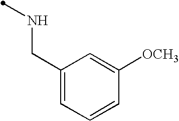 | 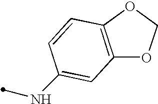 | 64 | 1 | |
| 157 | 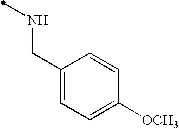 | 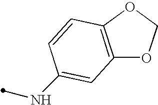 | 92 | 1 | |
| 158 | 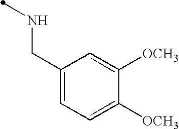 | 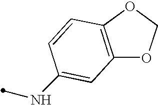 | 90 | | |
| 159 | 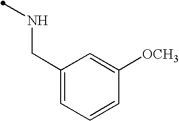 | 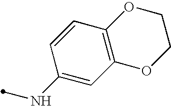 | 87 | 0.075 | |
| 160 | 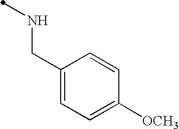 | 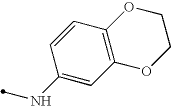 | 89 | 0.1 | |
| 161 | 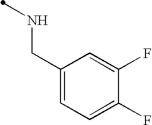 | 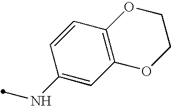 | 82 | 0.1 | |
| 162 | 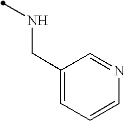 | 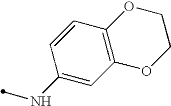 | 86 | 0.01 | |
| 163 | 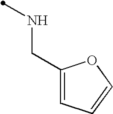 | 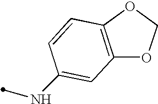 | 90 | 1 | |
| 164 | 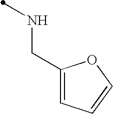 | 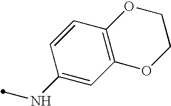 | 73 | 0.1 | |
| 165 | 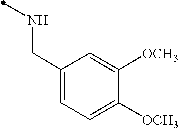 | 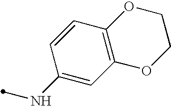 | 86 | 0.1 | |
| 166 | 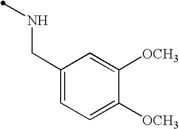 | 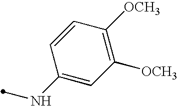 | 53 | 1 | |
| 167 | 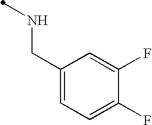 | 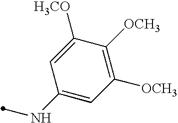 | 99 | | |
| 168 | 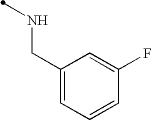 | 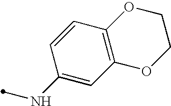 | 95 | 0.1 | |
| 169 | 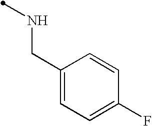 | 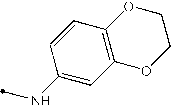 | 82 | 0.1 | |
| 170 | 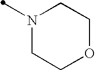 | 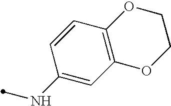 | 55 | 0.1 | |
| 171 | 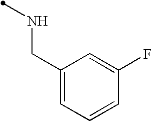 | 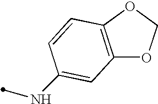 | 91 | | |
| 172 | 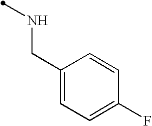 | 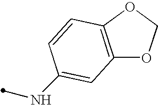 | 95 | 0.1 | |
| 172 | 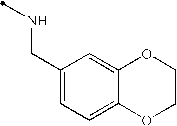 | 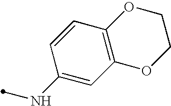 | 87 | 0.1 | |
| 174 | 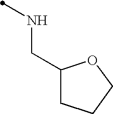 | 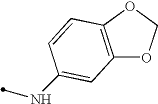 | 64 | | |
| 175 | 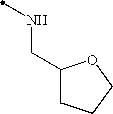 | 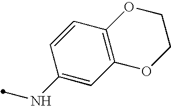 | 90 | 0.1 | |
| 176 | 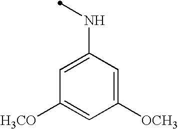 | 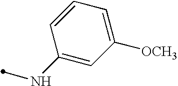 | 27 | 10 | 40 |
| 177 | 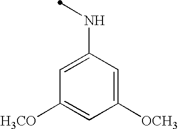 | 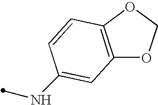 | 45 | 0.01 | 0.04 |
| 178 | 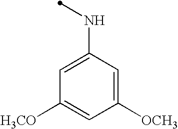 | 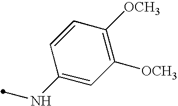 | 40 | | 0.3 |
| 179 | 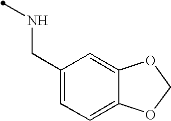 | 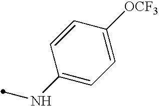 | 28 | | 30 |
| 180 | 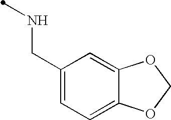 | 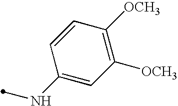 | 32 | | 1.5 |
| 181 | 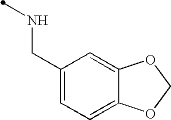 | 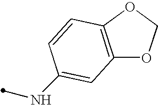 | 76 | 1 | 0.3 |
| 182 | 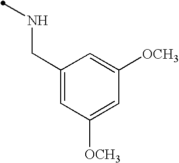 | 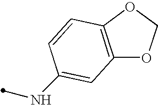 | 84 | 1 | 0.2 |
| 183 | 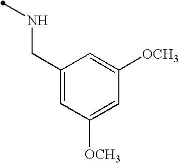 | 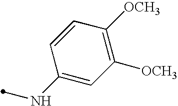 | 50 | 0.1 | 6 |
| 184 | 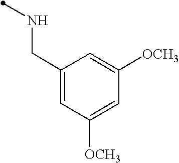 | 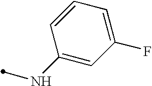 | 23 | | |
| 185 | 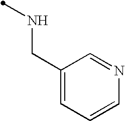 | 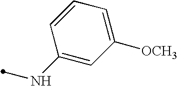 | 40 | | 30 |
| 186 | 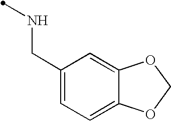 | 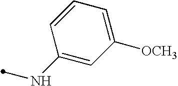 | 42 | | 100 |
| 187 | 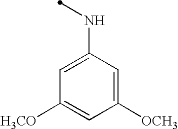 | 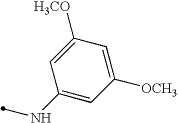 | 48 | | 80 |
| 188 | 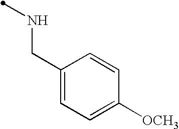 | 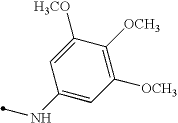 | 56 | | |
| 189 | 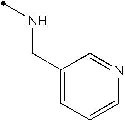 | 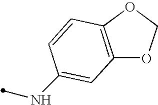 | 89 | 0.1 | 0.6 |
| 190 | 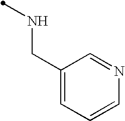 | 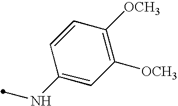 | 56 | | 15 |
| 191 | 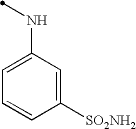 | 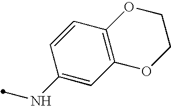 | 28 | | |
| 192 | 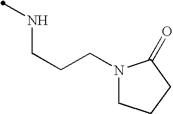 | 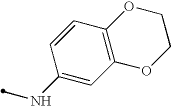 | 85 | 0.1 | 0.01 |
| 193 | 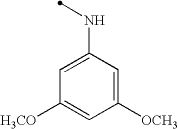 | 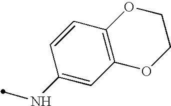 | 69 | 0.01 | 0.01 |
| 194 | 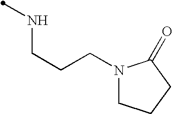 | 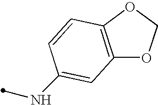 | 80 | | 3 |
| 195 | 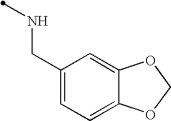 | 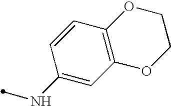 | 73 | 0.01 | |
| 196 | 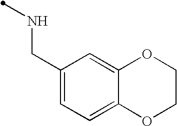 | 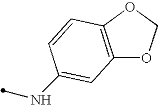 | 93 | 1 | |
| 197 | 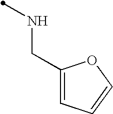 | 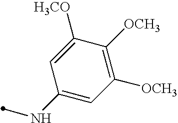 | 66 | 1 | |
| 198 | 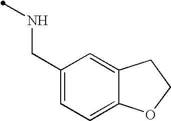 | 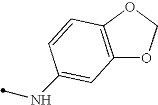 | 87 | 0.1 | |
| 199 | 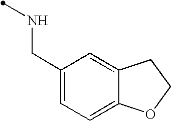 | 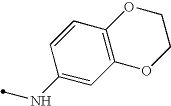 | 88 | 0.1 | |
| 200 | 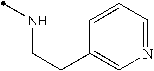 | 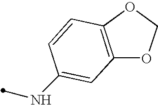 | 68 | | |
| 201 | 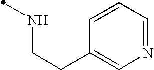 | 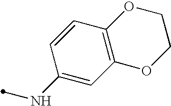 | 23 | | |
| 202 | 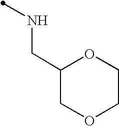 | 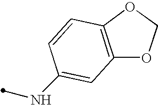 | 89 | 0.1 | |
| 203 | 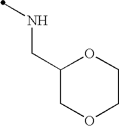 | 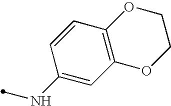 | 69 | 0.1 | |
| 204 | 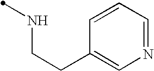 | 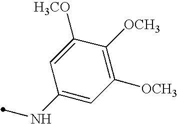 | 28 | | |
| 205 | 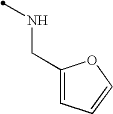 | 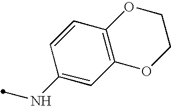 | 56 | 1 | |
| 206 | 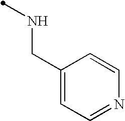 | 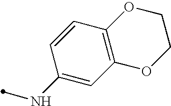 | 94 | 0.025 | |
| 207 | 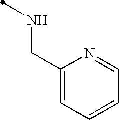 | 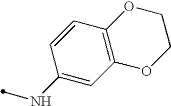 | 87 | 0.025 | |
| 208 | 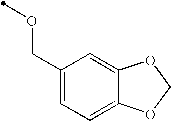 | 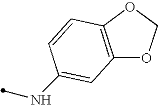 | 44 | | |
| 209 | 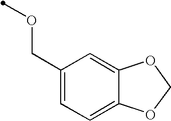 | 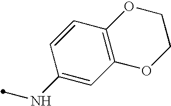 | 80 | 1 | |
| 210 | 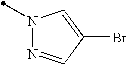 | 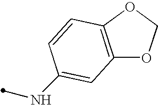 | 87 | 1 | |
| 211 | 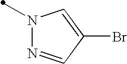 | 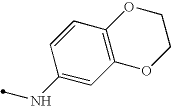 | 73 | | |
| 212 | 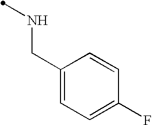 | 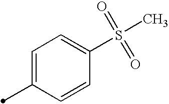 | 38 | | |
| 213 | 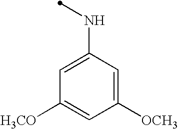 | 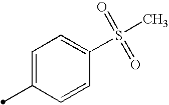 | 30 | | |
| 214 | 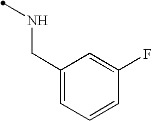 | 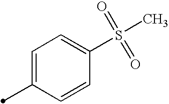 | 52 | | |

^{a}Measured as a percent inhibition at 10 μM based on 30 minute data.
^{b}Measured as concentration in μM required to achieve inhibition in FACS assay.
^{c}Measured as μM required to inhibit tumor cell growth by 50%.

TABLE 4
Compounds of Formula IIIB, phenyl-oxadiazoles.

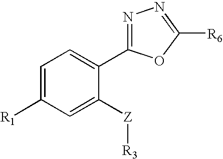

| Comp. | | | | Tubulin | FACS | SRB |
|---|---|---|---|---|---|---|
| No. | —Z—R₃ | —R₆ | —R₁ | Assay^{a} | Assay^{b} | Assay^{c} |
| 215 | 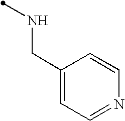 | 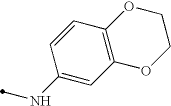 | H | 100 | 0.01 | 10 |
| 216 | 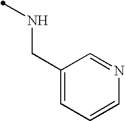 | 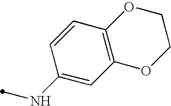 | H | 94 | 0.1 | |
| 217 | 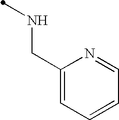 | 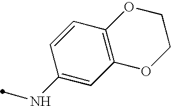 | H | 100 | 0.1 | |
| 218 | 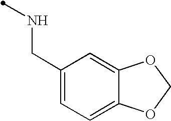 | 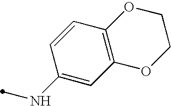 | H | 74 | 0.1 | |
| 219 | 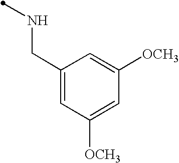 | 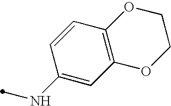 | H | 63 | 0.1 | |
| 220 | 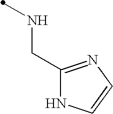 | 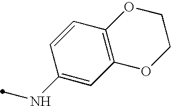 | H | 79 | | |
| 221 | 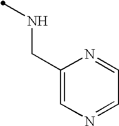 | 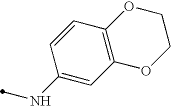 | H | 80 | | |
| 222 | 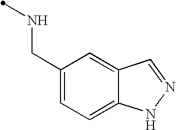 | 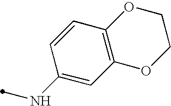 | H | 95 | | |
| 223 | 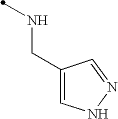 | 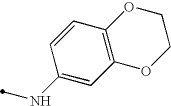 | H | 84 | 0.1 | 1.5 |
| 224 | 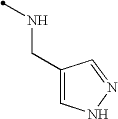 | 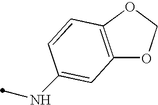 | H | 81 | 1 | |
| 225 | 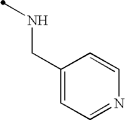 | 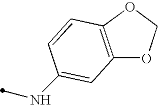 | H | 91 | | 15 |
| 226 | 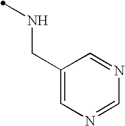 | 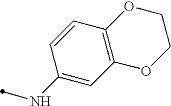 | H | 94 | | |
| 227 | 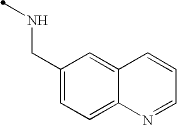 | 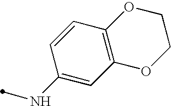 | H | 99 | | |
| 228 | 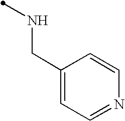 | 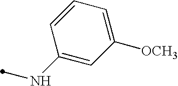 | H | 57 | 10 | 2 |
| 229 | 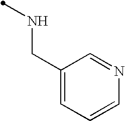 | 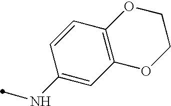 | N(CH₃)₂ | 32 | | |
| 230 | 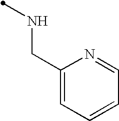 | 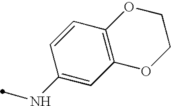 | N(CH₃)₂ | 45 | | |
| 231 | 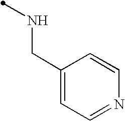 | 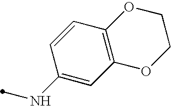 | N(CH₃)₂ | 28 | | |
| 232 | 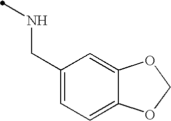 | 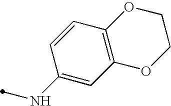 | N(CH₃)₂ | 26 | | |
| 233 | 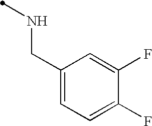 | 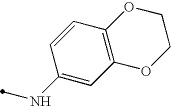 | N(CH₃)₂ | 40 | | |
| 234 | 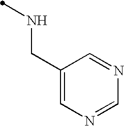 | 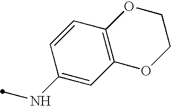 | N(CH₃)₂ | 30 | | |
| 235 | 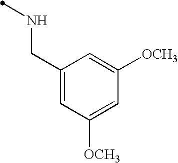 | 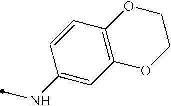 | 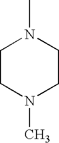 | 24 | | |
| 236 | —NO₂ | 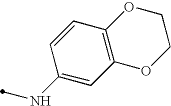 | 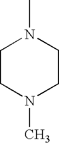 | 59 | | |
| 237a | —NH₂ | 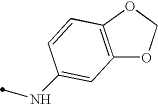 | H | 53 | | |
| 237 | 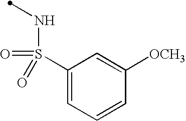 | 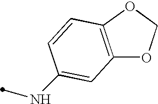 | H | 99 | 1 | |
| 238 | 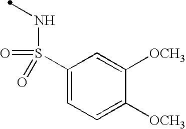 | 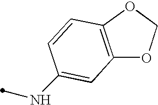 | H | 65 | | |
| 239 | 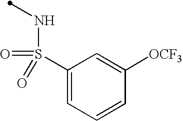 | 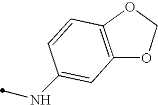 | H | 49 | | |
| 240 | 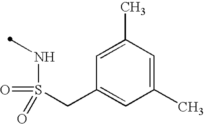 | 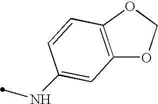 | H | 20 | | |
| 241 | —NH₂ | 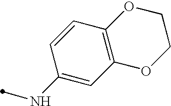 | H | 75 | 1 | |
| 242 | 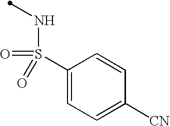 | 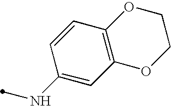 | H | 92 | | |
| 243a | 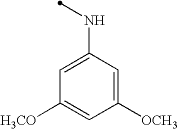 | —NH₂ | H | 46 | | |
| 243 | 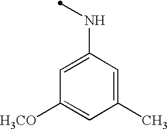 | 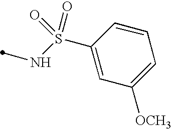 | H | 55 | | |

^{a}Measured as a percent inhibition at 10 μM based on 30 minute data.
^{b}Measured as concentration in μM required to achieve inhibition in FACS assay.
^{c}Measured as μM required to inhibit tumor cell growth by 50%.

TABLE 5
Compounds of Formula IIIC, phenyl-oxazoles.

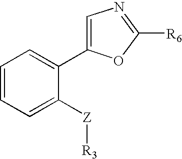

| Comp. | | | Tubulin | FACS | SRB |
|---|---|---|---|---|---|
| No. | —Z—R₃ | —R₆ | Assay^{a} | Assay^{b} | Assay^{c} |
| 244 | 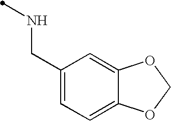 | 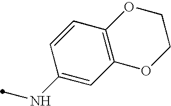 | 96 | 1 | |
| 245 | 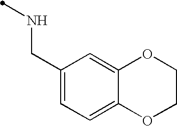 | 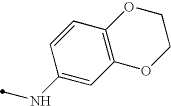 | 93 | | |
| 246 | 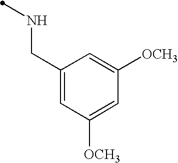 | 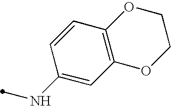 | 72 | 1 | |
| 247 | 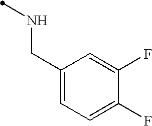 | 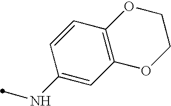 | 94 | | |
| 248 | 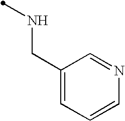 | 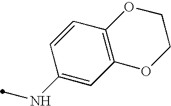 | 79 | | |
| 249 | 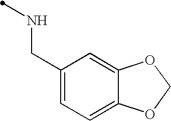 | 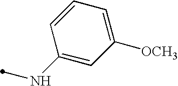 | 60 | 1 | |
| 250 | 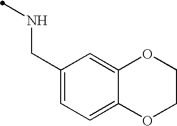 | 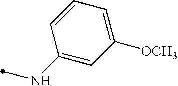 | 60 | 1 | |
| 251 | 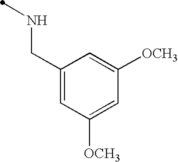 | 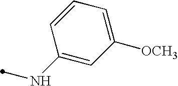 | 30 | | |
| 252 | 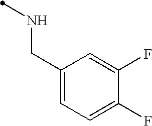 | 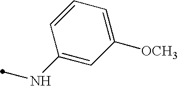 | 27 | | |
| 253 | 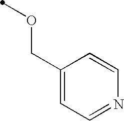 | 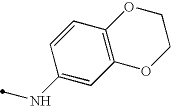 | 44 | | |
| 254 | 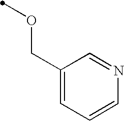 | 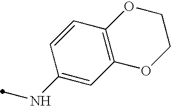 | 95 | | |
| 255 | 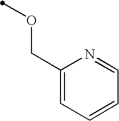 | 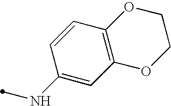 | 100 | | |
| 256 | 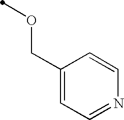 | 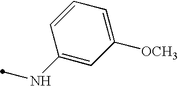 | 50 | | |
| 257 | 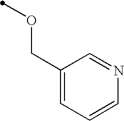 | 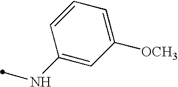 | 27 | | |
| 258 | 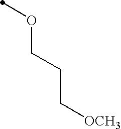 | 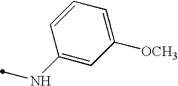 | 47 | | |

^{a}Measured as a percent inhibition at 10 μM based on 30 minute data.
^{b}Measured as concentration in μM required to achieve inhibition in FACS assay.
^{c}Measured as μM required to inhibit tumor cell growth by 50%.

The invention is further defined by reference to the following examples, describing in detail the preparation of the compound and the compositions of the present invention, as well as their utility. It will be apparent to those skilled in the art that many modifications, both to materials and methods, may be practiced without departing from the purpose and interest of this invention.

### EXAMPLES

The examples are intended to be illustrative only. In particular, the invention is not intended to be limited to the methods, protocols, conditions and the like specifically recited herein, insofar as those skilled in the art would be able to substitute other conditions, methods, amounts, materials, etc. based on the present disclosure to arrive at compounds within the scope of this disclosure. While the present invention is described with respect to particular examples and preferred embodiments, the present invention is not limited to these examples and embodiments. In particular, the compounds of the present invention are not limited to the exemplary species' recited herein. Moreover, the methods of the present invention are not limited to treating only the exemplified diseases and conditions, but rather any disease or condition that may be treated by regulation of tubulin. Additionally, the methods of synthesis of the present invention are not limited to the methods exemplified in the example. The methods of the present invention include methods of making any of the compounds set forth in the present invention that those skilled would be able to make in view of the present disclosure, and are not limited to the exemplified method. For example, methods encompassed by the present invention may involve the use of a different starting material depending on the desired final compound, different amounts of various ingredients, or substitution of different ingredients such as other reactants or catalysts that would be suitable depending on the starting material and result to be achieved.

### Example 1

### Synthesis of (3,5-Dimethoxy-phenyl)-{3-[5-(3,5-dimethoxy-phenylamino)-2H-[1,2,4]triazol-3-yl]-pyridin-2-yl}-amine (1)

### Step 1: Synthesis of 2-chloro-nicotinic Acid Ethyl Ester (1a)

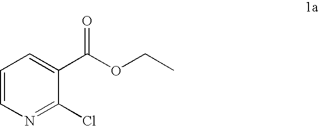

2-Chloropyridine-3-carboxylic acid (25 g, purchased from Aldrich) was refluxed in 200 ml of benzene and 150 ml of thionyl chloride over 3 hours. The solution was concentrated and chased with toluene. The residue obtained was refluxed in 100 ml of ethanol for 20 minutes. The solvents were removed in vacuum to give the product 1a, as light yellow oil in 72% yield by weight. The product 1a was identified by ¹HNMR and ¹³CNMR. ¹HNMR (CDCl₃) δ (ppm) 1.42 (t, J=6.6 Hz, 3H), 4.43 (q, J=6.8 Hz, 2H), 7.37 (br s, 1H), 8.18 (d, J=6.6 Hz, 1H), 8.54 (s, 1H). ¹³CNMR δ 13.8, 61.8, 122.0, 126.9, 140.0, 149.5, 151.4, 164.2.

### Step 2: Synthesis of 2-(3,5-dimethoxy-phenylamino)-nicotinic Acid Ethyl Ester (1b)

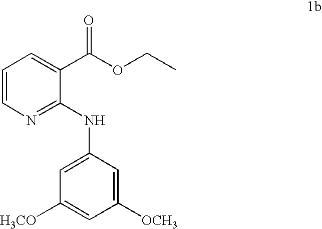

2-Chloro-nicotinic acid ethyl ester 1a (2 mmol, 0.343 g) and 3,5-dimethoxyaniline (2 mmol, 0.306 g, purchased from Aldrich) were dissolved in ethylene glycol (10 ml) and heated up to 160° C. with stirring. The reaction mixture was maintained at this temperature for 6 hours. Hydrogen chloride gas was formed during the course of the reaction. On cooling, the reaction mixture was poured into water (10 ml) and extracted with ether (4×100 ml). The ethereal layer was dried over magnesium sulfate, evaporated and the residue was distilled at 162-165° C./0.5 mm Hg to give a yellow oil in 63% yield by weight. The compound 1b was used in the next step without further purification. Product appeared as yellow oil, yield 63%. ¹HNMR (CDCl₃) δ (ppm) 1.24 (t, J=7.1 Hz, 3H), 3.78 (s, 6H), 4.11 (t, J=7.1 Hz, 2H), 6.17 (s, 1H), 6.68 (t, J=6.0 Hz, 1H), 6.97 (s, 2H), 8.20 (d, J=7.7 Hz, 1H), 8.36 (s, 1H), 10.24 (s, 1H). ¹³CNMR δ (ppm) 20.7, 55.0, 60.1, 94.8, 98.7, 103.2, 107.1, 113.1, 139.9, 141.2, 152.7, 155.8, 160.7, 167.2, 170.9.

### Step 3: Synthesis of 2-(3,5-dimethoxy-phenylamino)-nicotinic Acid Hydrazide (1c)

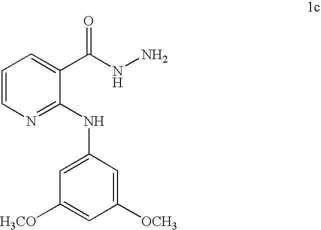

A mixture of 2-(3,5-dimethoxy-phenylamino)-nicotinic acid ethyl ester 1b (1.94 mmol, 0.59 g) and 85% hydrazine monohydrate (1.18 ml) in 2-propanol (2 ml) was refluxed for 3 hours and the solution turned red. After cooling to room temperature, the red solution deposited yellow solid that was filtered off and washed with 2-propanol. After drying in vacuum oven, the product 1c appeared as yellow solid in 84% yield by weight. ¹HNMR (DMSO-d₆) δ (ppm) 1.22 (br s, 2H), 4.00 (s, 6H), 6.18 (t, J=2.2 Hz, 1H), 6.66-6.70 (m, 1H), 6.94 (s, 2H), 7.64 (dd, J=7.7, 1.8 Hz, 1H), 7.70 (br s, 1H), 8.33 (dd, J=4.8, 1.5 Hz, 1H), 10.1 (br s, 1H). ¹³CNMR δ (ppm) 55.3, 94.8, 98.7, 100.2, 103.4, 100.4, 109.5, 113.1, 135.1, 141.5, 151.8, 160.9, 169.1.

### Step 4: Synthesis of 1-(3,5-dimethoxy-phenyl)-3-(4-fluoro-benzoyl)-thiourea (1d)

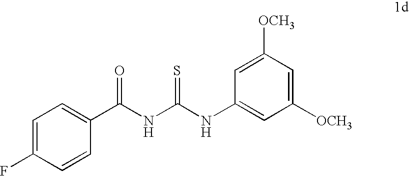

To a vigorously stirred hot solution of anhydrous ammonium thiocyanate (0.61 g, 7.8 mmol) in dry acetone (20 ml) was treated dropwise with 4-fluorobenzoyl chloride (1.03 g, 6.5 mmol, purchased from Aldrich). The reaction mixture was refluxed for 5 min. Then a solution of 3,5-dimethoxyaniline (1.0 g, 6.5 mmol) in dry acetone (10 ml) was added dropwise. The reaction mixture was heated for 1 hour. The solvent was evaporated and water (50 ml) was added to the residue. The precipitate was collected and recrystallized from ethyl alcohol to give the product 1d as white needles in 69% yield by weight. ¹HNMR (DMSO-d₆) δ (ppm) 3.76 (s, 6H), 6.43 (br s, 1H), 6.99 (br s, 2H), 7.35-7.41 (m, 2H), 8.04-8.09 (m, 2H), 11.62 (s, 1H). Anal. Calcd for C₁₆H₁₅FN₂O₃S: C, 57.47; H, 4.52; N, 8.38. Found: C, 57.49; H, 4.43; N, 8.26.

### Step 5: Synthesis of (3,5-dimethoxy-phenyl)-thiourea (1e)

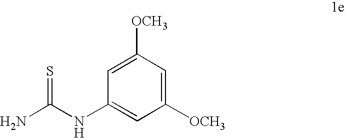

N-(3,5-Dimethoxyphenyl)-N′-(4-fluorobenzoyl)thiourea 1d (4.4 mmol, 1.5 g) was heated to reflux with 5% aqueous sodium hydroxide (10 ml) for 15 min. The cooled reaction mixture was treated with concentrated hydrochloric acid until acidic to precipitate both 4-fluorobenzoic acid and N-(3,5-dimethoxyphenyl)thiourea. The mixture was then made basic (pH=9) with concentrated ammonium hydroxide to dissolve the 4-fluorobenzoic acid. The product 1e was filtered and recrystallized from 95% ethyl alcohol to give white prisms in 75% yield. ¹HNMR (DMSO-d₆) δ (ppm) 3.72 (s, 6H), 6.27 (br s, 1H), 6.62 (br s, 2H), 7.53 (br s, 2H), 9.66 (s, 1H). ¹³CNMR (DMSO-d₆) δ (ppm) 55.2, 96.4, 100.8, 140.6, 160.4, 180.7. Anal. Calcd for C₉H₁₂N₂O₂S: C, 50.92; H, 5.70; N, 13.20. Found: C, 50.88; H, 5.66; N, 12.96.

### Step 6: Synthesis 1-(3,5-Dimethoxy-phenyl)-2-methyl-isothiourea Hydriodide (1f)

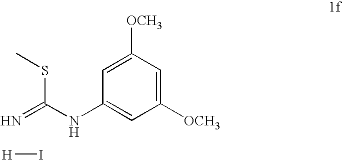

A solution of N-(3,5-dimethoxyphenyl)thiourea (2.5 mmol, 0.53 g) in freshly distilled dry methanol (10 ml) was treated with methyl iodide (2.5 mmol, 0.36 g). The solution was refluxed for 2 h, cooled, and evaporated to dryness in vacuo. The crystalline product was washed with several portion of ethyl ether and dried to give pure product 1f as white microcrystals in 92% yield. ¹HNMR (DMSO-d₆) δ (ppm) 2.70 (s, 3H), 3.78 (s, 6H), 6.53-6.56 (m, 3H), 9.30 (br s, 2H). ¹³CNMR (DMSO-d₆) δ (ppm) 55.6, 100.1, 103.7, 136.5, 161.1, 169.1.

### Step 7: Preparation of (3,5-Dimethoxy-phenyl)-{3-[5-(3,5-dimethoxy-phenylamino)-4H-[1,2,4]triazol-3-yl]-pyridin-2-yl}-amine (1)

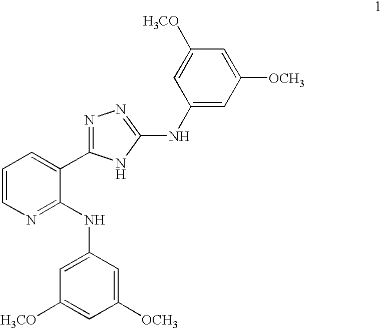

A mixture of 2-(3,5-dimethoxyphenylamino)nicotinic acid hydrazide (1c) (1 mmol, 0.29 g) and N-(3,5-dimethoxyphenyl)-S-methylisothiourea hydroiodide (1f) (1 mmol, 0.35 g) in 1 ml of pyridine were refluxed for 6 hours. The cooled mixture was poured into crushed ice and extracted with ether. The solvent was removed and the crude product was recrystallized from ethyl acetate (and two drops of ethanol) to give the pure product 1 as a brown solid in 25% yield. ¹H NMR (DMSO-d₆, 100° C.) δ (ppm) 3.75 (s, 6H), 3.76 (s, 6H), 6.11 (br s, 1H), 6.18 (t, J=2.2 Hz, 1H), 6.81 (d, J=2.2 Hz, 2H), 6.89-6.93 (m, 1H), 7.07 (d, J=1.8 Hz, 2H), 8.28-8.29 (m, 2H), 9.22 (br s, 1H), 10.7 (br s, 1H). MS m/z: 449 (M+1).

Compounds 2 to 59 were synthesized using method described in Example 1:

### Analytical Data:

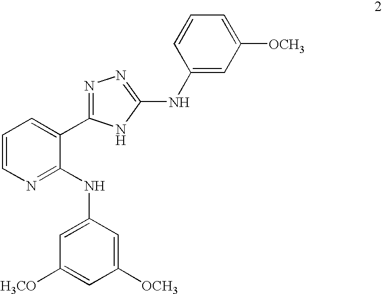

(3,5-Dimethoxy-phenyl)-{3-[5-(3-methoxy-phenylamino)-4H-[1,2,4]triazol-3-yl]-pyridin-2-yl}-amine (2): ¹HNMR (DMSO-d₆) (ppm) 11.00 (s, 1H), 9.50 (s, 1H), 8.28-8.32 (m, 2H), 7.10-7.28 (m, 5H), 6.92-6.97 (m, 1H), 6.47-6.50 (m, 1H), 6.17 (m, 1H), 3.76 (s, 6H), 3.75 (s, 3H). MS m/z: 419 (M+1).

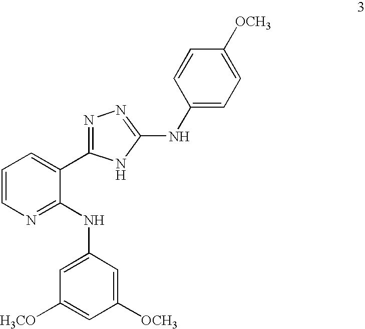

(3,5-Dimethoxy-phenyl)-{3-[5-(4-methoxy-phenylamino)-4H-[1,2,4]triazol-3-yl]-pyridin-2-yl}-amine (3): ¹HNMR (methanol-d₄) δ (ppm) 8.25-8.35 (br s, 1H), 8.19 (s, 1H), 7.43 (d, 2H), 6.83-6.96 (m, 5H), 6.16 (s, 1H), 3.80 (s, 6H), 3.30 (s, 3H). MS m/z: 419 (M+1).

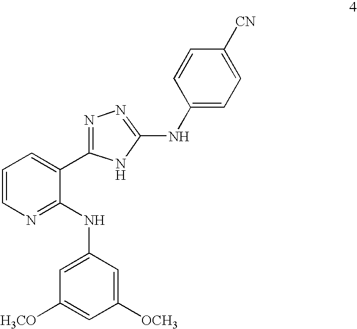

4-{5-[2-(3,5-Dimethoxy-phenylamino)-pyridin-3-yl]-4H-[1,2,4]triazol-3-ylamino}-benzonitrile (4): ¹HNMR (DMSO-d₆) δ (ppm) 10.30 (s, 1H), 8.32-8.46 (m, 2H), 7.78-7.93 (m, 4H), 7.00-7.77 (m, 3H), 6.26 (d, J=6.9 Hz, 1H), 3.82 (s, 3H), 3.80 (s, 3H). MS m/z: 414 (M+1).

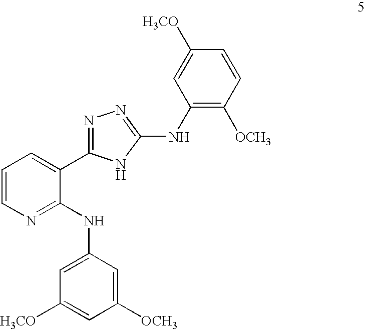

(3,5-Dimethoxy-phenyl)-{3-[5-(2,5-dimethoxy-phenylamino)-4H-[1,2,4]triazol-3-yl]-pyridin-2-yl}-amine (5): ¹HNMR (CDCl₃) δ (ppm) 10.34 (s, 1H), 8.27-8.29 (m, 2H), 7.77-7.78 (m, 1H), 7.36 (s, 1H), 7.00-7.01 (m, 2H), 6.73-6.82 (m, 2H), 6.45-6.49 (m, 1H), 6.14-6.15 (m, 1H), 3.77-3.83 (m, 12H). MS m/z: 449 (M+1).

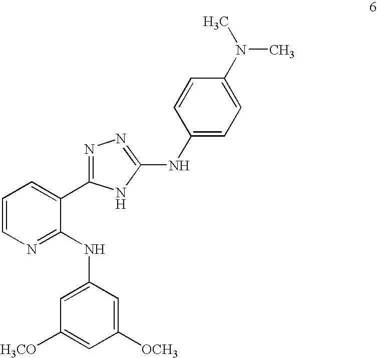

(3,5-Dimethoxy-phenyl)-{3-[5-(4-dimethylamino-phenylamino)-4H-[1,2,4]triazol-3-yl]-pyridin-2-yl}-amine (6): ¹HNMR (CDCl₃) δ (ppm) 10.56 (s, 1H), 8.43-8.50 (m, 2H), 7.47 (d, 3H), 6.94-6.98 (m, 4H), 6.36-6.38 (m, 1H), 4.01 (s, 6H), 3.19 (br s, 6H). MS m/z: 432 (M+1).

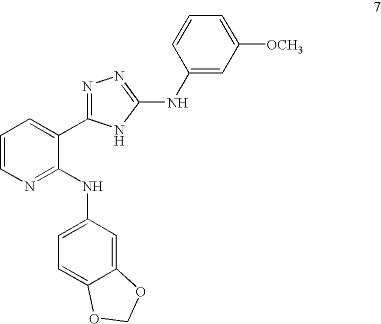

Benzo[1,3]dioxol-5-yl-{3-[5-(3-methoxy-phenylamino)-4H-[1,2,4]triazol-3-yl]-pyridin-2-yl}-amine (7): ¹HNMR (CDCl₃) δ (ppm) 10.19 (s, 1H), 8.22-8.24 (m, 1H), 8.11 (br s, 1H), 6.60-7.39 (m, 10H), 5.91 (s, 2H), 3.83 (s, 3H). MS m/z: 403 (M+1).

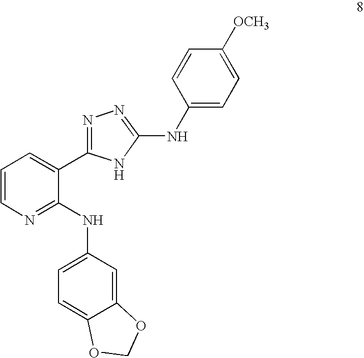

Benzo[1,3]dioxol-5-yl-{3-[5-(4-methoxy-phenylamino)-4H-[1,2,4]triazol-3-yl]-pyridin-2-yl}-amine (8): ¹HNMR (CDCl₃) δ (ppm) 10.07 (s, 1H), 8.09-8.16 (m, 2H), 7.31-7.35 (m, 1H), 7.22-7.30 (m, 3H), 6.88-6.95 (m, 3H), 6.62-6.75 (m, 3H), 5.84-5.86 (s, 2H), 3.78 (s, 3H). MS m/z: 403 (M+1).

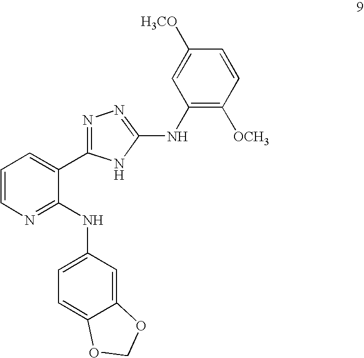

Benzo[1,3]dioxol-5-yl-{3-[5-(2,5-dimethoxy-phenylamino)-4H-[1,2,4]triazol-3-yl]-pyridin-2-yl}-amine (9): ¹HNMR (DMSO-d₆) δ (ppm) 10.49 (s, 1H), 8.88 (s, 1H), 8.25-8.40 (m, 2H), 7.95 (s, 1H), 7.64 (s, 1H), 6.91-7.13 (m, 4H), 6.49-6.58 (m, 1H), 6.03-6.06 (s, 2H), 3.89 (s, 3H), 3.69 (s, 3H). MS m/z: 433 (M+1).

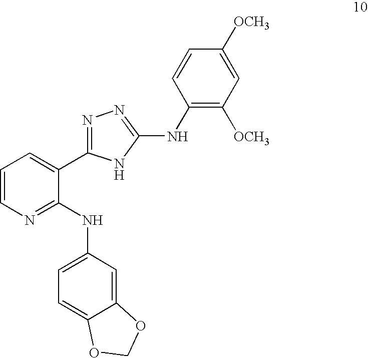

Benzo[1,3]dioxol-5-yl-{3-[5-(2,4-dimethoxy-phenylamino)-4H-[1,2,4]triazol-3-yl]-pyridin-2-yl}-amine (10): ¹HNMR (methanol-d₄) δ (ppm) 8.32 (s, 1H), 8.09 (s, 1H), 7.34 (s, 1H), 6.88-7.00 (m, 1H), 6.70-6.80 (m, 3H), 6.50-6.70 (m, 2H), 5.90 (s, 2H), 3.89 (s, 3H), 3.71 (s, 3H). MS m/z: 433 (M+1).

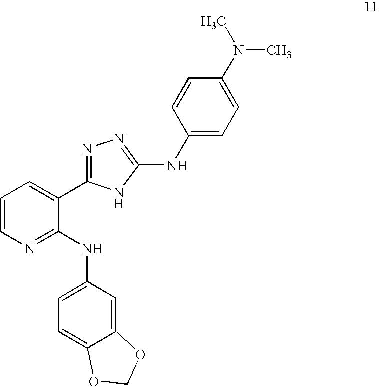

Benzo[1,3]dioxol-5-yl-{3-[5-(4-dimethylamino-phenylamino)-4H-[1,2,4]triazol-3-yl]-pyridin-2-yl}-amine (11): ¹HNMR (DMSO-d₆) δ (ppm) 13.28 (s, 1H), 11.07 (s, 1H), 9.27 (s, 1H), 8.23-8.34 (m, 2H), 6.79-7.60 (m, 8H), 6.03 (s, 2H), 2.71 (s, 6H). MS m/z: 416 (M+1).

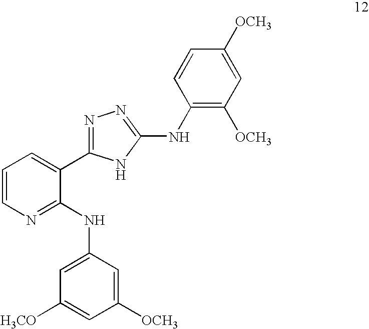

(3,5-Dimethoxy-phenyl)-{3-[5-(2,4-dimethoxy-phenylamino)-4H-[1,2,4]triazol-3-yl]-pyridin-2-yl}-amine (12): ¹HNMR (CDCl₃) δ (ppm) 10.41 (s, 1H), 8.19-8.29 (m, 2H), 7.77-7.80 (m, 1H), 7.03-7.04 (m, 3H), 6.72-6.76 (m, 1H), 6.51-6.55 (m, 2H), 6.14-6.16 (m, 1H), 3.64-3.90 (m, 12H). MS m/z: 449 (M+1).

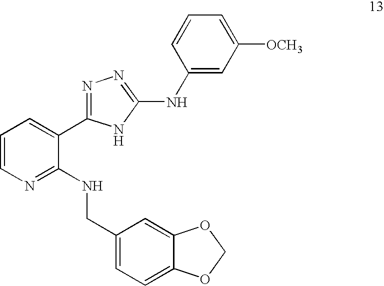

Benzo[1,3]dioxol-5-ylmethyl-{3-[5-(3-methoxy-phenylamino)-4H-[1,2,4]triazol-3-yl]-pyridin-2-yl}-amine (13): ¹HNMR (DMSO-d₆) δ (ppm) 9.64 (s, 1H), 9.29 (s, 1H), 8.91 (s, 1H), 8.14-8.33 (m, 2H), 6.73-7.28 (m, 7H), 6.44-6.56 (m, 1H), 6.01 (s, 2H), 4.70 (d, J=7.8 Hz, 2H), 3.77 (s, 3H). MS m/z: 417 (M+1).

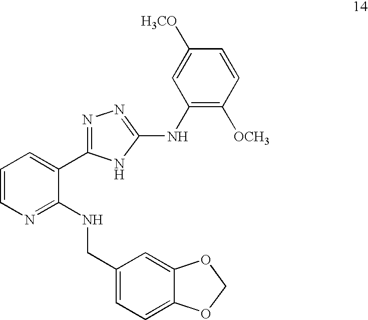

Benzo[1,3]dioxol-5-ylmethyl-{3-[5-(2,5-dimethoxy-phenylamino)-4H-[1,2,4]triazol-3-yl]-pyridin-2-yl}-amine (14): ¹HNMR (DMSO-d₆) δ (ppm) 8.60-8.68 (m, 2H), 8.35 (s, 1H), 8.20-8.25 (m, 1H), 7.90-7.96 (m, 2H), 7.50-7.58 (m, 2H), 6.51-7.02 (m, 3H), 6.50-6.55 (m, 1H), 5.96 (s, 2H), 4.67 (s, 2H), 3.92 (s, 3H), 3.83 (s, 3H). MS m/z: 447 (M+1).

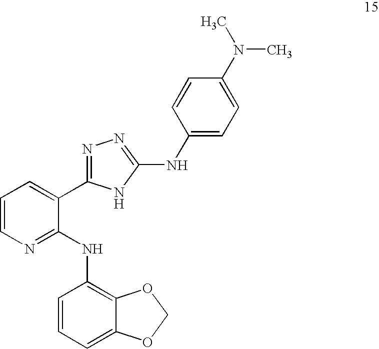

Benzo[1,3]dioxol-5-ylmethyl-{3-[5-(4-dimethylamino-phenylamino)-4H-[1,2,4]triazol-3-yl]-pyridin-2-yl}-amine (15): ¹HNMR (methanol-d₄) δ (ppm) 8.16-8.19 (m, 1H), 8.01-8.03 (m, 1H), 7.20-7.23 (m, 2H), 6.62-6.84 (m, 6H), 5.87 (s, 2H), 4.58 (s, 2H), 2.85 (d, 6H). MS m/z: 430 (M+1).

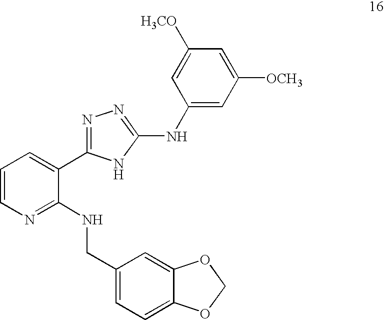

Benzo[1,3]dioxol-5-ylmethyl-{3-[5-(3,5-dimethoxy-phenylamino)-4H-[1,2,4]triazol-3-yl]-pyridin-2-yl}-amine (16): ¹HNMR (methanol-d₄) δ (ppm) 8.02-8.15 (m, 2H), 6.69-6.81 (m, 2H), 6.60-6.67 (m, 4H), 6.04 (s, 1H), 5.45-5.87 (m, 2H), 4.57 (d, 2H), 3.73 (s, 3H), 3.69 (s, 3H). MS m/z: 447 (M+1).

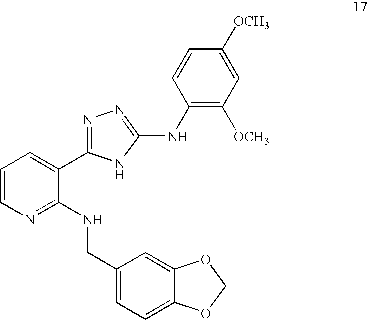

Benzo[1,3]dioxol-5-ylmethyl-{3-[5-(2,4-dimethoxy-phenylamino)-4H-[1,2,4]triazol-3-yl]-pyridin-2-yl}-amine (17): ¹HNMR (DMSO-d₆) δ (ppm) 12.20 (s, 1H), 8.04-8.23 (m, 4H), 7.81-7.90 (m, 1H), 6.82-6.97 (m, 3H), 6.67-6.78 (m, 2H), 6.44-6.48 (m, 1H), 6.01 (s, 2H), 4.57 (d, 2H), 3.88 (s, 3H), 3.67 (s, 3H). MS m/z: 447 (M+1).

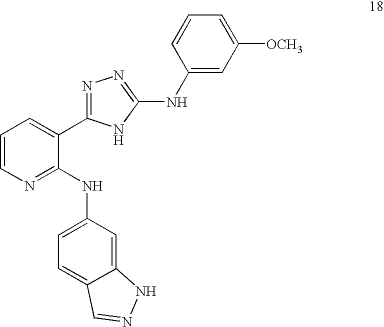

(1H-Indazol-6-yl)-{3-[5-(3-methoxy-phenylamino)-4H-[1,2,4]triazol-3-yl]-pyridin-2-yl}-amine (18): ¹HNMR (acetone-d₆) δ (ppm) 8.78-8.95 (m, 2H), 8.30-8.60 (m, 2H), 7.95 (s, 1H), 7.65-7.70 (m, 1H), 7.44-7.48 (m, 1H), 7.10-7.38 (m, 3H), 6.91-6.98 (m, 1H), 6.55-6.65 (m, 1H), 3.90 (s, 3H). MS m/z: 399 (M+1).

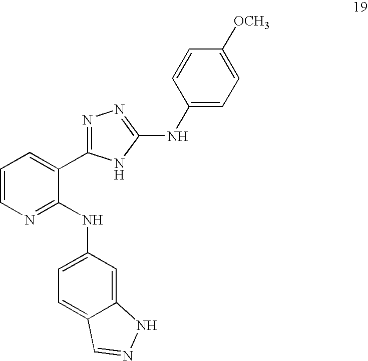

(1H-Indazol-6-yl)-{3-[5-(4-methoxy-phenylamino)-4H-[1,2,4]triazol-3-yl]-pyridin-2-yl}-amine (19): ¹HNMR (methanol-d₄) δ (ppm) 8.51 (s, 1H), 8.23-8.50 (m, 2H), 7.91-7.94 (s, 1H), 7.61-7.65 (m, 1H), 7.40-7.46 (m, 2H), 6.85-7.08 (m, 4H), 3.65 (s, 3H). MS m/z: 399 (M+1).

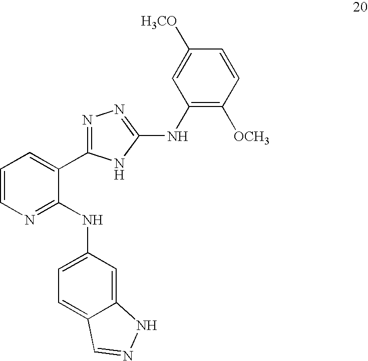

{3-[5-(2,5-Dimethoxy-phenylamino)-4H-[1,2,4]triazol-3-yl]-pyridin-2-yl}-(1H-indazol-6-yl)-amine (20): ¹HNMR (methanol-d₄) δ (ppm) 8.45 (s, 1H), 8.36 (d, 1H, J=5.7 Hz), 8.23-8.26 (m, 1H), 7.88-7.91 (m, 2H), 7.61-7.64 (m, 1H), 7.04-7.08 (m, 1H), 6.87-6.92 (m, 2H), 5.50-6.54 (m, 1H), 3.88 (s, 3H), 3.77 (s, 3H). MS m/z: 429 (M+1).

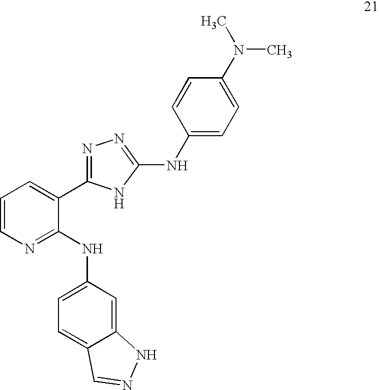

(1H-Indazol-6-yl)-{3-[5-(4-dimethylamino-phenylamino)-4H-[1,2,4]triazol-3-yl]-pyridin-2-yl}-amine (21): ¹HNMR (methanol-d₄) δ (ppm) 8.55 (s, 1H), 8.32-8.38 (m, 1H), 8.23-8.25 (m, 1H), 7.92 (s, 1H), 7.60-7.63 (m, 1H), 7.34-7.38 (m, 2H), 7.05-7.08 (m, 1H), 6.85-6.90 (m, 3H), 2.94 (s, 3H), 2.87 (s, 3H). MS m/z: 412 (M+1).

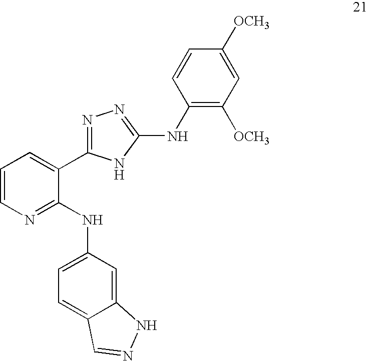

{3-[5-(2,4-Dimethoxy-phenylamino)-4H-[1,2,4]triazol-3-yl]-pyridin-2-yl}-(1H-indazol-6-yl)-amine (22): ¹HNMR (methanol-d₄) δ 8.50 (ppm) (s, 1H), 8.33-8.35 (m, 1H), 8.22-8.25 (m, 1H), 7.91 (d, 1H), 7.78 (d, 1H), 7.60 (d, 1H), 7.01-7.06 (m, 1H), 6.85-6.89 (m, 1H), 6.55-6.65 (m, 2H), 3.89 (s, 3H), 3.81 (s, 3H). MS m/z: 429 (M+1).

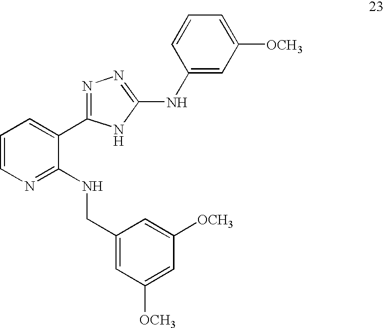

(3,5-Dimethoxy-benzyl)-{3-[5-(3-methoxy-phenylamino)-4H-[1,2,4]triazol-3-yl]-pyridin-2-yl}-amine (23): 1HNMR (DMSO-d₆) δ (ppm) 13.35 (s, 1H), 9.45 (s, 1H), 8.12 (s, 2H), 7.06-7.25 (m, 2H), 6.70-6.74 (m, 1H), 6.37-6.51 (m, 4H), 4.70-4.72 (d, 2H), 3.62-3.72 (m, 9H). MS m/z: 433 (M+1).

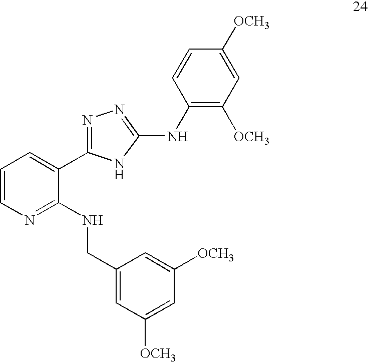

(3,5-Dimethoxy-benzyl)-{3-[5-(2,4-dimethoxy-phenylamino)-4H-[1,2,4]triazol-3-yl]-pyridin-2-yl}-amine (24): ¹HNMR (DMSO-d₆) δ (ppm) 12.17 (s, 1H), 8.09-8.66 (m, 4H), 7.85-7.98 (m, 1H), 6.27-6.70 (m, 6H), 4.68 (m, 2H), 3.65-3.90 (m, 12H). MS m/z: 463 (M+1).

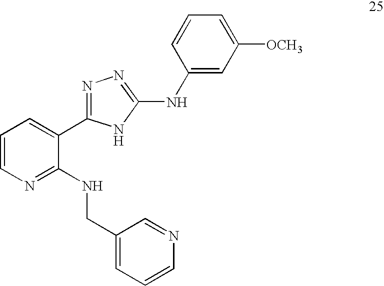

{3-[5-(3-Methoxy-phenylamino)-4H-[1,2,4]triazol-3-yl]-pyridin-2-yl}-pyridin-3-ylmethyl-amine (25): ¹HNMR (methanol-d₄) δ (ppm) 8.45 (s, 1H), 8.26-8.28 (m, 1H), 8.07-8.08 (m, 1H), 7.93-7.95 (m, 1H), 7.73 (d, 1H), 7.21-7.26 (m, 1H), 6.99-7.08 (m, 2H), 6.83-6.86 (m, 1H), 6.56-6.61 (m, 1H), 6.37-6.40 (m, 1H), 4.69 (d, 2H), 3.65 (s, 3H). MS m/z: 374 (M+1).

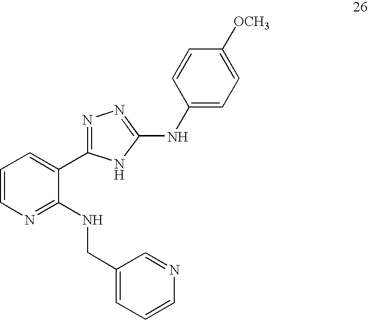

{3-[5-(4-Methoxy-phenylamino)-4H-[1,2,4]triazol-3-yl]-pyridin-2-yl}-pyridin-3-ylmethyl-amine (26): ¹HNMR (DMSO-d₆) δ (ppm) 9.19 (s, 1H), 8.61 (d, 1H), 8.43-8.48 (m, 1H), 8.12-8.20 (m, 2H), 7.74-7.77 (m, 1H), 7.33-7.44 (m, 3H), 6.70-6.85 (m, 3H) 4.78 (d, 2H), 3.71 (s, 3H). MS m/z: 374 (M+1).

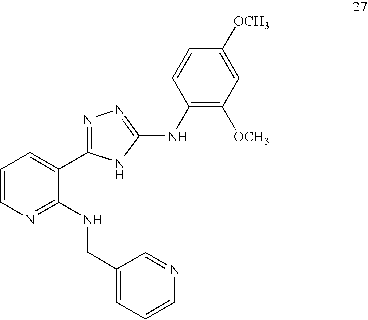

{3-[5-(2,4-Dimethoxy-phenylamino)-4H-[1,2,4]triazol-3-yl]-pyridin-2-yl}-pyridin-3-ylmethyl-amine (27): ¹HNMR (DMSO-d₆) δ (ppm) 8.59 (s, 1H), 8.44-8.46 (m, 2H), 8.21 (br, s, 1H), 8.09-8.10 (m, 1H), 7.85-7.88 (m, 1H), 7.73-7.88 (m, 1H), 7.31-7.36 (m, 1H), 6.63-6.73 (m, 2H), 6.44-6.69 (m, 1H), 4.77 (m, 2H), 3.84 (s, 3H), 3.67 (s, 3H). MS m/z: 404 (M+1).

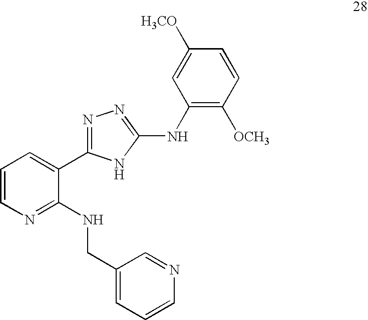

{3-[5-(2,5-Dimethoxy-phenylamino)-4H-[1,2,4]triazol-3-yl]-pyridin-2-yl}-pyridin-3-ylmethyl-amine (28): ¹HNMR (DMSO-d₆) δ (ppm) 8.65-8.58 (m, 2H), 8.43-8.46 (m, 1H), 8.19-8.23 (m, 1H), 8.10-8.12 (m, 2H), 7.93-7.94 (m, 2H), 7.71-7.74 (m, 1H), 7.30-7.36 (m, 1H), 6.92-6.95 (m, 1H), 6.71-6.76 (m, 1H), 6.43-6.47 (m, 1H), 4.79 (d, 2H), 3.83 (s, 3H), 3.71 (s, 3H). MS m/z: 404 (M+1).

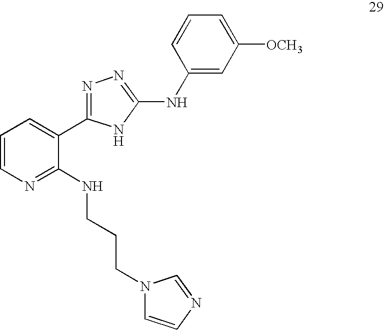

(3-imidazol-1-yl-propyl)-{3-[5-(3-methoxy-phenylamino)-4H-[1,2,4]triazol-3-yl]-pyridin-2-yl}-amine (29): ¹HNMR (CDCl₃) δ (ppm) 8.07-8.16 (m, 3H), 7.50-7.52 (m, 1H), 7.06-7.12 (m, 2H), 6.83-6.93 (m, 3H), 6.40-6.54 (m, 2H), 3.94-3.99 (m, 2H), 3.40 (s, 3H), 3.42-3.48 (m, 2H), 2.05-2.10 (m, 2H). MS m/z 391 (M+1).

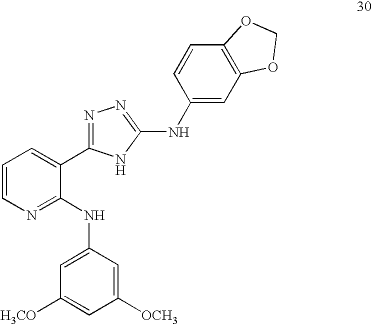

{3-[5-(Benzo[1,3]dioxol-5-ylamino)-4H-[1,2,4]triazol-3-yl]-pyridin-2-yl}-(3,5-dimethoxy-phenyl)-amine (30): ¹HNMR (DMSO-d₆) δ (ppm) 11.05 (s, 1H), 9.38 (s, 1H), 8.22-8.40 (m, 2H), 7.32 (s, 1H), 6.82-7.08 (m, 5H), 6.18 (s, 1H), 5.97 (s, 2H), 3.93 (s, 6H). MS m/z 434 (M+1).

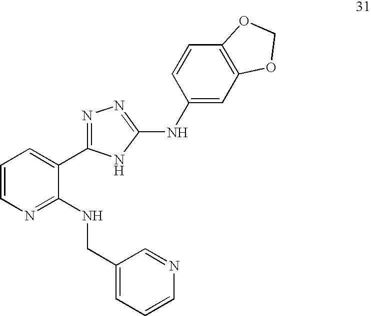

{3-[5-(Benzo[1,3]dioxol-5-ylamino)-4H-[1,2,4]triazol-3-yl]-pyridin-2-yl}-pyridin-3-ylmethyl-amine (31): ¹HNMR (DMSO-d₆) δ (ppm) 9.30 (s, 1H), 8.72-8.74 (m, 1H), 8.62 (s, 1H), 8.46 (d, 1H), 8.12-8.22 (m, 2H), 8.72-8.78 (m, 1H), 7.22-7.38 (m, 2H), 6.92-6.96 (m, 1H), 6.70-7.94 (m, 2H), 5.96 (s, 2H), 4.79 (d, 2H). MS m/z 388 (M+1).

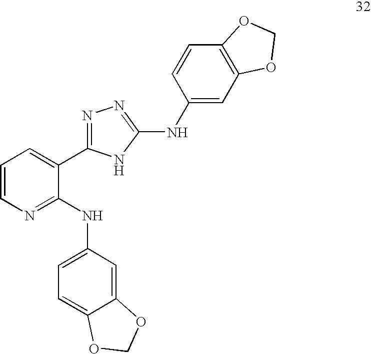

Benzo[1,3]dioxol-5-yl-{3-[5-(benzo[1,3]dioxol-5-ylamino)-4H-[1,2,4]triazol-3-yl]-pyridin-2-yl}-amine (32): ¹HNMR (DMSO-d₆) δ (ppm) 10.10 (s, 1H), 8.00-8.24 (m, 2H), 7.28 (s, 1H), 6.85-6.96 (m, 2H), 6.56-6.75 (m, 3H), 5.90 (s, 2H), 5.80 (s, 2H). MS m/z 417 (M+1).

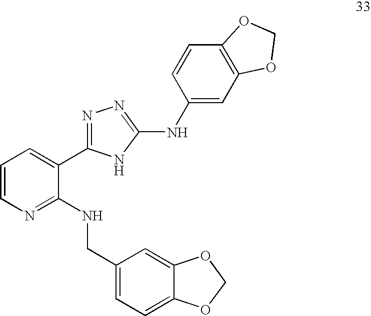

{3-[5-(Benzo[1,3]dioxol-5-ylamino)-4H-[1,2,4]triazol-3-yl]-pyridin-2-yl}-benzo[1,3]dioxol-5-ylmethyl-amine (33): ¹HNMR (DMSO-d₆) δ (ppm) 8.22 (m, 1H) 8.05-8.09 (m, 1H), 7.88-7.92 (m, 1H), 7.04 (s, 1H), 6.75-6.87 (m, 3H), 6.54-6.60 (m, 3H), 6.42-6.48 (m, 1H), 5.83 (s, 2H), 5.80 (s, 2H), 4.61 (d, 2H). MS m/z 431 (M+1).

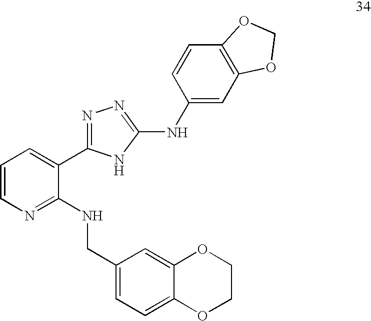

{3-[5-(Benzo[1,3]dioxol-5-ylamino)-4H-[1,2,4]triazol-3-yl]-pyridin-2-yl}-(2,3-dihydro-benzo[1,4]dioxin-6-ylmethyl)-amine (34): ¹HNMR (DMSO-d₆) δ (ppm) 13.6 (s, 1H), 8.10-8.20 (m, 2H), 7.21 (s, 2H), 6.95-6.99 (m, 1H), 6.74-6.88 (m, 5H), 5.98 (s, 2H), 4.74 (d, 2H), 4.18-4.20 (m, 4H). MS m/z: 445 (M+1).

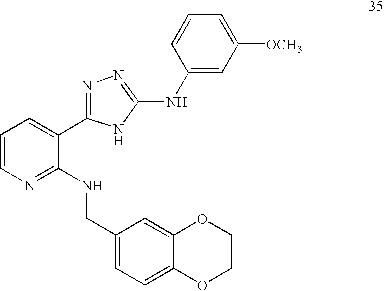

(2,3-Dihydro-benzo[1,4]dioxin-6-ylmethyl)-{3-[5-(3-methoxy-phenylamino)-4H-[1,2,4]triazol-3-yl]-pyridin-2-yl}-amine (35): ¹HNMR (DMSO-d₆) δ (ppm) 8.60-8.62 (m, 1H), 8.11-8.16 (m, 2H), 7.76-7.81 (m, 1H), 7.37-7.41 (m, 1H), 7.05-7.23 (m, 2H), 6.71-6.88 (m, 5H), 4.79 (d, 2H), 4.18-4.19 (s, d, 4H), 3.74 (s, 3H). MS m/z: 431 (M+1).

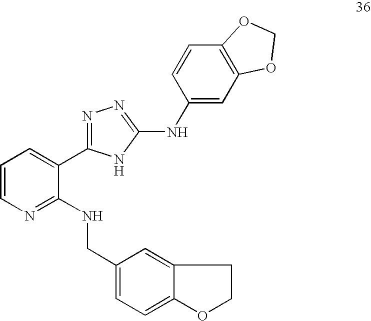

{3-[5-(Benzo[1,3]dioxol-5-ylamino)-4H-[1,2,4]triazol-3-yl]-pyridin-2-yl}-(2,3-dihydro-benzofuran-5-ylmethyl)-amine (36): ¹HNMR (DMSO-d₆) δ (ppm) 13.30 (s, 1H), 9.02 (s, 1H), 8.10-8.24 (m, 2H), 6.95-7.20 (m, 4H), 6.72-6.85 (m, 3H), 5.98 (s, 2H), 4.71 (d, 2H), 4.10-4.16 (m, 2H), 3.14-3.18 (m, 2H). MS m/z: 429 (M+1).

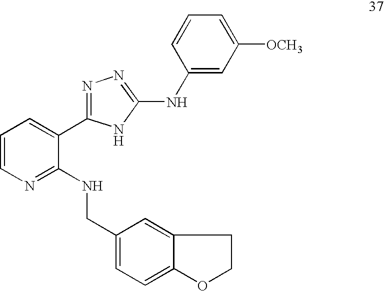

(2,3-Dihydro-benzofuran-5-ylmethyl)-{3-[5-(3-methoxy-phenylamino)-4H-[1,2,4]triazol-3-yl]-pyridin-2-yl}-amine (37): ¹HNMR (DMSO-d₆) δ (ppm) 13.45 (s, 1H), 9.79 (s, 1H), 9.10 (s, 1H), 8.12-8.18 (m, 2H), 7.11-7.24 (m, 5H), 6.65-6.73 (m, 2H), 6.55-6.66 (m, 1H), 4.48 (d, 2H), 4.08-4.16 (m, 2H), 3.74 (s, 3H), 3.12-3.18 (m, 2H). MS m/z: 415 (M+1).

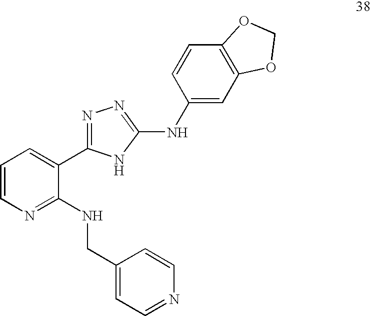

{3-[5-(Benzo[1,3]dioxol-5-ylamino)-4H-[1,2,4]triazol-3-yl]-pyridin-2-yl}-pyridin-4-ylmethyl-amine (38): ¹HNMR (DMSO-d₆) δ (ppm) 13.40 (s, 1H), 9.30 (s, 1H), 8.48-8.50 (m, 2H), 8.03-8.20 (m, 2H), 7.22-7.29 (m, 3H), 6.94-6.97 (m, 1H), 6.73-6.82 (m, 2H), 5.96 (s, 2H), 4.80 (d, 2H). MS m/z: 388 (M+1).

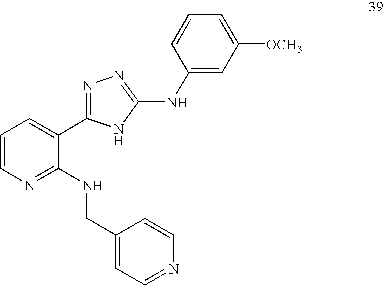

{3-[5-(3-Methoxy-phenylamino)-4H-[1,2,4]triazol-3-yl]-pyridin-2-yl}-pyridin-4-ylmethyl-amine (39): ¹HNMR (DMSO-d₆) δ (ppm) 8.54-8.56 (m, 2H), 8.10-8.23 (m, 2H), 7.30-7.39 (m, 3H), 7.05-7.19 (m, 2H), 6.78-6.81 (m, 1H), 6.48-6.51 (m, 1H), 4.86 (d, 2H), 3.78 (s, 3H). MS m/z: 374 (M+1).

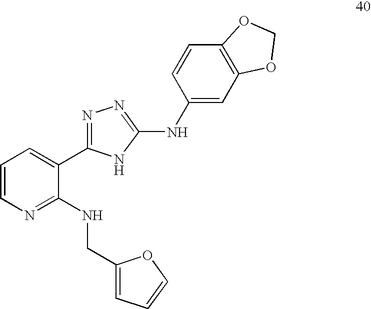

{3-[5-(Benzo[1,3]dioxol-5-ylamino)-4H-[1,2,4]triazol-3-yl]-pyridin-2-yl}-furan-2-ylmethyl-amine (40): ¹HNMR (DMSO-d₆) δ (ppm) 13.39 (s, 1H), 9.30 (s, 1H), 8.10-8.32 (m, 3H), 7.62-7.63 (m, 1H), 7.29-7.30 (m, 1H), 6.82-7.05 (m, 3H), 6.32-6.48 (m, 2H), 6.00 (d, 2H), 4.77-4.83 (m, 2H). MS m/z: 377 (M+1).

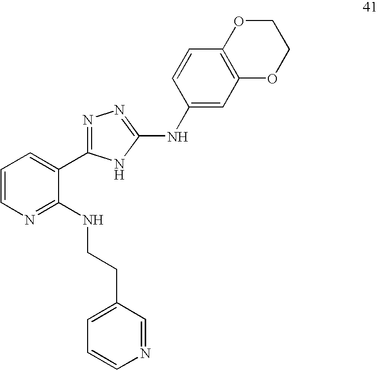

{3-[5-(2,3-Dihydro-benzo[1,4]dioxin-6-ylamino)-4H-[1,2,4]triazol-3-yl]-pyridin-2-yl}-(2-pyridin-3-yl-ethyl)-amine (41): ¹HNMR (DMSO-d₆) δ (ppm) 13.20 (s, 1H), 9.10 (s, 1H), 8.30-8.44 (m, 2H), 7.98-8.12 (m, 3H), 7.54-7.56 (m, 1H), 7.20-7.25 (m, 1H), 7.10 (s, 1H), 6.82-6.86 (m, 1H), 6.52-6.80 (m 2H), 4.12-4.22 (m, 6H), 3.64-3.68 (m, 2H). MS m/z: 416 (M+1).

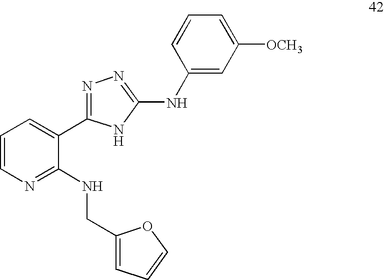

Furan-2-ylmethyl-{3-[5-(3-methoxy-phenylamino)-4H-[1,2,4]triazol-3-yl]-pyridin-2-yl}-amine (42): ¹HNMR (DMSO-d₆) δ (ppm) 13.41 (s, 1H), 9.50 (s, 1H), 8.05-8.25 (m, 3H), 7.40-7.44 (m, 1H), 7.10-7.30 (m, 3H), 6.75-6.83 (m, 1H), 6.25-6.40 (m, 2H), 4.78-4.81 (m, 2H), 3.78 (s, 3H). MS m/z: 363 (M+1).

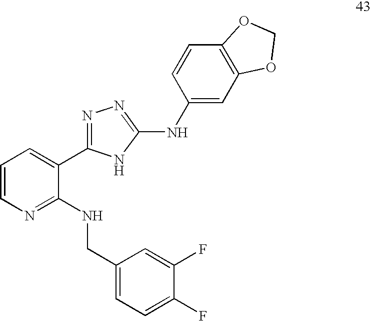

{3-[5-(Benzo[1,3]dioxol-5-ylamino)-4H-[1,2,4]triazol-3-yl]-pyridin-2-yl}-(3,4-difluoro-benzyl)-amine (43) ¹HNMR (DMSO-d₆) δ (ppm) 9.32 (br, s, 1H), 8.62-8.70 (br, m, 1H), 8.16-8.24 (m, 2H), 7.32-7.41 (m, 3H), 7.20-7.30 (m, 2H), 7.02-7.06 (m, 1H), 6.74-6.89 (m, 2H), 6.17 (s, 2H), 4.87 (d, 2H). MS m/z: 423 (M+1).

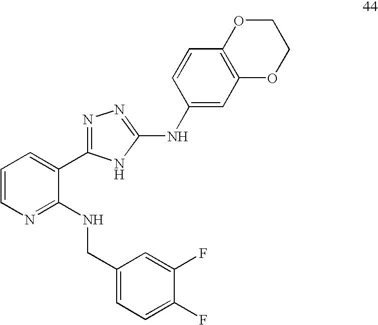

(3,4-Difluoro-benzyl)-{3-[5-(2,3-dihydro-benzo[1,4]dioxin-6-ylamino)-4H-[1,2,4]triazol-3-yl]-pyridin-2-yl}-amine (44): ¹HNMR (DMSO-d₆) δ (ppm) 12.85 (s, 1H), 9.33 (s, 1H), 8.81 (s, 1H), 8.20-8.28 (m, 2H), 7.45-7.52 (m, 2H), 7.34-7.38 (m, 2H), 6.98-7.02 (m, 1H), 6.77-6.81 (m, 2H), 4.81 (d, 2H), 4.22-4.31 (m, 4H). MS m/z: 437 (M+1).

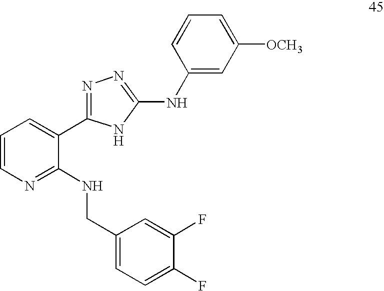

(3,4-Difluoro-benzyl)-{3-[5-(3-methoxy-phenylamino)-4H-[1,2,4]triazol-3-yl]-pyridin-2-yl}-amine (45): ¹HNMR (DMSO-d₆) δ (ppm) 9.45 (s, 1H), 7.05-7.47 (m, 7H), 6.77-6.83 (m, 1H), 6.48-6.52 (m, 1H), 4.88 (d, 2H), 3.72 (s, 3H). MS m/z: 409 (M+1).

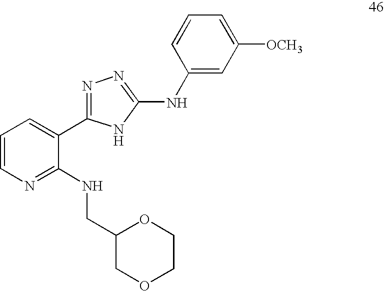

[1,4]Dioxan-2-ylmethyl-{3-[5-(3-methoxy-phenylamino)-4H-[1,2,4]triazol-3-yl]-pyridin-2-yl}-amine (46): ¹HNMR (DMSO-d₆) δ (ppm) 9.38 (br, s, 1H), 8.16-8.18 (m, 3H), 7.25-7.27 (m, 1H), 7.05-7.10 (m, 1H), 6.73-6.88 (m, 2H), 4.24-4.32 (m, 3H), 3.78-3.84 (m, 4H), 3.82 (s, 3H), 3.60-3.73 (m, 2H). MS m/z: 368 (M+1).

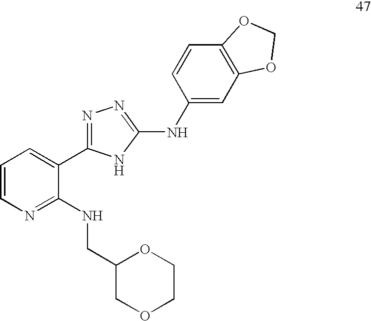

{3-[5-(Benzo[1,3]dioxol-5-ylamino)-4H-[1,2,4]triazol-3-yl]-pyridin-2-yl}-[1,4]dioxan-2-ylmethyl-amine (47): ¹HNMR (DMSO-d₆) δ (ppm) 9.33 (s, 1H), 8.35 (s, 1H), 8.17-8.21 (m, 2H), 7.30-7.32 (m, 1H), 7.03-7.10 (m, 1H), 6.92-6.96 (m, 1H), 6.77-6.81 (m, 1H), 6.03 (s, 2H), 3.35-3.88 (m, 9H). MS m/z: 382 (M+1).

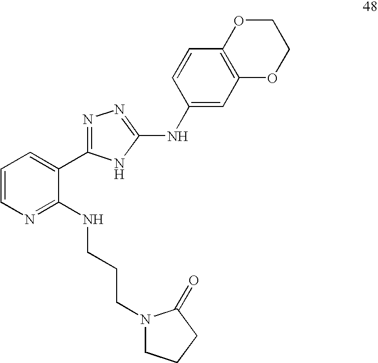

1-(3-{3-[5-(2,3-Dihydro-benzo[1,4]dioxin-6-ylamino)-4H-[1,2,4]triazol-3-yl]-pyridin-2-ylamino}-propyl)-pyrrolidin-2-one (48): ¹HNMR (DMSO-d₆) δ (ppm) 13.5 (s, 1H), 8.19 (s, 2H), 7.27-7.28 (m, 1H), 6.95-6.99 (m, 1H), 6.86-6.89 (m, 1H), 6.70-6.77 (m, 1H), 4.20-4.30 (m, 4H), 3.35-3.58 (m, H), 2.25-2.31 (m, 2H), 1.82-1.98 (m, 4H). MS m/z: 436 (M+1).

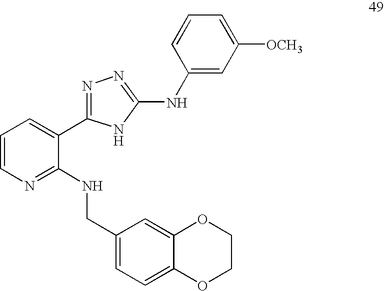

{3-[5-(2,3-Dihydro-benzo[1,4]dioxin-6-ylamino)-4H-[1,2,4]triazol-3-yl]-pyridin-2-yl}-(2,3-dihydro-benzo[1,4]dioxin-6-ylmethyl)-amine (49): ¹HNMR (DMSO-d₆) δ (ppm) 8.38-8.42 (m, 2H), 8.20-8.22 (m, 2H), 7.36 (s, 1H), 7.04-7.07 (m, 1H), 6.85-6.96 (m, 3H), 6.72-6.78 (m, 2H), 4.68-4.72 (m, 2H), 4.27-4.29 (m, 4H), 3.79 (s, 3H). MS m/z: 459 (M+1).

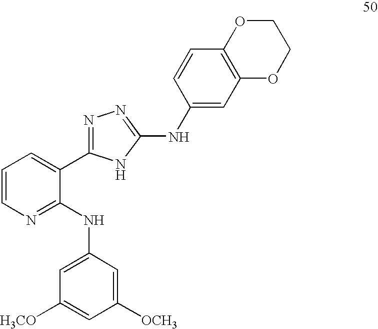

{3-[5-(2,3-Dihydro-benzo[1,4]dioxin-6-ylamino)-4H-[1,2,4]triazol-3-yl]-pyridin-2-yl}-(3,5-dimethoxy-phenyl)-amine (50): ¹HNMR (DMSO-d₆) δ (ppm) 10.72 (s, 1H), 9.52 (s, 1H), 8.22-8.41 (m, 2H), 6.82-7.28 (m, 6H), 6.15-6.22 (m, 1H), 4.22-4.31 (m, 4H), 3.78 (s, 3H), 3.74 (s, 3H). MS m/z: 447 (M+1).

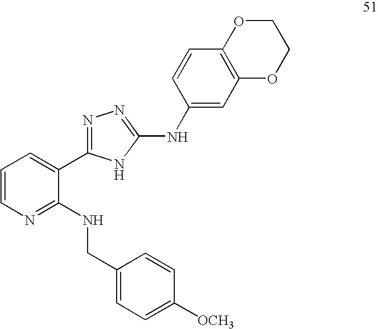

{3-[5-(2,3-Dihydro-benzo[1,4]dioxin-6-ylamino)-4H-[1,2,4]triazol-3-yl]-pyridin-2-yl}-(4-methoxy-benzyl)-amine (51): ¹HNMR (DMSO-d₆) δ (ppm) 9.31 (s, 1H), 9.02 (m, 1H), 8.20-8.22 (m, 1H), 8.05-8.11 (m, 1H), 7.40 (s, 1H), 7.28-7.32 (m, 2H), 7.15 (s, 1H), 6.88-6.98 (m, 3H), 6.62-6.73 (m, 2H), 4.72-4.80 (m, 2H), 4.20-4.28 (m, 4H), 3.76 (s, 3H). MS m/z: 431 (M+1).

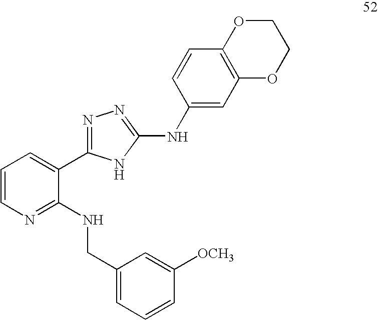

{3-[5-(2,3-Dihydro-benzo[1,4]dioxin-6-ylamino)-4H-[1,2,4]triazol-3-yl]-pyridin-2-yl}-(3-methoxy-benzyl)-amine (52): ¹HNMR (DMSO-d₆) δ (ppm) 13.28 (s, 1H), 9.19 (m, 1H), 8.18-8.26 (m, 2H), 7.43 (s, 1H), 7.20-7.26 (m, 2H), 6.65-6.95 (m, 5H), 4.77-4.79 (m, 2H), 4.20-4.24 (m, 4H), 3.78 (s, 3H). MS m/z: 431 (M+1).

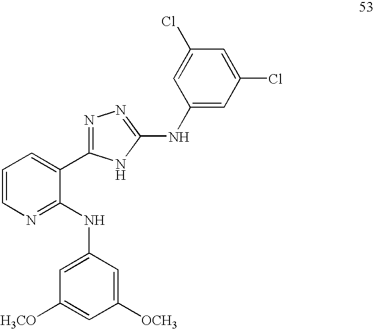

{3-[5-(3,5-Dichloro-phenylamino)-4H-[1,2,4]triazol-3-yl]-pyridin-2-yl}-(3,5-dimethoxy-phenyl)-amine (53). ¹HNMR (DMSO-d₆) δ (ppm) 8.3 (m, 2H), 7.7 (m, 2H), 7.0 (m, 4H), 6.2 (s, 1H), 5.8 (s, 1H), 3.7 (s, 6H). MS m/z: 458 (M+1).

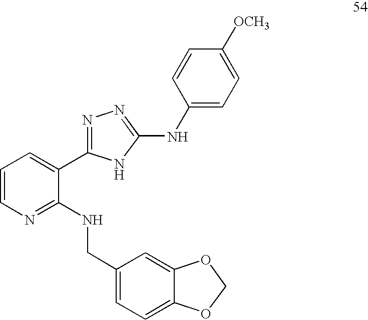

Benzo[1,3]dioxol-5-ylmethyl-{3-[5-(4-methoxy-phenylamino)-4H-[1,2,4]triazol-3-yl]-pyridin-2-yl}-amine (54). ¹HNMR (DMSO-d₆) δ (ppm) 9.63 (s, 1H), 8.97 (s, 1H), 8.38 (s, 1H), 8.15-8.28 (m, 2H), 7.49 (d, 2H), 6.90-6.98 (m, 5H), 6.84 (s, 1H), 6.02 (s, 2H), 4.73 (d, 2H), 3.78 (s, 3H). MS m/z: 417 (M+1). MS m/z: 417 (M+1).

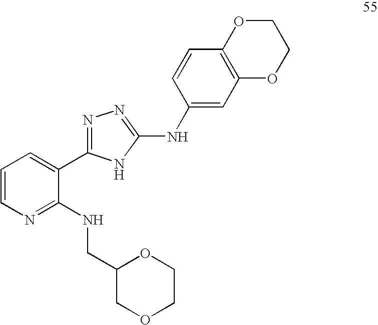

{3-[5-(2,3-Dihydro-benzo[1,4]dioxin-6-ylamino)-4H-[1,2,4]triazol-3-yl]-pyridin-2-yl}-[1,4]dioxan-2-ylmethyl-amine (55). ¹HNMR (DMSO-d₆) δ (ppm) 9.61 (s, 1H), 8.40 (br, s, 1H), 8.20-8.22 (m, 2H), 7.31 (s, 1H), 7.10-7.16 (m, 2H), 6.73-6.78 (m, 1H), 6.50-6.53 (m, 1H), 3.35-3.38 (m, 13H). MS m/z: 411 (M+1).

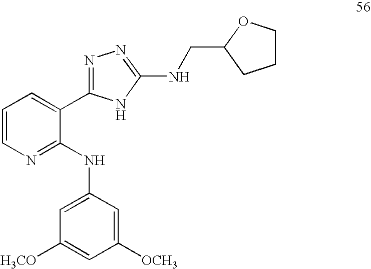

(3,5-Dimethoxy-phenyl)-(3-{5-[(tetrahydro-furan-2-ylmethyl)-amino]-4H-[1,2,4]triazol-3-yl}-pyridin-2-yl)-amine (56): ¹HNMR (DMSO-d₆) δ (ppm) 8.20-8.30 (m, 2H), 7.12 (s, 2H), 6.85-6.90 (m, 1H), 6.11 (s, 1H), 4.10-4.15 (m, 2H), 3.68 (s, 6H), 3.55-3.58 (m, 1H), 3.20-3.25 (m, 2H), 1.80-1.95 (m, 3H), 1.55-1.65 (m, 1H). MS m/z: 393 (M+1).

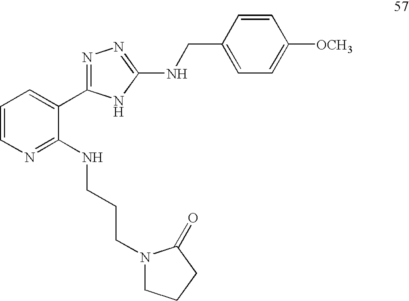

1-(3-{3-[5-(4-Methoxy-benzylamino)-4H-[1,2,4]triazol-3-yl]-pyridin-2-ylamino}-propyl)-pyrrolidin-2-one (57): ¹HNMR (DMSO-d₆) δ (ppm) 12.56 (s, 1H), 8.10-8.22 (m, 3H), 7.30-7.41 (m, 3H), 6.94-6.98 (m, 2H), 6.62-6.64 (m, 1H), 4.44-4.48 (m, 2H), 3.81 (s, 3H), 3.35-3.58 (m, 6H), 2.26-2.32 (m, 2H), 1.95-2.01 (m, 2H), 1.76-1.82 (m, 2H). MS m/z: 422 (M+1).

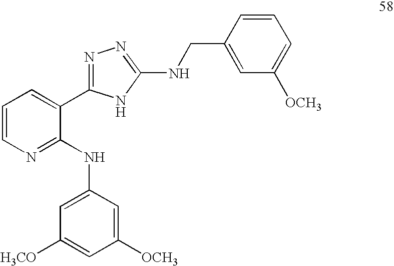

(3,5-Dimethoxy-phenyl)-{3-[5-(3-methoxy-benzylamino)-4H-[1,2,4]triazol-3-yl]-pyridin-2-yl}-amine (58): ¹HNMR (DMSO-d₆) δ (ppm) 12.72 (s, 1H), 10.91 (s, 1H), 8.28-8.32 (m, 2H), 7.56-7.59 (m, 1H), 7.24-7.30 (m, 1H), 6.78-6.99 (m 6H), 6.13 (s, 1H), 4.47 (d, 2H), 3.74 (s, 9H). MS m/z: 433 (M+1).

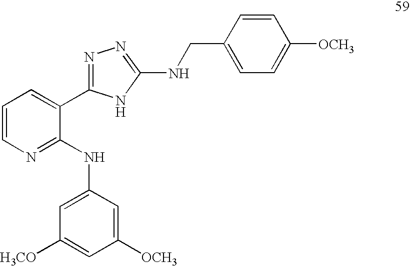

(3,5-Dimethoxy-phenyl)-{3-[5-(4-methoxy-benzylamino)-4H-[1,2,4]triazol-3-yl]-pyridin-2-yl}-amine (59): ¹HNMR (DMSO-d₆) δ (ppm) 12.72 (s, 1H), 10.89 (s, 1H), 8.26-8.34 (m, 2H), 7.46-7.47 (m, 1H), 7.24-7.28 (m, 2H), 7.03 (s, 2H), 6.80-6.87 (m, 3H), 6.12 (s, 1H), 4.37-4.42 (m, 2H), 3.72 (s, 9H). MS m/z: 433 (M+1).

### Example 2

### Synthesis of N-Benzo[1,3]dioxol-5-ylmethyl-N′-{5-[2-(3,5-dimethoxy-phenylamino)-pyridin-3-yl]-4H-[1,2,4]triazol-3-yl}-benzene-1,3-diamine (60)

### Step 1: synthesis of (3,5-dimethoxy-phenyl)-{3-[5-(3-nitro-phenylamino)-4H-[1,2,4]-triazol-3-yl]-pyridin-2-yl}-amine (60a)

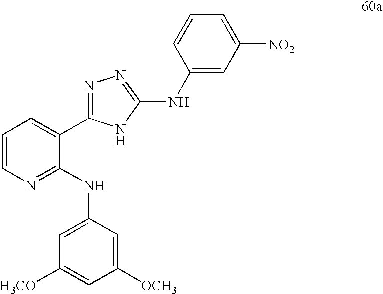

The reaction mixture of 2-(3,5-dimethoxy-phenylamino)-nicotinic acid hydrazide (1c from Example 1, 1.0 g, 3.47 mmol) and 2-methyl-1-(3-nitro-phenyl)-isothiourea (prepared using the method for the synthesis of 1f from Example 1, 1.42 g, 4.16 mmol) in pyridine (6 ml) was stirred at 140° C. under argon for 4 hours. The mixture was poured into 50 ml water, and extracted three times with ethyl acetate (40 ml). The organic layer was dried over sodium sulfate, filtered, and evaporated. The residue was subjected to silica gel column (CH₂Cl₂:methanol=110:1) to obtain 861 mg of 60a as white solid in 54% yield. ¹HNMR (DMSO-d₆) δ (ppm) 10.55 (s, 1H), 8.66 (s, 1H), 8.28-8.37 (m, 2H), 7.92-7.96 (m, 1H), 7.73-7.78 (m, 1H), 7.57-7.61 (m, 1H), 7.09 (s, 2H), 6.93-6.99 (m, 1H), 3.78 (s, 6H). MS m/z: 434 (M+1).

### Step 2: synthesis of N-{5-[2-(3,5-dimethoxy-phenylamino)-pyridin-3-yl]-4H-[1,2,4]triazol-3-yl}-benzene-1,3-diamine (60b)

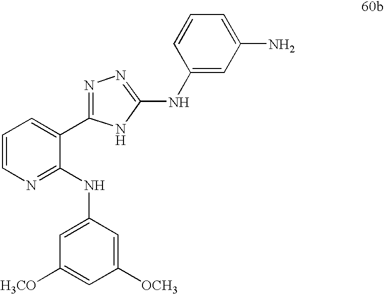

The reaction mixture of the nitro triazole compound (60a, 800 mg) and contain Pd—C 10% (120 mg) in ethanol (100 ml) was degassed and stirred under hydrogen at 60° C. for 4 hours. After filtering through Celite, the filtrate was evaporated to obtain 706 mg white solid (94.5% yield). ¹HNMR (DMSO-d₆) δ (ppm) 11.08 (s, 1H), 9.33 (s, 1H), 8.30-8.40 (m, 2H), 7.25 (s, 2H), 6.92-6.98 (m, 2H), 6.74-6.78 (m, 2H), 6.13 (m, 2H), 5.02 (m, 2H), 3.79 (s, 6H). MS m/z: 404 (M+1).

### Step 3: synthesis of N-Benzo[1,3]dioxol-5-ylmethyl-N′-{5-[2-(3,5-dimethoxy-phenylamino)-pyridin-3-yl]-4H-[1,2,4]triazol-3-yl}-benzene-1,3-diamine (60)

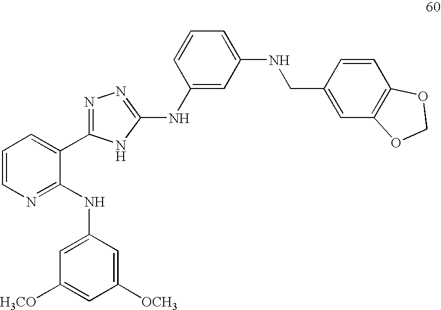

To a solution of (3,5-dimethoxy-phenyl)-{3-[5-(3-nitro-phenylamino)-4H-[1,2,4]triazol-3-yl}-amine (60b, 80 mg, 0.198 mmol) in anhydrous dichloroethane (20 ml) there was added benzo[1,3]dioxole-5-carboxaldehyde (33 mg, 0.218 mmol, from Aldrich), sodium triacetoxyborohydride (84 mg, 0.396 mmol, from Aldrich) and acetic acid (0.2 mmol). The reaction mixture was stirred at ambient temperature for 3 hours. The reaction was quenched with 10% NaOH (2 ml) and water (10 ml), then extracted three times with 15 ml ethyl acetate. The combined organic layer was washed with brine, and dried over anhydrous Na₂SO₄. After filtration and evaporation, the organic residue was subjected to preparative TLC (CH₂Cl₂:MeOH=25:1) to obtain 31 mg of compound 60 in 29% yield. ¹HNMR (DMSO-d₆) δ (ppm) 9.45 (s, 1H), 8.40-8.46 (m, 2H), 7.35-7.36 (m, 2H), 7.02-7.12 (m, 3H), 6.88-6.98 (m, 4H), 6.34-6.37 (m, 2H), 6.07 (s, 2H), 4.31 (d, 2H), 3.89 (s, 6H). MS m/z: 538 (M+1).

Compounds 61 and 62 were prepared using method described in Example 2:

### Analytical Data:

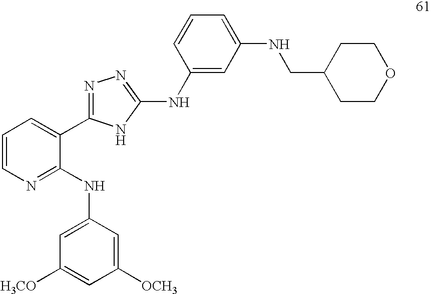

N-{5-[2-(3,5-Dimethoxy-phenylamino)-pyridin-3-yl]-4H-[1,2,4]triazol-3-yl}-N′-(tetrahydro-pyran-4-ylmethyl)-benzene-1,3-diamine (61): ¹HNMR (DMSO-d₆) δ (ppm) 8.30-8.32 (m, 2H), 6.80-7.18 (m, 6H), 6.20-6.23 (m, 2H), 5.84-5.91 (m, 2H), 3.79 (s, 6H), 3.20-3.30 (m, 3H), 2.90-2.97 (m, 2H), 1.60-1.88 (m, 4H), 1.10-1.26 (m, 2H). MS m/z: 502 (M+1).

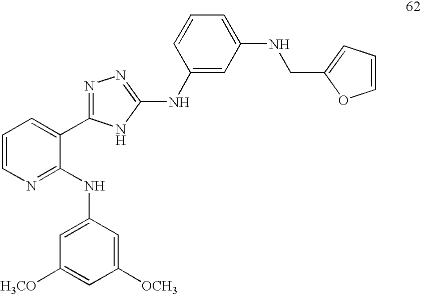

N-{5-[2-(3,5-Dimethoxy-phenylamino)-pyridin-3-yl]-4H-[1,2,4]triazol-3-yl}-N′-furan-2-ylmethyl-benzene-1,3-diamine (62): ¹HNMR (DMSO-d₆) δ (ppm) 13.40 (s, 1H), 10.85 (s, 1H), 9.21 (s, 1H), 7.51 (s, 1H), 6.81-7.14 (m, 6H), 6.05-6.32 (m, 5H), 4.21 (d, 2H), 3.84 (s, 6H). MS m/z: 484 (M+1).

### Example 3

### Synthesis of 2,3-Dihydro-benzofuran-5-sulfonic acid (3-{5-[2-(3,5-dimethoxy-phenylamino)-pyridin-3-yl]-4H-[1,2,4]triazol-3-ylamino}-phenyl)-amide (63)

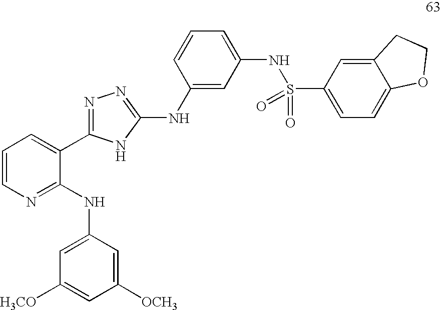

To a solution of N-{5-[2-(3,5-Dimethoxy-phenylamino)-pyridin-3-yl]-4H-[1,2,4]triazol-3-yl}-benzene-1,3-diamine (60b from Example 2, 60 mg, 0.148 mmol) in pyridine (1 ml), 2,3-dihydro-benzofuran-5-sulfonyl chloride (35.7 mg, 0.163 mmol, from Oakwood Products, Inc.) was slowly added. The reaction mixture was stirred at ambient temperature for 3 hours then poured into aqueous NaHCO₃ (10 ml), and extracted with ethyl acetate (3×10 ml). The combined organic layer was washed with brine, dried over anhydrous Na₂SO₄, filtered and evaporated. The organic residue was subjected to preparative thin layer chromatography (CH₂Cl₂:Methanol=20:1) to obtain 12 mg of 63 in 14% yield. ¹HNMR (DMSO-d₆) δ (ppm) 8.33-8.37 (m, 2H), 7.78 (s, 1H), 7.66-7.69 (m, 1H), 7.57 (s, 1H), 7.22-7.25 (m, 1H), 7.12-7.28 (m, 3H), 6.99-7.04 (m, 1H), 6.90 (d, 1H), 6.69-6.72 (m, 1H), 6.22 (s, 1H), 4.60-4.70 (t, 2H), 3.73 (s, 6H), 3.20-3.26 (t, 2H). MS m/z: 586 (M+1).

Compound 64 was prepared using method described in Example 3:

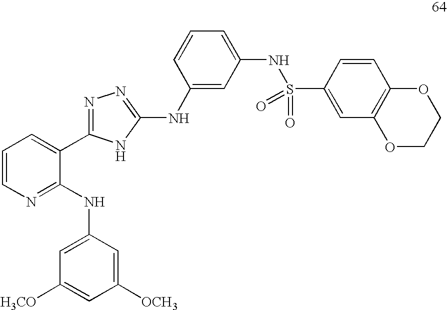

2,3-Dihydro-benzo[1,4]dioxine-6-sulfonic acid (3-{5-[2-(3,5-dimethoxy-phenylamino)-pyridin-3-yl]-4H-[1,2,4]triazol-3-ylamino}-phenyl)-amide (64): ¹HNMR (DMSO-d₆) δ (ppm) 9.63 (s, 1H), 8.28-8.37 (m, 2H), 7.50 (s, 1H), 7.29-7.34 (m, 3H), 7.08-7.17 (m 3H), 6.92-7.00 (m, 2H), 6.60-6.63 (d, 1H), 6.27-6.28 (m, 1H), 4.29-4.33 (m, 4H), 3.77 (s, 6H). MS m/z: 602 (M+1).

### Example 4

### Synthesis of (3,5-Dimethoxy-phenyl)-{3-[5-(3-methoxy-phenylamino)-1-methyl-1H-[1,2,4]triazol-3-yl]-pyridin-2-yl}-amine (65)

### Step 1: Synthesis of 2-(3,5-Dimethoxy-phenylamino)-nicotinic acid N′-methyl-hydrazide (65a)

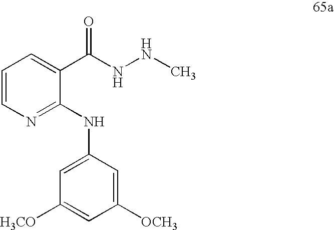

A reaction mixture containing 2-(3,5-dimethoxy-phenylamino)-nicotinic ethyl ester (1b, 2.0 g, 6.6 mmol), methylhydrazine (1.39 ml, 25.4 mmol, from Aldrich) and 2-propanol (5 ml) in seal tube was heated at 120° C. for 12 hours. After concentration, ethyl acetate was added to the crude mixture, and a solid precipitated. After filtration, the solid was dried in vacuum oven to give 1.3 g of 65a in 65% yield. ¹HNMR (DMSO-d₆) δ (ppm) 10.80 (s, 1H), 10.28 (s, 1H), 8.35-8.37 (m, 1H), 8.03 (d, 1H), 6.98 (s, 2H), 6.80-6.85 (m, 1H), 6.02 (t, 1H), 5.25 (s, br, 1H), 3.76 (s, 6H), 2.60 (s, 3H). MS m/z: 303 (M+1).

### Step 2: Synthesis {3-[5-(Benzo[1,3]dioxol-5-ylamino)-1-methyl-1H-[1,2,4]triazol-3-yl]-pyridin-2-yl}-(3,5-dimethoxy-phenyl)-amine (65)

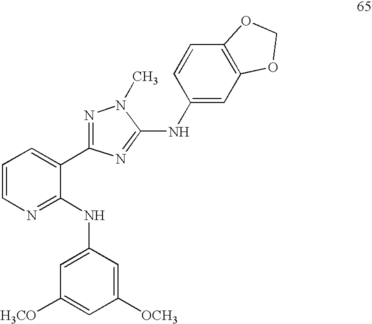

To a solution of 2-(3,5-Dimethoxy-phenylamino)-nicotinic acid N′-methyl-hydrazide (65a, 302 mg, 1.0 mmol) in pyridine (2 ml), 1-benzo[1,3]dioxol-6-yl)-2-methyl-isothiourea (synthesized according to the procedure for making 1f, from Example 1, 439.4 mg, 1.3 mmol) and triethylamine (0.30 ml) were added. The reaction mixture was heated at 160° C. for 12 hours under argon. The mixture was poured into water (30 ml), and extracted with ethyl acetate (30 ml×3). The combined organic layer was dried over anhydrous Na₂SO₄, filtered, and evaporated. The organic residue was subjected to silica gel column (CH₂Cl₂:MeOH=125:1) to obtain 150 mg compound 65 in 33.6% yield. ¹HNMR (DMSO-d₆) δ (ppm) 10.60 (s, 1H), 9.02 (s, 1H), 8.20-8.30 (m, 2H), 7.30 (s, 1H), 7.05-7.09 (m, 1H), 6.98 (s, 2H), 6.86-6.90 (m, 2H), 6.13 (s, 1H), 5.98 (s, 2H), 3.86 (s, 3H), 3.75 (s, 6H). MS m/z: 447 (M+1).

Compounds 66 to 70 were synthesized using the described method from Example 4:

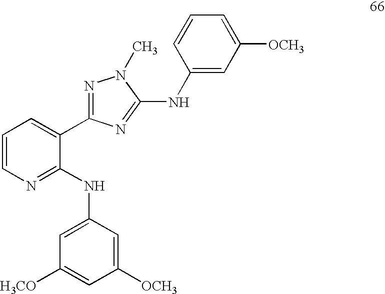

(3,5-Dimethoxy-phenyl)-{3-[5-(3-methoxy-phenylamino)-1-methyl-1H-[1,2,4]triazol-3-yl]-pyridin-2-yl}-amine (66). ¹HNMR (DMSO-d₆) δ (ppm) 10.75 (s, 1H), 9.23 (s, 1H), 8.39-8.41 (m, 1H), 8.28-8.30 (m, 1H), 7.38 (s, 1H), 7.30-7.32 (m, 2H), 7.06 (m, 2H), 6.03-6.09 (m 1H), 6.62-6.65 (m, 1H), 6.20 (s, 1H), 3.95 (s, 3H), 3.81 (s, 6H), 3.77 (s, 3H). MS m/z: 433 (M+1).

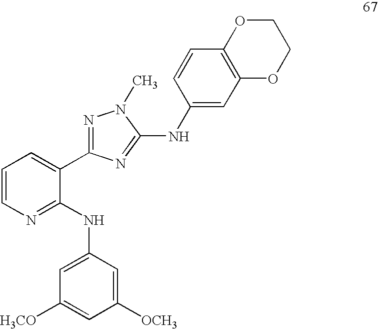

{3-[5-(2,3-Dihydro-benzo[1,4]dioxin-6-ylamino)-1-methyl-1H-[1,2,4]triazol-3-yl]-pyridin-2-yl}-(3,5-dimethoxy-phenyl)-amine (67). ¹HNMR (DMSO-d₆) δ (ppm) 10.69 (s, 1H), 9.02 (s, 1H), 8.30-8.40 (m, 2H), 7.31-7.33 (m, 1H), 7.12-7.16 (m, 1H), 7.05 (s, 2H), 6.87-6.96 (m, 2H), 6.20 (t, 1H), 4.28-4.31 (m, 4H), 3.90 (s, 3H), 3.79 (s, 6H). MS m/z: 461 (M+1).

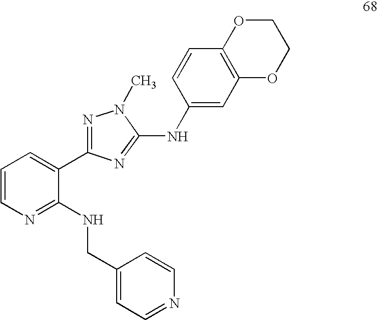

{3-[5-(2,3-Dihydro-benzo[1,4]dioxin-6-ylamino)-1-methyl-1H-[1,2,4]triazol-3-yl]-pyridin-2-yl}-pyridin-4-ylmethyl-amine (68). ¹HNMR (CDCl₃) δ (ppm) 8.45-8.47 (m, 2H), 8.27-8.29 (m, 2H), 8.05-8.07 (m, 1H), 7.22-7.26 (m, 2H), 6.97 (s, 1H), 6.69-6.72 (m, 2H), 6.58-6.63 (m, 1H), 5.93 (m, 1H), 4.70-4.74 (m, 2H), 4.20-4.22 (m, 4H), 3.65 (s, 3H). MS m/z: 415.18.

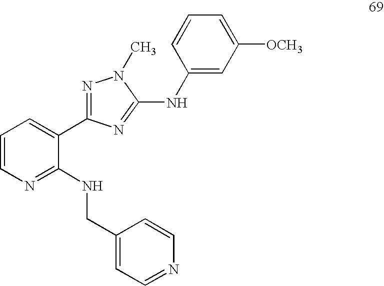

{3-[5-(3-Methoxy-phenylamino)-1-methyl-1H-[1,2,4]triazol-3-yl]-pyridin-2-yl}-pyridin-4-ylmethyl-amine (69). ¹HNMR (CDCl₃) δ (ppm) 8.46-8.48 (m, 2H), 8.25-8.29 (m, 2H), 8.05-8.08 (m, 1H), 7.24-7.26 (m, 2H), 7.10 (t, 1H), 6.99 (s, 1H), 6.79-6.81 (m, 1H), 6.61-6.65 (m, 1H), 6.50-6.54 (m, 1H), 6.32 (s, 1H), 4.30 (d, 2H), 3.85 (s, 3H), 3.69 (s, 3H). MS m/z: 388 (M+1).

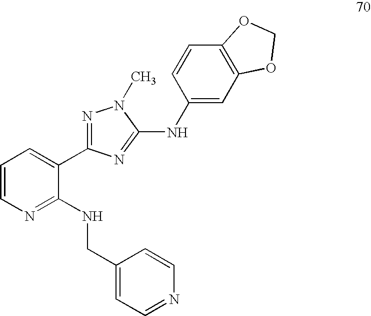

{3-[5-(Benzo[1,3]dioxol-5-ylamino)-1-methyl-1H-[1,2,4]triazol-3-yl]-pyridin-2-yl}-pyridin-4-ylmethyl-amine (70). ¹HNMR (CDCl₃) δ (ppm) 8.47-8.50 (m, 2H), 8.20-8.27 (m, 2H), 8.06-8.08 (m, 1H), 7.28-7.31 (m, 2H), 7.02 (s, 1H), 6.65-6.68 (m, 4H), 6.02-6.03 (m, 1H), 5.93 (s, 2H), 4.82 (d, 2H), 3.64 (s, 3H). MS m/z: 402 (M+1).

### Example 5

### Synthesis of {5-[2-(Benzo[1,3]dioxol-5-ylmethoxy)-pyridin-3-yl]-4H-[1,2,4]triazol-3-yl}-(2,3-dihydro-benzo[1,4]dioxin-6-yl)-amine (71)

### Step-1: synthesis of 2-(Benzo[1,3]dioxol-5-ylmethoxy)-nicotinic Acid Ethyl Ester (71a)

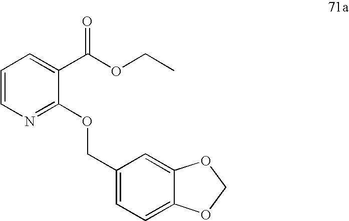

At ambient temperature, sodium hydride (0.682 g, 17.05 mmol, purchased from Aldrich, 60% oil suspension) was added slowly to a solution of benz[1,3]dioxol-5-yl-methanol (2.14 g, 15.5 mmol, purchased from Aldrich) in anhydrous DMF (10 ml) under argon. After 30 minutes, 2-chloro-nicotinic acid ethyl ester (1a, 3.0 g, 16.3 mmol) was added slowly to the reaction mixture. The resulting mixture was heated at 80° C. for 2 hours. Water (10 ml) was added slowly to quench the reaction. After dilution with 100 ml of water, the mixture was extracted with ether (60 ml×3). The combined organic layer were dried over anhydrous Na₂SO₄, filtered and evaporated. The organic residue was subjected to column chromatography (Hexane: EtOAc=15:1) to obtain 1.8 g of 71a in 41% yield. ¹HNMR (DMSO-d₆) δ (ppm) 8.49-8.52 (m, 1H), 8.15-8.19 (m, 1H), 7.05-7.16 (m, 2H), 6.88-6.97 (m, 2H), 6.01 (s, 2H), 4.27-4.32 (m, 2H), 1.21-1.30 (t, 3H).

### Step-2: synthesis of 2-(Benzo[1,3]dioxol-5-ylmethoxy)-nicotinic acid Hydrazide (71b)

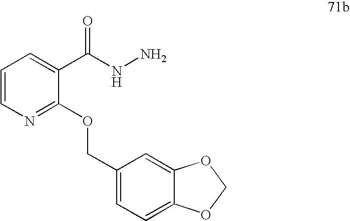

2-(Benzo[1,3]dioxol-5-ylmethoxy)-nicotinic acid ethyl ester (71a, 1.8 g, 6.2 mmol) was added to 2-propanol (10 ml) followed by hydrazine monohydrate (0.93 ml, 18.6 mmol), and the reaction mixture was heated at 80° C. under argon for two weeks. White solid precipitated from the reaction. After filtration and vacuum-drying, 1.1 g 71b was obtained in 55% yield. ¹HNMR (DMSO-d₆) δ (ppm) 9.31 (s, 1H), 8.29-8.3 9 (m, 1H), 7.11-7.16 (m, 2H), 6.96-7.01 (m, 1H), 6.92 (d, 2H), 6.02 (s, 2H), 5.39 (s, 2H), 4.57 (s, 2H).

### Step-3: synthesis of {5-[2-(Benzo[1,3]dioxol-5-ylmethoxy)-pyridin-3-yl]-4H-[1,2,4]triazol-3-yl}-(2,3-dihydro-benzo[1,4]dioxin-6-yl)-amine (71)

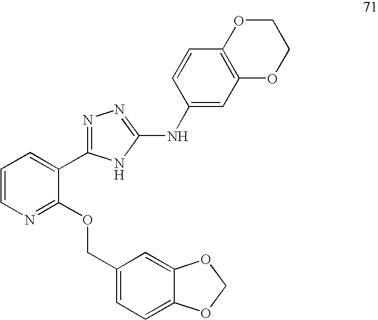

To a solution of 2-(benz[1,3]dioxol-5-ylmethoxy)-nicotinic acid hydrazide (71b, 120 mg, 0.34 mmol) in pyridine (2 ml), 1-(2,3-dihydro-benzo[1,4]dioxin-6-yl)-2-methyl-isothiourea (prepared according to 1f from Example, 144 mg, 0.406 mmol) and triethylamine (0.1 ml) were added. The reaction mixture stirred at ambient temperature for 30 minutes, then the temperature was raised to 120° C. for 5 hours. The mixture was poured into water (15 ml), and extracted with ethyl acetate (15 ml×3). The combined organic layer was dried over anhydrous Na₂SO₄, filtered and evaporated. Dichloromethane was added to the crude, and a yellow solid precipitated out. After filtration and vacuum-drying, 60 mg of 71 was obtained in 40% yield. ¹HNMR (DMSO-d₆) δ (ppm) 8.87 (s, 1H), 8.75 (s, 1H), 8.02-8.14 (m, 2H), 7.40-7.44 (m, 1H), 7.00-7.08 (m, 1H), 6.92-6.96 (m, 1H), 6.7-6.80 (m, 2H), 6.64-6.68 (m, 1H), 6.42-6.50 (m, 2H), 6.23-6.28 (m, 1H), 5.85 (s, 2H), 3.90-4.02 (m, 6H). MS m/z: 446 (M+1).

Compounds 72 and 73 were prepared using method described in Example 5:

### Analytical Data:

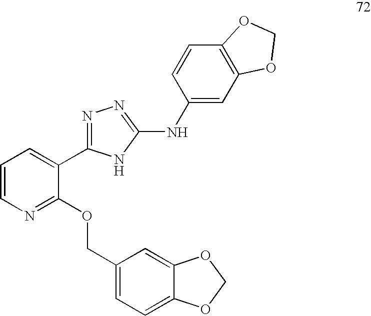

Benzo[1,3]dioxol-5-yl-{5-[2-(benzo[1,3]dioxol-5-ylmethoxy)-pyridin-3-yl]-4H-[1,2,4]triazol-3-yl}-amine (72): ¹HNMR (DMSO-d₆) δ (ppm) 13.25 (s, 1H), 9.23 (s, 1H), 8.25-8.40 (m, 2H), 7.39 (s, 1H), 7.22-7.28 (m, 2H), 7.05-7.10 (m, 2H), 6.85-6.94 (m, 2H), 6.01 (d, 4H), 5.60 (s, 2H). MS m/z: 432 (M+1).

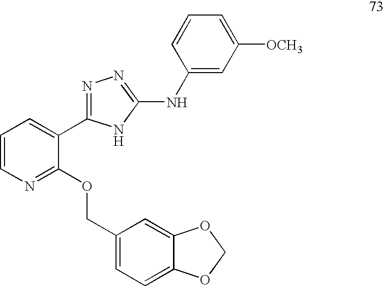

{5-[2-(Benzo[1,3]dioxol-5-ylmethoxy)-pyridin-3-yl]-4H-[1,2,4]triazol-3-yl}-(3-methoxy-phenyl)-amine (73): ¹HNMR (DMSO-d₆) (ppm) 9.33 (s, 1H), 8.30-8.40 (m, 2H), 7.41 (s, 1H), 7.20-7.32 (m, 4H), 7.07-7.11 (m, 1H), 6.90-6.94 (m, 1H), 6.45-6.48 (m, 1H), 6.03 (s, 2H), 5.60 (s, 2H), 3.77 (s, 3H). MS m/z: 418 (M+1).

### Example 6

### Synthesis of (3,5-Dimethoxy-phenyl)-{3-[5-(4-methanesulfonyl-phenyl)-4H-[1,2,4]triazol-3-yl]-pyridin-2-yl}-amine (74)

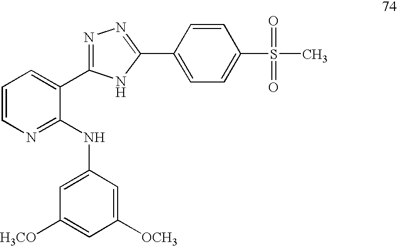

Compound 74 was synthesized by heating a mixture of 2-(3,5-dimethoxy-phenylamino)-nicotinic acid hydrazide (1c from Example 1, 180 mg, 0.743 mmol), 4-methanesulfonyl-benzamidine hydrochloride (179.8 mg, 0.766 mmol, purchased from J&W Pharmlab, PA), pyridine (2 ml) and triethylamine (0.15 ml) at 140° C. for 12 hours. The reaction solution was poured into water (15 ml), and extracted three times with ethyl acetate (15 ml). The combined organic layer was washed with brine, and dried over anhydrous Na₂SO4. After filtration, the organic phase was evaporated, then the residue was washed with hot methanol to give 188 mg (53% yield) of product. ¹HNMR (DMSO-d₆) δ (ppm) 15.15 (s, 1H), 11.00 (s, 1H), 8.35-8.44 (m, 4H), 8.05-8.12 (m, 2H), 7.10 (s, 2H), 6.92-6.96 (m, 1H), 6.14 (s, 1H), 3.81 (s, 6H), 3.21 (s, 3H). MS m/z: 452 (M+1).

Compounds 75 to 110 were synthesized using method described in Example 6:

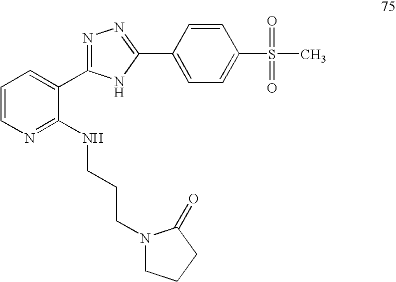

1-(3-{3-[5-(4-Methanesulfonyl-phenyl)-4H-[1,2,4]triazol-3-yl]-pyridin-2-ylamino}-propyl)-pyrrolidin-2-one (75): ¹HNMR (CDCl₃) δ (ppm) 8.45-8.52 (m, 1H), 8.04-8.09 (m, 2H), 7.81-7.98 (m, 4H), 6.22-6.27 (m, 1H), 3.35-3.68 (m, 6H), 2.95 (s, 3H), 2.37-2.43 (m, 2H), 1.90-2.02 (m, 4H). MS m/z: 441 (M+1).

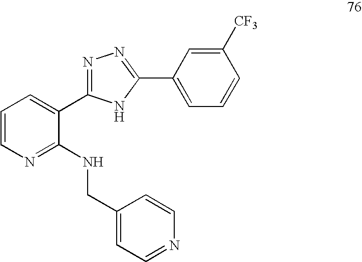

Pyridin-4-ylmethyl-{3-[5-(3-trifluoromethyl-phenyl)-4H-[1,2,4]triazol-3-yl]-pyridin-2-yl}-amine (76): ¹HNMR (DMSO-d₆) δ (ppm) 14.6 (br. s, 1H), 8.4 (m, 7H), 7.8 (m, 2H), 7.2 (d, 2H), 6.7 (m, 2H), 4.6 (d, 2H). ¹³C NMR (75 MHz, (CD3)₂SO) δ 153.6, 148.6, 148.5, 134.2, 129.2, 129.0, 128.9, 128.5, 128.1, 125.2, 124.8, 121.2, 121.0, 110.9, 42.3. MS m/z: 397 (M+1).

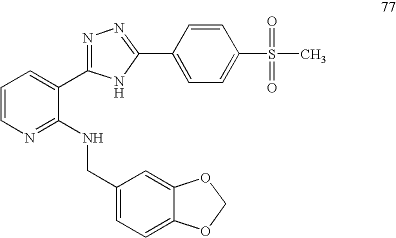

Benzo[1,3]dioxol-5-ylmethyl-{3-[5-(4-methanesulfonyl-phenyl)-4H-[1,2,4]triazol-3-yl]-pyridin-2-yl}-amine (77): ¹HNMR (DMSO-d₆) δ (ppm) 8.89 (s, 1H), 8.15-8.35 (m, 4H), 7.98-8.02 (m, 2H), 7.00 (s, 1H), 7.10 (s, 2H), 6.68-6.78 (m, 1H), 6.00 (s, 2H), 4.68 (d, 2H), 3.28 (s, 3H). MS m/z: 450 (M+1).

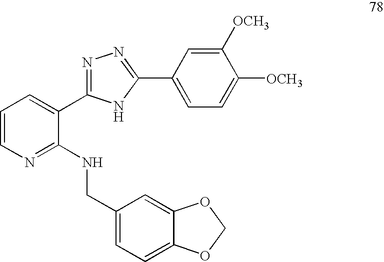

Benzo[1,3]dioxol-5-ylmethyl-{3-[5-(3,4-dimethoxy-phenyl)-4H-[1,2,4]triazol-3-yl]-pyridin-2-yl}-amine (78): ¹HNMR (DMSO-d₆) δ (ppm) 14.44 (s, 1H), 8.16-8.45 (m, 2H), 7.65-7.75 (m, 2H), 6.68-7.15 (m, 5H), 6.06 (s, 2H), 4.66 (d, 2H), 3.83-3.90 (d, 6H). MS m/z: 432 (M+1).

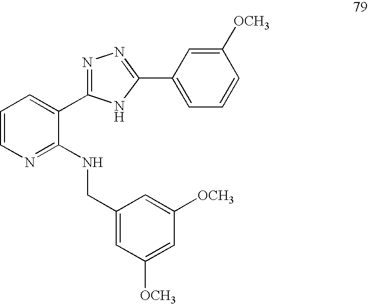

(3,5-Dimethoxy-benzyl)-{3-[5-(3-methoxy-phenyl)-4H-[1,2,4]triazol-3-yl]-pyridin-2-yl}-amine (79): ¹HNMR (DMSO-d₆) δ (ppm) 14.72 (s, 1H), 8.18-8.50 (m, 3H), 7.44-7.65 (m, 3H), 7.08 (s, 1H), 6.72-6.77 (m, 1H), 6.61 (d, 2H), 6.42 (d, 1H), 4.71 (d, 2H), 3.71-3.81 (m, 9H). MS m/z: 418 (M+1).

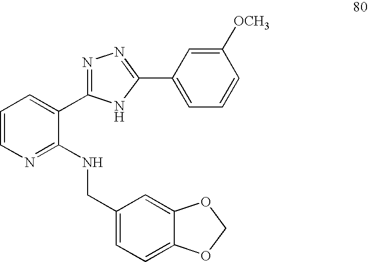

Benzo[1,3]dioxol-5-ylmethyl-{3-[5-(3-methoxy-phenyl)-4H-[1,2,4]triazol-3-yl]-pyridin-2-yl}-amine (80): ¹HNMR (DMSO-d₆) δ (ppm) 8.75 (s, 1H), 8.27-8.30 (m, 1H), 8.16-8.18 (m, 1H), 7.46-7.64 (m, 2H), 7.41-7.43 (m, 1H), 7.00-7.07 (m, 2H), 6.87-6.93 (m, 2H), 6.71-6.75 (m, 1H), 6.00 (s, 2H), 4.66 (d, 2H), 3.81 (s, 3H). MS m/z: 402 (M+1).

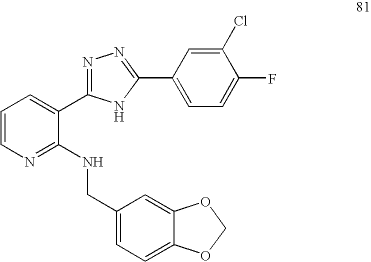

Benzo[1,3]dioxol-5-ylmethyl-{3-[5-(3-chloro-4-fluoro-phenyl)-4H-[1,2,4]triazol-3-yl]-pyridin-2-yl}-amine (81): ¹HNMR (DMSO-d₆) δ (ppm) 8.87 (s, 1H), 8.10-8.25 (m, 5H), 7.50-7.60 (m, 1H), 6.70-6.95 (m, 4H), 6.00 (s, 2H), 4.69 (d, 2H). MS m/z: 424 (M+1).

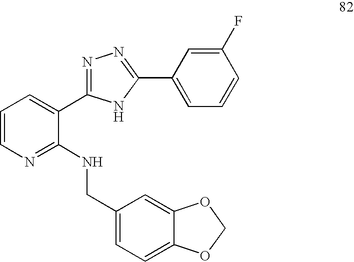

Benzo[1,3]dioxol-5-ylmethyl-{3-[5-(3-fluoro-phenyl)-4H-[1,2,4]triazol-3-yl]-pyridin-2-yl}-amine (82): ¹HNMR (DMSO-d₆) δ (ppm) 14.80 (s, br, 1H), 9.10 (s, br, 1H), 8.31-8.38 (m, 2H), 7.35-7.85 (m, 4H), 6.70-6.95 (m, 3H), 6.00 (s, 2H), 4.68 (d, 2H). MS m/z: 390 (M+1).

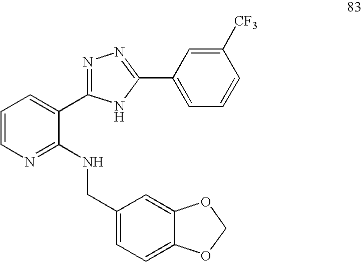

Benzo[1,3]dioxol-5-ylmethyl-{3-[5-(3-trifluoromethyl-phenyl)-4H-[1,2,4]triazol-3-yl]-pyridin-2-yl}-amine (83): ¹HNMR (DMSO-d₆) δ (ppm) 8.80 (s, 1H), 8.15-8.38 (m, 4H), 7.70-7.85 (m, 2H), 7.00 (s, 1H), 6.85-6.90 (m, 2H), 6.72-6.78 (m, 1H), 6.00 (s, 2H), 4.66 (d, 2H). MS m/z: 440 (M+1).

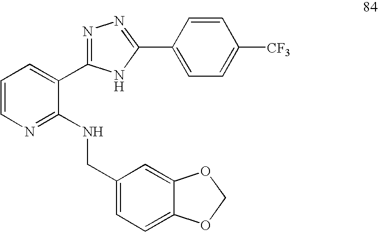

Benzo[1,3]dioxol-5-ylmethyl-{3-[5-(4-trifluoromethyl-phenyl)-4H-[1,2,4]triazol-3-yl]-pyridin-2-yl}-amine (84): ¹HNMR (DMSO-d₆) δ (ppm) 14.85 (s, br, 1H), 8.80 (s, br, 1H), 8.15-8.25 (m, 4H), 7.85-7.92 (m, 2H), 7.00 (s, 1H), 7.11 (s, 2H), 6.68-6.72 (m, 1H), 6.00 (s, 2H), 4.67 (d, 2H). MS m/z: 440 (M+1).

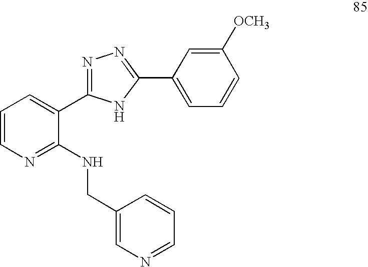

{3-[5-(3-Methoxy-phenyl)-4H-[1,2,4]triazol-3-yl]-pyridin-2-yl}-pyridin-3-ylmethyl-amine (85): ¹HNMR (DMSO-d₆) δ (ppm) 14.71 (s, 1H), 8.65 (s, 1H), 8.48-8.59 (m, 1H), 8.10-8.30 (m, 2H), 7.78-7.82 (m, 1H), 7.50-7.65 (m, 2H), 7.30-7.45 (m, 2H), 7.02-7.08 (m, 1H), 6.70-6.80 (m, 1H), 4.80-4.82 (m, 2H), 3.82 (s, 3H). MS m/z: 359 (M+1).

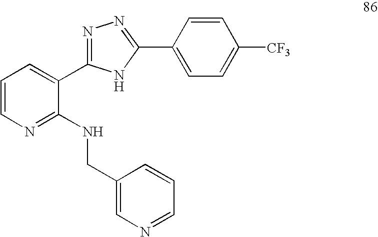

Pyridin-3-ylmethyl-{3-[5-(4-trifluoromethyl-phenyl)-4H-[1,2,4]triazol-3-yl]-pyridin-2-yl}-amine (86): ¹HNMR (DMSO-d₆) δ (ppm) 14.90 (s, 1H), 8.91 (s, 1H), 8.68 (s, 1H), 8.47 (d, 1H), 8.15-8.30 (m, 4H), 7.72-7.95 (m, 3H), 7.35-7.40 (m, 1H), 6.70-6.79 (m, 1H), 4.83 (d, 2H). MS m/z: 397 (M+1).

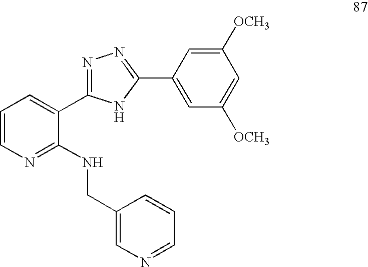

{3-[5-(3,5-Dimethoxy-phenyl)-4H-[1,2,4]triazol-3-yl]-pyridin-2-yl}-pyridin-3-ylmethyl-amine (87): ¹HNMR (DMSO-d₆) δ (ppm) 14.72 (s, 1H), 8.66 (s, 1H), 8.47-8.48 (m, 1H), 8.17-8.31 (m, 2H), 7.82 (d, 1H), 7.35-7.39 (m, 1H), 7.20-7.21 (d, 2H), 6.63-6.78 (m, 2H), 4.80-4.82 (m, 2H), 3.80 (s, 6H). MS m/z: 389 (M+1).

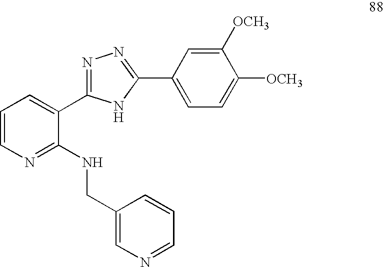

{3-[5-(3,4-Dimethoxy-phenyl)-4H-[1,2,4]triazol-3-yl]-pyridin-2-yl}-pyridin-3-ylmethyl-amine (88): ¹HNMR (DMSO-d₆) δ (ppm) 14.50 (s, 1H), 8.65-8.66 (m, 1H), 8.47-8.49 (m, 1H), 8.16-8.18 (m, 1H), 7.80-7.83 (m, 2H), 7.61-7.65 (m, 2H), 7.35-7.40 (m, 1H), 7.11 (d, 1H), 6.73-6.77 (m, 1H), 4.80 (d, 2H), 3.83 (s, 3H), 3.80 (s, 3H). MS m/z: 389 (M+1).

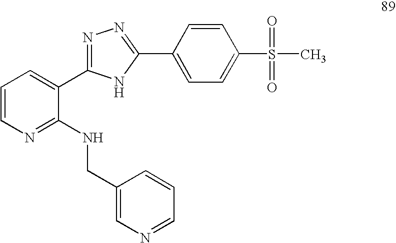

{3-[5-(4-Methanesulfonyl-phenyl)-4H-[1,2,4]triazol-3-yl]-pyridin-2-yl}-pyridin-3-ylmethyl-amine (89): ¹HNMR (DMSO-d₆) δ (ppm) 14.90 (s, 1H), 8.91 (s, 1H), 8.66 (s, 1H), 8.02-8.35 (m, 6H), 7.81-7.84 (m, 1H), 7.32-7.39 (m, 1H), 6.75-6.80 (m, 1H), 4.83 (d, 2H), 3.33 (s, 3H). MS m/z: 407 (M+1).

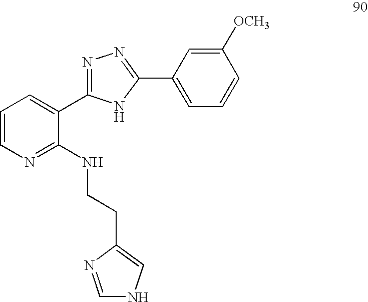

[2-(1H-Imidazol-4-yl)-ethyl]-{3-[5-(3-methoxy-phenyl)-4H-[1,2,4]triazol-3-yl]-pyridin-2-yl}-amine (90): ¹HNMR (DMSO-d₆) δ (ppm) 14.40 (s, br, 1H), 1.57 (s, br, 1H), 8.55 (s, 1H), 8.15-8.22 (m, 2H), 7.42-7.65 (m, 4H), 7.07-7.10 (m, 1H), 6.66-6.68 (m, 1H), 6.65-6.74 (m, 1H), 3.92 (s, 3H), 3.78-3.86 (m, 1H), 2.85-2.92 (m, 2H). MS m/z: 362 (M+1).

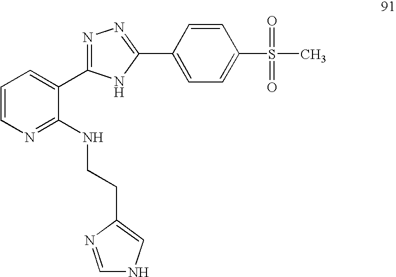

[2-(1H-Imidazol-4-yl)-ethyl]-{3-[5-(4-methanesulfonyl-phenyl)-4H-[1,2,4]triazol-3-yl]-pyridin-2-yl}-amine (91): ¹HNMR (DMSO-d₆) δ (ppm) 8.66 (s, 1H), 8.15-8.30 (m, 6H), 7.65 (m, 1H), 6.96 (s, 1H), 6.70-6.76 (m, 1H), 3.89-3.95 (m, 2H), 3.43 (s, 3H), 2.91-3.00 (m 2H). MS m/z: 410 (M+1).

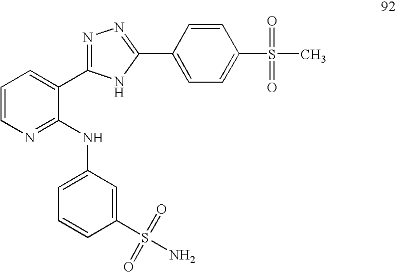

3-{3-[5-(4-Methanesulfonyl-phenyl)-4H-[1,2,4]triazol-3-yl]-pyridin-2-ylamino}-benzenesulfonamide (92): ¹HNMR (DMSO-d₆) δ (ppm) 15.16 (s, 1H), 11.14 (s, 1H), 8.30-8.48 (m, 4H), 8.10-8.14 (m, 2H), 7.89-7.92 (m, 1H), 7.35-7.56 (m, 2H), 7.25 (s, 2H), 6.95-7.00 (m, 1H), 3.28 (s, 3H). MS m/z: 471 (M+1).

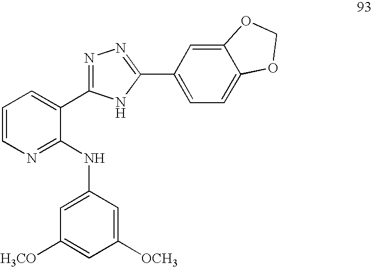

[3-(5-Benzo[1,3]dioxol-5-yl-4H-[1,2,4]triazol-3-yl)-pyridin-2-yl]-(3,5-dimethoxy-phenyl)-amine (93): ¹HNMR (DMSO-d₆) δ (ppm) 14.75 (s, 1H), 11.00 (s, 1H), 8.50-8.54 (m, 1H), 8.30-8.32 (m, 1H), 7.70-7.74 (m, 1H), 7.62 (s, 1H), 7.88-8.20 (m, 4H), 6.05 (s, 2H), 3.80 (s, 6H). MS m/z: 418 (M+1).

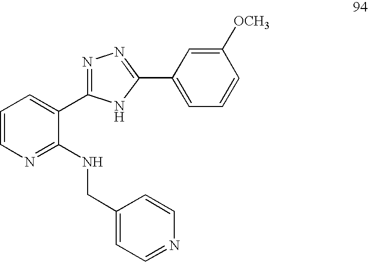

{3-[5-(3-Methoxy-phenyl)-4H-[1,2,4]triazol-3-yl]-pyridin-2-yl}-pyridin-4-ylmethyl-amine (94): ¹HNMR (DMSO-d₆) δ (ppm) 14.43 (s, 1H), 8.66 (s, 1H), 8.21-8.24 (m, 2H), 7.98-8.01 (m, 1H), 7.81-7.83 (m, 1H), 7.37-7.41 (m, 2H), 7.10-7.21 (m, 3H), 6.80-6.83 (m, 1H), 6.48-6.51 (m, 1H), 4.61 (d, 2H), 3.60 (s, 3H). MS m/z: 359 (M+1).

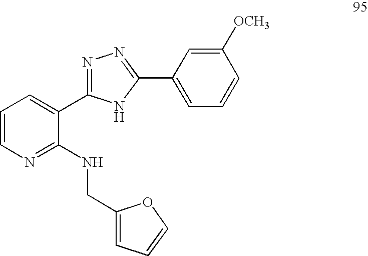

Furan-2-ylmethyl-{3-[5-(3-methoxy-phenyl)-4H-[1,2,4]triazol-3-yl]-pyridin-2-yl}-amine (95): ¹HNMR (DMSO-d₆) δ (ppm) 14.5 (s, 1H), 8.65 (s, 1H), 8.20-8.22 (m, 1H), 8.04-8.05 (m, 1H), 7.51-7.59 (m, 3H), 7.35-7.40 (m, 1H), 6.92-7.00 (m, 1H), 6.60-6.64 (m, 1H), 6.20-6.28 (m, 2H), 4.66 (d, 2H), 3.77 (s, 3H). MS m/z: 348 (M+1).

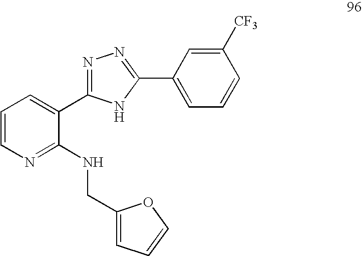

Furan-2-ylmethyl-{3-[5-(3-trifluoromethyl-phenyl)-4H-[1,2,4]triazol-3-yl]-pyridin-2-yl}-amine (96): ¹HNMR (DMSO-d₆) δ (ppm) 8.20-8.28 (m, 4H), 8.10-8.12 (m, 1H), 7.90-7.96 (m, 1H), 7.81-7.85 (m, 1H), 7.55-7.64 (m, 1H), 7.30 (s, 1H), 6.63-6.70 (m, 1H), 6.00-6.10 (m, 2H), 4.55 (d, 2H). MS m/z: 386 (M+1).

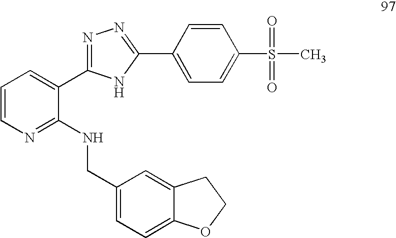

(2,3-Dihydro-benzofuran-5-ylmethyl)-{3-[5-(4-methanesulfonyl-phenyl)-4H-[1,2,4]triazol-3-yl]-pyridin-2-yl}-amine (97): ¹HNMR (DMSO-d₆) δ (ppm) 15.00 (s, 1H), 8.90 (s, 1H), 8.30-8.41 (m, 4H), 8.05-8.12 (m, 2H), 7.40 (s, 1H), 7.24-7.26 (m, 1H), 6.80-6.9 (m, 2H), 4.79 (d, 2H), 4.53-4.60 (m, 2H), 3.30 (s, 3H), 3.15-3.21 (m, 2H). MS m/z: 448 (M+1).

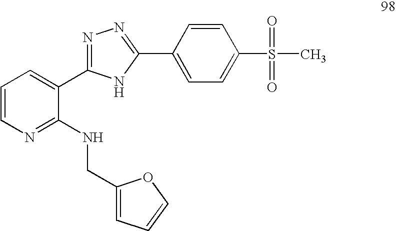

Furan-2-ylmethyl-{3-[5-(4-methanesulfonyl-phenyl)-4H-[1,2,4]triazol-3-yl]-pyridin-2-yl}-amine (98): ¹HNMR (DMSO-d₆) δ (ppm) 14.83 (s, 1H), 8.85 (s, 1H), 8.20-8.30 (m, 4H), 8.05-8.10 (m, 2H), 7.70 (s, 1H), 6.78-6.83 (m, 1H), 6.33-6.41 (m, 2H), 4.77 (d, 2H), 3.23 (s, 3H). MS m/z: 396 (M+1).

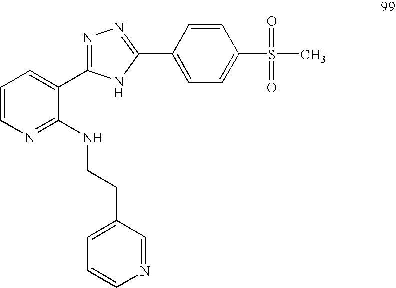

{3-[5-(4-Methanesulfonyl-phenyl)-4H-[1,2,4]triazol-3-yl]-pyridin-2-yl}-(2-pyridin-3-yl-ethyl)-amine (99): ¹HNMR (DMSO-d₆) δ (ppm) 14.91 (s, 1H), 8.44-8.59 (m, 3H), 8.05-8.21 (m, 6H), 7.83-7.86 (m, 1H), 7.33-7.37 (m, 1H), 6.73-6.78 (m, 1H), 3.87-3.92 (m, 2H), 3.28 (s, 3H), 3.02-3.08 (m, 2H). MS m/z: 421 (M+1).

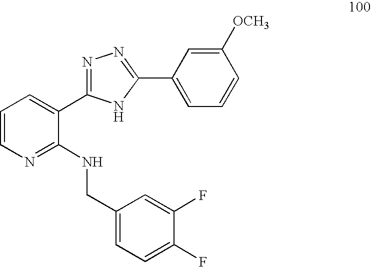

(3,4-Difluoro-benzyl)-{3-[5-(3-methoxy-phenyl)-4H-[1,2,4]triazol-3-yl]-pyridin-2-yl}-amine (100): ¹HNMR (DMSO-d₆) δ (ppm) 8.90 (s, 1H), 8.32-8.36 (m, 1H), 8.18-8.20 (m, 1H), 7.65-7.73 (m, 2H), 7.35-7.51 (m, 3H), 7.28-7.31 (m, 1H), 7.10-7.14 (m, 1H), 6.73-6.84 (m, 1H), 4.80 (d, 2H), 3.84 (s, 3H). MS m/z: 394 (M+1).

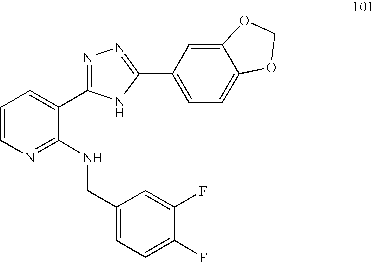

[3-(5-Benzo[1,3]dioxol-5-yl-4H-[1,2,4]triazol-3-yl)-pyridin-2-yl]-(3,4-difluoro-benzyl)-amine (101): ¹HNMR (DMSO-d₆) δ (ppm) 8.98 (s, 1H), 8.42-8.48 (m, 1H), 8.29-8.33 (m, 1H), 7.76-7.86 (m, 2H), 7.50-7.60 (m, 2H), 7.36-7.40 (m, 1H), 7.20-7.24 (m, 1H), 6.86-6.91 (m, 1H), 6.22 (s, 2H). MS m/z: 408 (M+1).

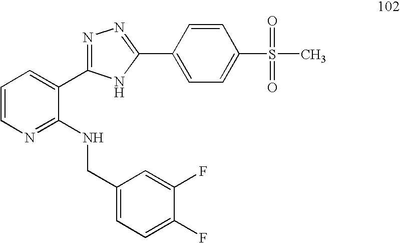

(3,4-Difluoro-benzyl)-{3-[5-(4-methanesulfonyl-phenyl)-4H-[1,2,4]triazol-3-yl]-pyridin-2-yl}-amine (102): ¹HNMR (DMSO-d₆) δ (ppm) 8.92 (s, 1H), 8.00-8.31 (m, 6H), 7.32-7.41 (m, 2H), 7.22-7.24 (m, 1H), 6.65-6.72 (m, 1H), 4.69 (d, 2H), 3.25 (s, 3H). MS m/z: 442 (M+1).

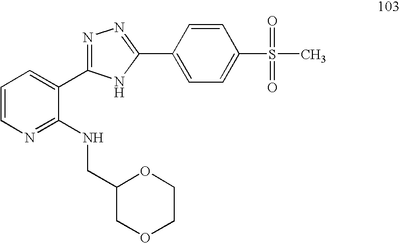

[1,4]Dioxan-2-ylmethyl-{3-[5-(4-methanesulfonyl-phenyl)-4H-[1,2,4]triazol-3-yl]-pyridin-2-yl}-amine (103): ¹HNMR (DMSO-d₆) δ (ppm) 8.85 (s, 1H), 8.10-8.41 (m, 6H), 6.70-6.76 (m, 1H), 3.80-3.95 (m, 6H), 3.35-3.55 (m, 3H), 3.26 (s, 3H). MS m/z: 431 (M+1).

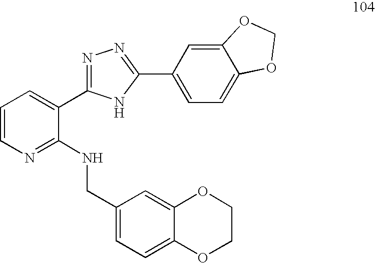

[3-(5-Benzo[1,3]dioxol-5-yl-4H-[1,2,4]triazol-3-yl)-pyridin-2-yl]-(2,3-dihydro-benzo[1,4]dioxin-6-ylmethyl)-amine (104): ¹HNMR (DMSO-d₆) δ (ppm) 8.71 (s, 1H), 8.06-0.23 (m, 2H), 7.40-7.52 (m, 2H), 6.94-7.01 (m, 1H), 6.75-6.89 (m, 3-H), 6.58-6.63 (m, 1H), 6.03 (s, 2H), 4.54-4.56 (m, 2H), 4.10-4.16 (m, 4H). MS m/z: 430 (M+1).

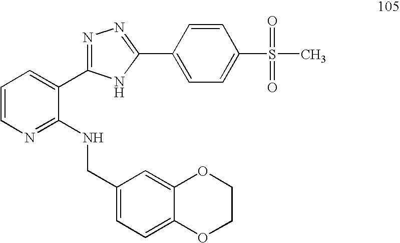

(2,3-Dihydro-benzo[1,4]dioxin-6-ylmethyl)-{3-[5-(4-methanesulfonyl-phenyl)-4H-[1,2,4]triazol-3-yl]-pyridin-2-yl}-amine (105): ¹HNMR (DMSO-d₆) δ (ppm) 10.10 (s, 1H), 8.10-8.30 (m, 5H), 6.71-6.90 (m, 4H), 4.64 (d, 2H), 4.14-4.18 (m, 4H), 3.20 (s, 3H). MS m/z: 464 (M+1).

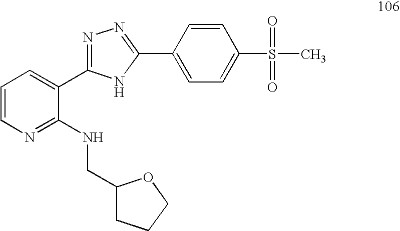

{3-[5-(4-Methanesulfonyl-phenyl)-4H-[1,2,4]triazol-3-yl]-pyridin-2-yl}-(tetrahydro-furan-2-ylmethyl)-amine (106): ¹HNMR (DMSO-d₆) δ (ppm) 14.95 (s, 1H), 8.89 (s, 1H), 8.10-8.45 (m, 6H), 6.77-6.83 (m, 1H), 4.18-4.22 (m, 1H), 3.99-4.01 (m, 1H), 3.80-3.90 (m, 2H), 3.55-3.65 (m, 1H), 3.35 (s, 3H), 1.95-2.14 (m, 3H), 1.62-1.75 (m, 1H). MS m/z: 400 (M+1).

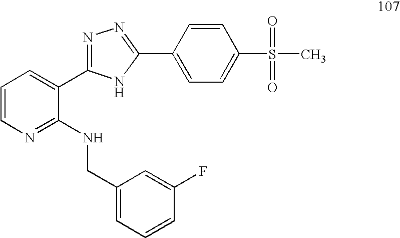

(3-Fluoro-benzyl)-{3-[5-(4-methanesulfonyl-phenyl)-4H-[1,2,4]triazol-3-yl]-pyridin-2-yl}-amine (107): ¹HNMR (DMSO-d₆) δ (ppm) 8.15-8.62 (m, 7H), 7.05-7.40 (m, 4H), 6.78-6.83 (m, 1H), 4.87 (m, 2H), 3.45 (s, 3H). MS m/z: 424 (M+1).

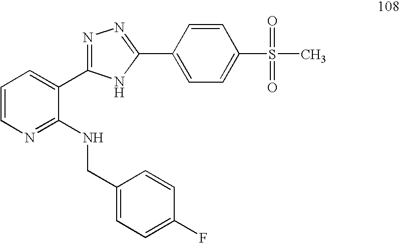

(4-Fluoro-benzyl)-{3-[5-(4-methanesulfonyl-phenyl)-4H-[1,2,4]triazol-3-yl]-pyridin-2-yl}-amine (108): ¹HNMR (DMSO-d₆) δ (ppm) 14.65 (s, 1H), 9.00 (s, 1H), 8.05-8.33 (m, 6H), 7.44-7.46 (m, 2H), 7.25-7.28 (m, 2H), 6.81-6.83 (m, 1H), 4.86-4.90 (m, 2H), 3.27 (s, 3H). MS m/z: 424 (M+1).

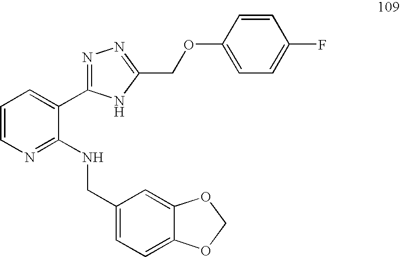

Benzo[1,3]dioxol-5-ylmethyl-{3-[5-(4-fluoro-phenoxymethyl)-4H-[1,2,4]triazol-3-yl]-pyridin-2-yl}-amine (109) synthesized from 2-(4-Fluoro-phenoxy)-acetamidine (purchased from J&W Pharmlab). ¹HNMR (ethanol-d₄) δ (ppm) 8.18 (s, 1H), 7.97-8.01 (m, 1H), 7.90-7.96 (m, 5H), 6.24-6.27 (m, 2H), 6.55-6.60 (m, 2H), 5.81 (s, 2H), 5.12 (s, 2H), 4.50 (s, 1H). MS m/z: 420 (M+1).

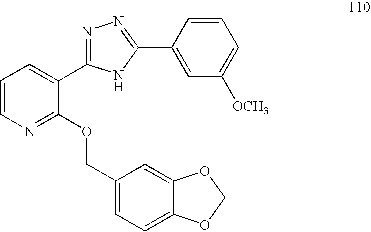

2-(Benzo[1,3]dioxol-5-ylmethoxy)-3-[5-(3-methoxy-phenyl)-4H-[1,2,4]triazol-3-yl]-pyridine (110): prepared using 71b (from Example 5) as starting material: ¹HNMR (DMSO-d₆) δ (ppm) 14.13 (s, 1H), 8.47-8.52 (m, 1H), 8.30-8.32 (m, 1H), 7.70-7.80 (m, 2H), 7.47-7.53 (m, 1H), 7.23-7.26 (m, 2H), 7.04-7.08 (m, 2H), 6.90-6.92 (m, 1H), 6.03 (s, 2H), 3.90 (s, 3H). MS m/z: 403 (M+1).

### Example 7

### synthesis of benzo[1,3]dioxol-5-yl-(5-{2-[(pyridin-4-ylmethyl)-amino]-phenyl}-4H-[1,2,4]triazol-3-yl)-amine (111)

### Step 1—synthesis of 2-[(pyridin-4-ylmethyl)-amino]-benzoic acid Methyl Ester (111a)

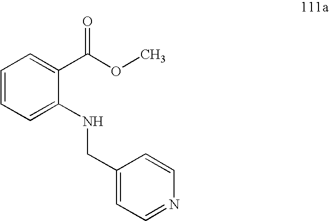

A mixture of methyl anthranilate (7.5 g, purchased from Aldrich) and 4-pyridylaldehyde (8.6 g, purchased from Aldrich) in methanol (300 ml) and acetic acid (3 ml) was stirred at room temperature for 12 hours. NaBH₃CN (6.9 g) was added to the reaction, and the resulting solution was stirred at ambient temperature for 12 hr. The reaction mixture was concentrated and the residue was dissolved in ethyl acetate and washed with saturated aqueous NaHCO₃ and brine. The organic layer was dried with MgSO₄, filtered, concentrated and purified by flash chromatography over silica gel (1:1 ethyl acetate/hexane) to give 10.2 g of 111a as yellow oil in 85% yield. ¹HNMR (DMSO-d₆) δ (ppm) 8.50 (d, J=6.0 Hz, 2H), 8.22 (t, J=6.0 Hz, 1H), 7.83 (d, J=8.0 Hz, 1H), 7.32 (d, J=5.7 Hz, 3H), 6.60 (t, J=9.0 Hz, 2H), 4.56 (d, J=6.3 Hz, 2H), 3.84 (s, 3H). MS m/z: 243 (M+1).

### Step 2—preparation of 2-[(Pyridin-4-ylmethyl)-amino]-benzoic acid Hydrazide (111b)

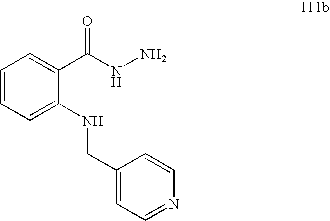

A mixture of 2-[(Pyridin-4-ylmethyl)-amino]-benzoic acid methyl ester (111a, 10 g) in hydrazine (50 ml) was refluxed. After 2 hr. the excess hydrazine was removed and the remaining mixture was dissolved in dichloromethane, washed with brine and dried with MgSO₄ and concentrated. The crude residue was purified by flash chromatography to give 9.2 g of 111b as white solid in 87% yield. ¹HNMR (DMSO-d₆) δ (ppm) 9.64 (s, 1H), 8.42-8.66 (m, 2H), 8.20 (t, J=6.0 Hz, 1H), 7.50 (d, J=7.8 Hz, 1H), 7.33 (d, J=4.80 Hz, 2H), 7.04-7.25 (t, J=7.8 Hz, 1H), 6.42-6.70 (m, 2H), 4.33-4.61 (m, 4H).

### Step 3—preparation of Benzo[1,3]dioxol-5-yl-(5-{2-[(pyridin-4-ylmethyl)-amino]-phenyl}-4H-[1,2,4]triazol-3-yl)-amine (111)

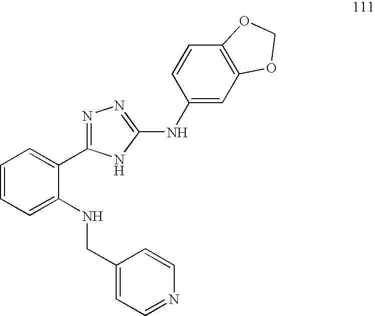

To a solution of 2-[(pyridin-4-yl)-amino]-benzoic acid hydrazide (111b, 200 mg, 0.82 mmol) in pyridine (2 ml), was added 1-benzo[1,3]dioxol-6-yl)-2-methyl-isothiourea (304.2 mg, 0.90 mmol, prepared according to the method for the preparation of compound 1f) and triethylamine (0.15 ml). The reaction mixture was stirred at 140° C. for 4 hours under argon. The reaction solution was poured into water (15 ml), then extracted with ethyl acetate (15 ml×3). The combined organic layer was dried over anhydrous Na₂SO₄. After filtration and evaporation, the organic residue was purified by silica gel column (dichloromethane:MeOH=125:1). Collected product 140 mg. Yield: 44.3%. ¹HNMR (DMSO-d₆) δ (ppm) 13.31 (s, 1H), 9.18 (s, 1H), 8.64-8.66 (m, 3H), 7.89 (s, 1H), 7.25-7.33 (m, 3H), 7.14-7.19 (m, 1H), 6.96-6.99 (m, 1H), 6.74-6.79 (m, 1H), 6.60-6.71 (m, 2H), 5.98 (s, 2H), 4.59 (d, 2H). MS m/z: 387 (M+1).

Compounds 112 to 123 were prepared using the method described in Example 7:

### Analytical Data:

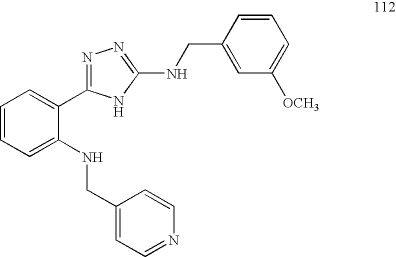

(3-Methoxy-benzyl)-(5-{2-[(pyridin-4-ylmethyl)-amino]-phenyl}-4H-[1,2,4]triazol-3-yl)-amine (112): ¹HNMR (DMSO-d₆) δ (ppm) 12.43 (s, 1H), 8.66-8.68 (m, 2H), 8.29-8.32 (m, 1H), 7.96-8.00 (m, 1H), 7.20-7.41 (m, 4H), 7.10-7.18 (m, 1H), 6.96-7.04 (m, 2H), 6.84-6.88 (m, 1H), 6.65-6.70 (m, 2H), 4.61-4.63 (m, 2H), 4.46-4.48 (m, 2H), 3.79 (s, 3H). MS m/z: 387 (M+1).

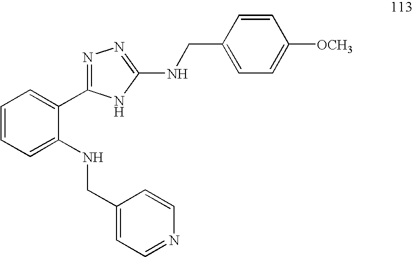

(4-Methoxy-benzyl)-(5-{2-[(pyridin-4-ylmethyl)-amino]-phenyl}-4H-[1,2,4]triazol-3-yl)-amine (113): ¹HNMR (DMSO-d₆) δ (ppm) 12.30 (s, 1H), 8.56-8.58 (m, 2H), 8.20-8.22 (m, 1H), 7.88-7.93 (m, 1H), 7.17-7), 7.00-7.06 (m, 1H), 6.80-6.87 (m, 2H), 6.40-6.60 (m, 2H), 4.51-4.54 (m, 2H), 4.31-4.33 (m, 2H), 3.71 (s, 3H). MS m/z: 387 (M+1).

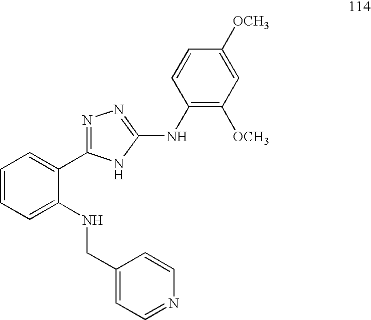

(2,4-Dimethoxy-phenyl)-(5-{2-[(pyridin-4-ylmethyl)-amino]-phenyl}-4H-[1,2,4]triazol-3-yl)-amine (114): ¹HNMR (DMSO-d₆) δ (ppm) 13.41 (s, 1H), 12.12 (s, 1H), 8.75 (s, 1H), 7.82-8.55 (m, 4H), 7.73-7.33 (m, 2H), 7.15 (s, 1H), 6.40-6.75 (m, 4H), 4.55 (s, 2H), 3.96 (s, 3H), 3.82 (s, 3H). MS m/z: 403 (M+1).

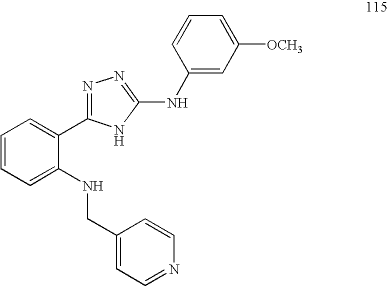

(3-Methoxy-phenyl)-(5-{2-[(pyridin-4-ylmethyl)-amino]-phenyl}-4H-[1,2,4]triazol-3-yl)-amine (115): ¹HNMR (methanol-d₄) δ (ppm) 8.40-8.45 (m, 2H), 7.74-7.80 (m, 1H), 7.42-7.45 (d, 2H), 7.08-7.35 (m, 3H), 6.94-6.98 (m, 3H), 6.95-6.98 (m, 1H), 4.59 (s, 2H), 3.75 (s, 3H). MS m/z: 373 (M+1).

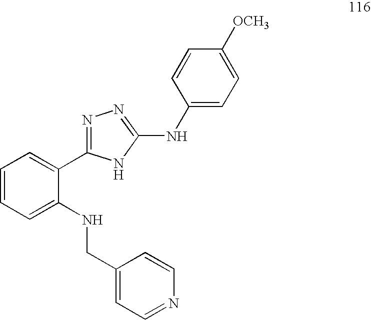

(4-Methoxy-phenyl)-(5-{2-[(pyridin-4-ylmethyl)-amino]-phenyl}-4H-[1,2,4]triazol-3-yl)-amine (116): ¹HNMR (DMSO-d₆) δ (ppm) 8.55-9.35 (m, 3H), 7.80-8.20 (m, 2H), 7.20-7.55 (m, 5H), 6.61-6.96 (m, 4H), 4.79 (s, 2H), 3.80 (s, 3H). MS m/z: 373 (M+1).

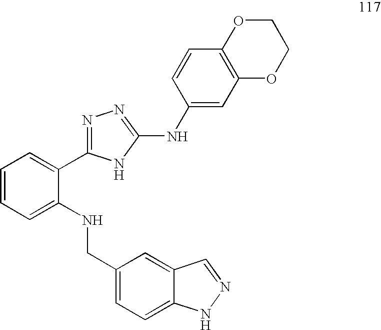

(2,3-Dihydro-benzo[1,4]dioxin-6-yl)-(5-{2-[(1H-indazol-5-ylmethyl)-amino]-phenyl}-4H-[1,2,4]triazol-3-yl)-amine (117): ¹HNMR (DMSO-d₆) δ (ppm) 13.53 (s, 1H), 13.05 (s, 1H), 9.00 (s, 1H), 8.08-8.09 (m, 2H), 7.80-7.84 (m, 1H), 7.60-7.65 (m, 1H), 7.40-7.43 (m, 1H), 7.25-7.30 (m, 2H), 6.96-7.01 (m, 1H), 6.72-6.85 (m, 3H), 4.68-4.70 (m, 2H), 4.20-4.25 (m, 4H). MS m/z: 440 (M+1).

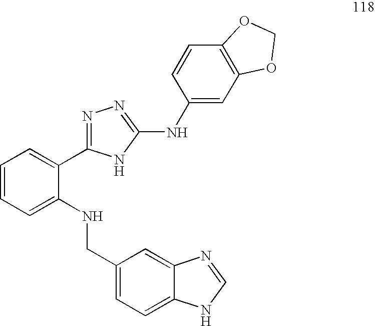

Benzo[1,3]dioxol-5-yl-(5-{2-[(1H-benzoimidazol-5-ylmethyl)-amino]-phenyl}-4H-[1,2,4]triazol-3-yl)-amine (118): ¹HNMR (DMSO-d₆) δ (ppm) 13.60 (s, 1H), 12.54 (s, 1H), 8.11 (m, 2H), 7.84-7.88 (m, 1H), 7.52-7.60 (m, 2H), 7.15-7.30 (m, 3H), 6.95-7.02 (m, 1H), 6.65-6.88 (m, 3H), 5.98 (s, 2H), 4.62 (s, 2H). MS m/z: 426 (M+1).

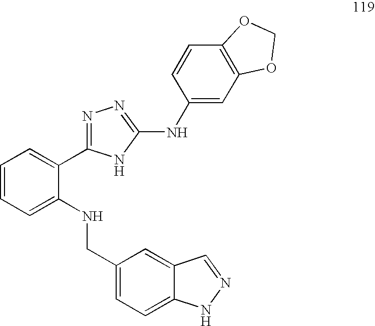

Benzo[1,3]dioxol-5-yl-(5-{2-[(1H-indazol-5-ylmethyl)-amino]-phenyl}-4H-[1,2,4]triazol-3-yl)-amine (119): ¹HNMR (DMSO-d₆) δ (ppm) 8.05 (s, 2H), 7.70 (s, 2H), 7.55-7.57 (m, 1H), 7.36-7.39 (m, 1H), 7.20-7.30 (m, 2H), 7.02-7.07 (m, 1H), 6.84-6.92 (m, 3H), 6.00 (s, 2H), 4.76 (s, 2H). MS m/z: 426 (M+1).

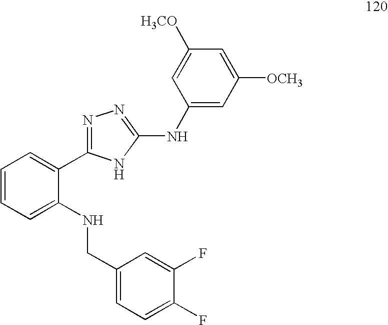

{5-[2-(3,4-Difluoro-benzylamino)-phenyl]-4H-[1,2,4]triazol-3-yl}-(3,5-dimethoxy-phenyl)-amine (120): ¹HNMR (DMSO-d₆) δ (ppm) 8.15-8.32 (m, 2H), 7.40-7.47 (m, 2H), 7.20 (s, 1H), 6.73-6.88 (m, 3H), 6.81-6.89 (m, 3H), 6.10 (s, 1H), 4.78-4.80 (m, 2H), 3.73-3.75 (m, 6H). MS m/z: 438 (M+1).

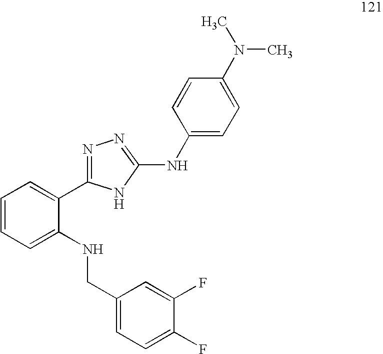

{5-[2-(3,4-Difluoro-benzylamino)-phenyl]-4H-[1,2,4]triazol-3-yl}-(4-dimethylamino-phenyl)-amine (121): ¹HNMR (DMSO-d₆) δ (ppm) 13.21 (s, 1H), 9.12 (s, 1H), 8.67 (m, 1H), 8.15-8.32 (m, 3H), 7.27-7.65 (m, 6H), 6.72-6.76 (m, 3H), 4.82-4.86 (m, 2H), 2.85-2.90 (m, 6H). MS m/z: 421 (M+1).

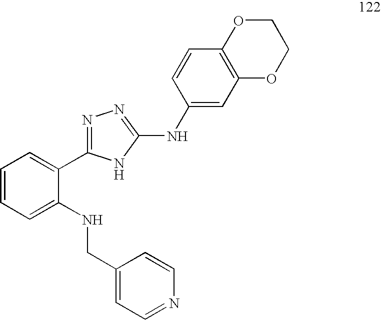

(2,3-Dihydro-benzo[1,4]dioxin-6-yl)-(5-{2-[(pyridin-4-ylmethyl)-amino]-phenyl}-4H-[1,2,4]triazol-3-yl)-amine (122): ¹HNMR (DMSO-d₆) δ (ppm) 13.50 (s, 1H), 12.62 (s, 1H), 9.02 (s, 1H), 8.48-8.52 (m, 2H), 7.80-7.83 (m, 1H), 7.30-7.32 (m, 2H), 7.18-7.21 (m, 2H), 6.90-6.93 (m, 2H), 6.65-6.79 (m, 2H), 4.61 (d, 2H), 4.20-4.32 (m, 4H). MS m/z: 401 (M+1).

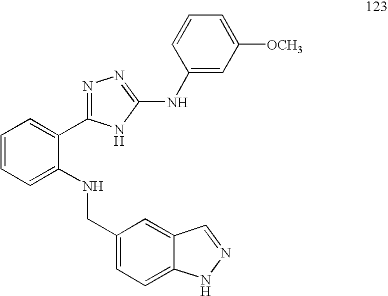

(5-{2-[(1H-Indazol-5-ylmethyl)-amino]-phenyl}-4H-[1,2,4]triazol-3-yl)-(3-methoxy-phenyl)-amine (123): ¹HNMR (DMSO-d₆) δ (ppm) 9.59 (s, 1H), 9.3 (s, 1H), 8.68-8.70 (m, 1H), 8.05-8.08 (m, 2H), 7.72-7.78 (m, 2H), 7.55-7.59 (m, 1H), 7.41-7.44 (m, 1H), 7.30 (s, 1H), 7.10-7.23 (m, 2H), 6.80-6.83 (m, 1H), 6.65-6.70 (m, 1H), 6.40-6.42 (m, 1H), 4.65 (m, 2H), 3.74 (m, 3H). MS m/z: 412 (M+1).

### Example 8

### synthesis of Benzo[1,3]dioxol-5-yl-(5-{2-[(pyridin-4-ylmethyl)-amino]-4-trifluoromethyl-phenyl}-4H-[1,2,4]triazol-3-yl)-amine (124)

### Step 1: synthesis of 2-Nitro-4-trifluoromethyl-benzoic acid Hydrazide (124a)

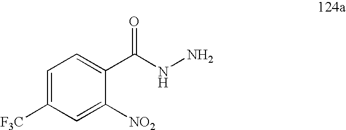

The reaction mixture of 2-nitro-4-trifluoromethyl-benzoic acid methyl ester (5.74 g, 0.023 mol, purchased from Aldrich), hydrazine monohydrate (3.46 ml, 0.060 mol) and 2-propanol (40 ml) was stirred at 80° C. under argon for overnight. A white solid precipitated out. Filtered and dried to obtain 3.0 g of 124a in 52% yield. ¹HNMR (DMSO-d₆) δ (ppm) 9.92 (s, 1H), 8.40-8.44 (m, 1H), 8.15-8.20 (m, 1H), 7.80-7.85 (m, 1H), 4.78 (s, 1H).

### Step 2: synthesis of [5-(2-Amino-4-trifluoromethyl-phenyl)-4H-[1,2,4]triazol-3-yl]-benzo[1,3]dioxol-5-yl-amine (124b)

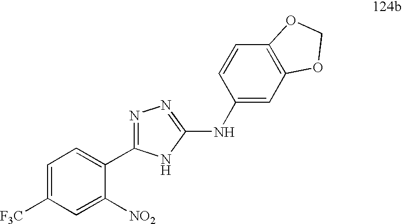

Above hydrazide (124a, 800 mg, 3.21 mmol) was put in sealed tube. Pyridine 10 ml, triethylamine (1.0 ml), and 1-benzo[1,3]dioxol-5-yl-2-methyl-isothiourea hydroiodide (1.41 g, 4.17 mmol) were added, then the reaction mixture was stirred at 130° C. for overnight. The reaction solution to cooled to room temperature then poured into water (60 ml), and extracted with ethyl acetate (50 ml×3). The organic layer was washed with brine, and dried over anhydrous Na₂SO₄. After filtration and evaporation, the organic residue was purified with silica gel column (CH₂Cl₂:methanol=100:1) to obtain 638 mg of 124b in 68% yield. ¹HNMR (DMSO-d₆) δ (ppm) 13.17 (s, 1H), 9.49 (s, 1H), 8.05-8.38 (m, 3H), 7.23 (s, 1H), 6.87-6.92 (m, 2H), 6.03 (s, 2H).

### Step 3: Synthesis of [5-(2-Amino-4-trifluoromethyl-phenyl)-4H-[1,2,4]triazol-3-yl]-benzo[1,3]dioxol-5-yl-amine (124c)

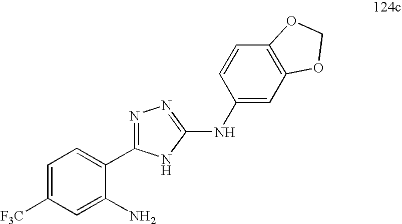

The nitro triazole compound 124b (800 mg), ethanol (60 ml), and 10% Pd—C (160 mg) was added in flask. The reaction mixture stirred at 60° C. for 3 hours, and a solid precipitated from the reaction solution. 60 ml chloroform was added, and the reaction mixture was stirred at 80° C. until the solid dissolved. The catalyst was filtered, and the filtrate was evaporated to obtain 643 mg of 124c in 88.4% yield. ¹HNMR (DMSO-d₆) δ (ppm) 9.15-9.25 (t, 1H), 8.03 (s, 1H), 7.34-7.36 (m, 1H), 7.15-7.17 (m, 1H), 6.85-7.10 (m, 4H), 6.00 (s, 2H). MS m/z: 364 (M+1).

### Step 4: synthesis of Benzo[1,3]dioxol-5-yl-(5-{2-[(pyridin-4-ylmethyl)-amino]-4-trifluoromethyl-phenyl}-4H-[1,2,4]triazol-3-yl)-amine (124)

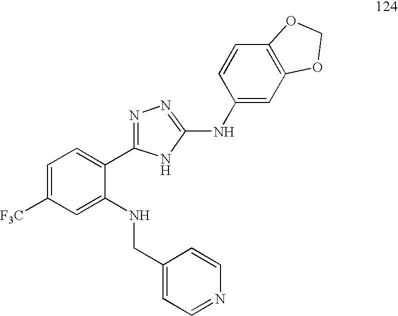

To a solution of [5-2-amino-4-trifluoromethyl-phenyl)-4H-[1,2,4]triazol-3-yl]-benzo[1,3]dioxol-5-yl-amine (124c, 57 mg, 0.157 mmol) in anhydrous dichloroethane (5 ml) was added pyridine-4-carbaldehyde (17 μl, 0.173 mmol), sodium triacetoxyborohydride (87.1 mg, 0.393 mmol), acetic acid (0.157 mmol). The reaction mixture was stirred at ambient temperature for 8 hours. The reaction was quenched with aqueous 2N NaOH then extracted with ethyl acetate (20 ml×3). The organic layer was washed with brine, then dried over anhydrous Na₂SO₄. After filtration and concentration, the residue was washed with hot methanol to obtain 30 mg of 124 in 42.3% yield. ¹HNMR (DMSO-d₆) δ (ppm) 9.34 (s, 1H), 8.53-8.57 (m, 2H), (m, 1H), 7.38-7.40 (m, 2H), 7.25 (s, 1H), 6.95-7.03 (m, 2H), 6.80-6.90 (m, 2H), 5.98 (s, 2H), 4.62 (d, 2H). MS m/z: 455 (M+1).

Compounds 125 and 126 were prepared using method described in Example 8:

### Analytical Data:

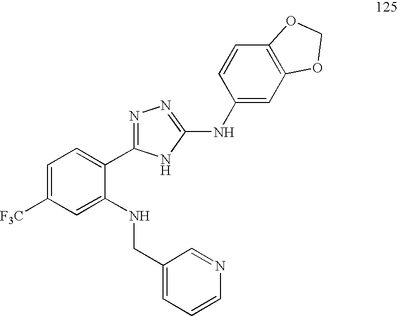

Benzo[1,3]dioxol-5-yl-(5-{2-[(pyridin-3-ylmethyl)-amino]-4-trifluoromethyl-phenyl}-4H-[1,2,4]triazol-3-yl)-amine (125): ¹HNMR (DMSO-d₆) δ (ppm) 9.44 (s, 1H), 8.71 (s, 1H), 8.53-8.56 (m, 1H), 7.78-7.83 (m, 1H), 7.42-7.48 (m, 1H), 26 (m, 1H), 6.97-7.08 (m, 3H), 6.80-6.84 (m, 1H), 6.00 (s, 2H), 4.66 (d, 2H). MS m/z: 455 (M+1).

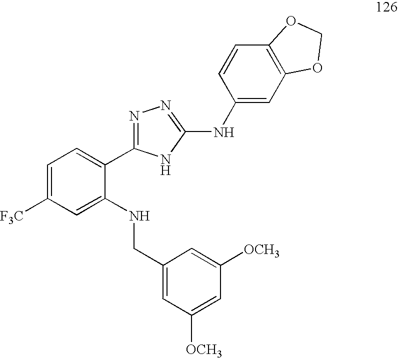

Benzo[1,3]dioxol-5-yl-{5-[2-(3,5-dimethoxy-benzylamino)-4-trifluoromethyl-phenyl]-4H-[1,2,4]triazol-3-yl}-amine (126): ¹HNMR (DMSO-d₆) δ (ppm) 9.39 (s, 1H), 8.58 (s, 1H), 8.16-8.20 (m, 1H), 7.44 (s, 1H), 7.05-7.13 (m, 3H), 6.89-6.95 (m, 1H), 6.61 (d, 2H), 6.48 (s, 1H), 6.02 (s, 2H), 4.60 (d, 2H), 3.78 (s, 6H). MS m/z: 514 (M+1).

### Example 9

### Synthesis of N-{2-[5-(2,3-Dihydro-benzo[1,4]dioxin-6-ylamino)-4H-[1,2,4]triazol-3-yl]-phenyl}-3-trifluoromethoxy-benzenesulfonamide (127)

### Step 1: synthesis of (2,3-Dihydro-benzo[1,4]dioxin-6-yl)-[5-(2-nitro-phenyl)-4H-[1,2,4]triazol-3-yl]-amine (127a)

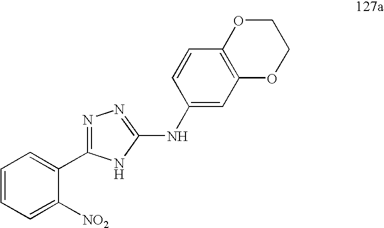

The reaction mixture of 2-nitrobenzoic hydrazide (1.0 g, 5.5 mmol, purchased from Aldrich) and 1-(2,3-dihydro-benzo[1,4]dioxin-6-yl)-2-methyl-isothiourea (2.34 g, 6.6 mmol, from Oakwood Products, Inc.) in pyridine (10 ml) was stirred at 130° C. under argon for 12 hours. The reaction was cooled down to room temperature and poured into water 50 ml. After extracting with ethyl acetate (40 ml×3), the combined organic layer was dried over anhydrous Na₂SO₄. After filtration and evaporation, the organic residue was purified by silica gel column (hexane:ethyl acetate=3:1). Compound 127a recovered as 1.6 g white solid in 85.8% yield. ¹HNMR (DMSO-d₆) δ (ppm) 9.31 (s, 1H), 7.60-7.98 (m, 4H), 7.12 (s, 1H), 6.90-6.93 (m, 1H), 6.69-6.73 (m, 1H), 4.15-4.22 (m, 4H).

### Step 2—synthesis of [5-(2-amino-phenyl)-4H-[1,2,4]triazol-3-yl]-(2,3-dihydro-benzo[1,4]dioxin-6-yl)-amine (127b) 091675

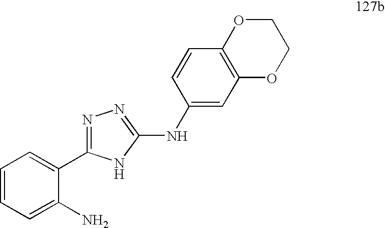

Triazole nitro compound 127a (1.6 g), ethanol (120 ml), and 10% Pd—C (240 mg) was added into a flask. The reaction mixture was degassed, and placed under hydrogen. The reaction mixture stirred at 60° C. for 4 hours. After filtration of catalyst, the colorless solution was evaporated to obtained 127b as a white solid 1.2 g (yield 82.1%). ¹HNMR (DMSO-d₆) δ (ppm) 13.20 (s, 1H), 9.04 (s, 1H), 7.82 (s, 1H), 7.31 (d, 1H), 7.15-7.20 (m, 1H), 6.97-7.00 (m, 1H), 6.60-6.78 (m, 4H), 4.20-4.28 (m, 4H).

### Step 3—synthesis of N-{2-[5-(2,3-Dihydro-benzo[1,4]dioxin-6-ylamino)-4H-[1,2,4]triazol-3-yl]-phenyl}-3-trifluoromethoxy-benzenesulfonamide (127)

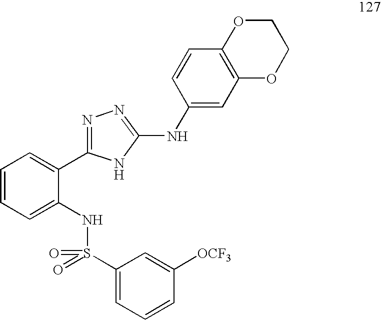

To a solution of amine triazole compound 127b (100 mg, 0.323 mmol) in pyridine (2 ml), 3-trifluoromethoxy-benzenesulfonyl chloride (109.5 mg, 0.42 mmol, purchased from Aldrich) was added. The reaction mixture was stirred at ambient temperature under argon for 12 hours. The reaction was quenched with saturated NaHCO₃. After adding additional 20 ml of water, the mixture was extracted with ethyl acetate (20 ml×3). The combined organic layer was dried over Na₂SO₄. After filtration and evaporation, CH₂Cl₂ was added to the organic resulting organic residue and a solid precipitated. The solid was separated from solvent by filtration to obtain 52 mg of 127 in 30% yield. ¹HNMR (DMSO-d₆) δ (ppm) 8.64-8.68 (m, 2H), 7.85-8.06 (m, 4H), 7.64-7.66 (m, 1H), 7.40-7.46 (m, 2H), 7.12-7.38 (m, 2H), 6.82-6.85 (m, 1H), 4.20-4.28 (m, 4H). MS m/z: 534 (M+1).

Compound 128 was prepared using method described in Example 9:

### Analytical Data:

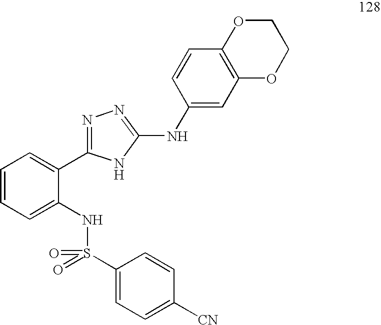

4-Cyano-N-{2-[5-(2,3-dihydro-benzo[1,4]dioxin-6-ylamino)-4H-[1,2,4]triazol-3-yl]-phenyl}-benzenesulfonamide (128): ¹HNMR (DMSO-d₆) δ (ppm) 8.62-8.66 (m, 2H), 7.81-8.00 (m, 2H), 7.63-7.65 (m, 3H), 7.40-7.44 (m, 3H), 6.92-7.00 (m, 1H), 6.80-6.84 (m, 1H), 4.18-4.24 (m, 4H). MS m/z: 475 (M+1).

### Example 10

### Synthesis of [2-(5-Phenyl-4H-[1,2,4]triazol-3-yl)-phenyl]-pyridin-4-ylmethyl-amine (129)

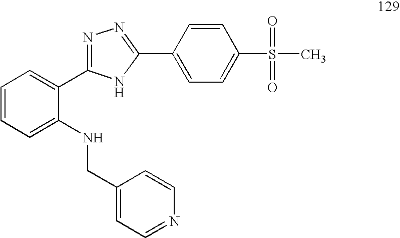

To a solution 2-[(pyridin-4-ylmethyl)-amino]-benzoic acid hydrazide (111b, from Example 7, 180 mg, 0.743 mmol) in pyridine (2 ml) was added 4-methanesulfonyl-benzamidine hydrochloride (179.8 mg, 0.766 mmol, purchased from J&W Pharmlab, PA), and triethylamine (0.15 ml). The reaction mixture heated at 140° C. for 12 hours. The reaction was poured into water (15 ml), then extracted with ethyl acetate (15 ml×3). The combined organic layer was washed with brine, and dried over anhydrous Na₂SO₄. After filtration, the organic phase was evaporated and the residue was washed with hot methanol to give 156 mg of product 129 in 51.8% yield. ¹HNMR (DMSO-d₆) δ (ppm) 8.68-8.74 (m, 1H), 8.50-8.56 (m, 2H), 8.30-8.35 (m, 2H), 8.04-8.08 (m, 2H), 7.91-7.96 (m, 1H), 7.41-7.43 (m, 2H), 7.20-7.28 (m, 1H), 6.62-6.71 (m, 2H), 4.61 (d, 2H), 3.28 (s, 3H). MS m/z: 406 (M+1).

Compound 130 to 152 were synthesized using the method described in Example 10:

### Analytical Data:

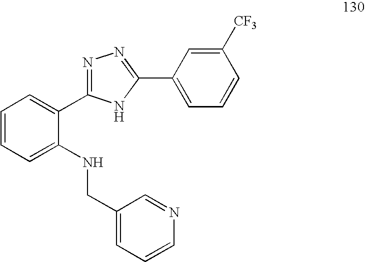

Pyridin-3-ylmethyl-{2-[5-(3-trifluoromethyl-phenyl)-4H-[1,2,4]triazol-3-yl]-phenyl}-amine (130): ¹HNMR (DMSO-d₆) δ (ppm) 8.7 (m, 2H), 8.6 (m, 1H), 8.4 (m, 1H), 8.2 (m, 2H), 7.7-8.0 (m, 4H), 7.5 (m, 1H), 7.3 (m, 1H), 6.7-7.0 (m, 2H), 4.6 (m, 2H). MS m/z: 396 (M+1).

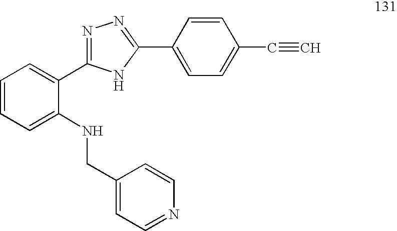

{2-[5-(4-Ethynyl-phenyl)-4H-[1,2,4]triazol-3-yl]-phenyl}-pyridin-4-ylmethyl-amine (131): ¹HNMR (DMSO-d₆) δ (ppm) 14.6 (br. s, 1H), 8 (m, 12H), 6.6 (m, 2H), 4.7 (d, 2H), 4.4 (s, 1H). ¹³CNMR (DMSO-d₆) δ (ppm) 150.1, 149.3, 146.4, 132.6, 131.5, 127.9, 126.5, 122.7, 122.5, 116.0, 111.9, 88.6, 82.5, 55.4, 45.7. MS m/z: 352.2 (M+1).

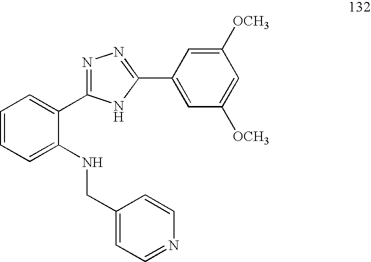

{2-[5-(3,5-Dimethoxy-phenyl)-4H-[1,2,4]triazol-3-yl]-phenyl}-pyridin-4-ylmethyl-amine (132): ¹HNMR (DMSO-d₆) δ (ppm) 10.59 (s, 1H), 8.50-8.53 (m, 2H), 7.97-8.00 (m, 1H), 7.62-7.64 (m, 1H), 7.24-7.36 (m, 3H), 6.89 (d, 2H), 6.67-6.68 (m, 2H), 6.20 (s, 1H), 4.67 (d, 2H), 3.76 (s, 6H). MS m/z: 388 (M+1).

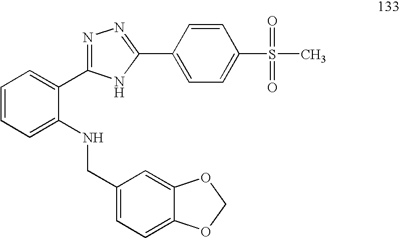

Benzo[1,3]dioxol-5-ylmethyl-{2-[5-(4-methanesulfonyl-phenyl)-4H-[1,2,4]triazol-3-yl]-phenyl}-amine (133): ¹HNMR (DMSO-d₆) δ (ppm) 8.78 (s, 1H), 8.34-8.39 (m, 2H), 8.10-8.17 (m, 2H), 7.92-7.96 (m, 1H), 7.35-7.40 (m, 1H), 7.97-7.10 (m 3H), 6.75-6.87 (m, 2H), 6.02 (s, 2H), 4.50 (d, 2H), 3.25 (s, 3H). MS m/z: 449 (M+1).

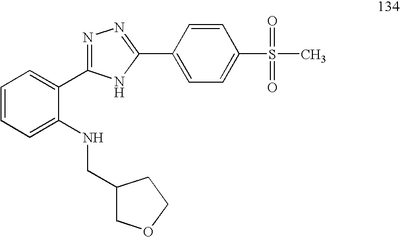

{2-[5-(4-Methanesulfonyl-phenyl)-4H-[1,2,4]triazol-3-yl]-phenyl}-(tetrahydro-furan-3-ylmethyl)-amine (134): ¹HNMR (DMSO-d₆) δ (ppm) 8.30-8.34 (m, 2H), 8.03-8.09 (m, 2H), 7.74-7.78 (m, 1H), 7.29-7.39 (m, 2H), 6.97-7.04 (m, 1H), 6.82-6.86 (m, 2H), 6.10-6.13 (m, 1H), 3.60-3.90 (m, 4H), 3.23 (s, 3H), 3.05-30.7 (m, 2H), 2.72-2.80 (m, 1H), 1.85-1.95 (m, 2H). MS m/z: 399 (M+1).

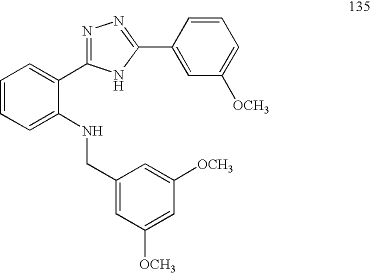

(3,5-Dimethoxy-benzyl)-{2-[5-(3-methoxy-phenyl)-4H-[1,2,4]triazol-3-yl]-phenyl}-amine (135): ¹HNMR (CDCl₃) δ (ppm) 11.00 (br s, 1H), 8.50 (br s, 1H), 7.69 (d, J=7.8 Hz, 1H), 7.62 (d, J=7.8 Hz, 1H), 7.58 (t, J=1.8 Hz, 1H), 7.35 (d, J=7.8 Hz, 1H), 7.31-7.24 (m, 1H), 6.97 (m, 1H), 6.75 (d, J=8.4 Hz, 1H), 6.70 (t, J=7.5 Hz, 1H), 6.63 (d, J=2.1 Hz, 2H), 6.38 (t, J=2.1 Hz, 1H), 4.47 (s, 2H), 3.87 (s, 3H), 3.76 (s, 6H). MS m/z: 417 (M+1).

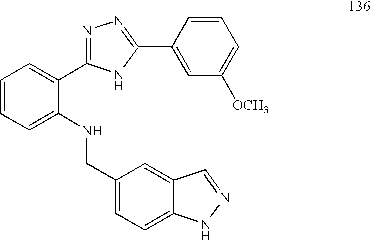

(1H-Indazol-5-ylmethyl)-{2-[5-(3-methoxy-phenyl)-4H-[1,2,4]triazol-3-yl]-phenyl}-amine (136): ¹HNMR (DMSO-d₆) δ (ppm) 14.38 (s, 1H), 13.07 (s, 1H), 8.78 (s, 1H), 8.20-8.15 (m, 1H), 8.04 (s, 1H), 7.84 (s, 1H), 7.58-7.42 (m, 4H), 7.32-7.23 (m, 2H), 7.10-6.87 (m, 2H), 6.71 (t, J=3.9 Hz, 1H), 4.58 (d, J=3.9 Hz, 2H), 3.63 (s, 3H). MS m/z: 397 (M+1).

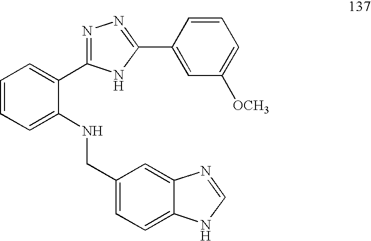

(1H-Benzoimidazol-5-ylmethyl)-{2-[5-(3-methoxy-phenyl)-4H-[1,2,4]triazol-3-yl]-phenyl}-amine (137): ¹HNMR (DMSO-d₆) δ (ppm) 14.38 (s, 1H), 12.40 (s, 1H), 8.80 (s, 1H), 8.21 (s, 1H), 8.20-8.15 (m, 1H), 7.86-7.84 (m, 1H), 7.54-7.20 (m, 6H), 7.02-6.68 (m, 3H), 4.60 (s, 2H), 3.64 (s, 3H). MS m/z: 397 (M+1).

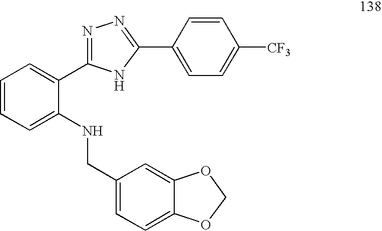

Benzo[1,3]dioxol-5-ylmethyl-{2-[5-(4-trifluoromethyl-phenyl)-4H-[1,2,4]triazol-3-yl]-phenyl}-amine (138): ¹HNMR (DMSO-d₆) δ (ppm) 14.65 (s, 1H), 8.74 (s, 1H), 8.31 (d, J=7.5 Hz, 2H), 8.04-8.00 (m, 1H), 7.93-7.88 (m, 2H), 7.35 (t, J=7.5 Hz, 1H), 7.08 (s, 1H), 7.00 (s, 2H), 6.85 (d, J=8.4 Hz, 1H), 6.78 (t, J=7.5 Hz, 1H), 6.07 (s, 2H), 4.51 (s, 2H). MS m/z: 439 (M+1).

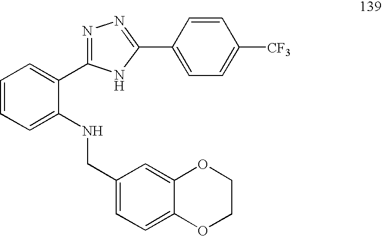

(2,3-Dihydro-benzo[1,4]dioxin-6-ylmethyl)-{2-[5-(4-trifluoromethyl-phenyl)-4H-[1,2,4]triazol-3-yl]-phenyl}-amine (139): ¹HNMR (DMSO-d₆) δ (ppm) 14.61 (s, 1H), 8.70 (s, 1H), 8.27 (d, J=8.1 Hz, 2H), 8.02-7.94 (m, 1H), 7.89-7.84 (m, 2H), 7.31 (t, J=7.5 Hz, 1H), 6.98-6.88 (m, 3H), 6.81 (d, J=8.4 Hz, 1H), 6.74 (t, J=7.5 Hz, 1H), 4.45 (s, 2H), 4.25 (s, 4H). MS m/z: 453 (M+1).

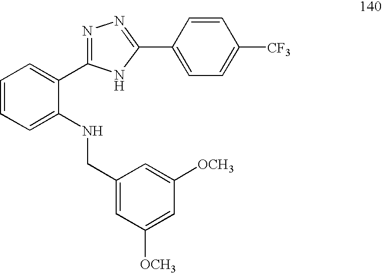

(3,5-Dimethoxy-benzyl)-{2-[5-(4-trifluoromethyl-phenyl)-4H-[1,2,4]triazol-3-yl]-phenyl}-amine (140): ¹HNMR (CDCl₃) δ (ppm) 11.00 (br s, 1H), 8.47 (br s, 1H), 8.10 (d, J=8.1 Hz, 2H), 7.59 (d, J=8.1 Hz, 2H), 7.49 (d, J=7.2 Hz, 1H), 7.24 (d, J=7.8, 1.5 Hz, 1H), 6.69 (d, J=8.1 Hz, 1H), 6.64 (t, J=7.5 Hz, 1H), 6.58 (d, J=2.1 Hz, 2H), 6.36 (t, J=2.4 Hz, 1H), 4.40 (d, J=4.2 Hz, 2H), 3.73 (s, 6H). MS m/z: 455 (M+1).

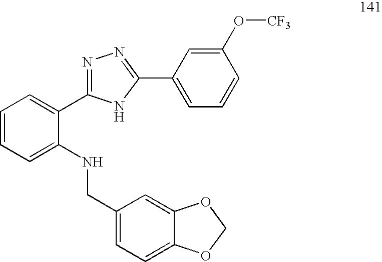

Benzo[1,3]dioxol-5-ylmethyl-{2-[5-(3-trifluoromethoxy-phenyl)-4H-[1,2,4]triazol-3-yl]-phenyl}-amine (141): ¹HNMR (DMSO-d₆) δ (ppm) 14.46 (s, 1H), 8.53 (br s, 1H), 8.00 (d, J=7.5 Hz, 1H), 7.79 (s, 2H), 7.62-7.56 (m, 1H), 7.43-7.38 (m, 1H), 7.26-7.19 (m, 1H), 6.95 (s, 1H), 6.90-6.82 (m, 2H), 6.74 (d, J=8.1 Hz, 1H), 6.65 (t, J=7.5 Hz, 1H), 5.94 (s, 2H), 4.35 (s, 2H). MS m/z: 455 (M+1).

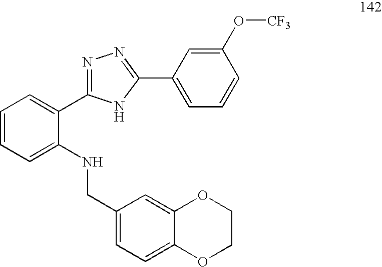

(2,3-Dihydro-benzo[1,4]dioxin-6-ylmethyl)-{2-[5-(3-trifluoromethoxy-phenyl)-4H-[1,2,4]triazol-3-yl]-phenyl}-amine (142): ¹HNMR (DMSO-d₆) δ (ppm) 14.53 (s, 1H), 8.60 (br s, 1H), 8.15-8.03 (m, 2H), 7.87 (s, 1H), 7.62 (t, J=7.5 Hz, 1H), 7.46-7.43 (m, 1H), 7.30 (t, J=7.8 Hz, 1H), 6.95-6.80 (m, 4H), 6.72 (d, J=8.1 Hz, 1H), 4.39 (s, 2H), 4.22 (s, 4H). MS m/z: 469 (M+1).

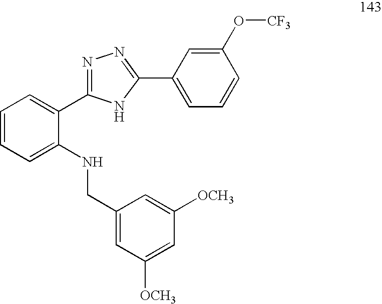

(3,5-Dimethoxy-benzyl)-{2-[5-(3-trifluoromethoxy-phenyl)-4H-[1,2,4]triazol-3-yl]-phenyl}-amine (143): ¹HNMR (DMSO-d₆) δ (ppm) 14.62 (s, 1H), 8.71 (t, J=5.1 Hz, 1H), 8.22-8.18 (m, 1H), 8.14 (d, J=7.8 Hz, 1H), 7.95 (s, 1H), 7.68 (t, J=7.8 Hz, 1H), 7.54 (d, J=7.5 Hz, 1H), 7.38 (t, J=7.5 Hz, 1H), 6.89 (d, J=8.7 Hz, 1H), 6.81 (t, J=7.5 Hz, 1H), 6.74-6.68 (m, 2H), 6.51 (s, 1H), 4.54 (s, 2H), 3.79 (s, 6H). MS m/z: 471 (M+1).

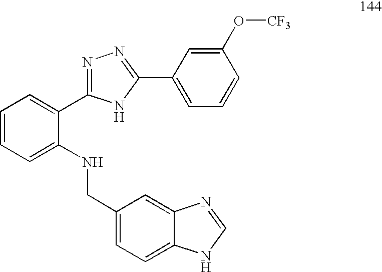

(1H-Benzoimidazol-5-ylmethyl)-{2-[5-(3-trifluoromethoxy-phenyl)-4H-[1,2,4]triazol-3-yl]-phenyl}-amine (144): ¹HNMR (DMSO-d₆) δ (ppm) 14.28 (s, 1H), 12.15 (s, 1H), 8.48 (s, 1H), 7.95 (s, 1H), 7.74-7.04 (m, 9H), 6.65-6.58 (m, 1H), 6.46 (t, J=7.5 Hz, 1H), 4.38 (s, 2H). MS m/z: 451 (M+1).

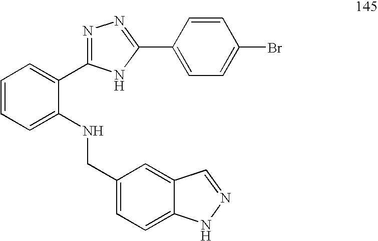

{2-[5-(4-Bromo-phenyl)-4H-[1,2,4]triazol-3-yl]-phenyl}-(1H-indazol-5-ylmethyl)-amine (145): ¹HNMR (DMSO-d₆) δ (ppm) 14.24 (s, 1H), 12.86 (s, 1H), 8.58 (s, 1H), 8.00-7.90 (m, 1H), 7.84 (s, 1H), 7.65-7.56 (m, 4H), 7.38-7.23 (m, 3H), 7.10-7.00 (m, 1H), 6.62 (d, J=8.1 Hz, 1H), 6.48 (t, J=7.5 Hz, 1H), 4.38 (s, 2H). MS m/z: 445 (M+1).

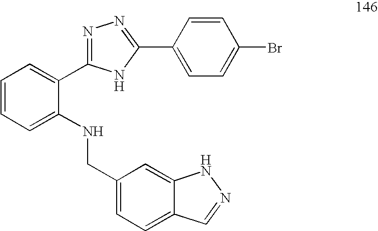

{2-[5-(4-Bromo-phenyl)-4H-[1,2,4]triazol-3-yl]-phenyl}-(1H-indazol-6-ylmethyl)-amine (146): ¹HNMR (DMSO-d₆) (ppm) δ 14.24 (br s, 1H), 12.84 (s, 1H), 8.59 (s, 1H), 7.93 (s, 1H), 7.82 (d, J=8.4 Hz, 2H), 7.78 (s, 1H), 7.65 (d, J=8.1 Hz, 1H), 7.50 (d, J=8.4 Hz, 2H), 7.43 (s, 1H), 7.10 (t, J=8.1 Hz, 2H), 6.66 (d, J=8.1 Hz, 1H), 6.57 (t, J=7.8 Hz, 1H), 4.55 (d, J=5.1 Hz, 2H). MS m/z: 445 (M+1).

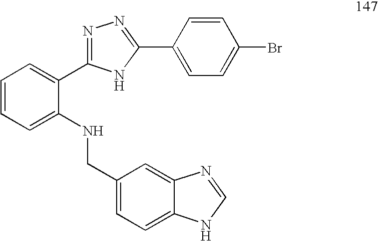

(1H-Benzoimidazol-5-ylmethyl)-{2-[5-(4-bromo-phenyl)-4H-[1,2,4]triazol-3-yl]-phenyl}-amine (147): ¹HNMR (DMSO-d₆) δ (ppm) 14.31 (s, 1H), 12.23 (s, 1H), 8.67 (s, 1H), 8.07 (s, 1H), 7.76-7.44 (m, 7H), 7.20-7.14 (m, 2H), 6.68 (d, J=7.2 Hz, 1H), 6.54 (t, J=7.2 Hz, 1H), 4.46 (s, 2H). MS m/z: 445 (M+1).

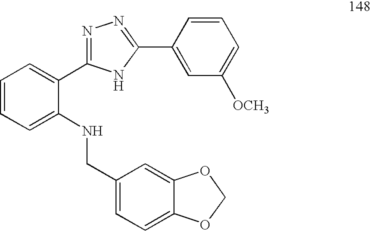

Benzo[1,3]dioxol-5-ylmethyl-{2-[5-(3-methoxy-phenyl)-4H-[1,2,4]triazol-3-yl]-phenyl}-amine (148): ¹HNMR (CDCl₃) δ (ppm) 10.90 (br s, 1H), 8.41 (br s, 1H), 7.69 (s, 1H), 7.61 (d, J=7.5 Hz, 1H), 7.58-7.57 (m, 1H), 7.36 (t, J=8.1 Hz, 1H), 7.32-7.27 (m, 1H), 6.99-6.91 (m, 3H), 6.80 (d, J=7.8 Hz, 1H), 6.77-6.70 (m, 2H), 5.95 (s, 2H), 4.44 (s, 2H), 3.84 (s, 3H). MS m/z: 401 (M+1).

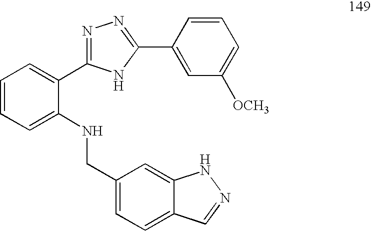

(1H-Indazol-6-ylmethyl)-{2-[5-(3-methoxy-phenyl)-4H-[1,2,4]triazol-3-yl]-phenyl}-amine (149). ¹HNMR (CDCl₃) δ (ppm) 10.40 (br s, 1H), 8.70 (br s, 1H), 8.15-8.07 (m, 1H), 7.80-7.60 (m, 3H), 7.42-7.10 (m, 5H), 7.10-6.72 (m, 4H), 4.71 (s, 2H), 3.87 (s, 3H). MS m/z: 397 (M+1).

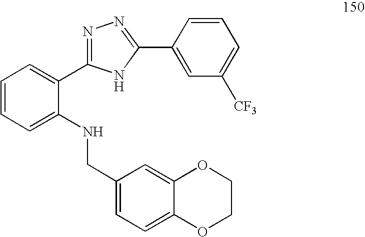

(2,3-Dihydro-benzo[1,4]dioxin-6-ylmethyl)-{2-[5-(3-trifluoromethyl-phenyl)-4H-[1,2,4]triazol-3-yl]-phenyl}-amine (150). ¹HNMR (DMSO-d₆) δ (ppm) 14.65 (br. s, 1H), 8.60 (br. s, 1H), 8.38 (d, J=7.8 Hz, 1H), 8.30 (br. s, 1H), 7.80-7.90 (m, 3H), 7.32 (m, 1H), 6.75-7.00 (m, 5H), 4.44 (d, J=4.5 Hz, 2H), 4.28 (s, 4H). MS m/z: 453 (M+1).

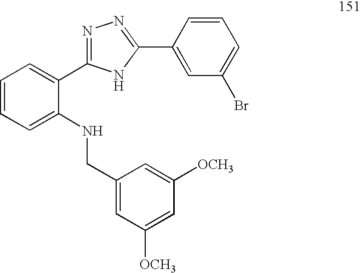

{2-[5-(3-Bromo-phenyl)-4H-[1,2,4]triazol-3-yl]-phenyl}-(3,5-dimethoxy-benzyl)-amine (151): ¹HNMR (DMSO-d₆) δ (ppm) 14.56 (br. s, 1H), 8.15 (br. s, 1H), 8.03 (d, J=8.1 Hz, 1H), 7.93 (br. s, 1H), 7.65 (d, J=7.5 Hz, 1H), 7.43 (t, J=7.7 Hz, 1H), 7.24 (t, J=7.7 Hz, 1H), 6.76 (d, J=8.4 Hz, 1H), 6.69 (t, J=7.4 Hz, 1H), 6.61 (m, 2H), 6.43 (m, 1H), 4.45 (d, J=4.8 Hz, 2H), 3.72 (s, 6H). MS m/z: 466 (M+1).

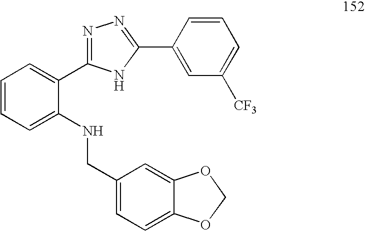

Benzo[1,3]dioxol-5-ylmethyl-{2-[5-(3-trifluoromethyl-phenyl)-4H-[1,2,4]triazol-3-yl]-phenyl}-amine (152): ¹HNMR (DMSO-d₆) δ (ppm) 14.62 (br. s, 1H), 8.64 (br. s, 1H), 8.38 (d, J=1.2 Hz, 1H), 8.26 (br. s, 1H), 7.78-7.94 (m, 3H), 7.36 (m, 1H), 7.09 (br. s, 1H), 6.95-7.03 (m, 2H), 6.87 (d, J=8.1 Hz, 1H), 6.73 (t, J=7.4 Hz, 1H), 6.07 (s, 2H), 4.47 (d, J=4.8 Hz, 2H). MS m/z: 439 (M+1).

### Example 11

### Synthesis of {2-[5-(4-Methanesulfonyl-phenyl)-4H-[1,2,4]triazol-3-yl]-5-trifluoromethyl-phenyl}-pyridin-4-ylmethyl-amine (153) (094309)

### Step 1: synthesis of 3-(4-Methanesulfonyl-phenyl)-5-(2-nitro-4-trifluoromethyl-phenyl-4H-[1,2,4]triazole (153a)

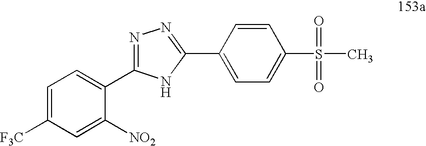

A reaction mixture of 2-nitro-4-trifluoromethyl-benzoic acid hydrazide (1.2 g, 4.82 mmol), 4-methanesulfonyl-benzamidine (1.2 g, 5.05 mmol), pyridine (10 ml), and triethylamine (1 ml) in a sealed tube was heated to 160° C. for 4 hours. The reaction solution was poured into 80 ml water, then extracted with ethyl acetate (80 ml×3). The combined organic layer was dried over anhydrous Na₂SO₄, then filtered and evaporated to yield a solid organic residue. Dichloromethane was added to the solid residue and left it for 30 minutes A solid precipitated. 826 mg yellow solid was recovered from filtration (yield. 41.6%). ¹HNMR (DMSO-d₆) δ (ppm) 15.26 (s, 1H), 8.52 (s, 1H), 8.30-8.33 (m, 1H), 8.20-8.28 (m, 3H), 8.02-8.09 (m, 2H), 3.40 (s, 3H). MS m/z: 413 (M+1).

### Step 2: synthesis of 2-[5-(4-Methanesulfonyl-phenyl)-4H-[1,2,4]triazol-3-yl]-5-trifluoromethyl-phenylamine (153b)

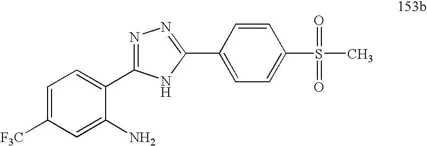

The nitro triazole compound (153a, 800 mg), ethanol (60 ml), Pd—C 10% (120 mg) were added into a flask. The reaction mixture stirred at 60° C. for 3 hours. A solid precipitated from the solution. The reaction was diluted with 60 ml chloroform, and heated at 80° C. until the precipitation disappeared. After filtered out the catalyst, the filtrate was evaporated to obtain compound 153b (643 mg, yield 86.7%). ¹HNMR (DMSO-d₆) δ (ppm) 14.88 (s, 1H), 8.42-8.46 (m, 2H), 8.04-8.09 (m, 3H), 7.24 (s, 1H), 7.15 (s, 1H), 6.93-6.98 (m, 1H), 3.32 (s, 3H). MS m/z: 383 (M+1).

### Step 3: synthesis of {2-[5-(4-Methanesulfonyl-phenyl)-4H-[1,2,4]triazol-3-yl]-5-trifluoromethyl-phenyl}-pyridin-4-ylmethyl-amine (153)

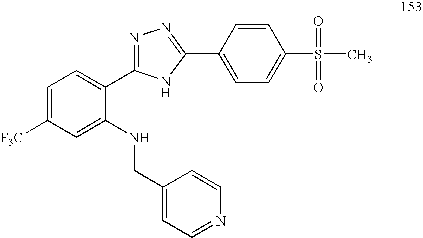

To a solution of 2-[5-(4-methanesulfonyl-phenyl)-4H-[1,2,4.]triazol-5-trifluoromethyl-phenylamine (153b, 60 mg, 0.16 mmol) in anhydrous dichloromethane (5 ml) was added pyridine-4-carbaldehyde (17 μL, 0.172 mmol), sodium triacetoxyborohydride (87.1 mg, 0.392 mmol), acetic acid (0.8 mmol). The reaction mixture was stirred at ambient temperature for 6 hours. The reaction was quenched with aqueous 2N NaOH. After the addition of water, the mixture was extracted with ethyl acetate (20 ml×3). The combined organic layer was washed with brine, and dried over anhydrous Na₂SO₄. After filtration and evaporated, the organic residue was added to ethanol (5 ml) followed by sodium borohydride (50 mg). The reaction mixture was stirred at 60° C. for 2 hours. The reaction solution was quenched with water, and white solid precipitated out. After filtration, the solid was washed with hot methanol to yield 10 mg of 153 (Yield 13.5%). ¹HNMR (DMSO-d₆) δ (ppm) 9.00 (s, 1H), 8.70-8.72 (m, 2H), 8.43-8.48 (m, 2H), 8.30-8.33 (m, 1H), 8.14-8.21 (m, 2H), 7.53-7.59 (m, 2H), 7.14-7.19 (m, 1H), 7.07 (s, 1H), 4.85 (d, 2H), 3.41 (s, 3H). MS m/z: 474 (M+1).

### Example 12

### synthesis of 2,3-Dihydro-benzo[1,4]dioxine-6-carboxylic acid {2-[5-(4-trifluoromethyl-phenylamino)-4H-[1,2,4]triazol-3-yl]-phenyl}-amide (154)

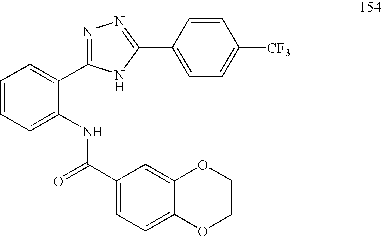

2[5-(2-Amino-phenyl)-4H-[1,2,4]triazol-3-yl]-(4-trifluoromethyl-phenyl)-amine (50 mg, 0.16 mmol, synthesized from 2-aminobenzoic acid hydrazide using method from Example 10), 2,3-Dihydro-benzo[1,4]dioxine-6-carbonyl chloride (39 mg, 0.20 mmol), and triethylamine (40 mg, 0.50 mmol) were dissolved in CH₂Cl₂ and left to stir at room temperature for 12 hours. The reaction mixture was concentrated and the crude material was purified by flash chromatography (1:99 methanol/CH₂Cl₂). Yield 30%. ¹HNMR (CDCl₃) δ (ppm) 15.11 (br s, 1H), 12.47 (br s, 1H), 8.84 (d, J=8.1 Hz, 1H), 8.32 (d, J=8.1 Hz, 2H), 8.19 (d, J=6.9 Hz, 1H), 7.93-7.84 (m, 2H), 7.65-7.49 (m, 3H), 7.30 (t, J=7.2 Hz, 1H), 7.09 (d, J=7.2 Hz, 1H), 4.37-4.31 (m, 4H). MS m/z: 467 (M+1).

### Example 13

### synthesis of {3-[5-(Benzo[1,3]dioxol-5-ylamino)-[1,3,4]oxadiazol-2-yl]-pyridin-2-yl}-(3,4-difluoro-benzyl)-amine (155)

### Step 1: Synthesis of 2-(3,4-Difluoro-benzylamino)-nicotinic acid Ethyl Ester (155a)

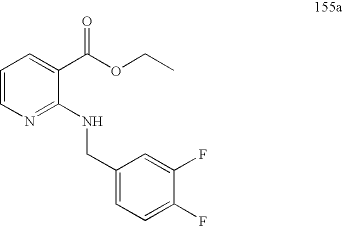

Ethyl-2-chloronicotinate (2.0 ml, 13.9 mmol) was added to a solution of triethylamine (2.5 ml, 17.8 mmol) in dimethylsulfoxide (10 ml) and stirred for five minutes. 3,4-Difluorobenzylamine (2.1 ml, 17.8 mmol) was added to the mixture and heated to 70° C. Upon disappearance of starting material, the reaction mixture was diluted with ethyl acetate (20 ml) and washed 2×20 ml of de-ionized water. The aqueous wash was re-extracted 3×20 ml of ethyl acetate. The organic layers were combined and dried over anhydrous sodium sulfate. The sodium sulfate was filtered, and the organic solvent was removed in vacuo. The yellow oil was purified with silica gel flash column chromatography (Hexane:Dichloromethane=2:1) to yield a yellow oil (2.2 g, 54%). ¹HNMR (Acetone-d₆) δ (ppm): 8.37-8.62 (br, 1H), 8.34-8.21 (m, 1H), 8.09-8.21 (m, 1H), 7.30-7.46 (m, 1H), 7.19-7.30 (m, 2H), 6.58-6.73 (m, 1H), 4.78 (d, 2H), 4.23-4.44 (q, 2H), 1.23-1.46 (t, 3H). MS m/z: 293 (M+1).

### Step 2: Synthesis of 2-(3,4-Difluoro-benzylamino)-nicotinic acid Hydrazide (155b)

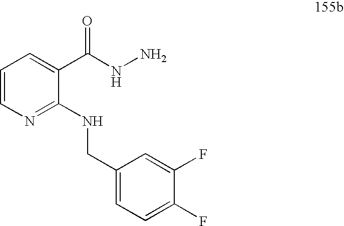

Isopropyl alcohol (7 ml) was added to a round bottom flask containing 2-(3,4-difluoro-benzylamino)-nicotinic acid ethyl ester 155a (1.5 g, 5.13 mmol). Hydrazine monohydrate (2 ml, 41.2 mmol) was added to the mixture and heated to 70° C. Upon disappearance of starting material, the reaction mixture was diluted with ethyl acetate (15 ml) and washed 2×15 ml of de-ionized water. The organic layer was dried over anhydrous sodium sulfate and filtered. The organic solvent was removed in vacuo to yield yellow oil. The yellow oil was purified with silica gel flash column chromatography (Hexane:Ethyl Acetate=1:2.5) to yield 1.3 g white solid in 90% yield. ¹HNMR (DMSO-d₆) δ (ppm) 9.68-9.84 (br, 1H), 8.50-8.66 (m, 1H), 8.03-8.15 (m, 1H), 7.75-7.89 (m, 1H), 7.24-7.40 (m, 2H), 7.05-7.18 (br, 1H), 6.47-6.64 (m, 1H), 4.58 (d, 2H), 4.35-4.61 (br, 2H). MS m/z: 279.03 (M+1).

### Step 3: Synthesis of Hydrazide Thiourea Intermediate 155c

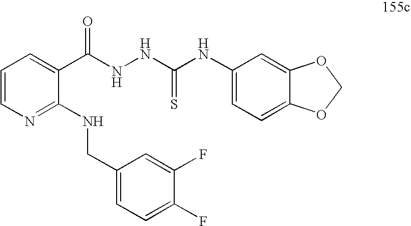

2-(3,4-Difluoro-benzylamino)-nicotinic acid hydrazide (155b, 106 mg, 0.38 mmol) was dissolved in dichloromethane (10 ml) and stirred for five minutes under argon. 5-Isothiocyano-benzo[1,3]dioxole (82.6 mg, 0.46 mmol) was added to the reaction mixture and heated to 30° C. Upon disappearance of starting material, the reaction was cooled and filtered. The white solid was washed diethyl ether (3×5 ml) and dichloromethane (2×5 ml) (144 mg, 83%). ¹HNMR (DMSO-d₆) δ (ppm) 10.38-10.62 (br, 1H), 9.63-9.86 (br, 1H), 9.43-9.61 (br, 1H), 8.49-8.75 (br, 1H), 8.10-8.25 (m, 1H), 7.99-8.10 (m, 1H), 7.21-7.46 (m, 2H), 7.07-7.21 (br, 1H), 6.92-7.05 (br, 1H), 6.85 (d, 1H), 6.68-6.78 (m, 1H), 6.55-6.68 (m, 1H), 5.91 (s, 2H), 4.52-4.74 (d, 2H). MS m/z: 457.94 (M+1).

### Step 4: Synthesis of {3-[5-(Benzo[1,3]dioxol-5-ylamino)-[1,3,4]oxadiazol-2-yl]-pyridin-2-yl}-(3,4-difluoro-benzyl)-amine (155)

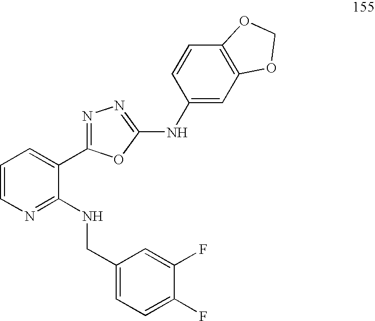

1,3-Dicyclohexylcarbodiimide (50.3 mg, 0.24 mmol) was added to a solution of 155c (73.9 mg, 0.16 mmol) in anhydrous toluene (5 ml) and heated under argon atmosphere to 100° C. Upon disappearance of starting material, the reaction was cooled and diluted with ethyl acetate (10 ml). The reaction mixture was washed with a saturated aqueous solution of sodium bicarbonate (10 ml) and saturated aqueous solution of sodium chloride (2×10 ml). The organic layer was separated and dried over anhydrous sodium sulfate. After filtration and removal of the organic solvent in vacuo, methanol (10 ml) was added to the white solid and heated to 60° C. for 10 minutes. Methanol was removed in vacuo to a volume of approximately 2 ml. The mixture was cooled in an ice bath and the white precipitate was filtered and washed with diethyl ether (3×5 ml) (53.4 mg, 78%). ¹HNMR (DMSO-d₆) δ (ppm) 10.58 (s, 1H), 8.24-8.37 (m, 2H), 7.90-7.99 (m, 1H), 7.22-7.53 (m, 4H), 7.03-7.12 (m, 1H), 7.00 (d, 1H), 6.79-6.90 (m, 1H), 6.03 (s, 2H), 4.83 (d, 2H). MS m/z: 424.02 (M+1).

Compounds 156 to 207 were synthesized using the method described in Example 13:

### Analytical Data:

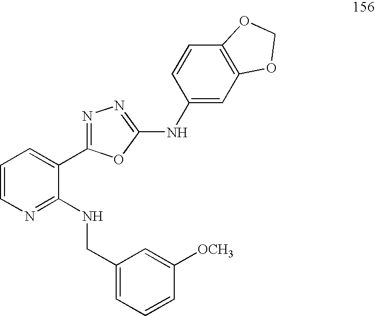

{3-[5-(Benzo[1,3]dioxol-5-ylamino)-[1,3,4]oxadiazol-2-yl]-pyridin-2-yl}-(3-methoxy-benzyl)-amine (156): ¹HNMR (DMSO-d₆) δ (ppm) 10.48 (s, 1H), 8.06-8.16 (m, 1H), 7.95-8.04 (m, 1H), 7.65-7.78 (m, 1H), 7.02-7.18 (m, 2H), 6.82-6.95 (m, 1H), 6.70-6.80 (m, 3H), 6.57-6.70 (m, 2H), 5.82 (s, 2H), 4.61 (d, 2H), 3.58 (s, 3H). MS m/z: 418.01 (M+1).

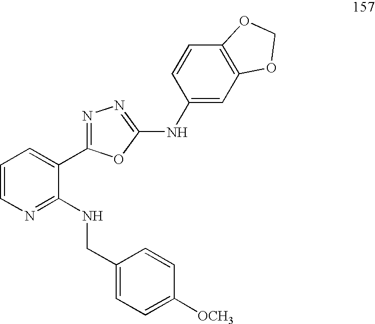

{3-[5-(Benzo[1,3]dioxol-5-ylamino)-[1,3,4]oxadiazol-2-yl]-pyridin-2-yl}-(4-methoxy-benzyl)-amine (157): ¹HNMR (DMSO-d₆) δ (ppm) 10.49 (s, 1H), 8.21-8.34 (m, 1H), 8.03-8.16 (m, 1H), 7.83-7.94 (m, 1H), 7.23-7.42 (m, 3H), 6.99-7.09 (m, 1H), 6.89-6.99 (m, 3H), 6.74-6.85 (m, 1H), 6.03 (s, 2H), 4.72 (d, 2H), 3.70 (s, 3H). MS m/z: 418.01 (M+1).

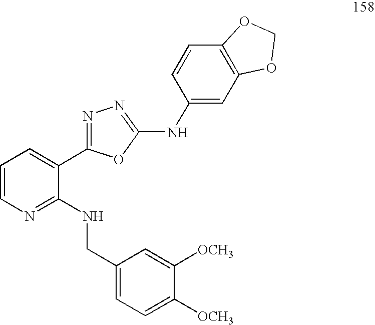

{3-[5-(Benzo[1,3]dioxol-5-ylamino)-[1,3,4]oxadiazol-2-yl]-pyridin-2-yl}-(3,4-dimethoxy-benzyl)-amine (158): ¹HNMR (DMSO-d₆) δ (ppm) 10.65 (s, 1H), 8.32-8.43 (m, 1H), 8.14-8.25 (m, 1H), 7.95-8.03 (m, 1H), 7.36-7.42 (m, 1H), 7.09-7.18 (m, 2H), 6.98-7.06 (m, 3H), 6.86-6.93 (m, 1H), 6.07 (s, 2H), 4.80 (d, 2H), 3.80 (s, 6H). MS m/z: 448.08 (M+1).

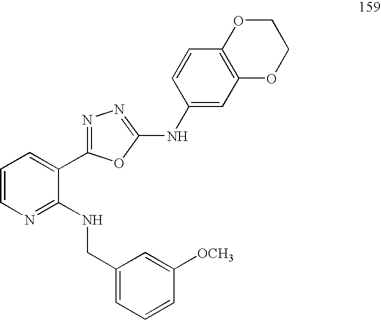

{3-[5-(2,3-Dihydro-benzo[1,4]dioxin-6-ylamino)-[1,3,4]oxadiazol-2-yl]-pyridin-2-yl}-(3-methoxy-benzyl)-amine (159): ¹HNMR (DMSO-d₆) δ (ppm) 10.55 (s, 1H), 8.11-8.32 (m, 2H), 7.78-7.94 (m, 1H), 7.15-7.31 (m, 2H), 6.91-7.08 (m, 3H), 6.70-6.90 (m, 3H), 4.74 (d, 2H), 4.10-4.32 (m, 4H), 3.63 (s, 3H). MS m/z: 431.95 (M+1).

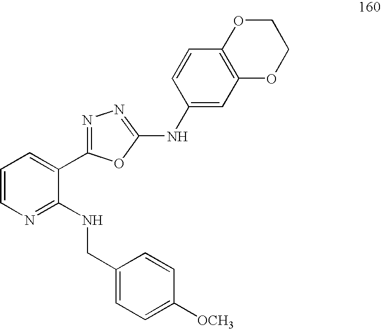

{3-[5-(2,3-Dihydro-benzo[1,4]dioxin-6-ylamino)-[1,3,4]oxadiazol-2-yl]-pyridin-2-yl}-(4-methoxy-benzyl)-amine (160): ¹HNMR (DMSO-d₆) δ (ppm) 10.35 (s, 1H), 7.98-8.11 (m, 1H), 7.83-7.96 (m, 1H), 7.61-7.73 (m, 1H), 7.14 (d, 2H), 6.96-7.09 (d, 1H), 6.78-6.89 (m, 1H), 6.50-6.78 (m, 4H), 4.50 (d, 2H), 3.94-4.13 (m, 4H), 3.50 (s, 3H). MS m/z: 431.96 (M+1).

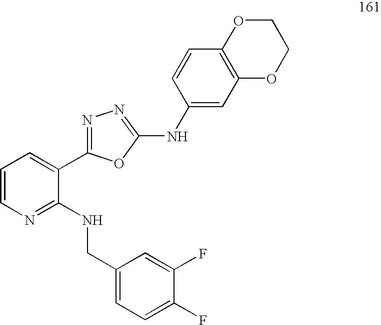

(3,4-Difluoro-benzyl)-{3-[5-(2,3-dihydro-benzo[1,4]dioxin-6-ylamino)-[1,3,4]oxadiazol-2-yl]-pyridin-2-yl}-amine (161): ¹HNMR (DMSO-d₆) δ (ppm) 10.49 (s, 1H), 8.13-8.38 (m, 2H), 7.80-7.98 (m, 1H), 7.30-7.53 (m, 2H), 7.14-7.30 (m, 2H), 6.94-7.09 (m, 1H), 6.73-6.94 (m, 2H), 4.79 (d, 2H), 4.22 (d, 4H). MS m/z: 437.89.

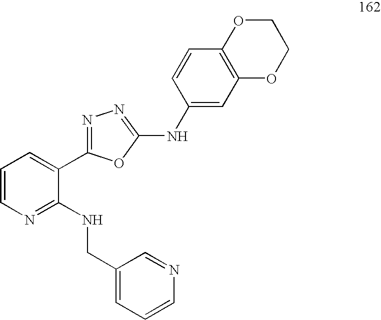

{3-[5-(2,3-Dihydro-benzo[1,4]dioxin-6-ylamino)-[1,3,4]oxadiazol-2-yl]-pyridin-2-yl}-pyridin-3-ylmethyl-amine (162): ¹HNMR (DMSO-d₆) δ (ppm) 10.52 (s, 1H), 8.48 (d, 2H), 8.26-8.36 (m, 1H), 8.13-8.21 (m, 1H), 7.83-7.96 (m, 1H), 7.29-7.36 (m, 2H), 7.21-7.29 (m, 1H), 6.95-7.07 (m, 1H), 6.72-6.92 (m, 2H), 4.85 (d, 2H), 4.14-4.31 (m, 4H). MS m/z: 402.99 (M+1).

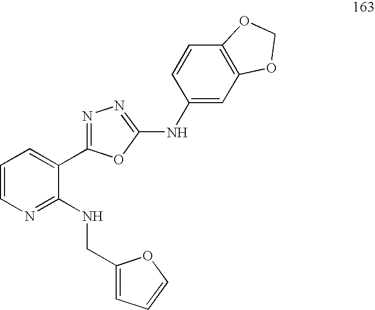

{3-[5-(Benzo[1,3]dioxol-5-ylamino)-[1,3,4]oxadiazol-2-yl]-pyridin-2-yl}-furan-2-ylmethyl-amine (163): ¹HNMR (DMSO-d₆) δ (ppm) 10.82 (s, 1H), 8.40-8.51 (m, 1H), 8.20-8.32 (m, 1H), 8.01-8.13 (m, 1H), 7.75-7.85 (m, 1H), 7.42-7.53 (m, 1H), 7.17-7.30 (m, 1H), 7.05-7.14 (m, 1H), 6.93-7.05 (m, 1H), 6.56-6.66 (m, 1H), 6.12 (s, 2H), 4.97 (d, 2H). MS m/z: 378 (M+1).

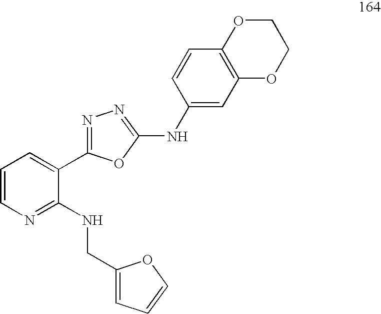

{3-[5-(2,3-Dihydro-benzo[1,4]dioxin-6-ylamino)-[1,3,4]oxadiazol-2-yl]-pyridin-2-yl}-furan-2-ylmethyl-amine (164): ¹HNMR (DMSO-d₆) δ (ppm) 10.50 (s, 1H), 8.22-8.33 (m, 1H), 8.01-8.15 (t, 1H), 7.81-7.94 (m, 1H), 7.56 (s, 1H), 7.16-7.30 (m, 1H), 6.94-7.07 (m, 1H), 6.75-6.91 (m, 2H), 6.38-6.49 (m, 1H), 6.26-6.36 (m, 1H), 4.78 (d, 2H), 4.12-4.34 (m, 4H). MS m/z: 392 (M+1).

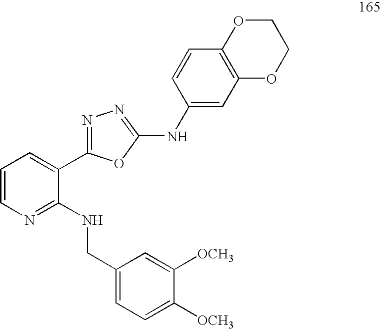

{3-[5-(2,3-Dihydro-benzo[1,4]dioxin-6-ylamino)-[1,3,4]oxadiazol-2-yl]-pyridin-2-yl}-(3,4-dimethoxy-benzyl)-amine (165): ¹HNMR (DMSO-d₆) δ (ppm) 10.45 (s, 1H), 8.18-8.35 (m, 1H), 8.02-8.17 (m, 1H), 7.79-7.95 (m, 1H), 7.13-7.35 (m, 1H), 6.68-7.11 (m, 6H), 4.69 (d, 2H), 4.23 (d, 4H), 3.62 (s, 6H). MS m/z: 462 (M+1).

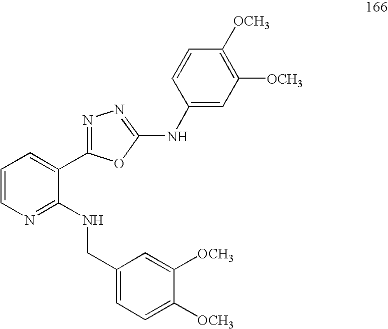

(3,4-Dimethoxy-benzyl)-{3-[5-(3,4-dimethoxy-phenylamino)-[1,3,4]oxadiazol-2-yl]-pyridin-2-yl}-amine (166): ¹HNMR (DMSO-d₆) δ (ppm) 10.55 (s, 1H), 8.20-8.30 (m, 1H), 8.10 (t, J=5.1, 1H), 7.82-7.92 (m, 1H), 7.34 (d, J=2.4, 1H), 7.01-7.10 (m, 2H), 6.87-6.99 (m, 3H), 6.72-6.83 (m, 1H), 4.68 (d, J=5.4, 2H), 3.66-3.80 (m, 12H). MS m/z: 464 (M+1).

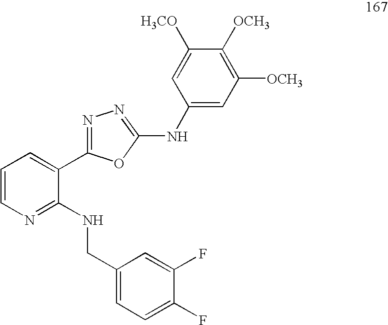

(3,4-Difluoro-benzyl)-{3-[5-(3,4,5-trimethoxy-phenylamino)-[1,3,4]oxadiazol-2-yl]-pyridin-2-yl}-amine (167): ¹HNMR (DMSO-d₆) δ (ppm) 10.65 (s, 1H), 8.16-8.37 (m, 2H), 7.83-7.97 (m, 1H), 7.30-7.49 (m, 2H), 6.93 (s, 2H), 6.74-6.86 (m, 1H), 4.79 (d, 2H), 3.68 (s, 6H), 3.55 (s, 3H). MS m/z: 470 (M+1).

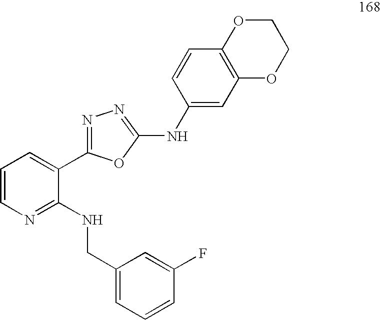

{3-[5-(2,3-Dihydro-benzo[1,4]dioxin-6-ylamino)-[1,3,4]oxadiazol-2-yl]-pyridin-2-yl}-(3-fluoro-benzyl)-amine (168): ¹HNMR (DMSO-d₆) δ (ppm) 10.47 (s, 1H), 8.07-8.34 (m, 2H), 7.78-7.98 (m, 1H), 7.32-7.54 (m, 2H), 7.09-7.30 (m, 3H), 6.93-7.07 (m, 1H), 6.68-6.91 (m, 2H), 4.67 (s, 2H), 4.23 (d, 4H). MS m/z: 420.15 (M+1).

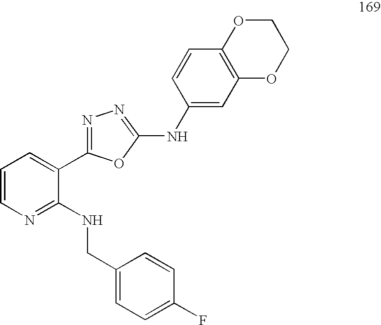

{3-[5-(2,3-Dihydro-benzo[1,4]dioxin-6-ylamino)-[1,3,4]oxadiazol-2-yl]-pyridin-2-yl}-(4-fluoro-benzyl)-amine (169): ¹HNMR (DMSO-d₆) δ (ppm) 10.38 (s, 1H), 7.95-8.20 (m, 2H), 7.61-7.79 (m, 1H), 7.12-7.32 (m, 1H), 6.77-7.11 (m, 5H), 6.52-6.74 (m, 2H), 4.53 (s, 2H), 4.08 (d, 4H). MS m/z: 420.15 (M+1)

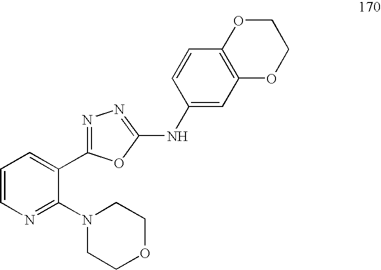

(2,3-Dihydro-benzo[1,4]dioxin-6-yl)-[5-(2-morpholin-4-yl-pyridin-3-yl)-[1,3,4]oxadiazol-2-yl]-amine (170): ¹HNMR (DMSO-d₆) δ (ppm) 10.23 (s, 1H), 8.07-8.35 (m, 1H), 7.72-7.96 (m, 1H), 7.08 (d, 1H), 6.74-6.96 (m, 2$, 6.63 (d, 1H), 3.88-4.20 (m, 4H), 3.34-3.63 (br, 4H), 2.85-3.07 (br, 4H). MS m/z: 382.23 (M+1).

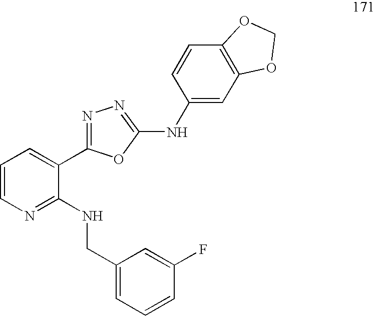

{3-[5-(Benzo[1,3]dioxol-5-ylamino)-[1,3,4]oxadiazol-2-yl]-pyridin-2-yl}-(3-fluoro-benzyl)-amine (171): ¹HNMR (DMSO-d₆) δ (ppm) 10.63 (s, 1H), 8.09-8.37 (m, 2H), 7.89 (d, 1H), 6.70-7.51 (m, 8H), 5.89 (s, 2H), 4.81 (d, 2H). MS m/z: 406.16 (M+1).

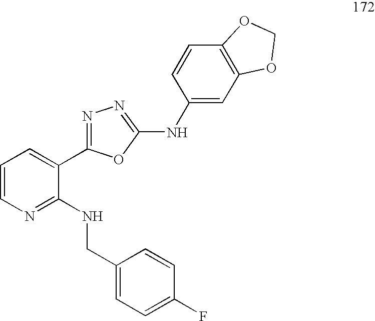

{3-[5-(Benzo[1,3]dioxol-5-ylamino)-[1,3,4]oxadiazol-2-yl]-pyridin-2-yl}-(4-fluoro-benzyl)-amine (172): ¹HNMR (DMSO-d₆) δ (ppm) 10.58 (s, 1H), 8.10-8.30 (m, 2H), 7.88 (d, 1H), 7.34-7.51 (m, 2H), 7.23 (s, 1H), 7.16 (t, 2H), 6.97-7.08 (m, 1H), 6.91 (d, 1H), 6.69-6.84 (m, 1H), 5.94 (s, 2H), 4.78 (d, 2H). MS m/z: 406.16 (M+1).

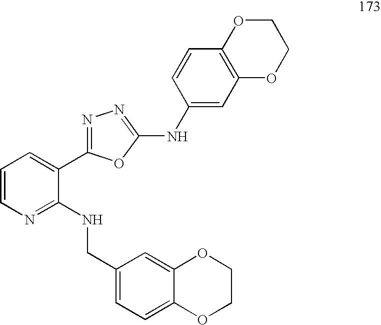

{3-[5-(2,3-Dihydro-benzo[1,4]dioxin-6-ylamino)-[1,3,4]oxadiazol-2-yl]-pyridin-2-yl}-(2,3-dihydro-benzo[1,4]dioxin-6-ylmethyl)-amine (173): ¹HNMR (DMSO-d₆) δ (ppm) 10.52 (s, 1H), 8.20-8.23 (m, 1H), 8.02-8.06 (m, 1H), 7.80-7.84 (m, 1H), 7.18-7.21 (m, 1H), 6.94-6.98 (m, 1H), 6.70-6.87 (m, 5H), 4.73 (d, 2H), 4.16-4.22 (m, 8H). MS m/z: 460 (M+1).

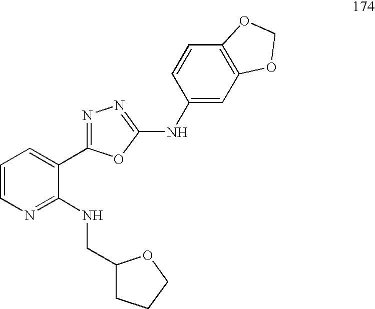

{3-[5-(Benzo[1,3]dioxol-5-ylamino)-[1,3,4]oxadiazol-2-yl]-pyridin-2-yl}-(tetrahydro-furan-2-ylmethyl)-amine (174): ¹HNMR (DMSO-d₆) δ (ppm) 10.53 (s, 1H), 8.14-8.41 (m, 2H), 7.77-8.11 (m, 1H), 7.33 (d, 1H), 6.66-7.15 (m, 3H), 5.91 (s, 2H), 3.97-4.25 (m, 1H), 3.42-3.96 (m, 4H), 1.72-2.11 (m, 3H), 1.44-1.72 (m, 1H). MS m/z: 382 (M+1).

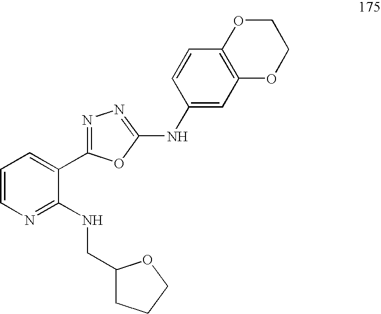

{3-[5-(2,3-Dihydro-benzo[1,4]dioxin-6-ylamino)-[1,3,4]oxadiazol-2-yl]-pyridin-2-yl}-(tetrahydro-furan-2-ylmethyl)-amine (175): ¹HNMR (DMSO-d₆) δ (ppm) 10.42 (s, 1H), 8.12-8.35 (m, 1H), 7.73-8.12 (m, 2H), 7.26 (d, 1H), 6.68-7.13 (m, 3H), 3.97-4.42 (m, 4H), 3.44-3.96 (m, 4H), 1.72-2.12 (m, 3H), 1.46-1.72 (m, 1H). MS m/z: 396 (M+1).

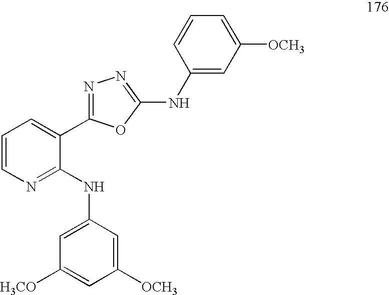

(3,5-Dimethoxy-phenyl)-{3-[5-(3-methoxy-phenylamino)-[1,3,4]oxadiazol-2-yl]-pyridin-2-yl}-amine (176): ¹HNMR (DMSO-d₆) δ (ppm) 10.89 (s, 1H), 10.20 (s, 1H), 8.33-8.38 (m, 1H), 8.01-8.04 (m, 1H), 7.22-7.33 (m, 2H), 7.10-7.22 (m, 1H), 7.01-7.09 (m, 3H), 6.62-6.69 (m, 1H), 6.21-6.25 (m, 1H), 3.78-3.87 (m, 9H). MS m/z: 420 (M+1).

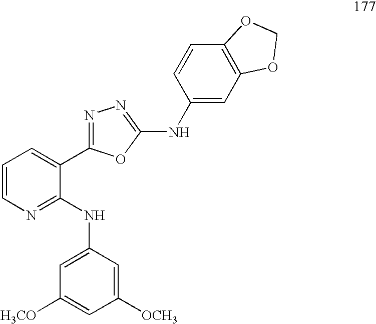

{3-[5-(Benzo[1,3]dioxol-5-ylamino)-[1,3,4]oxadiazol-2-yl]-pyridin-2-yl}-(3,5-dimethoxy-phenyl)-amine (177): ¹HNMR (DMSO-d₆) δ (ppm) 10.76 (s, 1H), 10.07 (s, 1H), 8.40-8.43 (m, 1H), 8.02-8.05 (m, 1H), 7.34 (d, 1H), 6.94-7.11 (m, 5H), 6.25 (t, 1H), 6.05 (s, 2H), 3.79 (d, 6H). MS m/z: 434 (M+1).

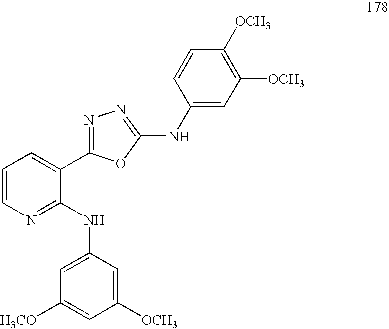

(3,5-Dimethoxy-phenyl)-{3-[5-(3,4-dimethoxy-phenylamino)-[1,3,4]oxadiazol-2-yl]-pyridin-2-yl}-amine (178): ¹HNMR (DMSO-d₆) δ (ppm) 10.68 (s, 1H), 10.04 (s, 1H), 8.41 (t, 1H), 8.05 (t, 1H), 7.36 (d, 1H), 7.00-7.19 (m, 5H), 6.25 (s, 1H), 3.78-3.82 (m, 12H). MS m/z: 450 (M+1).

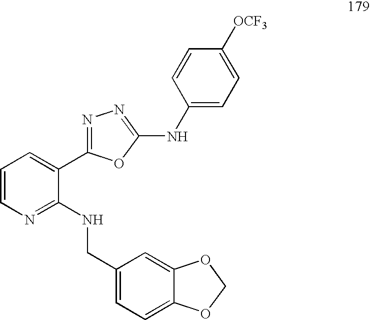

Benzo[1,3]dioxol-5-ylmethyl-{3-[5-(4-trifluoromethoxy-phenylamino)-[1,3,4]oxadiazol-2-yl]-pyridin-2-yl}-amine (179): ¹HNMR (DMSO-d₆) δ (ppm) 11.05 (s, 1H), 8.28-8.30 (s, 1H), 8.14 (t, 1H), 7.92-7.96 (m, 1H), 7.75 (d, 2H), 7.17-7.29 (m, 1H), 7.00 (s, 1H), 6.91 (s, 2H), 6.80-6.84 (m, 1H), 6.02 (s, 2H), 4.71 (d, 2H). MS m/z: 472 (M+1).

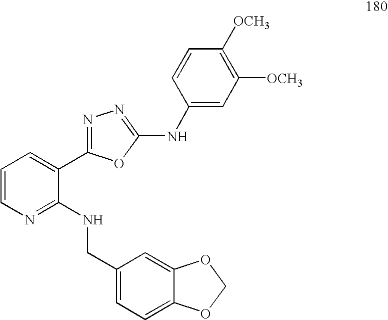

Benzo[1,3]dioxol-5-ylmethyl-{3-[5-(3,4-dimethoxy-phenylamino)-[1,3,4]oxadiazol-2-yl]-pyridin-2-yl}-amine (180): ¹HNMR (DMSO-d₆) δ (ppm) 10.60 (s, 1H), 8.26-8.29 (m, 1H), 8.16 (t, 1H), 7.89-7.93 (m, 1H), 7.39 (d, 1H), 6.79-7.12 (m, 6H), 6.02 (s, 2H), 4.71 (d, 2H), 3.78 (d, 6H). MS m/z: 448 (M+1).

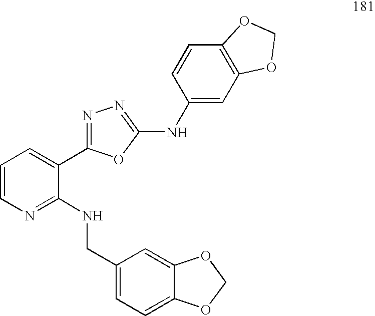

{3-[5-(Benzo[1,3]dioxol-5-ylamino)-[1,3,4]oxadiazol-2-yl]-pyridin-2-yl}-benzo[1,3]dioxol-5-ylmethyl-amine (181): ¹HNMR (DMSO-d₆) δ (ppm) 10.62 (s, 1H), 8.23-8.25 (m, 1H), 8.09 (t, 1H), 7.84-7.88 (m, 1H), 7.27-7.28 (d, 1H), 6.75-7.03 (m, 6H), 6.00 (s, 2H), 5.99 (s, 2H), 4.67 (d, 2H). MS m/z: 432 (M+1).

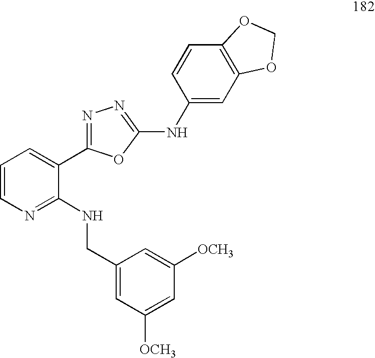

{3-[5-(Benzo[1,3]dioxol-5-ylamino)-[1,3,4]oxadiazol-2-yl]-pyridin-2-yl}-(3,5-dimethoxy-benzyl)-amine (182): ¹HNMR (DMSO-d₆) δ (ppm) 10.64 (s, 1H), 8.12-8.24 (m, 2H), 7.86-7.89 (m, 1H), 7.29 (d, 1H), 6.76-7.04 (m, 3H), 6.55 (d, 2H), 6.40 (t, 1H), 6.00 (s, 2H), 4.71 (d, 2H), 3.72 (s, 6H). MS m/z: 448 (M+1).

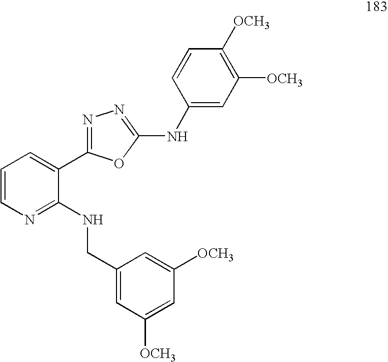

(3,5-Dimethoxy-benzyl)-{3-[5-(3,4-dimethoxy-phenylamino)-[1,3,4]oxadiazol-2-yl]-pyridin-2-yl}-amine (183): ¹HNMR (DMSO-d₆) δ (ppm) 10.57 (s, 1H), 8.15-8.24 (m, 2H), 7.87-7.90 (m, 1H), 7.35-7.36 (m, 1H), 7.06-7.10 (m, 1H), 6.95 (d, 1H), 6.76-6.81 (m, 1H), 6.55 (d, 2H), 6.40 (t, 1H), 4.72 (d, 2H), 3.72-3.77 (m, 12H). MS m/z: 464 (M+1).

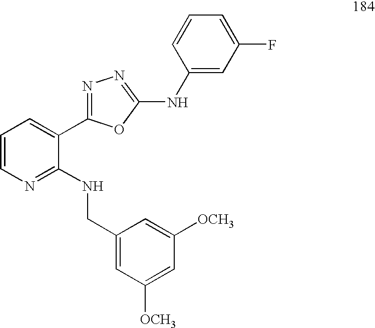

(3,5-Dimethoxy-benzyl)-{3-[5-(3-fluoro-phenylamino)-[1,3,4]oxadiazol-2-yl]-pyridin-2-yl}-amine (184): ¹HNMR (DMSO-d₆) δ (ppm) 11.05 (s, 1H), 8.14-8.26 (m, 2H), 7.90 (d, 1H), 7.37-7.57 (m, 3H), 7.09 (d, 1H), 6.80-6.88 (m, 2H), 6.54 (s, 2H), 6.40 (s, 1H), 3.72 (s, 6H). MS m/z: 422 (M+1).

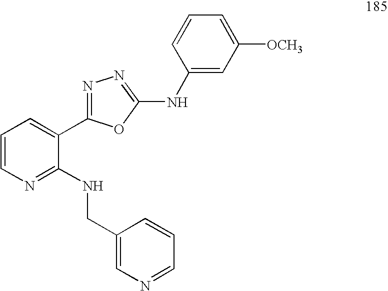

{3-[5-(3-Methoxy-phenylamino)-[1,3,4]oxadiazol-2-yl]-pyridin-2-yl}-pyridin-3-ylmethyl-amine (185): ¹HNMR (DMSO-d₆) δ (ppm) 8.61 (s, 1H), 8.46 (d, 1H), 8.22-8.30 (m, 2H), 7.89-7.91 (m, 1H), 7.76-7.79 (m, 1H), 7.13-7.36 (m, 4H), 6.72-6.83 (m, 1H), 6.59-6.61 (m, 1H), 4.83 (d. 2H), 3.76 (s. 3H). MS m/z: 375 (M+1).

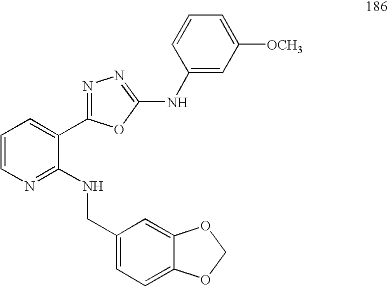

Benzo[1,3]dioxol-5-ylmethyl-{3-[5-(3-methoxy-phenylamino)-[1,3,4]oxadiazol-2-yl]-pyridin-2-yl}-amine (186): ¹HNMR (DMSO-d₆) δ (ppm) 10.60 (s, 1H), 8.10-8.15 (m, 1H), 7.88-7.92 (t, 1H), 7.61-7.70 (m, 1H), 6.41-7.14 (m, 8H), 5.85 (s, 2H), 4.50 (d, 2H), 3.62 (s, 3). MS m/z: 418 (M+1).

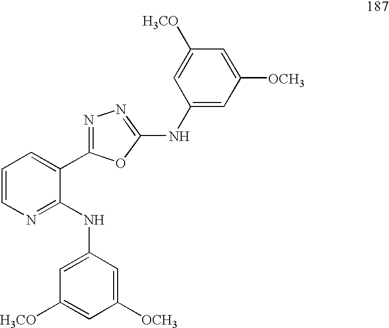

(3,5-Dimethoxy-phenyl)-{3-[5-(3,5-dimethoxy-phenylamino)-[1,3,4]oxadiazol-2-yl]-pyridin-2-yl}-amine (187): ¹HNMR (DMSO-d₆) δ (ppm) 11.00 (s, 1H), 10.10 (s, 1H), 8.53-8.55 (m, 1H), 8.15-8.18 (m, 1H), 7.15-7.17 (m, 3H), 7.01 (s, 1H), 6.31-6.38 (m, 2H), 3.91-3.97 (m, 12H). MS m/z: 450 (M+1).

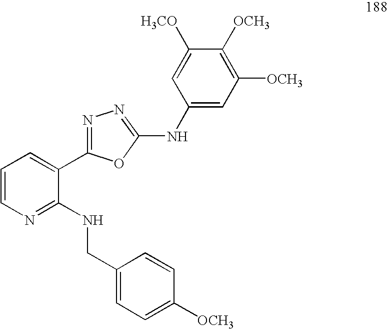

(4-Methoxy-benzyl)-{3-[5-(3,4,5-trimethoxy-phenylamino)-[1,3,4]oxadiazol-2-yl]-pyridin-2-yl}-amine (188): ¹HNMR (DMSO-d₆) δ (ppm) 10.68 (s, 1H), 8.17-8.31 (m, 1H), 8.01-8.16 (m, 1H), 7.80-7.93 (m, 1H), 7.33 (d, 2H), 6.94 (s, 2H), 6.89 (d, 2H), 6.71-6.83 (m, 1H), 4.73 (d, 2H), 3.67-3.88 (m, 9H), 3.54 (s, 3H). LCMS m/z: 464 (M+1).

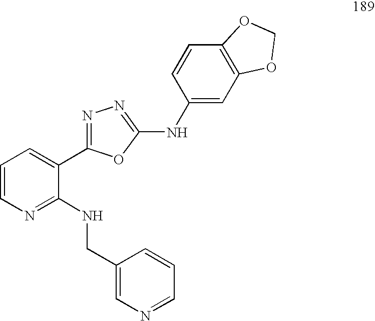

{3-[5-(Benzo[1,3]dioxol-5-ylamino)-[1,3,4]oxadiazol-2-yl]-pyridin-2-yl}-pyridin-3-ylmethyl-amine (189): ¹HNMR (DMSO-d₆) δ (ppm) 10.69 (br, s, 1H), 8.62 (d, 1H), 8.45-8.47 (m, 1H), 8.21-8.28 (m, 2H), 7.86-7.89 (m, 1H), 7.78 (d, 1H), 7.31-7.38 (m, 2H), 7.02-7.06 (m, 1H), 6.89-6.92 (m, 1H), 6.77-6.81 (m, 1H), 6.00 (s, 2H), 4.82 (d, 2H). MS m/z: 389 (M+1).

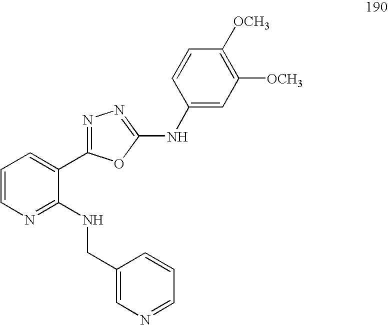

{3-[5-(3,4-Dimethoxy-phenylamino)-[1,3,4]oxadiazol-2-yl]-pyridin-2-yl}-pyridin-3-ylmethyl-amine (190): ¹HNMR (DMSO-d₆) δ (ppm) 8.62 (s, 1H), 8.44-8.46 (m, 1H), 8.21-8.31 (m, 2H), 7.76-7.90 (m, 2H), 7.32-7.40 (m, 2H), 7.06-7.10 (m, 1H), 6.93-6.96 (m, 1H), 6.77-6.93 (m, 1H), 4.82 (d, 2H), 3.67-3.77 (m, 6H). MS m/z: 405 (M+1).

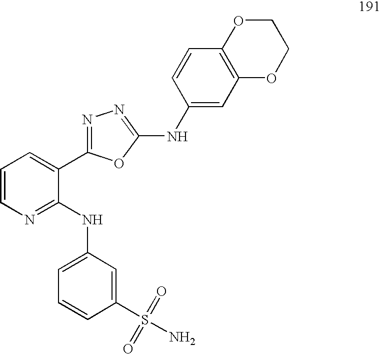

3-{3-[5-(2,3-Dihydro-benzo[1,4]dioxin-6-ylamino)-[1,3,4]oxadiazol-2-yl]-pyridin-2-ylamino}-benzenesulfonamide (191): ¹HNMR (DMSO-d₆) δ (ppm) 10.81 (s, 1H), 10.39 (s, 1H), 8.56-8.60 (m, 1H), 8.40-8.43 (m, 1H), 8.16-8.18 (m, 2H), 7.48-7.83 (m, 4H), 7.39 (d, 1H), 7.12-7.20 (m, 2H), 6.95-7.00 (m, 1H), 4.46 (d, 4H). MS m/z: 467 (M+1).

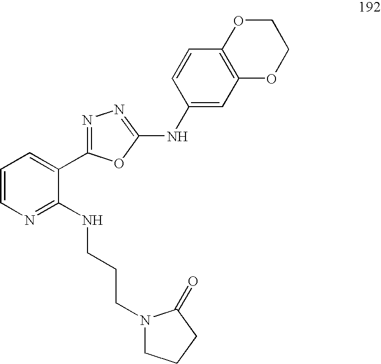

1-(3-{3-[5-(2,3-Dihydro-benzo[1,4]dioxin-6-ylamino)-[1,3,4]oxadiazol-2-yl]-pyridin-2-ylamino}-propyl)-pyrrolidin-2-one (192): ¹HNMR (DMSO-d₆) δ (ppm) 10.39 (s, 1H), 8.02-8.05 (m, 1H), 7.63-7.68 (m, 2H), 7.08 (d, 1H), 6.82-6.86 (m, 1H), 6.65-6.68 (m, 1H), 6.48-6.54 (m, 1H), 4.04-4.08 (m, 4H), 3.35-3.38 (m. 2H), 3.05-3.18 (m, 4H), 1.96-2.04 (m, 2H), 1.56-1.78 (m, 4H). MS m/z: 437 (M+1).

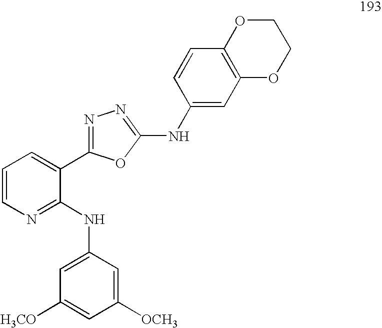

{3-[5-(2,3-Dihydro-benzo[1,4]dioxin-6-ylamino)-[1,3,4]oxadiazol-2-yl]-pyridin-2-yl}-(3,5-dimethoxy-phenyl)-amine (193): ¹HNMR (DMSO-d₆) δ (ppm) 10.82 (s, 1H), 10.22 (s, 1H), 8.56-8.58 (m, 1H), 8.06-8.09 (m, 1H), 7.42 (d, 1H), 7.20-7.23 (m, 4H), 7.02-7.06 (m, 1H), 6.40 (t, 1H), 4.38-4.43 (m, 4H), 3.95 (s, 6H). MS m/z: 448 (M+1).

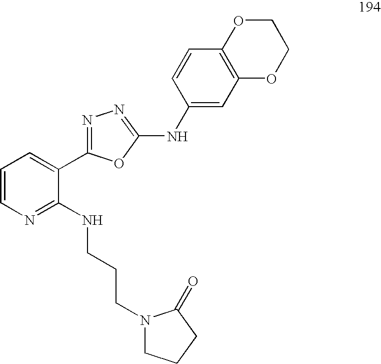

1-(3-{3-[5-(Benzo[1,3]dioxol-5-ylamino)-[1,3,4]oxadiazol-2-yl]-pyridin-2-ylamino}-propyl)-pyrrolidin-2-one (194): ¹HNMR (DMSO-d₆) δ (ppm) 9.58 (s, br, 1H), 8.24-8.26 (m, 1H), 7.80-7.86 (m, 2H), 7.31 (d, 1H), 6.98-7.04 (m, 1H), 6.97-6.92 (m, 1H), 6.72-6.76 (m, 1H), 6.03 (s, 2H), 3.50-3.54 (m, 2H), 3.24-3.40 (m, 4H), 2.20-2.24 (m, 2H), 1.80-1.95 (m, 4H). MS m/z: 423 (M+1).

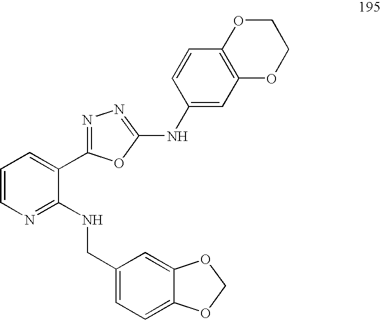

Benzo[1,3]dioxol-5-ylmethyl-{3-[5-(2,3-dihydro-benzo[1,4]dioxin-6-ylamino)-[1,3,4]oxadiazol-2-yl]-pyridin-2-yl}-amine (195): ¹HNMR (DMSO-d₆) δ (ppm) 10.48 (s, 1H), 8.19-8.30 (m, 1H), 8.03-8.16 (m, 1H), 7.79-7.91 (m, 1H), 7.14-7.28 (d, 1H), 6.92-7.06 (m, 2H), 6.72-6.91 (m, 4H), 5.92 (s, 2H), 4.68 (d, 2H), 4.14-4.32 (m, 4H). MS m/z: 446.10 (M+1).

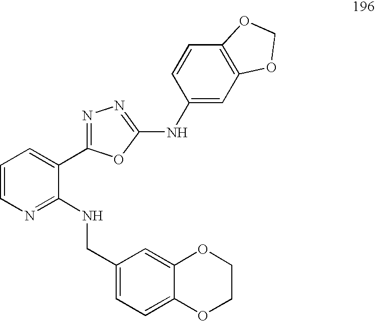

{3-[5-(Benzo[1,3]dioxol-5-ylamino)-[1,3,4]oxadiazol-2-yl]-pyridin-2-yl}-(2,3-dihydro-benzo[1,4]dioxin-6-ylmethyl)-amine (196): ¹HNMR (DMSO-d₆) δ (ppm) 10.58 (s, 1H), 8.14-8.18 (m, 1H), 7.96 (t, 1H), 7.72-7.76 (m, 1H), 7.21 (d, 1H), 6.90-6.94 (m, 1H), 6.65-6.85 (m, 5H), 5.92 (s, 2H), 4.57 (d, 2H), 4.11 (s, 4H). MS m/z: 446 (M+1).

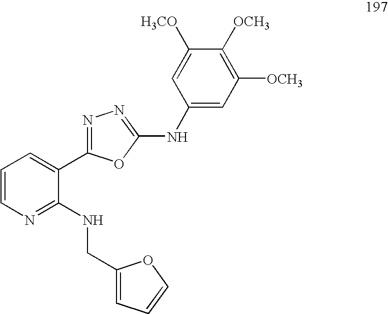

Furan-2-ylmethyl-{3-[5-(3,4,5-H trimethoxy-phenylamino)-[1,3,4]oxadiazol-2-yl]-pyridin-2-yl}-amine (197): ¹HNMR (DMSO-d₆) δ (ppm) 10.77 (br s, 1H), 8.34 (dd, J=3.3, 0.9 Hz, 1H), 8.17 (t, J=5.4 Hz, 1H), 7.96-7.94 (m, 1H), 7.70-7.67 (m, 1H), 7.05 (s, 2H), 6.90 (dd, J=4.8, 2.7 Hz, 1H), 6.48 (t, 2.4 Hz, 1H), 6.41-6.38 (m, 1H), 4.88-4.84 (m, 2H), 3.85 (s, 6H), 3.69 (s, 3H). MS m/z: 424 (M+1).

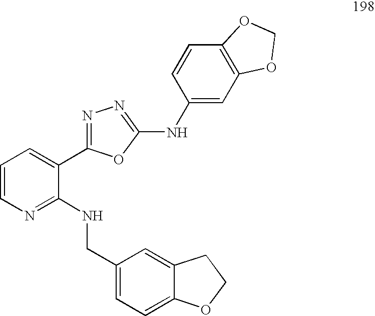

{3-[5-(Benzo[1,3]dioxol-5-ylamino)-[1,3,4]oxadiazol-2-yl]-pyridin-2-yl}-(2,3-dihydro-benzofuran-5-ylmethyl)-amine (198): ¹HNMR (DMSO-d₆) δ (ppm) 10.85 (s, 1H), 8.40-8.46 (m, 1H), 7.95-8.00 (m, 1H), 7.34-7.38 (m, 2H), 7.26-7.30 (m, 1H), 7.20-7.23 (m, 1H), 7.02-7.06 (m, 1H), 6.82-6.92 (m, 2H), 6.12 (s, 2H), 4.86-4.88 (m, 2H), 4.68-4.73 (m, 2H), 3.20-3.30 (m, 12H). MS m/z: 430 (M+1).

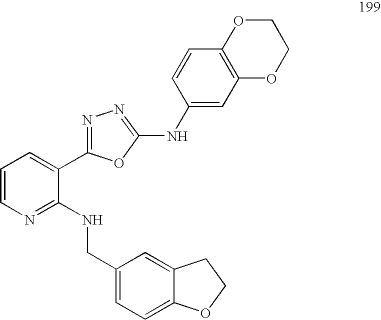

{3-[5-(2,3-Dihydro-benzo[1,4]dioxin-6-ylamino)-[1,3,4]oxadiazol-2-yl]-pyridin-2-yl}-(2,3-dihydro-benzofuran-5-ylmethyl)-amine (199): ¹HNMR (DMSO-d₆) δ (ppm) 10.30 (s, 1H), 8.02-8.06 (m, 1H), 7.82-7.86 (m, 1H), 7.62-7.68 (m, 1H), 6.95-7.02 (m, 2H), 6.90-6.94 (m, 1H), 6.81-6.85 (m, 1H), 6.46-6.66 (m, 2H), 4.47-4.49 (m, 2H), 4.25-4.35 (m, 4H), 4.00-4.08 (m, 4H). MS m/z: 444 (M+1).

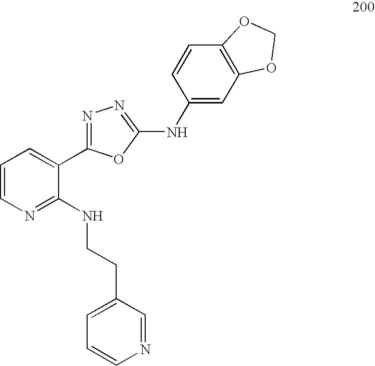

{3-[5-(Benzo[1,3]dioxol-5-ylamino)-[1,3,4]oxadiazol-2-yl]-pyridin-2-yl}-(2-pyridin-3-yl-ethyl)-amine (200): ¹HNMR (DMSO-d₆) δ (ppm) 10.74 (s, 1H), 8.44-8.58 (m, 2H), 8.26-8.30 (m, 1H), 7.85-7.92 (m, 2H), 7.74-7.79 (m, 1H), 7.28-7.36 (m, 2H), 7.04-7.08 (m, 1H), 6.92-6.96 (m, 1H), 6.80-6.86 (m, 1H), 6.09 (s, 2H), 3.80-3.90 (m, 2H), 3.00-3.06 (m, 2H), MS m/z: 403 (M+1).

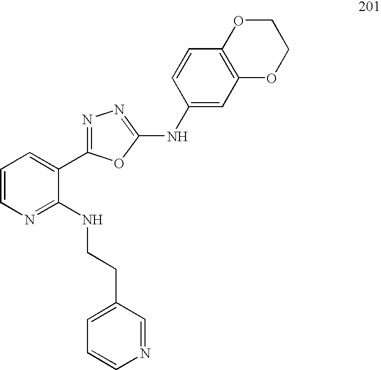

{3-[5-(2,3-Dihydro-benzo[1,4]dioxin-6-ylamino)-[1,3,4]oxadiazol-2-yl]-pyridin-2-yl}-(2-pyridin-3-yl-ethyl)-amine (201): ¹HNMR (DMSO-d₆) δ (ppm) 10.50 (s, 1H), 8.30-8.45 (m, 2H), 8.15-8.20 (m, 1H), 7.80-7.90 (m, 2H), 7.61-7.66 (m, 1H), 7.12-7.28 (m, 2H), 7.92-7.98 (m, 1H), 6.65-6.80 (m, 2H), 4.05-4.20 (m, 4H), 3.71-3.76 (m, 2H), 2.85-2.92 (m, 2H). MS m/z: 417 (M+1).

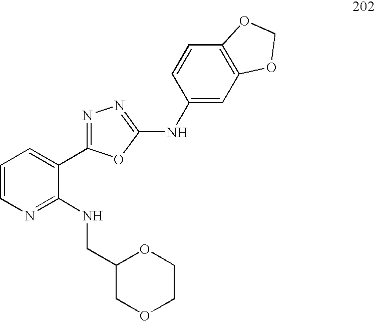

{3-[5-(Benzo[1,3]dioxol-5-ylamino)-[1,3,4]oxadiazol-2-yl]-pyridin-2-yl}-[1,4]dioxan-2-ylmethyl-amine (202): ¹HNMR (DMSO-d₆) δ (ppm) 10.60 (s, 1H), 8.17-8.33 (m, 1H), 7.79-8.01 (m, 2H), 7.25-7.38 (m, 1H), 6.98-7.12 (m, 1H), 6.93 (d, 1H), 6.69-6.85 (m, 1H), 5.92 (s, 2H), 3.39-3.88 (m, 7H), 3.24-3.39 (m, 2H). MS m/z: 398 (M+1).

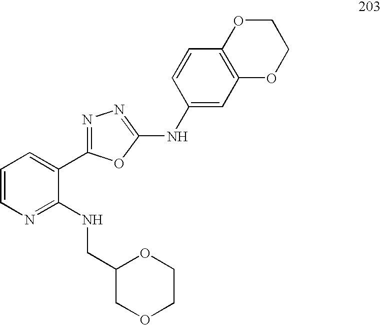

{3-[5-(2,3-Dihydro-benzo[1,4]dioxin-6-ylamino)-[1,3,4]oxadiazol-2-yl]-pyridin-2-yl}-[1,4]dioxan-2-ylmethyl-amine (203): ¹HNMR (DMSO-d₆) δ (ppm) 10.49 (s, 1H), 8.17-8.31 (m, 1H), 7.76-8.01 (m, 2H), 7.17-7.33 (m, 1H), 6.95-7.08 (m, 1H), 6.88 (d, 1H), 6.69-6.81 (m, 1H), 4.11-4.37 (m, 4H), 3.40-3.87 (m, 7H), 3.22-3.38 (m, 2H). MS m/z: 411.

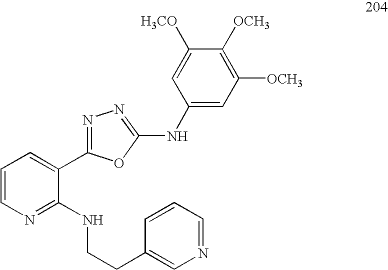

(2-Pyridin-3-yl-ethyl)-{3-[5-(3,4,5-trimethoxy-phenylamino)-[1,3,4]oxadiazol-2-yl]-pyridin-2-yl}-amine (204): ¹HNMR (DMSO-d₆) δ (ppm) 10.68 (s, 1H), 8.45-8.55 (m, 1H), 8.35-8.45 (m, 1H), 8.21-8.31 (m, 1H), 7.81-7.98 (m, 2H), 7.69 (d, 1H), 7.26-7.37 (m, 1H) 6.99 (s, 2H), 6.68-6.84 (m, 1H), 3.69-3.92 (m, 8H), 3.63 (s, 3H), 2.89-3.03 (t, 2H). MS m/z: 449.06 (M+1).

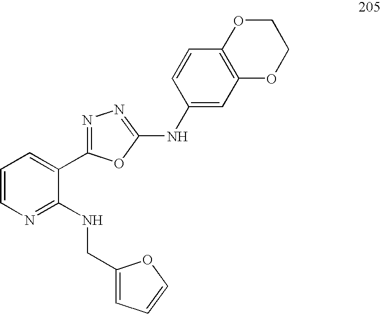

{3-[5-(2,3-Dihydro-benzo[1,4]dioxin-6-ylamino)-[1,3,4]oxadiazol-2-yl]-pyridin-2-yl}-furan-2-ylmethyl-amine (205): ¹HNMR (DMSO-d₆) δ (ppm) 13.40 (s, 1H), 9.23 (s, 1H), 8.59-8.60 (m, 1H), 8.10-8.25 (m, 2H), 7.69-7.71 (m, 1H), 7.48-7.51 (m, 1H), 6.96-7.00 (m, 1H), 6.78-6.82 (m, 2H), 6.30-6.39 (m, 2H), 4.80 (d, 2H), 4.22-4.31 (m, 4H). MS m/z: 391 (M+1).

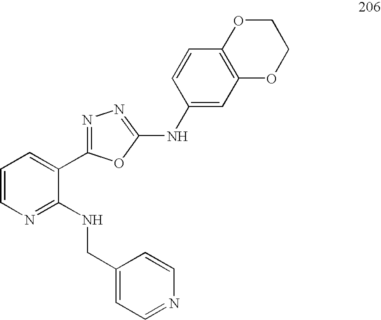

{3-[5-(2,3-Dihydro-benzo[1,4]dioxin-6-ylamino)-[1,3,4]oxadiazol-2-yl]-pyridin-2-yl}-pyridin-4-ylmethyl-amine (206): ¹HNMR (DMSO-d₆) δ (ppm) 10.61 (s, 1H), 8.52 (d, J=5.7, 2H), 8.24-8.40 (t, J=6.0, 1H), 8.10-8.24 (m, 1H), 7.81-7.99 (m, 1H), 7.18-7.45 (m, 3H), 6.94-7.10 (m, 1H), 6.732-6.92 (m, 2H), 4.76-4.93 (d, J=6.0, 2H), 4.14-4.31 (m, 4H). MS m/z: 403.12 (M+1).

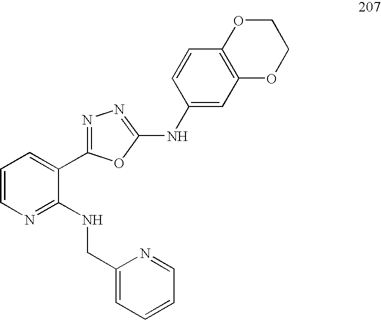

{3-[5-(2,3-Dihydro-benzo[1,4]dioxin-6-ylamino)-[1,3,4]oxadiazol-2-yl]-pyridin-2-yl}-pyridin-2-ylmethyl-amine (207): ¹HNMR (DMSO-d₆) δ (ppm) 10.55 (s, 1H), 8.44-8.66 (d, J=4.8, 2H), 8.16-8.27 (t, J=5.4, 1H), 7.84-7.95 (m, 1H), 7.69-7.81 (m, 1H), 7.19-7.43 (m, 3H), 6.95-7.07 (m, 1H), 6.73-6.91 (m, 2H), 4.83-4.93 (d, J=5.1, 2H), 4.16-4.29 (m, 4H). MS m/z: 403.09 (M+1).

### Example 14

### Synthesis of Benzo[1,3]dioxol-5-yl-{5-[2-(benzo[1,3]dioxol-5-ylmethoxy)-pyridin-3-yl]-[1,3,4]oxadiazol-2-yl}-amine (208)

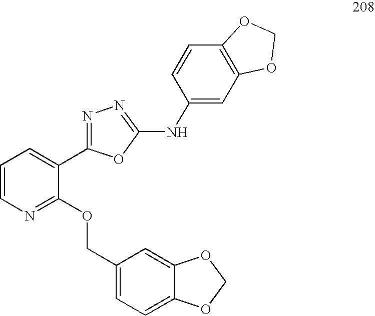

Benzo[1,3]dioxol-5-yl-{5-[2-(benzo[1,3]dioxol-5-ylmethoxy)-pyridin-3-yl]-[1,3,4]oxadiazol-2-yl}-amine (208): This compound was synthesized by reacting 71b (from Example 5) with 5-isothiocyanato-benzo[1,3]dioxole (from Oakwood products) and follow the procedures listed in Example 13 (step-3 and step-4): ¹HNMR (DMSO-d₆) δ (ppm) 10.29 (s, 1H), 8.09-8.26 (m, 1H), 7.89-8.04 (m, 1H), 6.89-7.18, (m, 3H), 6.76-6.89 (m, 2H), 6.61-6.76 (m, 2H), 5.71 (s, 4H), 5.13 (s, 2H). MS m/z: 432.91 (M+1).

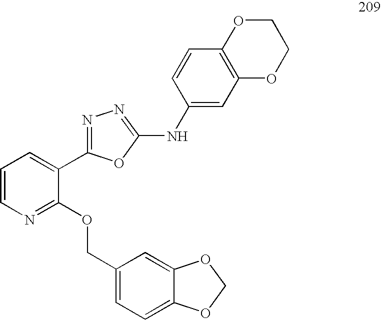

{5-[2-(Benzo[1,3]dioxol-5-ylmethoxy)-pyridin-3-yl]-[1,3,4]oxadiazol-2-yl}-(2,3-dihydro-benzo[1,4]dioxin-6-yl)-amine (209): ¹HNMR (DMSO-d₆) δ (ppm) 10.37 (s, 1H), 8.30-8.44 (m, 1H), 8.09-8.22 (m, 1H), 7.14-7.29 (m, 2H), 7.05-7.14 (m, 1H), 6.94-7.05 (m, 2H), 6.76-6.94 (m, 2H), 5.93 (s, 2H), 5.34 (s, 2H), 4.10-4.33 (m, 4H). MS m/z: 446.89 (M+1).

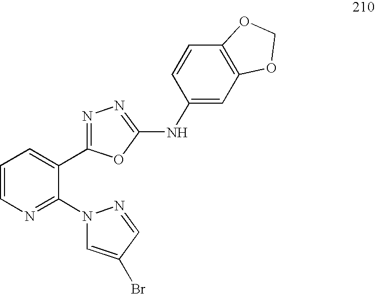

Benzo[1,3]dioxol-5-yl-{5-[2-(4-bromo-pyrazol-1-yl)-pyridin-3-yl]-[1,3,4]oxadiazol-2-yl}-amine (210): ¹HNMR (DMSO-d₆) δ (ppm) 10.48 (s, 1H), 8.83-9.03 (m, 2H), 8.43-8.60 (m, 1H), 8.01 (s, 1H), 7.75-7.90 (m, 1H), 7.29-7.42 (m, 1H), 6.97-7.18 (m, 2H), 6.08 (s, 2H). MS m/z: 428.75 (M+1).

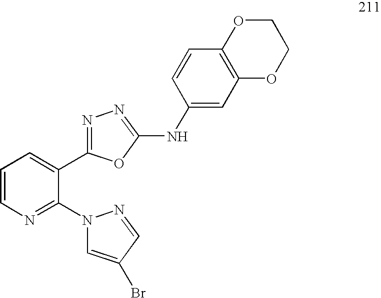

{5-[2-(4-Bromo-pyrazol-1-yl)-pyridin-3-yl]-[1,3,4]oxadiazol-2-yl}-(2,3-dihydro-benzo[1,4]dioxin-6-yl)-amine (211): ¹H NMR (Methanol-d₄) δ (ppm) 10.48 (s, 1H), 8.82-8.98 (m, 2H), 8.41-8.58 (m, 1H), 7.94-8.10 (s, 1H), 7.74-7.90 (m, 1H), 7.23-7.37 (m, 1H), 7.01-7.14 (m, 1H), 6.86-7.00 (m, 1H), 4.22-4.48 (m, 4H). MS m/z: 442.77 (M+1).

### Example 15

### synthesis of (4-Fluoro-benzyl)-{3-[5-(4-methanesulfonyl-phenyl)-[1,3,4]oxadiazol-2-yl]-pyridin-2-yl}-amine (212)

### Step 1: synthesis of 2-(4-fluoro-benzylamino)-nicotinic acid Ethyl Ester (212a)

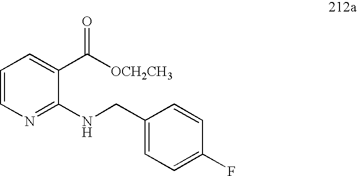

Ethyl-2-chloronicotinate (1.5 ml, 10.4 mmol) was added to a solution of triethylamine (3.5 ml, 24.9 mmol) in dimethylsulfoxide (7 ml) and stirred for five minutes. 4-Fluorobenzylamine (1.5 ml, 13.1 mmol) was added to the mixture and heated to 70° C. Upon disappearance of starting material, the reaction mixture was diluted with ethyl acetate (20 ml) and washed with deionized water (2×20 ml). The aqueous wash was re-extracted with ethyl acetate (3×20 ml). The organic layers were combined and dried over anhydrous sodium sulfate. The sodium sulfate was filtered, and the organic solvent was removed in vacuo. The yellow oil was purified with silica gel flash column chromatography (Hexane:Dichloromethane→1:2) to yield a yellow oil (2.0 g, 71%). ¹HNMR (Acetone-d₆) δ (ppm) 8.34-8.59 (br, 1H), 8.24-8.33 (m, 1H), 8.10-8.24 (m, 1H), 7.35-7.53 (m, 2H), 7.01-7.20 (m, 2H), 6.56-6.71 (m, 1H), 4.71 (d, T=5.7, 2H), 4.33 (q, J=7.2, 2H), 1.35 (t, J=7.2, 3H). MS m/z: 275.01 (M+1).

### Step 2: synthesis of 2-(4-Fluoro-benzylamino)-nicotinic acid Hydrazide (212b)

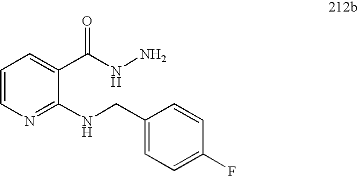

Isopropyl alcohol (10 ml) was added to a round bottom flask containing intermediate 212a (1.75 g, 6.38 mmol). Hydrazine monohydrate (3 ml, 61.8 mmol) was added to the mixture and heated to 70° C. Upon disappearance of starting material, the reaction mixture was diluted with ethyl acetate (15 ml) and washed with deionized water (2×15 ml). The organic layer was dried over anhydrous sodium sulfate and filtered. The organic solvent was removed in vacuo to yield a yellow oil. The flask was placed in an ice water bath, and the white solid was filtered and washed with diethyl ether (3×15 ml). (1.43 g, 86%). ¹HNMR (DMSO-d₆) δ (ppm) 9.79 (s, 1H), 8.60 (t, J=5.4, 1H), 8.04-8.25 (m, 1H), 7.77-7.97 (m, 1H), 7.28-7.48 (m, 2H), 7.04-7.22 (m, 2H), 6.49-6.66 (m, 1H), 4.64 (d, J=6.0, 2H), 4.46 (s, 1H). MS m/z: 261 (M+1).

### Step 3 Preparation of (4-Fluoro-benzyl)-{3-[5-(4-methanesulfonyl-phenyl)-[1,3,4]oxadiazol-2-yl]-pyridin-2-yl}-amine (212)

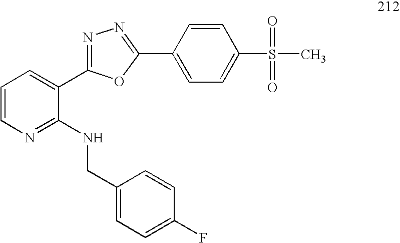

Intermediate 212b (51.4 mg, 0.20 mmol) was dissolved in methylene chloride (7 ml) and stirred at room temperature. 4-Methanesulfonyl-benzoic acid (40.9 mg, 0.20 mmol, from Peakdale Molecular), 2-chloro-1,3-dimethylimidazolinium chloride (DMC) (65.7 mg, 3.89 mmol), and anhydrous triethylamine (0.11 ml, 0.78 mmol) were added, and the reaction was monitored with TLC. Upon completion, the reaction was diluted with methylene chloride and washed 3×10 ml 5% citric acid, 3×10 ml saturated aqueous sodium bicarbonate, and 3×10 ml of saturated aqueous sodium chloride. The organic phase was dried over anhydrous magnesium sulfate, then filtered and concentrated. Methanol was added and the mixture was heated to 50° C. After 15 minutes, methanol was removed in vacuo to approximately 2 ml. The mixture was cooled in an ice water bath, then the white solid was filtered and washed with 3×5 ml diethyl ether (62.4 mg, 74.4%). ¹HNMR (DMSO-d₆) δ (ppm) 8.12-8.48 (m, 7H), 7.37-7.51 (m, 2H), 7.16 (t, J=8.7, 2H), 6.91 (m, 1H), 4.82 (d, J=5.4, 2H), 3.32 (s, 3H). MS m/z: 425 (M+1).

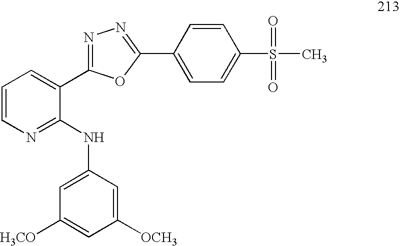

(3,5-Dimethoxy-phenyl)-{3-[5-(4-methanesulfonyl-phenyl)-[1,3,4]oxadiazol-2-yl]-pyridin-2-yl}-amine (213): ¹HNMR (DMSO-d₆) δ (ppm) 10.00 (s, 1H), 8.36-8.60 (m, 4H), 8.20 (d, J=8.4, 2H), 6.94-7.17 (m, 3H), 6.38 (s, 1H), 3.77 (s, 6H), 3.34 (s, 3H). MS m/z: 453 (M+1).

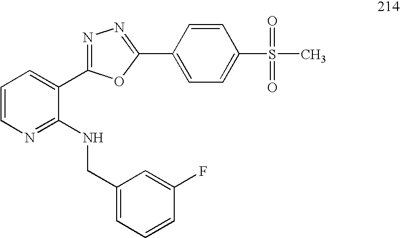

(3-Fluoro-benzyl)-{3-[5-(4-methanesulfonyl-phenyl)-[1,3,4]oxadiazol-2-yl]-pyridin-2-yl}-amine (214): ¹HNMR (DMSO-d₆) δ (ppm) 8.24-8.49 (m, 5H), 8.18 (d, J=8.4, 2H), 7.31-7.45 (m, 1H), 7.14-7.28 (m, 2H), 7.00-7.13 (m, 1H), 6.79-6.91 (m, 1H), 4.86 (d, J=6.0, 2H), 3.33 (s, 3H). MS m/z: 425 (M+1).

### Example 16

### Synthesis of (2,3-Dihydro-benzo[1,4]dioxin-6-yl)-(5-{2-[(pyridin-4-ylmethyl)-amino]-phenyl}-[1,3,4]oxadiazol-2-yl)-amine (215)

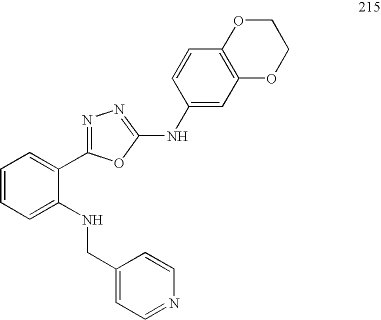

(2,3-Dihydro-benzo[1,4]dioxin-6-yl)-(5-{2-[(pyridin-4-ylmethyl)-amino]-phenyl}-[1,3,4]oxadiazol-2-yl)-amine (215): synthesized by reacting 111b (from Example 7) with 6-isothiocyanato-2,3-dihydro-benzo[1,4]dioxine (from Oakwood Products) and follow step-3 and step-4 in Example 13. ¹HNMR (DMSO-d₆) δ (ppm) 10.58 (s, 1 h), 8.57-8.63 (m, 2H), 7.86 (t, 1H), 7.58-7.64 (m, 1H), 7.20-7.33 (m, 4H), 6.96-7.02 (m, 1H), 6.67-6.88 (m, 3H), 4.65-4.67 (m, 2H), 4.16-4.25 (m, 4H). MS m/z: 402 (M+1).

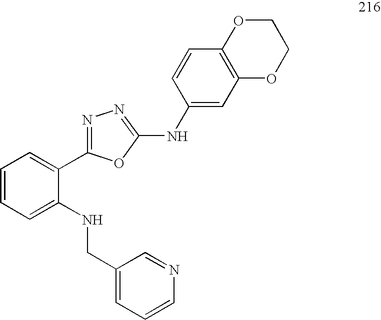

(2,3-Dihydro-benzo[1,4]dioxin-6-yl)-(5-{2-[(pyridin-3-ylmethyl)-amino]-phenyl}-[1,3,4]oxadiazol-2-yl)-amine (216): ¹HNMR (DMSO-d₆) δ (ppm) 10.37 (s, 1H), 8.48 (s, 1H), 8.40-8.41 (m, 1H), 7.62-7.73 (m, 2H), 7.46-7.50 (m, 1H), 7.08-7.25 (m, 3H), 6.85-6.91 (m, 1H), 6.58-6.72 (m, 3H), 4.48-4.51 (m, 1H), 4.05-4.10 (m, 4H). MS m/z: 402 (M+1).

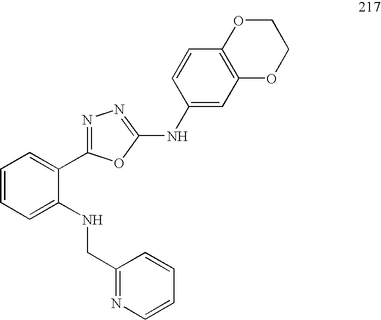

(2,3-Dihydro-benzo[1,4]dioxin-6-yl)-(5-{2-[(pyridin-2-ylmethyl)-amino]-phenyl}-[1,3,4]oxadiazol-2-yl)-amine (217): ¹HNMR (DMSO-d₆) δ (ppm) 10.52 (s, 1H), 8.62-8.65 (m, 1H), 8.18-8.22 (m, 1H), 7.75-7.81 (m, 1H), 7.25-7.36 (m, 4H), 7.10-7.16 (m, 1H), 6.75-6.98 (m, 3H), 4.68-4.70 (m, 2H), 4.22-4.28 (m, 4H). MS m/z: 402 (M+1).

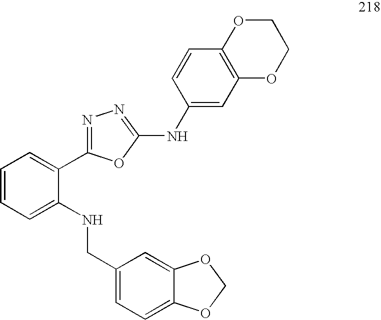

(5-{2-[(Benzo[1,3]dioxol-5-ylmethyl)-amino]-phenyl}-[1,3,4]oxadiazol-2-yl)-(2,3-dihydro-benzo[1,4]dioxin-6-yl)-amine (218): ¹HNMR (DMSO-d₆) δ (ppm) 10.46 (s, 1H), 7.79 (t, 1H), 7.58-7.61 (m, 1H), 7.21-7.25 (m, 2H), 6.96-7.02 (m, 2H), 6.81-6.88 (m, 3H), 6.71-6.76 (m 1H), 5.98 (s, 2H), 4.49 (d, 2H), 4.20-4.26 (m, 4H). MS m/z: 445 (M+1).

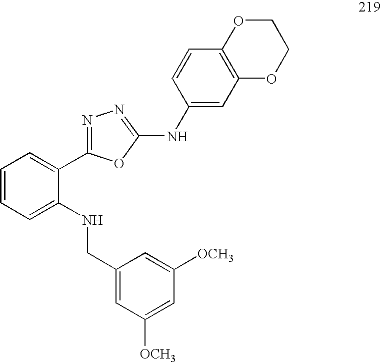

(2,3-Dihydro-benzo[1,4]dioxin-6-yl)-{5-[2-(3,5-dimethoxy-benzylamino)-phenyl]-[1,3,4]oxadiazol-2-yl}-amine (219): ¹HNMR (DMSO-d₆) δ (ppm) 10.48 (s, 1H), 7.81-7.86 (t, 1H), 7.61 (d, 1H), 7.39 (t, 1H), 7.22 (s, 1H), 6.98-7.04 (m, 1H), 6.61-6.75 (m, 3H), 6.65 (s, 2H), 6.41 (s, 1H), 4.52 (d, 2H), 4.23-4.27 (m, 4H), 3.73 (s, 6H). MS m/z: 491 (M+1).

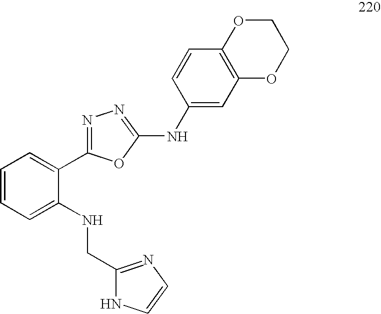

(2,3-Dihydro-benzo[1,4]dioxin-6-yl)-(5-{2-[(1H-imidazol-2-ylmethyl)-amino]-phenyl}-[1,3,4]oxadiazol-2-yl)-amine (220): ¹HNMR (DMSO-d₆) δ (ppm) 10.45 (s, 1H), 7.91 (s, 1H), 7.64 (d, 2H), 7.31-7.39 (m, 1H), 7.23 (s, 1H), 6.92-7.06 (m, 3H), 6.90-6.95 (m, 2H), 6.75-6.81 (m, 1H), 4.59 (d, 2H), 4.24-4.28 (m, 4H). MS m/z: 391 (M+1).

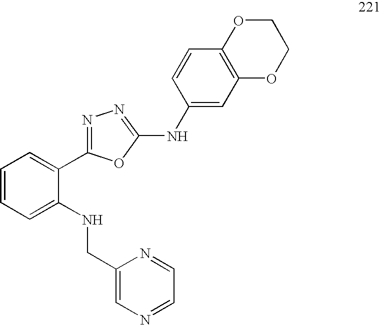

(2,3-Dihydro-benzo[1,4]dioxin-6-yl)-(5-{2-[pyrazin-2-ylmethyl)-amino]-phenyl}-[1,3,4]oxadiazol-2-yl)-amine (221): ¹HNMR (DMSO-d₆) δ (ppm) 10.46 (s, 1H), 9.14 (s, 1H), 8.85 (s, 2H), 7.96 (t, 1H), 7.74-7.78 (m, 1H), 7.38-7.41 (m 1H), 7.29 (s, 1H), 7.04-7.09 (m, 1H), 6.82-6.96 (m, 3H), 4.77 (d, 2H), 4.22-4.26 (m, 4H). MS m/z: 403 (M+1).

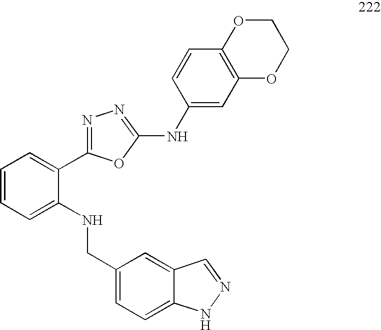

(2,3-Dihydro-benzo[1,4]dioxin-6-yl)-(5-{2-[(1H-indazol-5-ylmethyl)-amino]-phenyl}-[1,3,4]oxadiazol-2-yl)-amine (222): ¹HNMR (DMSO-d₆) δ (ppm) 13.27 (s, 1H), 10.49 (s, 1H), 8.08 (s, 1H), 7.88 (s, 1H), 7.75 (s, 1H), 7.52-7.63 (m, 2H), 7.20-7.39 (m, 3H), 6.75-6.99 (m, 4H), 4.66 (s, 2H), 4.28 (s, 4H). MS m/z: 441 (M+1).

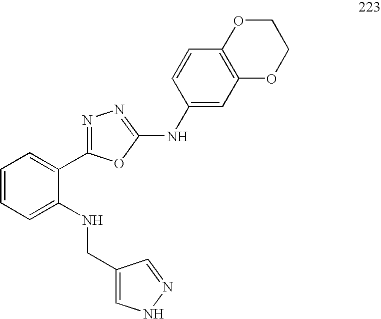

(2,3-Dihydro-benzo[1,4]dioxin-6-yl)-(5-{2-[(1H-pyrazol-4-ylmethyl)-amino]-phenyl}-[1,3,4]oxadiazol-2-yl)-amine (223): ¹HNMR (DMSO-d₆) δ (ppm) 10.36 (s, 1H), 7.68 (s, 1H), 7.07-7.47 (m, 4H), 6.59-6.89 (m, 4H), 4.26-4.28 (m, 2H), 4.04-4.14 (m, 4H). MS m/z: 392 (M+1).

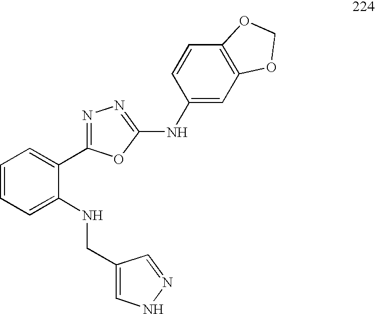

Benzo[1,3]dioxol-5-yl-(5-{2-[(1H-pyrazol-4-ylmethyl)-amino]-phenyl}-[1,3,4]oxadiazol-2-yl)-amine (224): ¹HNMR (DMSO-d₆) δ (ppm) 12.83 (s, 1H), 7.60-7.75 (m, 3H), 7.48-7.51 (m, 1H), 7.32-7.38 (m, 1H), 7.29 (d, 1H), 6.90-7.05 (m, 3H), 6.72-6.76 (m, 1H), 6.02 (s. 2H), 4.38-4.40 (m, 2H). MS m/z: 377 (M+1).

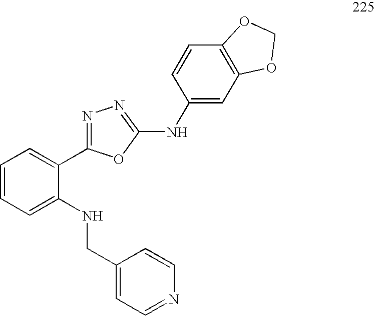

Benzo[1,3]dioxol-5-yl-(5-{2-[(pyridin-4-ylmethyl)-amino]-phenyl}-[1,3,4]oxadiazol-2-yl)-amine (225): ¹HNMR (DMSO-d₆) δ (ppm) 10.58 (s, 1H), 8.51-8.53 (m, 2H), 7.93-7.97 (m, 1H), 7.62-7.66 (m, 1H), 7.23-7.38 (m, 4H), 7.03-7.10 (m, 1H), 6.90-6.94 (m, 1H), 6.65-6.75 (m, 2H), 6.03 (s, 2H), 4.74-4.76 (m, 2H). MS m/z: 388 (M+1).

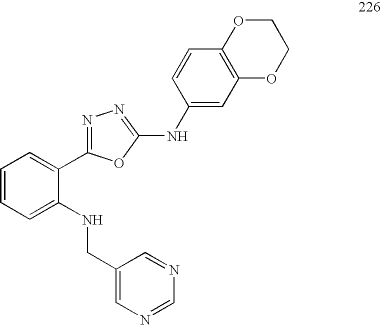

(2,3-Dihydro-benzo[1,4]dioxin-6-yl)-(5-{2-[(pyrimidin-5-ylmethyl)-amino]-phenyl}-[1,3,4]oxadiazol-2-yl)-amine (226): ¹HNMR (DMSO-d₆) δ (ppm) 10.40 (s, 1H), 8.74 (s, 1H), 8.58 (s, 1H), 8.16 (t, 1H), 7.50-7.52 (m, 1H), 7.24-7.26 (m, 2H), 6.98-7.02 (m, 1H), 6.75-6.81 (m 2H), 6.65-6.68 (m, 2H), 4.79 (d, 2H), 4.24-4.28 (m 4H). MS m/z: 403 (M+1).

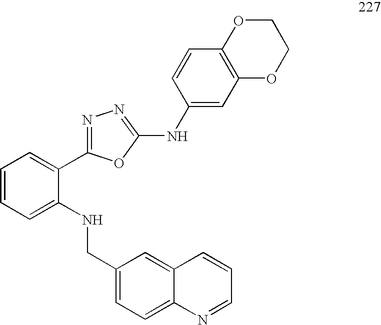

(2,3-Dihydro-benzo[1,4]dioxin-6-yl)-(5-{2-[(quinolin-6-ylmethyl)-amino]-phenyl}-[1,3,4]oxadiazol-2-yl)-amine (227): ¹HNMR (DMSO-d₆) δ (ppm) 9.98 (br s, 1H), 8.80 (s, 1H), 8.39 (d, 1H), 8.00-8.11 (m, 3H), 7.89-7.93 (m, 1H), 7.68-7.70 (m, 1H), 7.59-7.69 (m, 1H), 7.28 (s, 2H), 7.15-7.17 (m, 1H), 6.68-6.89 (m, 3H), 4.89 (d, 2H), 4.24-4.28 (m, 4H). MS m/z: 452 (M+1).

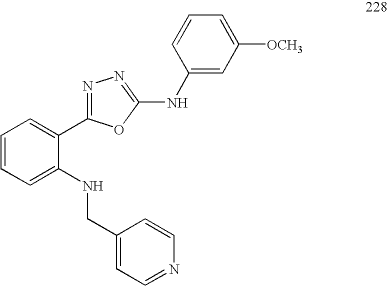

(3-Methoxy-phenyl)-(5-{2-[(pyridin-4-ylmethyl)-amino]-phenyl}-[1,3,4]oxadiazol-2-yl)-amine (228): ¹HNMR (DMSO-d₆) δ (ppm) 10.73 (s, 1H), 8.51 (d, J=6 Hz, 2H), 7.97 (t, J=6 Hz, 1H), 7.63 (d, J=8 Hz, 1H), 7.36 (m, 3H), 7.27 (t, J=7.8 Hz, 2H), 7.13 (m, 1H), 6.74 (m, 2H), 6.60 (d, J=7.8 Hz, 1H), 4.66 (d, J=5.7 Hz, 2H), 3.77 (s, 3H). MS m/z: 374 (M+1).

### Example 17

### Synthesis of (2,3-Dihydro-benzo[1,4]dioxin-6-yl)-(5-{4-aminodimethyl-2-[(Pyridin-3-ylmethyl)-amino]-phenyl}-4H-[1,2,4]triazol-3-yl)-amine (229)

### Step 1: Synthesis of 4-fluoro-2-nitro-benzoic acid Methyl Ester (229a)

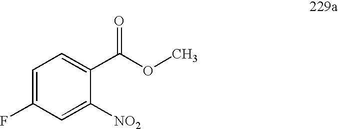

Iodomethane (2.29 ml, 36.7 mmol) was added to a solution of 4-fluoro-2-nitro-benzoic acid (6.18 g, 33 mmol, from Aldrich) in DMF (15 ml) and K₂CO₃ (6.91 g, 50 mmol). The reaction mixture was stirred at room temperature under argon for 12 hours, then poured into water (100 ml) and extracted with ether (100 ml×3). The combined organic layer was washed with brine, dried over anhydrous sodium sulfate, filtered and the filtrate was evaporated. The organic residue was purified by silica gel column chromatography (hexane:ether=5:1) to 5.3 g of compound 229a. Yield: 80.3%. ¹HNMR (DMSO-d₆) δ (ppm) 7.68-7.73 (m, 1H), 7.39-7.44 (m, 1H), 7.15-7.22 (m, 1H), 3.80 (s, 6H).

### Step 2: Synthesis of 4-dimethylamino-2-nitro-benzoic acid Methyl Ester (229b)

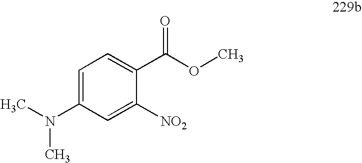

To a solution of 4-fluoro-2-nitro-benzoic acid methyl ester 229a (2.0 g, 10 mmol) in DMF (10 ml), dimethylamine hydrochloride (1.64 g, 20 mmol), K₂CO₃ (2.78 g, 20 mmol) were added. The reaction mixture was stirred at 80° C. under argon for 12 hours. The reaction was poured into 100 ml water, and a solid precipitated out. The solid was separated by filtration to, obtain 2.19 g of 229b. Yield: 97.3%. ¹HNMR (DMSO-d₆) δ (ppm) 7.75 (d, 1H), 7.06 (d, 1H), 6.86-6.90 (m 1H), 3.78 (s, 3H), 3.03 (s, 6H).

### Step 3: Synthesis of 4-dimethylamino-2-nitro-benzoic acid Methyl Ester (229c)

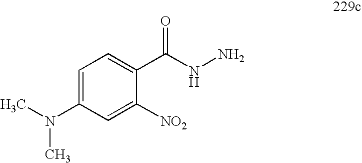

Compound 229b (2.19 g, 9.78 mmol) was added to a solution of 2-propanol (15 ml) and 85% hydrazine monohydrate (1.46 ml, 30.15 mmol). The reaction mixture was stirred at 80° C. for 72 hours. Upon the reaction was done, the solid precipitated out. 1.027 mg of 229c recovered as a solid upon filtration (yield 46.9%). ¹HNMR (DMSO-d₆) δ (ppm) 7.41 (d, 1H), 7.05 (d, 1H), 6.86-6.91 (m, 1H), 4.38 (s, 1H), 2.95 (s, 6H). MS m/z: 225 (M+1).

### Step 4: Synthesis of (2,3-Dihydro-benzo[1,4]dioxin-6-yl)-[5-(4-aminodimethyl-2-nitro-phenyl)-4H-[1,2,4]triazol-3-yl]-amine (229d)

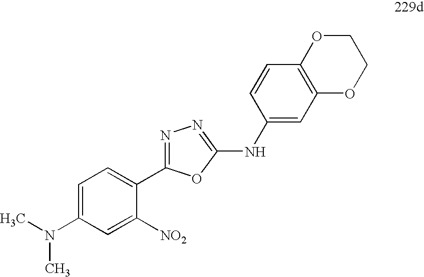

To a solution of 4-dimethylamino-2-nitrobenzoic hydrazide (1.0 g, 4.58 mmol) in dichloromethane (20 ml), 5-isothiocyanato-benzo[1,3]dioxin (0.93 g, 4.81 mmol) was added. The reaction mixture stirred at 45° C. under argon for 3 hours and a solid formed in the reaction. After filtration, the solid was washed with dichloromethane, then ether. The solid was added into toluene (20 ml), the DCC (1.2 g, 5.5 mmol) was added. The resulting mixture was stirred at 105° C. under argon overnight. The reaction was cooled, the solid was filtered and washed with hot methanol to provide 856 mg of compound 229d. Yield: 48.8%. ¹HNMR (DMSO-d₆) δ (ppm) 10.41 (s, 1H), 7.83-7.86 (m, 1H), 7.32-7.28 (m, 2H), 7.14-7.20 (m, 1H), 7.05-7.19 (m, 1H), 6.89-6.93 (m, 1H), 4.29-4.32 (m, 4H), 3.15 (s, 6H). MS m/z: 384 (M+1).

### Step 5: Synthesis of (2,3-Dihydro-benzo[1,4]dioxin-6-yl)-[5-(4-aminodimethyl-2-amino-phenyl)-[1,3,4]oxadiazol-2-yl]-amine (229e)

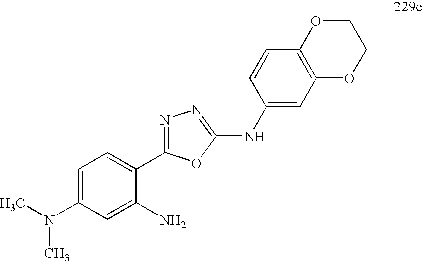

The corresponding nitro compound 229d (840 mg) was dissolved in ethanol (50 ml), then palladium, 10% wt, on activated carbon (140 mg) was added. The reaction mixture was degassed and then stirred under hydrogen at 50° C. for 3 hours. After the catalyst was filtered out, the filtrate was evaporated to obtain 700 mg of 229e in 90.4% yield. ¹HNMR (DMSO-d₆) δ (ppm) 10.23 (s, 1H), 7.35 (d, 1H), 7.24 (s, 1H), 6.96 (d, 1H), 6.84-6.88 (m, 1H), 6.41 (s, 2H), 6.16 (d, 1H), 6.10 (s, 1H), 4.16-4.21 (m, 4H), 3.05 (s, 6H). MS m/z: 354 (M+1).

### Step 6: Synthesis of (2,3-Dihydro-benzo[1,4]dioxin-6-yl)-(5-{4-dimethylamino-2-[(pyridin-3-ylmethyl)-amino]-phenyl}-[1,3,4]oxadiazol-2-yl)-amine (229)

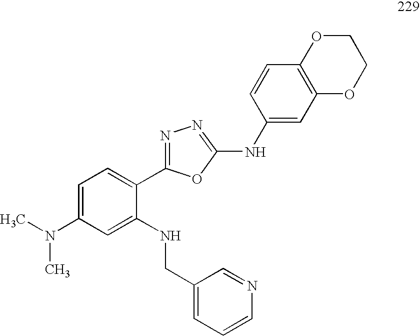

To a solution of amino compound (71 mg, 0.2 mmol) in the dichloroethane (5 ml), pyridine-3-carboxaldehyde (from Aldrich, 33 mg, 0.22 mmol) was added, followed by sodium triacetoxyborohydride (106 mg, 0.50 mmol), and acetic acid (0.2 mmol). The reaction mixture was stirred at 40° C. for 3 hours. 10% NaOH (2 ml) was added, followed by water (10 ml). A solid precipitated out. After filtration, the solid was washed with hot methanol to give 45 mg of 229 in 51% yield. ¹HNMR (DMSO-d₆) δ (ppm) 10.30 (s, 1H), 8.73 (s, 1H), 8.55 (d, 1H), 7.78-8.84 (m, 2H), 7.41 (d, 2H), 7.25 (s, 2H), 7.12 (d, 1H), 6.93 (d, 1H), 6.23 (d, 1H), 5.93 (s, 1H), 4.77 (d, 2H), 4.21-4.23 (m, 4H), 2.94 (s, 6H). MS m/z: 445 (M+1).

The compounds 230 to 236 were made using the process described in Example 17.

### Analytical Data:

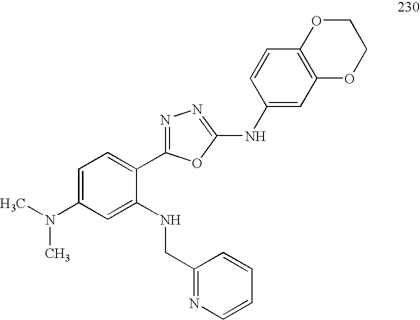

(2,3-Dihydro-benzo[1,4]dioxin-6-yl)-(5-{4-dimethylamino-2-[(pyridin-2-ylmethyl)-amino]-phenyl}-[1,3,4]oxadiazol-2-yl)-amine (230): ¹HNMR (DMSO-d₆) δ (ppm) 10.28 (s, 1H), 8.58-8.62 (m, 2H), 7.91 (t, 1H), 7.81 (t, 1H), 7.42-7.48 (m, 2H), 7.32-7.37 (m, 1H), 7.24 (d, 1H), 7.00-7.03 (m, 1H), 6.74-7.78 (m, 1H), 6.16-6.22 (m, 1H), 5.93 (s, 1H), 4.67 (d, 2H), 4.20-4.24 (m, 4H), 2.91 (s, 6H). MS m/z: 445 (M+1).

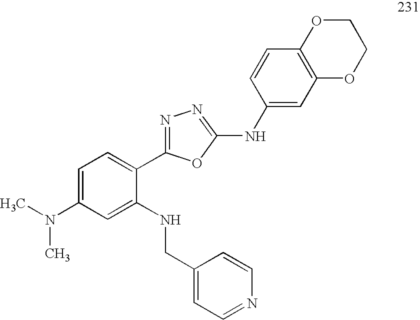

(2,3-Dihydro-benzo[1,4]dioxin-6-yl)-(5-{4-dimethylamino-2-[(pyridin-4-ylmethyl)-amino]-phenyl}-[1,3,4]oxadiazol-2-yl)-amine (231): ¹HNMR (DMSO-d₆) δ (ppm) 10.30 (s, 1H), 8.55 (d, 2H), 7.86 (t, 1H), 7.38-7.44 (m, 3H), 7.24 (s, 1H), 7.00-7.04 (m, 1H), 6.84-6.88 (m, 1H), 6.17-6.21 (m, 1H), 5.81 (s, 1H), 4.65 (d, 1H), 4.20-4.23 (m, 4H), 2.87 (s, 6H). MS m/z: 445 (M+1).

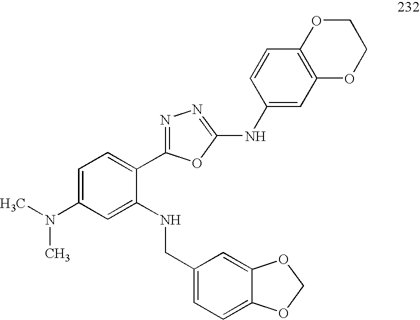

(5-{2-[(Benzo[1,3]dioxol-5-ylmethyl)-amino]-4-dimethylamino-phenyl}-[1,3,4]oxadiazol-2-yl)-(2,3-dihydro-benzo[1,4]dioxin-6-yl)-amine (232): ¹HNMR (DMSO-d₆) δ (ppm) 10.25 (s, 1H), 7.62-7.63 (m, 1H), 7.37-7.41 (m, 1H), 7.23 (s, 1H), 7.81-7.98 (m, 5H), 6.20-6.23 (m, 1H), 6.01 (s, 2H), 5.95 (s, 1H), 4.49 (d, 2H), 4.22-4.25 (m, 4H), 2.92 (s, 6H). MS m/z: 488 (M+1).

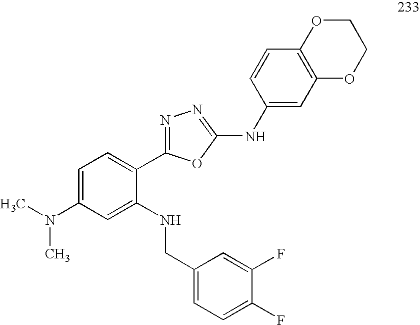

{5-[2-(3,4-Difluoro-benzylamino)-4-dimethylamino-phenyl]-[1,3,4]oxadiazol-2-yl}-(2,3-dihydro-benzo[1,4]dioxin-6-yl)-amine (233): ¹HNMR (DMSO-d₆) δ (ppm) 10.35 (s, 1H), 7.84 (t, 1H), 7.45-7.51 (m, 2H), 7.23-7.29 (m, 2H), 7.03-7.06 (m, 1H), 6.81-6.91 (m 1H), 6.02-6.07 (m, 1H), 5.91 (s, 1H), 4.30-4.34 (m, 4H), 4.05 (d, 2H), 2.94 (s, 6H). MS m/z: 480 (M+1).

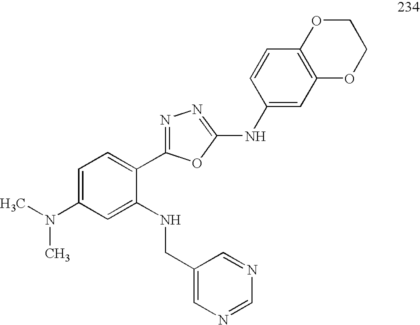

(2,3-Dihydro-benzo[1,4]dioxin-6-yl)-(5-{4-dimethylamino-2-[(pyrimidin-5-ylmethyl)-amino]-phenyl}-[1,3,4]oxadiazol-2-yl)-amine (234): ¹HNMR (DMSO-d₆) δ (ppm) 10.21 (s, 1H), 8.13 (s, 1H), 8.57 (s, 1H), 8.45 (s, 1H), 7.92 (t, 1H), 7.30-7.34 (m, 1H), 7.12 (s, 1H), 6.88-6.92 (m, 1H), 6.70-6.73 (m, 1H), 6.06-6.13 (m, 1H), 5.85 (s, 1H), 4.65 (d, 2H), 4.12-4.18 (m, 4H), 2.80 (s, 6H). MS m/z: 446 (M+1).

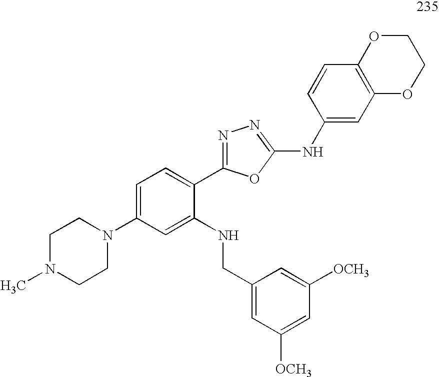

(2,3-Dihydro-benzo[1,4]dioxin-6-yl)-{5-[2-(3,5-dimethoxy-benzylamino)-4-(4-methyl-piperazin-1-yl)-phenyl]-[1,3,4]oxadiazol-2-yl}-amine (235): ¹HNMR (DMSO-d₆) δ (ppm) 10.40 (s, 1H), 7.88 (t, 1H), 7.62-7.68 (m, 1H), 7.36-7.38 (m, 1H), 7.12-7.17 (m, 1H), 6.94-7.00 (m, 1H), 6.77-6.79 (m, 2H), 6.48-6.57 (m, 2H), 6.35 (s, 1H), 4.63 (d, 2H), 4.34-4.38 (m, 4H), 3.89 (s, 6H), 3.35 (s, 4H), 2.38 (s, 4H), 2.31 (s, 3H). MS m/z: 559 (M+1).

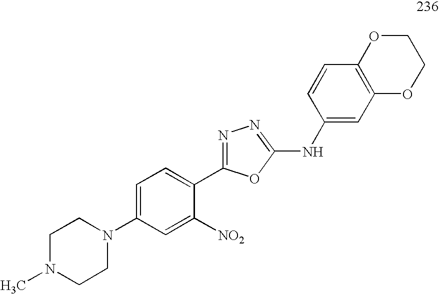

(2,3-Dihydro-benzo[1,4]dioxin-6-yl)-{5-[4-(4-methyl-piperazin-1-yl)-2-nitro-phenyl]-[1,3,4]oxadiazol-2-yl}-amine (236)): ¹HNMR (DMSO-d₆) δ (ppm) 7.77-7.82 (m, 1H), 7.66 (s, 1H), 7.37-7.41 (m, 1H), 7.23 (s, 1H), 7.01-7.05 (m, 1H), 6.88-6.90 (m, 1H), 4.28-4.31 (m, 4H), 3.52-3.54 (m, 4H), 2.40-2.42 (m, 4H), 2.26 (s, 3H). MS m/z: 439 (M+1).

### Example 18

### synthesis of N-{2-[5-(Benzo[1,3]dioxol-5-ylamino)-[1,3,4]oxadiazol-2-yl]-phenyl}-3-methoxy-benzenesulfonamide (237)

### Step 1: Synthesis of [5-(2-Amino-phenyl)-[1,3,4]oxadiazol-2-yl]-benzo[1,3]dioxol-5-yl-amine (237a)

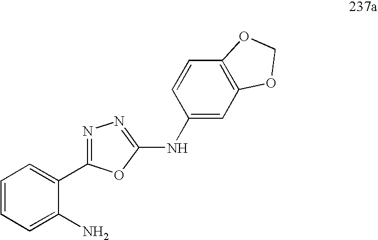

To a solution of 2-nitrobenzoic hydrazide (2.0 g, 11 mmol, from Aldrich) in dichloromethane (50 ml) there was added 5-isothiocyanato-benzo[1,3]dioxole (2.17 g, 12.7 mmol, from Oakwood Products, Inc.). The reaction mixture was stirred at 45° C. under argon for 3 hours. After filtration, the formed solid was washed with dichloromethane and ether. The resulting solid was placed in toluene (40 ml), then DCC (3.3 g, 16.5 mmol) was added and heated at 105° C. under argon for 12 hours. The reaction mixture was cooled down to room temperature, and the precipitated solid was washed with hot methanol to provide 1.7 g benzo[1,3]dioxol-5-yl-[5-(2-nitro-phenyl)-[1,3,4]oxadiazol-2-yl]-amine in 50% yield by weight. This compound was dissolved in ethanol (50 ml), then palladium (10% wt, on activated carbon) (170 mg) was added. The reaction mixture was degassed, and stirred under hydrogen at 50° C. for 4 hours. After the catalyst was filtered out, the filtrate was evaporated to yield 1.2 g of 237a. Yield: 72.3%. ¹HNMR (DMSO-d₆) δ (ppm) 10.51 (s, 1H), 7.58-7.60 (m, 1H), 7.25-7.27 (s, 1H), 7.12-7.20 (m, 1H), 6.96-7.01 (m, 1H), 6.83-6.90 (m, 2H), 6.67-6.74 (m, 2H), 5.99 (s, 2H). MS m/z: 297 (M+1).

### Step 2: Synthesis of N-{2-[5-(Benzo[1,3]dioxol-5-ylamino)-[1,3,4]oxadiazol-2-yl]-phenyl}-3-methoxy-benzenesulfonamide (237)

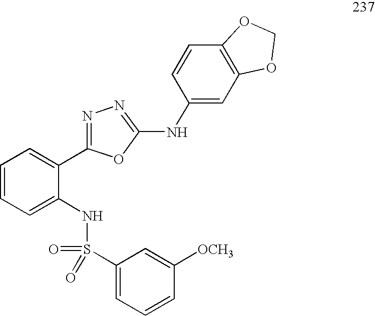

To a solution of [5-(2-amino-phenyl)-[1,3,4,]oxadiazol-2-yl]-benzo[1,3]dioxol-5-yl-amine (70 mg, 0.236 mmol) in pyridine (1.0 ml), 3-methoxylbenesulfonyl chloride (58.5 mg, 0.283 mmol) and DMAP (10 mg) were added. The reaction mixture was stirred at room temperature under argon for 1 hour, then heated to 60° C. for 12 hours. The reaction was quenched with 5% NaHCO₃ aqueous solution, poured into water (10 ml), then extracted with ethyl acetate (3×15 ml). The combined organic layer was washed with brine, and dried over anhydrous Na₂SO₄. After filtration and evaporation, the organic residue was subjected prepared TLC (Dichloromethane:Methanol=50:1), to yield 46 mg of 237. Yield: 42.2%. ¹HNMR (DMSO-d₆) δ (ppm) 10.75 (s, 1H), 10.57 (s, 1H), 7.20-7.68 (m, 9H), 7.03-7.08 (m, 1H), 6.90-6.94 (m, 1H), 6.00 (s, 2H), 3.74 (s, 3H). MS m/z: 467 (M+1).

Compounds 238 to 240 were synthesized using the method described in Example 18:

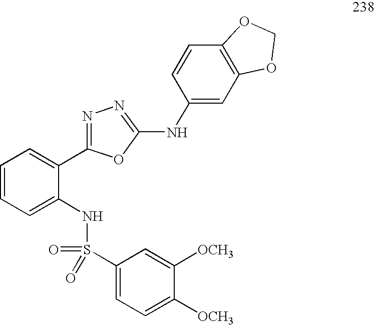

N-{2-[5-(Benzo[1,3]dioxol-5-ylamino)-[1,3,4]oxadiazol-2-yl]-phenyl}-3,4-dimethoxy-benzenesulfonamide (238): ¹HNMR (DMSO-d₆) δ (ppm) 10.79 (s, 1H), 10.49 (s, 1H), 7.60-7.66 (m, 2H), 7.44-7.52 (m, 1H), 7.35-7.40 (m, 1H), 7.16-7.23 (m, 3H), 6.95-7.00 (m, 2H), 6.88-6.95 (m, 1H), 6.00 (s, 2H), 3.77 (s, 3H), 3.68 (s, 3H). MS m/z: 497 (M+1).

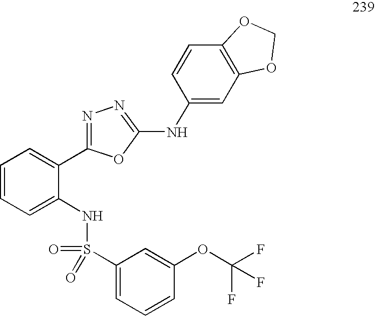

N-{2-[5-(Benzo[1,3]dioxol-5-ylamino)-[1,3,4]oxadiazol-2-yl]-phenyl}-3-trifluoromethoxy-benzenesulfonamide (239): ¹HNMR (DMSO-d₆) δ (ppm) 10.75 (d, 2H), 7.88-7.92 (m, 1H), 7.74-7.78 (m, 2H), 7.55-7.60 (m, 1H), 7.30-7.40 (m, 2H), 7.06-7.10 (m, 1H), 6.96-7.00 (m, 1H), 6.86-6.91 (m, 2H), 6.02 (s, 2H). MS m/z: 521 (M+1).

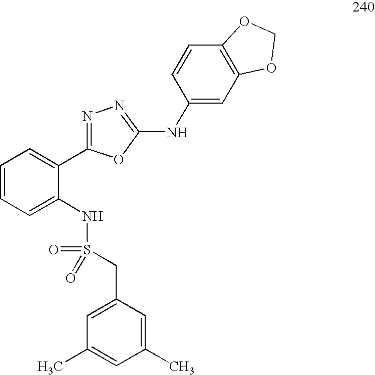

N-{2-[5-(Benzo[1,3]dioxol-5-ylamino)-[1,3,4]oxadiazol-2-yl]-phenyl}-C-(3,5-dimethyl-phenyl)-methanesulfonamide (240): ¹HNMR (DMSO-d₆) δ (ppm) 11.00 (d, 2H), 7.97-8.00 (m, 1H), 7.70-7.76 (m, 1H), 7.48-7.56 (m, 3H), 7.30-7.34 (m, 3H), 7.18-7.20 (m, 1H), 6.29 (s, 2H), 2.75 (s, 2H), 2.84 (s, 6H). MS m/z: 479 (M+1).

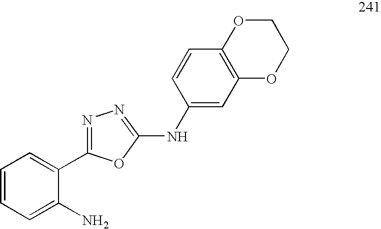

[5-(2-Amino-phenyl)-[1,3,4]oxadiazol-2-yl]-(2,3-dihydro-benzo[1,4]dioxin-6-yl)-amine (241): synthesized according to the method for 237a. ¹HNMR (DMSO-d₆) δ (ppm) 10.44 (s, 1H), 8.03-8.06 (m, 1H), 7.88-7.91 (m, 1H), 7.68-7.83 (m, 2H), 7.08-7.11 (m, 1H), 6.87-6.90 (m, 1H), 6.71-6.75 (m, 1H), 4.08-4.20 (m, 4H). MS m/z: 311 (M+1).

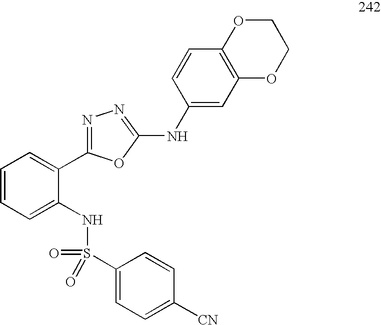

4-Cyano-N-{2-[5-(2,3-dihydro-benzo[1,4]dioxin-6-ylamino)-[1,3,4]oxadiazol-2-yl]-phenyl}-benzenesulfonamide (242): ¹HNMR (DMSO-d₆) δ (ppm) 10.90 (s, 1H), 10.68 (s, 1H), 8.02-8.10 (m, 4H), 7.76-7.80 (m, 1H), 7.53-7.60 (m, 2H), 7.29-7.38 (m, 2H), 7.07-7.11 (m, 1H), 6.92-6.96 (m, 1H), 4.30-4.35 (m, 4H). MS m/z: 476 (M+1).

### Example 19

### synthesis of N-{5-[2-(3,5-Dimethoxy-phenylamino)-pyridin-3-yl]-[1,3,4]oxadiazol-2-yl}-3-methoxy-benzenesulfonamide (243)

### Step 1: Synthesis of [3-(5-Amino-[1,3,4]oxadiazol-2-yl)-pyridin-2-yl]-(3,5-dimethoxy-phenyl)-amine (243a)

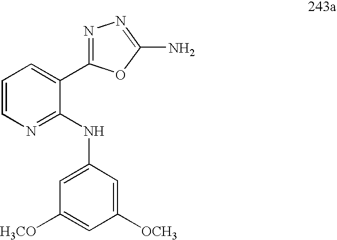

A reaction mixture of 2-(3,5-dimethoxy-phenylamino)-nicotinic acid hydrazide (1c, Example 1, 1.0 g, 3.47 mmol) and benzotrizol-1-yl-C— (2,3-dihydro-benzotriazol-1-yl)-methyleneamine (0.913 g, 3.47 mmol) in THF (50 ml) was stirred at 70° C. for 3 hours. The reaction was cooled and the precipitate was filtered and washed with THF to obtain 900 mg of 243a in 83% yield. ¹HNMR (DMSO-d₆) δ (ppm) 9.99 (s, 1H), 8.28-8.32 (m, 1H), 7.88-7.91 (m, 1H), 7.43 (s, 2H), 6.97-6.99 (m, 3H), 6.12 (s, 1H), 3.68 (s, 6H). MS m/z: 314 (M+1).

### Step 2: synthesis of N-{5-[2-(3,5-Dimethoxy-phenylamino)-pyridin-3-yl]-[1,3,4]oxadiazol-2-yl}-3-methoxy-benzenesulfonamide (243)

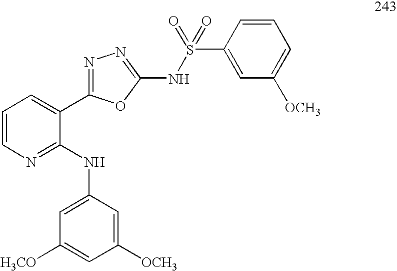

To a solution of [3-(5-amino-[1,3,4]oxadizol-2-yl)-pyridin-2-yl]-(3,5-dimethoxy-phenyl)-amine (63 mg, 0.2 mmol) in pyridine (1 ml) was added meta-methoxysulfonyl chloride (50 μL, 4 mmol). The reaction mixture stirred at 100° C. for 6 hours. The reaction solution was poured into water (10 ml), and a white solid precipitated out. The solid was filtered and washed with hot methanol To obtain 21 mg of 243 in 21.8% yield. ¹HNMR (DMSO-d₆) δ (ppm) 10.21 (s, 1H), 8.49-8.52 (m, 1H), 7.71 (s, 3H), 7.10-7.15 (m, 4H), 6.43 (s, 1H), 3.91 (s, 9H). MS m/z: 484 (M+1).

### Example 20

### synthesis of (5-{2-[(Benzo[1,3]dioxol-5-ylmethyl)-amino]-phenyl}-oxazol-2-yl)-(2,3-dihydro-benzo[1,4]dioxin-6-yl)-amine (244)

### Step 1: synthesis of 2-Azido-1-(2-nitro-phenyl)-ethanone (244a)

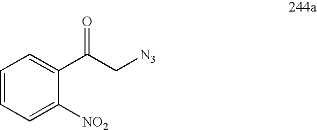

A mixture of 2-bromo-1-(2-nitro-phenyl)-ethanone (12 mmol, 2.93 g, from Aldrich) and NaN₃ (14.4 mmol, 0.94 g) in CH₃COCH₃/H₂O (15/5 ml) was stirred at 50° C. for 30 minutes. Most of solvent was removed in vacuo. Et₂O was added, and the organic phase was washed with H₂O, brine, and dried over Na₂SO₄. Removal of solvent in vacuo gave 2-azido-1-(2-nitro-phenyl)-ethanone (2.18 g, 88%) as a brown solid. ¹HNMR (CDCl₃) δ (ppm) 8.23 (d, J=8.1 Hz, 1H), 7.81 (t, J=7.5 Hz, 1H), 7.69 (t, J=7.5 Hz, 1H), 7.41 (d, J=7.5 Hz, 1H), 4.32 (s, 2H).

### Step 2: synthesis of (2,3-Dihydro-benzo[1,4]dioxin-6-yl)-[5-(2-nitro-phenyl)-oxazol-2-yl]-amine (244b)

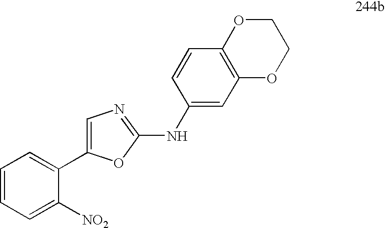

To a mixture of 2-azido-1-(2-nitro-phenyl)-ethanone (244a, 10 mmol, 2.06 g) and 6-isothiocyanato-2,3-dihydro-benzo[1,4]dioxine (10 mmol, 1.93 g, from Maybridge) in dry dioxane (20 ml), was added Ph₃P (10 mmol, 2.62 g) in one portion. The flask was immersed into a pre-heated oil bath (95° C.), and stirred for 20 minutes. (Caution: Although we did not experience any explosions while doing this reaction, extreme caution must be exercised when heating an azide solution due to the possibility of an explosion). After removal of solvent in vacuo, the residue was subjected to the flash column chromatography (silica gel) with Hexanes/EtOAc (2:1 to 1:1) as an eluent to give mixture of (2,3-dihydro-benzo[1,4]dioxin-6-yl)-[5-(2-nitro-phenyl)-oxazol-2-yl]-amine and some by-products (containing Ph₃P═S and Ph₃P═O). The solid was triturated with EtOAc to furnish pure (2,3-dihydro-benzo[1,4]dioxin-6-yl)-[5-(2-nitro-phenyl)-oxazol-2-yl]-amine (0.51 g, 15%). ¹HNMR (DMSO-d₆) δ (ppm) 10.26 (s, 1H), 7.98 (dd, J=8.1, 0.6 Hz, 1H), 7.84 (td, J=7.8, 1.5 Hz, 1H), 7.80 (td, J=7.8, 1.5 Hz, 1H), 7.60 (td, J=7.5, 1.5 Hz, 1H), 7.47 (s, 1H), 7.29 (d, J=2.4 Hz, 1H), 7.05 (dd, J=9.0, 2.4 Hz, 1H), 6.86 (d, J=9.0 Hz, 1H), 4.30-4.25 (m, 4H). MS m/z: 340 (M+1).

### Step 3: synthesis of [5-(2-Amino-phenyl)-oxazol-2-yl]-(2,3-dihydro-benzo[1,4]dioxin-6-yl)-amine (244c)

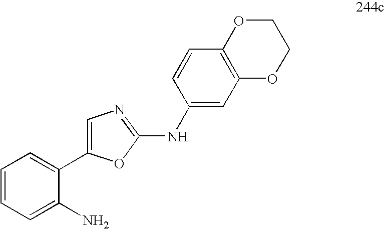

A mixture of (2,3-dihydro-benzo[1,4]dioxin-6-yl)-[5-(2-nitro-phenyl)-oxazol-2-yl]-amine (1 mmol, 0.34 g) and Pd/C (50 mg) in dry methanol (10 ml) was stirred at 40° C. under hydrogen (using a balloon). After 4 h, the reaction mixture was filtered through silica gel, and washed with EtOAc. The combined solution was concentrated in vacuo to give [5-(2-amino-phenyl)-oxazol-2-yl]-(2,3-dihydro-benzo[1,4]dioxin-6-yl)-amine (0.27 g, 87%). ¹H NMR (DMSO-d₆) δ (ppm) 10.03 (s, 1H), 7.38 (d, J=7.8 Hz, 1H), 7.35 (d, J=2.4 Hz, 1H), 7.26 (s, 1H), 7.07-7.04 (m, 2H), 6.87-6.84 (m, 2H), 6.71 (t, J=7.5 Hz, 1H), 5.23 (s, 2H), 4.30-4.24 (m, 4H). MS m/z: 310 (M+1).

### Step 4: synthesis of (5-{2-[(Benzo[1,3]dioxol-5-ylmethyl)-amino]-phenyl}-oxazol-2-yl)-(2,3-dihydro-benzo[1,4]dioxin-6-yl)-amine (244)

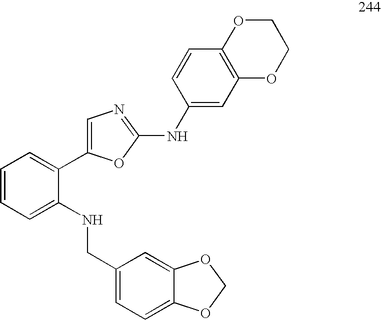

To a mixture of [5-(2-amino-phenyl)-oxazol-2-yl]-(2,3-dihydro-benzo[1,4]dioxin-6-yl)-amine (0.15 mmol, 46 mg) and piperonal (0.18 mmol, 27 mg, from Aldrich) in dry benzene (4 ml), was added NaBH(OAc)₃ (0.45 mmol, 95 mg) and one drop of CH₃COOH. The reaction mixture was stirred at 70° C. in a sealed tube. After 16 hours, additional NaBH(OAc)₃ (0.3 mmol, 64 mg) was added, and the reaction continued at 70° C. for an additional 6 hours. After cooling, ethyl acetate and water were added. The separated organic phase was washed with saturated aqueous NaHCO₃, H₂O, brine, and dried over anhydrous Na₂SO₄. After removal of solvent in vacuo, the residue was purified by column chromatography (silica gel) with Hexanes/EtOAc (8:1 to 1:1) as an eluent to give (5-{2-[(benzo[1,3]dioxol-5-ylmethyl)-amino]-phenyl}-oxazol-2-yl)-(2,3-dihydro-benzo[1,4]dioxin-6-yl)-amine (20 mg, 30%). ¹HNMR (CDCl₃) δ (ppm) 7.39 (dd, J=7.8, 1.5 Hz, 1H), 7.24-7.19 (m, 1H), 7.06-7.05 (m, 2H), 6.96-6.91 (m, 2H), 6.88-6.78 (m, 3H), 6.78-6.66 (m, 2H), 5.96 (s, 2H), 4.28 (s, 2H), 4.26-4.21 (m, 4H). MS m/z: 444 (M+1).

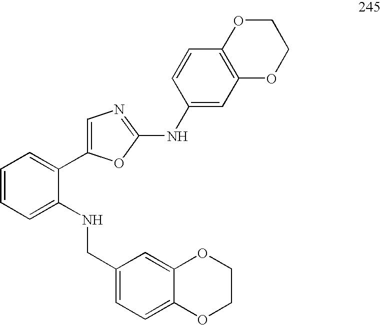

(2,3-Dihydro-benzo[1,4]dioxin-6-yl)-(5-{2-[(2,3-dihydro-benzo[1,4]dioxin-6-ylmethyl)-amino]-phenyl}-oxazol-2-yl)-amine (245): ¹HNMR (CDCl₃) δ (ppm) 7.40 (dd, J=7.5, 1.2 Hz, 1H), 7.18 (td, J=7.5, 1.2 Hz, 1H), 7.05 (d, J=2.4 Hz, 1H), 7.03 (s, 1H), 6.89-6.70 (m, 7H), 4.25 (s, 10H). MS m/z: 458 (M+1).

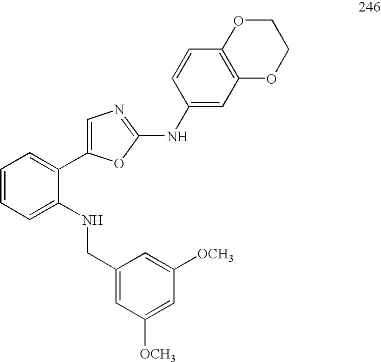

(2,3-Dihydro-benzo[1,4]dioxin-6-yl)-{5-[2-(3,5-dimethoxy-benzylamino)-phenyl]-oxazol-2-yl}-amine (246): ¹H NMR (CDCl₃) δ (ppm) 7.40 (dd, J=7.5, 1.2 Hz, 1H), 7.17 (td, J=7.5, 1.2 Hz, 1H), 7.06 (d, J=2.4 Hz, 1H), 7.04 (s, 1H), 6.89 (dd, J=8.7, 2.4 Hz, 1H), 6.83-6.76 (m, 2H), 6.69 (d, J=8.4 Hz, 1H), 6.56-6.53 (m, 2H), 6.38-6.35 (m, 1H), 4.32 (s, 2H), 4.25-4.22 (m, 4H), 3.77 (s, 6H). MS m/z: 460 (M+1).

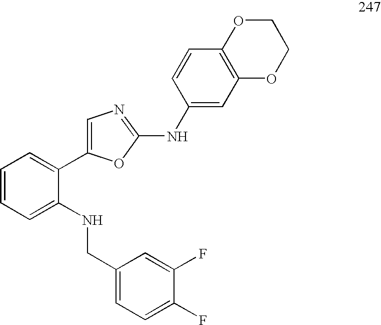

{5-[2-(3,4-Difluoro-benzylamino)-phenyl]-oxazol-2-yl}-(2,3-dihydro-benzo[1,4]dioxin-6-yl)-amine (247): ¹H NMR (CDCl₃) δ (ppm) 7.40 (dd, J=7.5, 1.5 Hz, 1H), 7.22-7.05 (m, 6H), 6.90-6.77 (m, 3H), 6.61 (d, J=8.1 Hz, 1H), 4.36 (s, 2H), 4.26-4.22 (m, 4H). MS m/z: 436 (M+1).

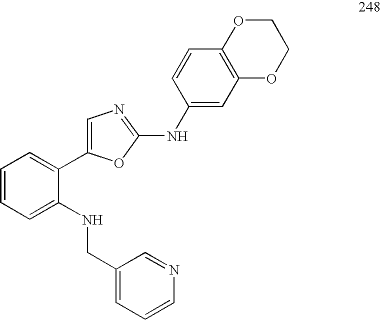

(2,3-Dihydro-benzo[1,4]dioxin-6-yl)-(5-{2-[(pyridin-3-ylmethyl)-amino]-phenyl}-oxazol-2-yl)-amine (248): ¹HNMR (CDCl₃) δ (ppm) 8.66 (d, J=1.5 Hz, 1H), 8.53 (dd, J=4.8, 0.9 Hz, 1H), 7.74 (d, J=7.8 Hz, 1H), 7.41 (dd, J=7.5, 1.2 Hz, 1H), 7.29 (dd, J=7.5, 4.8 Hz, 1H), 7.16 (td, J=7.8, 1.5 Hz, 1H), 7.08 (d, J=2.4 Hz, 1H), 7.04 (s, 1H), 6.89 (dd, J=8.7, 2.4 Hz, 1H), 6.82 (s, 1H), 6.81-6.78 (m, 1H), 6.64 (d, J=8.1 Hz, 1H), 4.43 (s, 2H), 4.25-4.20 (m, 4H). MS m/z: 401 (M+1).

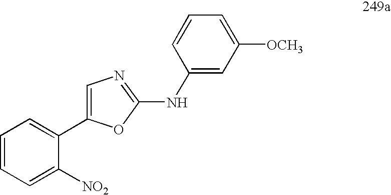

(3-Methoxy-phenyl)-[5-(2-nitro-phenyl)-oxazol-2-yl]-amine (249a): ¹HNMR (DMSO-d₆) δ (ppm) 10.42 (s, 1H), 7.95 (dd, J=7.8, 0.6 Hz, 1H), 7.80 (td, J=7.8, 1.5 Hz, 1H), 7.76 (td, J=7.8, 1.5 Hz, 1H), 7.56 (td, J=7.5, 1.8 Hz, 1H), 7.45 (s, 1H), 7.30 (t, J=2.1 Hz, 1H), 7.22 (t, J=8.1 Hz, 1H), 7.15-7.12 (m, 1H), 6.58-6.54 (m, 1H), 3.75 (s, 3H). MS m/z: 312 (M+1).

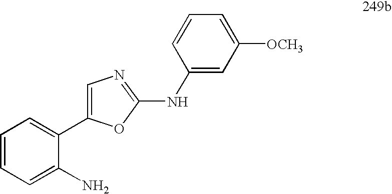

[5-(2-Amino-phenyl)-oxazol-2-yl]-(3-methoxy-phenyl)-amine (249b): ¹H NMR (DMSO-d₆) δ (ppm) 10.24 (s, 1H), 7.41-7.37 (m, 2H), 7.28 (s, 1H), 7.23 (d, J=8.1 Hz, 1H), 7.16 (d, J=8.1 Hz, 1H), 7.06 (td, J=7.5, 1.2 Hz, 1H), 6.85 (d, J=8.1 Hz, 1H), 6.69 (t, J=7.5 Hz, 1H), 6.57 (dd, J=7.8, 1.2 Hz, 1H), 5.24 (s, 2H), 3.79 (s, 3H). MS m/z: 282 (M+1).

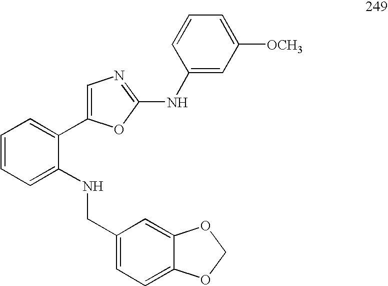

(5-{2-[(Benzo[1,3]dioxol-5-ylmethyl)-amino]-phenyl}-oxazol-2-yl)-(3-methoxy-phenyl)-amine (249): ¹H NMR (CDCl₃) δ (ppm) 7.42 (dd, J=7.8, 1.5 Hz, 1H), 7.22 (d, J=8.1 Hz, 1H), 7.18-7.16 (m, 2H), 7.07 (s, 1H), 7.01 (d, J=8.1 Hz, 1H), 6.87 (s, 1H), 6.85-6.73 (m, 4H), 6.61 (dd, J=8.1, 1.8 Hz, 1H), 5.95 (s, 2H), 4.29 (s, 2H), 3.84 (s, 3H). MS m/z: 416 (M+1).

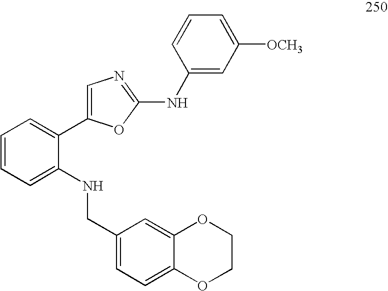

(5-{2-[(2,3-Dihydro-benzo[1,4]dioxin-6-ylmethyl)-amino]-phenyl}-oxazol-2-yl)-(3-methoxy-phenyl)-amine (250): ¹HNMR (DMSO-d₆) δ (ppm) 10.25 (s, 1H), 7.40 (t, J=2.1 Hz, 1H), 7.36 (dd, J=7.5, 1.2 Hz, 1H), 7.32 (s, 1H), 7.22-7.17 (m, 2H), 7.07 (t, J=7.5 Hz, 1H), 6.87-6.78 (m, 3H), 6.67 (t, J=7.5 Hz, 1H), 6.59 (d, J=8.1 Hz, 1H), 6.55 (dd, J=8.1, 1.2 Hz, 1H), 5.72 (t, J=2.7 Hz, 1H), 4.31 (d, J=2.7 Hz, 2H), 4.20 (s, 4H), 3.76 (s, 3H). MS m/z: 430 (M+1).

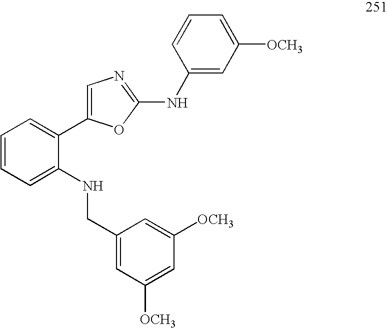

{5-[2-(3,5-Dimethoxy-benzylamino)-phenyl]-oxazol-2-yl}-(3-methoxy-phenyl)-amine (251): ¹H NMR (DMSO-d₆) δ (ppm) 10.30 (s, 1H), 7.46 (t, J=2.1 Hz, 1H), 7.42 (dd, J=7.5, 1.5 Hz, 1H), 7.39 (s, 1H), 7.28 (t, J=8.1 Hz, 1H), 7.22 (t, J=8.1 Hz, 1H), 7.12 (td, J=7.5, 1.2 Hz, 1H), 6.74 (t, J=7.5 Hz, 1H), 6.64-6.54 (m, 4H), 6.42-6.40 (m, 1H), 5.82 (t, J=5.7 Hz, 1H), 4.42 (d, J=5.7 Hz, 2H), 3.81 (s, 3H), 3.76 (s, 6H). MS m/z: 432 (M+1).

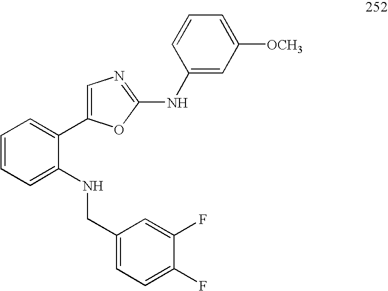

{5-[2-(3,4-Difluoro-benzylamino)-phenyl]-oxazol-2-yl}-(3-methoxy-phenyl)-amine (252): ¹HNMR (CDCl₃) δ (ppm) 7.43 (dd, J=7.5, 1.5 Hz, 1H), 7.25-7.10 (m, 6H), 7.09 (s, 1H), 6.98 (dd, J=7.5, 2.1 Hz, 1H), 6.81 (td, J=7.5, 0.9 Hz, 1H), 6.64-6.58 (m, 2H), 4.36 (s, 2H), 3.82 (s, 3H). MS m/z: 408 (M+1).

### Example 21

### Synthesis of (2,3-Dihydro-benzo[1,4]dioxin-6-yl)-{5-[2-(pyridin-4-ylmethoxy)-phenyl]-oxazol-2-yl}-amine (253)

### Step 1: synthesis of 2-Azido-1-(2-hydroxy-phenyl)-ethanone (253a)

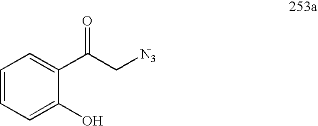

A mixture of 2-bromo-1-(2-hydroxy-phenyl)-ethanone (12 mmol, 2.57 g, from Aldrich) and NaN₃ (14.4 mmol, 0.94 g) in CH₃COCH₃/H₂O (15/5 ml) was stirred at 50° C. for 30 minutes. Most of solvent was removed in vacuo. Et₂O was added, and the organic phase was washed with H₂O, brine, and dried over Na₂SO₄. Removal of solvent in vacuo gave 2-azido-1-(2-hydroxy-phenyl)-ethanone (1.91 g, 90%) as a light-yellow solid. ¹HNMR (CDCl₃) δ (ppm) 11.66 (s, 1H), 7.58-7.50 (m, 2H), 7.04 (d, J=8.7 Hz, 1H), 6.92 (t, J=7.8 Hz, 1H), 4.59 (s, 2H).

### Step 2: synthesis of 2-[2-(2,3-Dihydro-benzo[1,4]dioxin-6-ylamino)-oxazol-5-yl]-phenol. hydrochloride (253b)

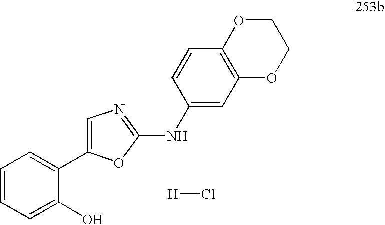

To a mixture of 2-azido-1-(2-hydroxy-phenyl)-ethanone (10 mmol, 1.77 g) and 6-isothiocyanato-2,3-dihydro-benzo[1,4]dioxine (10 mmol, 1.93 g) in dry dioxane (20 ml), was added Ph₃P (10 mmol, 2.62 g) in one portion. The flask was immersed into a pre-heated oil bath (95° C.), and stirred for 20 minutes. (Caution: Although we did not experience any explosions while doing this reaction, extreme caution must be exercised when heating an azide solution due to the possibility of an explosion). After removal of solvent in vacuo, the residue was subjected to the flash column chromatography (silica gel) with Hexanes/EtOAc (2:1 to 1:1) as an eluent to give mixture of (2,3-dihydro-benzo[1,4]dioxin-6-yl)-[5-(2-nitro-phenyl)-oxazol-2-yl]-amine and some by-products (containing Ph₃P═S and Ph₃P═O). The mixture was suspended in EtOAc and treated with excess HCl/Et₂O. The solid was collected and washed with EtOAc to furnish (2,3-dihydro-benzo[1,4]dioxin-6-yl)-[5-(2-hydroxy-phenyl)-oxazol-2-yl]-ammonium chloride (1.15 g, 33%). ¹HNMR (DMSO-d₆) δ (ppm) 10.40 (s, 1H), 10.23 (s, 1H), 7.30 (dd, J=7.8, 1.2 Hz, 1H), 7.20 (s, 1H), 7.09 (d, J=2.4 Hz, 1H), 6.99 (td, J=7.8, 1.2 Hz, 1H), 6.90-6.69 (m, 4H), 4.12-4.06 (m, 4H). MS m/z: 311 (M+1).

### Step 3: synthesis of (2,3-Dihydro-benzo[1,4]dioxin-6-yl)-{5-[2-(pyridin-4-ylmethoxy)-phenyl]-oxazol-2-yl}-amine (253)

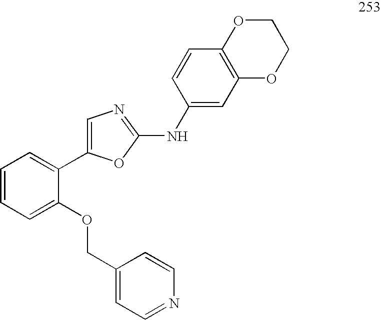

A mixture of (2,3-dihydro-benzo[1,4]dioxin-6-yl)-[5-(2-hydroxy-phenyl)-oxazol-2-yl]-ammonium chloride (0.2 mmol, 69 mg) and 4-bromomethyl-pyridine hydrobromide (0.2 mmol, 51 mg, from Aldrich) in dry DMF (3 ml) was stirred at 40° C. in the presence of K₂CO₃ (1 mmol, 0.14 g). After 3 h, most of DMF was removed in vacuo. EtOAc and H₂O were added. The separated organic phase was washed with H₂O, brine, and dried over Na₂SO₄. After removal of solvent in vacuo, the residue was purified by column chromatography (silica gel) with Hexanes/EtOAc (6:1 to 1:1) as an eluent to give (2,3-dihydro-benzo[1,4]dioxin-6-yl)-{5-[2-(pyridin-4-ylmethoxy)-phenyl]-oxazol-2-yl}-amine (37 mg, 46%). ¹HNMR (CDCl₃) δ (ppm) 8.64 (d, J=5.7 Hz, 2H), 7.65 (d, J=7.5 Hz, 1H), 7.41 (d, J=7.2 Hz, 2H), 7.31 (s, 1H), 7.20 (t, J=7.2 Hz, 1H), 7.11-7.02 (m, 2H), 6.93 (d, J=8.1 Hz, 2H), 6.85 (d, J=8.7 Hz, 1H), 5.23 (s, 2H), 4.26-4.23 (m, 4H). MS m/z: 402 (M+1).

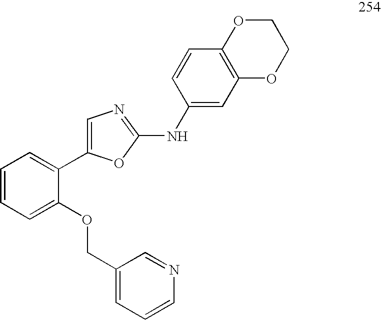

(2,3-Dihydro-benzo[1,4]dioxin-6-yl)-{5-[2-(pyridin-3-ylmethoxy)-phenyl]-oxazol-2-yl}-amine (254): ¹HNMR (CDCl₃) (ppm) 8.72 (d, J=1.8 Hz, 1H), 8.63 (dd, J=4.8, 1.2 Hz, 1H), 7.84 (d, J=7.2 Hz, 1H), 7.63 (dd, J=7.5, 1.5 Hz, 1H), 7.36 (dd, J=7.8, 4.8 Hz, 1H), 7.22 (td, J=8.1, 1.5 Hz, 1H), 7.19 (s, 1H), 7.08-7.01 (m, 3H), 6.90 (dd, J=8.7, 2.7 Hz, 1H), 6.83 (d, J=8.7 Hz, 1H), 5.20 (s, 2H), 4.27-4.22 (m, 4H). MS m/z: 402 (M+1).

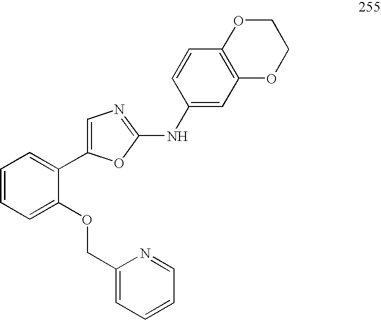

(2,3-Dihydro-benzo[1,4]dioxin-6-yl)-{5-[2-(pyridin-3-ylmethoxy)-phenyl]-oxazol-2-yl}-amine (255): ¹HNMR (CDCl₃) δ (ppm) 8.62 (d, J=4.5 Hz, 1H), 7.22 (td, J=7.5, 1.5 Hz, 1H), 7.64 (dd, J=7.5, 1.5 Hz, 1H), 7.53 (d, J=7.8 Hz, 1H), 7.41 (s, 1H), 7.27-7.23 (m, 1H), 7.18 (td, J=7.8, 1.5 Hz, 1H), 7.11 (d, J=2.4 Hz, 1H), 7.05-6.98 (m, 2H), 6.93 (dd, J=8.7, 2.4 Hz, 1H), 6.83 (d, J=8.7 Hz, 1H), 5.35 (s, 2H), 4.27-4.23 (m, 4H). MS m/z: 402 (M+1).

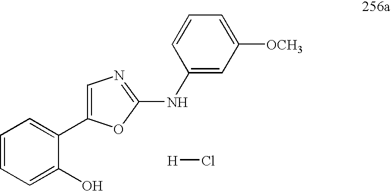

[5-(2-Hydroxy-phenyl)-oxazol-2-yl]-(3-methoxy-phenyl)-ammonium chloride (256a): ¹HNMR (DMSO-d₆) δ (ppm) 10.74 (s, 1H), 10.50 (br s, 1H), 7.47 (dd, J=7.8, 1.5 Hz, 1H), 7.37 (s, 1H), 7.32 (t, J=2.1 Hz, 1H), 7.25 (t, J=8.1 Hz, 1H), 7.17-7.11 (m, 2H), 7.01 (d, J=7.8 Hz, 1H), 6.91 (t, J=7.5 Hz, 1H), 6.60 (dd, J=7.5, 1.8 Hz, 1H), 3.76 (s, 3H). MS m/z: 283 (M+1).

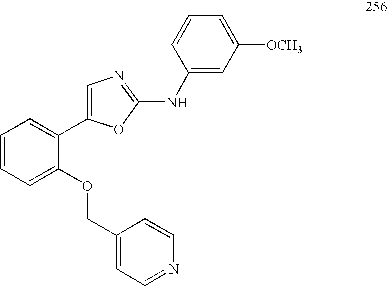

(3-Methoxy-phenyl)-{5-[2-(pyridin-4-ylmethoxy)-phenyl]-oxazol-2-yl}-amine (256): ¹HNMR (CDCl₃) δ (ppm) 8.65 (d, J=5.7 Hz, 2H), 7.68 (dd, J=7.5, 1.2 Hz, 1H), 7.41 (d, J=5.4 Hz, 2H), 7.35 (s, 1H), 7.28-7.19 (m, 3H), 7.10-7.02 (m, 2H), 6.94 (d, J=8.1 Hz, 1H), 6.61 (dd, J=8.1, 2.1 Hz, 1H), 5.24 (s, 2H), 3.83 (s, 3H). MS m/z: 374 (M+1).

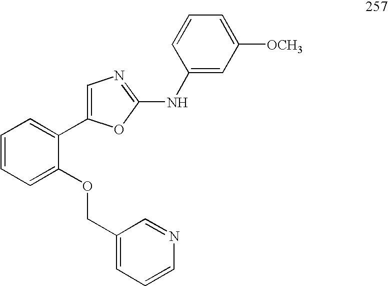

(3-Methoxy-phenyl)-{5-[2-(pyridin-3-ylmethoxy)-phenyl]-oxazol-2-yl}-amine (257): ¹HNMR (CDCl₃) δ (ppm) 8.74 (d, J=1.5 Hz, 1H), 8.65 (dd, J=4.8, 1.5 Hz, 1H), 7.86 (d, J=7.8 Hz, 1H), 7.66 (dd, J=7.8, 1.5 Hz, 1H), 7.39 (dd, J=7.8, 4.8 Hz, 1H), 7.28-7.25 (m, 2H), 7.22 (s, 1H), 7.18 (t, J=2.1 Hz, 1H), 7.10-7.01 (m, 3H), 6.22 (dd, J=7.8, 1.8 Hz, 1H), 5.22 (s, 2H), 3.83 (s, 3H). MS m/z: 374 (M+1).

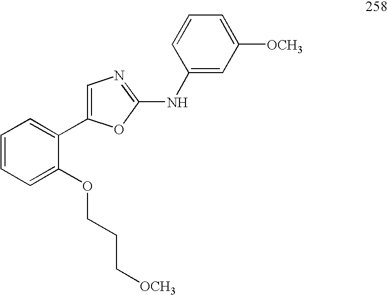

(3-Methoxy-phenyl)-{5-[2-(3-methoxy-propoxy)-phenyl]-oxazol-2-yl}-amine (258): ¹HNMR (CDCl₃) δ (ppm) 7.66 (dd, J=7.8, 1.2 Hz, 1H), 7.44 (s, 1H), 7.30-7.20 (m, 3H), 7.07 (dd, J=8.7, 1.2 Hz, 1H), 7.03-6.97 (m, 2H), 6.62 (dd, J=8.1, 1.8 Hz, 1H), 4.23 (t, J=6.0 Hz, 2H), 3.86 (s, 3H), 3.65 (t, J=6.0 Hz, 2H), 3.38 (s, 3H), 2.23-2.15 (m, 2H). MS m/z: 355 (M+1).

### Example 22

### In Vitro Tubulin Polymerization Assay

Tubulin polymerization is a kinetic process that is temperature-dependent and requires GTP. Soluble tubulin dimers polymerize into microtubules upon warming, and polymerization in vitro correlates with an increase in turbidity (measured at 340 nm). Lyophilized bovine tubulin (HTS Tubulin—97% tubulin, <3% MAPs—Cytoskeleton Inc.) was resuspended in G-PEM buffer (80 mM PIPES pH 7, 1 mM EGTA, 1 mM MgCl₂, 1 mM GTP, 5% glycerol) to a final concentration of 3 mg/ml and kept at 4° C. Compounds in 100× stock solutions in DMSO were dotted to pre-warmed 96-well plates (Corning Costar 3696), the plates were immediately transferred to a 37° C. plate reader (SPECTRAmax Plus, Molecular Devices), cold tubulin was added to the wells, plates were shaken for mixing, and absorbance at 340 nm was read every minute for 30 minutes. Kinetic curves with 30 points each were collected for each compound, and the dynamic range was between 0 and 0.4 OD units. Percentage inhibition values were calculated using the 30 minute data point, based on control samples (treated with 1% DMSO only). This assay is a modified version of the HTS kit sold by Cytoskeleton, adapted to maximize throughput and reduce time, without reduction in dynamic range or sensitivity, while retaining the ability to detect compounds that inhibit or enhance tubulin polymerization.

### Example 23

### Cell Cycle Analysis

Cancer cells (A431, human epidermoid cells) were maintained in culture in D-MEM media with 10% FBS and 1 mg/ml glutamate. Prior to experiment, cells are plated onto 6-well plates for a final density of 500,000 cells/well at the time of treatment. Cells were treated with compounds at 0.01-1 μM final concentrations (final 0.1% DMSO) for 24 hours, then trypsinized, collected, rinsed in PBS (phosphate buffered saline), and fixed in 70% cold ethanol overnight at 4° C. Cells were then rinsed with PBS, resuspended in PBS with 0.2% Tween, RNAse was added (final 1 μg/ml), cells were incubated at 37° C. for 15 min, followed by addition of Propidium Iodide (final 50 μg/ml), and a 30 minute incubation at room temperature. DNA ploidy was analyzed using cell sorters (Epics Excel, Beckman-Coulter, or Guava PCA-96, Guava Technologies) and mitotic arrest characterized by massive accumulation of cells in the G2/M phase of cell cycle.

### Example 24

The in vitro growth inhibition activity of the compounds was determined using a Sulphorhodamine B assay. See, Skehan et al., “New colorimetric cytotoxicity assay for anticancer-drug screening,” J. Natl. Cancer Inst., 82, 1107-1112, (1990). Sulphorhodamine B binds to basic amino acids and stains proteins which can be eluted and detected spectrophotometrically by measuring absorbance at 515 nm. The absorbance indicates the total protein content of the cells fixed to the walls of the plate well at a given time by trichloroacetic acid, which is a measure of the viable cell concentration.

The reagents used in the assay can be purchased from commercial sources and include Sulphorhodamine B 0.4% (w/v) in 1% (v/v) acetic acid (Sigma Cat#S-1402); trichloroacetic acid 50% (w/v) in deionized water, working solution (Sigma Cat#T-9159); and trizma base (Tris) 10 mM working solution, pH 7.5 (Sigma Cat#T-7693).

The procedure was carried out over four days. In Day 1, the cells were seeded in a seed 10,000 cells/100 μL/well in 96 well plate in duplicates as per template. Also, seed cells in extra plate for time zero (To plate). Thereafter, the cells were incubated for 24 hours at 37° C. with 5% CO₂.

On Day 2, the test compound was added to the cells at five log doses from 100 μM to 0.01 μM (Volume of addition=100 μL in 1% DMSO for all compound concentrations, Control treatment=1% DMSO).

The compounds were prepared by weighing the test compounds in 1.5 ml eppendorf tubes and calculating the volume of DMSO to be added to bring the concentration of the compound to 20 mM. Thereafter, a 20 mM stock was made and diluted by four 10 fold dilutions in DMSO to get 2, 0.2, 0.02 and 0.002 mM solutions. Each solution was diluted 100 times (10 μL to 1 ml medium) and a further addition to the culture plate (100 μL) to half the concentration of cells was made. The final well concentration for 20 mM stock was 100 μM and similarly with other test concentrations.

The cells were incubated for 48 hours at 37° C. with 5% CO₂ and terminated by adding 50 μL of 50% cold trichloroacetic acid (10% to final). Thereafter, the cells were incubated for one hour at 4° C.

On Day 4, the cells were fixed to the wells by the addition of 50 μL of 50% cold trichloroacetic acid (10% to final) and incubated for 1 hour at 4° C. The supernatant was discarded by force inverting the plate into the sink followed by washing thrice with tap water and the plates are then air-dried. 100 μL SRB (0.4% in 1% acetic acid) was added to each well and the plates were incubated for 10 minutes at room temperature. Unbound dye was removed by force inverting the plate into the sink and washing thrice with 1% acetic acid. Thereafter, the plates were allowed to air dry.

Bound SRB was solubilized with 100 μL of 10 mM Tris, pH 7.4 and the absorbance was measured at a wavelength of 515 nm.

A sample set of calculations was performed as follows. The percentage growth was calculated by T−To/C−To×100 if T>To and T−To/To×100 if T<To, wherein T is Test OD (with compound), C is Control OD, To is Time Zero OD (cell growth at the time of drug addition). A plot was made with concentrations on X axis and percentage growth on Y axis, the intercept at 50 on the scale gave the GI50 (growth inhibition to 50%) values.

GI50 stands for the concentration of compound required to inhibit 50% tumor cell growth. The in vitro growth inhibition activities of the compounds were determined in A431 human cancer cell line.

What is claimed is:

1. A compound having Formula II: 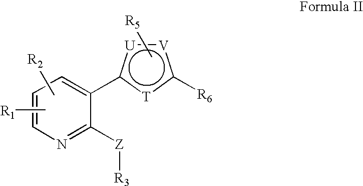
or pharmaceutically acceptable salts, stereoisomers, hydrates or pro-drugs thereof,
wherein,
the ring formed by T, U, V is 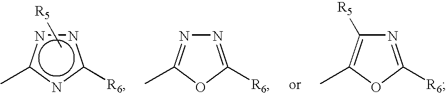
Z is O, S, nitro, or NR₄;
R₁, R₂, or R₅ each independently is:
1) hydrogen, hydroxyl, halo, nitro, or cyano;
2) C₁-C₈ alkyl;
3) C₂-C₈ alkenyl;
4) C₂-C₈ alkynyl;
5) C₁-C₈ alkoxy;
6) C₃-C₈ cycloalkyl or heterocyclyl;
7) C₄-C₈ cycloalkylalkyl or heterocyclylalkyl;
8) C₃-C₁₀ aryl;
9) C₅-C₁₀ aralkyl;
10) C₆-C₁₀ aryloxy;
11) NH₂, NHR₇, or NR₇R₇; or
12) —SO₂R₇,
wherein R₇ is independently H, hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally, R₁ and R₂ taken together form a ring structure including cycloalkyl, heterocyclyl, or aryl ring;
R₃ is:
1) hydrogen;
2) C₁-C₈ alkyl;
3) C₂-C₈ alkenyl;
4) C₂-C₈ alkynyl;
5) C₁-C₈ alkoxy;
6) C₃-C₁₀ cycloalkyl or heterocyclyl;
7) C₄-C₁₀ cycloalkylalkyl or heterocyclylalkyl;
8) C₃-C₁₀ aryl;
9) C₄-C₁₀ aralkyl;
10) carbonyl; or
11) —SO₂R₈, —CO₂R₈, —SR₈, or —SOR₈;
wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,
R₄ is:
1) hydrogen;
2) C₁-C₈ alkyl;
3) C₂-C₈ alkenyl;
4) C₂-C₈ alkynyl;
5) C₃-C₈ cycloalkyl or heterocyclyl;
6) C₄-C₈ cycloalkylalkyl or heterocyclylalkyl;
7) C₃-C₁₀ aryl;
8) C₅-C₁₀ aralkyl;
9) carbonyl; or
10) —SO₂R₁₂, or —SOR₁₂;
wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; optionally, R₃ and R₄ are taken together to form a C₄-C₆ heterocyclyl optionally substituted with R₁₃, or aryl; and
R₆ is:
1) C₁-C₈ alkyl;
2) C₂-C₈ alkenyl;
3) C₂-C₈ alkynyl;
4) C₁-C₈ alkoxy;
5) C₃-C₁₀ cycloalkyl or heterocyclyl;
6) C₄-C₁₀ cycloalkylalkyl or heterocyclylalkyl;
7) C₄-C₁₀ aryl;
8) C₅-C₁₀ aralkyl; or
9) NH₂, NHR₉ or NR₉R₉,
wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

2. The compounds according to claim 1, wherein Z is O or NH.

3. The compounds according to claim 1, wherein R₁, R₂, or R₅ is substituted with R₇, wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

4. The compounds according to claim 1, wherein R₁ and R₂ taken together form a ring structure including cycloalkyl, heterocyclyl or aryl rings.

5. The compound according to claim 1, wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

6. The compound according to claim 1, wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

7. The compound according to claim 1, wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉, cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄) alkyl]₂, —NH(C₅-C₈ heterocyclylalkyl), —NH(C₆-C₈ aryl), or —NH(C₆-C₈ heterocyclyl).

8. A compound of Formula III: 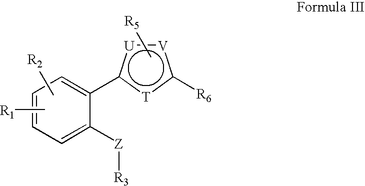
wherein,
the ring formed by T, U, V is 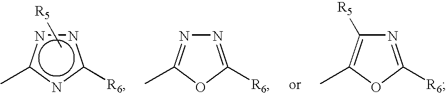
Z is O, S, nitro, or NR₄;
R₁, R₂, or R₅ each independently is:
1) hydrogen, hydroxyl, halo, nitro, or cyano;
2) C₁-C₈ alkyl;
3) C₂-C₈ alkenyl;
4) C₂-C₈ alkynyl;
5) C₁-C₈ alkoxy;
6) C₃-C₈ cycloalkyl or heterocyclyl;
7) C₄-C₈ cycloalkylalkyl or heterocyclylalkyl;
8) C₃-C₁₀ aryl;
9) C₅-C₁₀ aralkyl;
10) C₆-C₁₀ aryloxy;
11) NH₂, NHR₇, or NR₇R₇; or
12) —SO₂R₇,
wherein R₇ is independently H, hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂; optionally, R₁ and R₂ taken together form a ring structure including cycloalkyl, heterocyclyl, or aryl ring;
R₃ is:
1) hydrogen;
2) C₁-C₈ alkyl;
3) C₂-C₈ alkenyl;
4) C₂-C₈ alkynyl;
5) C₁-C₈ alkoxy;
6) C₃-C₁₀ cycloalkyl or heterocyclyl;
7) C₄-C₁₀ cycloalkylalkyl or heterocyclylalkyl;
8) C₃-C₁₀ aryl;
9) C₄-C₁₀ aralkyl;
10) carbonyl; or
11) —SO₂R₈, —CO₂R₈, —SR₈, or —SOR₈;
wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,
R₄ is:
1) hydrogen;
2) C₁-C₈ alkyl;
3) C₂-C₈ alkenyl;
4) C₂-C₈ alkynyl;
5) C₃-C₈ cycloalkyl or heterocyclyl;
6) C₄-C₈ cycloalkylalkyl or heterocyclylalkyl;
7) C₃-C₁₀ aryl;
8) C₅-C₁₀ aralkyl;
9) carbonyl; or
10) —SO₂R₁₂, or —SOR₁₂;
wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; optionally, R₃ and R₄ are taken together to form a C₄-C₆ heterocyclyl optionally substituted with R₁₃, or aryl; and
R₆ is:
1) C₁-C₈ alkyl;
2) C₂-C₈ alkenyl;
3) C₂-C₈ alkynyl;
4) C₁-C₈ alkoxy;
5) C₃-C₁₀ cycloalkyl or heterocyclyl;
6) C₄-C₁₀ cycloalkylalkyl or heterocyclylalkyl;
7) C₄-C₁₀ aryl;
8) C₅-C₁₀ aralkyl; or
9) NH₂, NHR₉ or NR₉R₉,
wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloakyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

9. The compound according to claim 8, wherein Z is O or NR₄.

10. The compound according to claim 8, wherein R₁, R₂, or R₅ is substituted with R₇ wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

11. The compound according to claim 8, wherein when taken together R₁ and R₂ form a ring structure including cycloalkyl, heterocyclyl, or aryl.

12. The compound according to claim 8, wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

13. The compound according to claim 8, wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

14. The compound according to claim 8, wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

15. A method for treating cancer comprising administering a therapeutically effective amount of a compound of Formula n to a subject in need of such treatment, wherein the compound of Formula II has the formula: 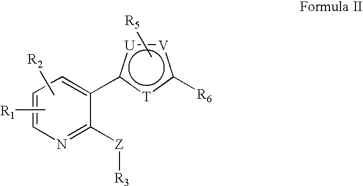
or pharmaceutically acceptable salts, stereoisomers, hydrates or pro-drugs thereof,
wherein,
the ring formed by T, U, V is 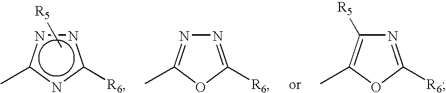
Z is O, S, nitro, or NR₄;
R₁, R₂, or R₅ each independently is:
1) hydrogen, hydroxyl, halo, nitro, or cyano;
2) C₁-C₆ alkyl;
3) C₂-C₆ alkenyl;
4) C₂-C₆ alkynyl;
5) C₁-C₆ alkoxy;
6) C₃-C₈ cycloalkyl or heterocyclyl;
7) C₄-C₈ cycloalkylalkyl or heterocyclylalkyl;
8) C₄-C₁₀ aryl;
9) C₅-C₁₀ aralkyl;
10) C₆-C₁₀ aryloxy;
11) NH₂, NHR₇, or NR₇R₇; or
12) —SO₂R₇,
wherein R₇ is independently H, hydroxyl, halo, C₁-C₄ alkyl optionally substituted with at least one R₁₀, C₁-C₄ alkoxy optionally substituted with at least one R₁₀, C₃-C₈ cycloalkyl optionally substituted with at least one R₁₀, C₄-C₈ heterocycloalkyl optionally substituted with at least one R₁₀, C₆-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂, wherein when taken together R₁ and R₂ form a ring structure including heterocyclyl or aryl rings;
R₃ is.
1) hydrogen;
2) C₁-C₆ alkyl;
3) C₂-C₆ alkenyl;
4) C₂-C₆ alkynyl;
5) C₁-C₆ alkoxy;
6) C₃-C₁₀ cycloalkyl or heterocyclyl;
7) C₄-C₁₀ cycloalkylalkyl or heterocyclylalkyl;
8) C₄-C₁₀ aryl;
9) C₄-C₁₀ aralkyl;
10) carbonyl; or
11) —SO₂R₈, —CO₂R₈, —SR₈, or —SOR₈;
wherein R₈ is independently H, halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂,
R₄ is:
1) hydrogen;
2) C₁-C₆ alkyl;
3) C₂-C₆ alkenyl;
4) C₂-C₆ alkynyl;
5) C₃-C₈ cycloalkyl or heterocyclyl;
6) C₄-C₈ cycloalkylalkyl or heterocyclylalkyl;
7) C₄-C₁₀ aryl;
8) C₅-C₁₀ aralkyl;
9) carbonyl; or
10) —SO₂R₁₂, or —SOR₁₂;
wherein R₁₂ is independently H, halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₆-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂; and
R₆ is:
1) C₁-C₆ alkyl;
2) C₂-C₆ alkenyl;
3) C₂-C₆ alkynyl;
4) C₁-C₆ alkoxy;
5) C₃-C₈ cycloalkyl or heterocyclyl;
6) C₄-C₈ cycloalkylalkyl or heterocyclylalkyl;
7) C₄-C₁₀ aryl;
8) C₅-C₁₀ aralkyl; or
9) —NH₂, —NHR₉, or —NR₉R₉,
wherein R₉ is independently hydroxyl, halo, nitro, C₁-C₄ alkyl optionally substituted with at least one R₁₄, C₂-C₄ alkynyl optionally substituted with at least one R₁₄, C₁-C₄ alkoxy optionally substituted with at least one R₁₄, C₃-C₈ cycloalkyl optionally substituted with at least one R₁₄, C₂-C₈ heterocyclyl optionally substituted with at least one R₁₄, C₆-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₆-C₁₀ aryl, C₄-C₉ heterocycloalkyl, —SO₂(C₆-C₁₀ aryl), NH₂, —NH[(C₁-C₄)alkyl], —N[(C₁-C₄)alkyl]₂, —NH(C₅-C₉ heterocyclylalkyl), —NH(C₆-C₈ aryl), or —NH(C₆-C₈ heterocyclyl) or a pharmaceutically acceptable salt, hydrate or pro-drug thereof, in combination with a pharmaceutically acceptable carrier.

16. The method according to claim 15, wherein Z is O or NH.

17. The method according to claim 15, wherein R₁, R₂, or R₅ is substituted with R₇ wherein R₇ is independently hydroxyl, halo, C₁-C₆ alkyl optionally substituted with at least one R₁₀, C₁-C₆ alkoxy optionally substituted with at least one R₁₀, C₃-C₈ cycloalkyl optionally substituted with at least one R₁₀, C₄-C₈ heterocycloalkyl optionally substituted with at least one R₁₀, C₃-C₁₀ aryl optionally substituted with at least one R₁₀, NH₂, NHR₁₀, NR₁₀R₁₀, or SO₂R₁₀, wherein R₁₀ is independently halo, cyano, nitro, C₁-C₄ alkyl, C₁-C₄ alkoxy, or NH₂.

18. The method according to claim 15, wherein R₁ and R₂ taken together form a ring structure including cycloalkyl, heterocyclyl, or aryl.

19. The method according to claim 15, wherein R₃ is substituted with R₈ wherein R₈ is independently halo, cyano, nitro, C₁-C₄ alkyl optionally substituted with at least one R₁₁, C₁-C₄ alkoxy optionally substituted with at least one R₁₁, C₃-C₈ cycloalkyl optionally substituted with at least one R₁₁, C₃-C₈ heterocyclyl optionally substituted with at least one R₁₁, C₆-C₁₀ aryl optionally substituted with at least one R₁₁, C₆-C₁₀ aralkyl optionally substituted with at least one R₁₁, NH₂, NHR₁₁, NR₁₁R₁₁, or SO₂R₁₁, wherein R₁₁ is independently halo, cyano, nitro, C₁-C₄ alkyl, C₁-C₄ alkoxy, C₆-C₁₀ aryl, C₃-C₈ aralkyl, C₃-C₈ heterocyclyl, or NH₂.

20. The method according to claim 15, wherein R₄ is substituted with R₁₂ wherein R₁₂ is independently halo, cyano, nitro, C₁-C₆ alkyl optionally substituted with at least one R₁₃, C₁-C₄ alkoxy optionally substituted with at least one R₁₃, C₃-C₈ cycloalkyl optionally substituted with at least one R₁₃, C₂-C₈ heterocyclyl optionally substituted with at least one R₁₃, C₃-C₁₀ aryl optionally substituted with at least one R₁₃, NH₂, NHR₁₃, NR₁₃R₁₃, or SO₂R₁₃, wherein R₁₃ is independently halo, cyano, nitro, C₁-C₄ alkyl, C₁-C₄ alkoxy, C₃-C₉ aryl, C₃-C₈ heterocyclylalkyl, or NH₂.

21. The method according to claim 15, wherein R₆ is substituted with R₉ wherein R₉ is independently hydroxyl, halo, nitro, C₁-C₆ alkyl optionally substituted with at least one R₁₄, C₂-C₆ alkynyl optionally substituted with at least one R₁₄, C₁-C₆ alkoxy optionally substituted with at least one R₁₄, C₃-C₁₀ cycloalkyl optionally substituted with at least one R₁₄, C₂-C₈ heterocyclyl optionally substituted with at least one R₁₄, C₄-C₈ cycloalkylalkyl optionally substituted with R₁₄, heterocyclylalkyl optionally substituted with R₁₄, C₄-C₁₀ aryl optionally substituted with at least one R₁₄, C₅-C₁₀ aralkyl optionally substituted with at least one R₁₄, —NH₂, —NHR₁₄, —NR₁₄R₁₄, or —SO₂—R₁₄, wherein R₁₄ is independently halo, cyano, nitro, C₁-C₆ alkyl, C₁-C₆ alkoxy, C₄-C₉ cycloalkyl, C₄-C₉ heterocycloalkyl, C₄-C₁₀ aryl, —SO₂(C₆-C₁₀ aryl), —NH₂, —NH[(C₁-C₄)alkyl], —N[(C₁-C₄)alkyl]₂, —NH(C₅-C₈ heterocyclylalkyl), —NH(C₆-C₈ aryl), or —NH(C₆-C₈ heterocyclyl).

22. The method according to claim 15, wherein the dosage form is a tablet, caplet, troche, lozenge, dispersion, suspension, suppository, solution, capsule, or patch.

23. The method according to claim 15, wherein the compound is administered in about 0.001 mg/kg to about 100 mg/kg.

24. The method according to claim 15, wherein the compound is administered by oral administration.
